SEQUENCE LISTING (33 sequences)

SEQ ID NO: 1 - RNA, 11 nt, Artificial Sequence
  Synthetic
cccguaccccc

SEQ ID NO: 2 - RNA, 12 nt, Artificial Sequence
  Synthetic
ggcuucugacca

SEQ ID NO: 3 - RNA, 15 nt, Artificial Sequence
  Synthetic
ggccacucguuaaga

SEQ ID NO: 4 - RNA, 20 nt, Artificial Sequence
  Synthetic
ggccacugaagccauugaag

SEQ ID NO: 5 - RNA, 24 nt, Artificial Sequence
  Synthetic
ggccacugaagccauugucaagga

SEQ ID NO: 6 - RNA, 25 nt, Artificial Sequence
  Synthetic
ggccacugaagccauugucaccgaa

SEQ ID NO: 7 - RNA, 25 nt, Artificial Sequence
  Synthetic
ggcgaagcacucguggccauucgca

SEQ ID NO: 8 - RNA, 10 nt, Artificial Sequence
  Synthetic
ggccaaggaa

SEQ ID NO: 9 - RNA, 11 nt, Artificial Sequence
  Synthetic
ggccaaggaaa

SEQ ID NO: 10 - RNA, 12 nt, Artificial Sequence
  Synthetic
ggccacugaaga

SEQ ID NO: 11 - RNA, 16 nt, Artificial Sequence
  Synthetic
ggccacugaagccauu

SEQ ID NO: 12 - RNA, 15 nt, Artificial Sequence
  Synthetic
ggccacugaaggaag

SEQ ID NO: 13 - RNA, 12 nt, Artificial Sequence
  Synthetic
ccggacuagaga

SEQ ID NO: 14 - RNA, 13 nt, Artificial Sequence
  Synthetic
gaauagagaggaa

SEQ ID NO: 15 - RNA, 16 nt, Artificial Sequence
  Synthetic
ggccacugaaggaaga

SEQ ID NO: 16 - RNA, 17 nt, Artificial Sequence
  Synthetic
gaacacugaucguagaa

SEQ ID NO: 17 - RNA, 18 nt, Artificial Sequence
  Synthetic
ggccacugaaggaagaga

SEQ ID NO: 18 - RNA, 14 nt, Artificial Sequence
  Synthetic
ggauagauagcgaa

SEQ ID NO: 19 - RNA, 14 nt, Artificial Sequence
  Synthetic
ggagugagagagaa

SEQ ID NO: 20 - RNA, 14 nt, Artificial Sequence
  Synthetic
ggccacauagcgaa

SEQ ID NO: 21 - RNA, 22 nt, Artificial Sequence
  Synthetic
agccccuccuccccuuccugca

SEQ ID NO: 22 - RNA, 22 nt, Artificial Sequence
  Synthetic
guggucuuugaauaaagucuga

SEQ ID NO: 23 - RNA, 127 nt, Artificial Sequence
  Synthetic
ugauaauaggcuggagccucgguggccuagcuucuugccccuugggccuccccccagccccuccuccccuuccugcaaguggucacccguacccccguggucuuugaauaaagucugagugggcggc

SEQ ID NO: 24 - RNA, 22 nt, Artificial Sequence
  Synthetic
  modified_base (1)..(2): Is a 2′ OMe RNA nucleotide
  modified_base (7)..(22): Is a 2′ OMe RNA nucleotide
ugcaggaaggggaggaggggcu

SEQ ID NO: 25 - RNA, 22 nt, Artificial Sequence
  Synthetic
  modified_base (1)..(18): Is a 2′ OMe RNA nucleotide
ucagacuuuauucaaagaccac

SEQ ID NO: 26 - RNA, 22 nt, Artificial Sequence
  Synthetic
  modified_base (1)..(17): Is a 2′ OMe RNA nucleotide
  modified_base (22)..(22): Is a 2′ OMe RNA nucleotide
ucagacuuuauucaaagaccac

SEQ ID NO: 27 - RNA, 22 nt, Artificial Sequence
  Synthetic
  modified_base (1)..(16): Is a 2′ OMe RNA nucleotide
  modified_base (21)..(22): Is a 2′ OMe RNA nucleotide
ucagacuuuauucaaagaccac

SEQ ID NO: 28 - RNA, 22 nt, Artificial Sequence
  Synthetic
  modified_base (1)..(13): Is a 2′ OMe RNA nucleotide
  modified_base (18)..(22): Is a 2′ OMe RNA nucleotide
ucagacuuuauucaaagaccac

SEQ ID NO: 29 - RNA, 22 nt, Artificial Sequence
  Synthetic
  modified_base (1)..(12): Is a 2′ OMe RNA nucleotide
  modified_base (17)..(22): Is a 2′ OMe RNA nucleotide
ucagacuuuauucaaagaccac

SEQ ID NO: 30 - RNA, 22 nt, Artificial Sequence
  Synthetic
  modified_base (1)..(5): Is a 2′ OMe RNA nucleotide
  modified_base (10)..(22): Is a 2′ OMe RNA nucleotide
ugcaggaaggggaggaggggcu

SEQ ID NO: 31 - RNA, 22 nt, Artificial Sequence
  Synthetic
  modified_base (1)..(17): Is a 2′ OMe RNA nucleotide
  modified_base (22)..(22): Is a 2′ OMe RNA nucleotide
ucagacuuuauucaaagaccac

SEQ ID NO: 32 - RNA, 20 nt, Artificial Sequence
  Synthetic
cccguacccccguggucuuu

SEQ ID NO: 33 - RNA, 23 nt, Artificial Sequence
  Synthetic
cccguacccccguggucuuugaa

(19) United States
(12) Patent Application Publication
(10) Pub. No.: US 2024/0229109 A1
(43) Pub. Date: Jul. 11, 2024

(54) METHODS FOR IDENTIFICATION AND RATIO DETERMINATION OF RNA SPECIES IN MULTIVALENT RNA COMPOSITIONS

(71) Applicant: ModernaTX, Inc., Cambridge, MA (US)

(72) Inventors: Amy E. Rabideau, Waltham, MA (US); David Reid, Cambridge, MA (US); Huijuan Li, Cambridge, MA (US); Kristian Link, Cambridge, MA (US); Tao Jiang, Quincy, MA (US); Eva-Maria Schneeberger, Cambridge, MA (US)

(73) Assignee: ModernaTX, Inc., Cambridge, MA (US)

(21) Appl. No.: 18/284,938

(22) PCT Filed: Mar. 31, 2022
(86) PCT No.: PCT/US2022/022839

§ 371 Date: Sep. 29, 2023

Related U.S. Application Data
(60) Provisional application No. 63/287,722, filed on Dec. 9, 2021.
(60) Provisional application No. 63/248,083, filed on Sep. 24, 2021.
(60) Provisional application No. 63/228,957, filed on Aug. 3, 2021.
(60) Provisional application No. 63/169,398, filed on Apr. 1, 2021.

(51) Int. Cl. C12Q 1/6825; C12Q 1/6806; C12Q 1/6876

(52) U.S. Cl. CPC C12Q 1/6825; C12Q 1/6876; C12Q 1/6806; C12Q 2600/16

(57) ABSTRACT

Aspects of the disclosure relate to methods for analyzing compositions comprising RNA species with unique nucleotide sequences for identification and/or ratio determination of RNAs. The disclosure is based, in part, on methods of cleaving identifying sequences from RNAs in a composition, and detecting the abundance of each identifying sequence to quantify the abundance of corresponding RNA species. Other aspects relate to methods for producing compositions comprising more than two RNA species, such as multivalent RNA compositions. The disclosure is based, in part, on methods of determining the proper amount of input DNA for in vitro transcription (IVT) reactions that will result in RNA being transcribed in a predetermined ratio. In some aspects, the disclosure relates to pharmaceutical compositions comprising multivalent RNA compositions produced by methods described, by the disclosure.

### RELATED APPLICATIONS

This application claims the benefit under 35 U.S.C. § 119(e) of U.S. provisional Application No. 63/169,398 filed Apr. 1, 2021, U.S. provisional Application No. 63/228,957 filed Aug. 3, 2021, U.S. provisional Application No. 63/248,083 filed Sep. 24, 2021, and U.S. provisional Application No. 63/287,722 filed Dec. 9, 2021, each of which is incorporated by reference herein in its entirety.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. The ASCII file, created on Mar. 29, 2022, is named M137870168WO00-SEQ-NTJ, and is 7,917 bytes in size.

### BACKGROUND

Multivalent mRNA constructs are typically produced by transcribing one mRNA product at a time, purifying each mRNA product, and then mixing the purified mRNA products together prior to formulation. This type of process incurs significant time and monetary investment especially at the Good Manufacturing Practice (GMP) scale.

### SUMMARY

Aspects of the disclosure relate to RNA compositions comprising one or more distinct RNA species (e.g., RNAs encoding different proteins), where each RNA species comprises a unique nucleotide sequence that can be used to identify the RNA species, and methods of analyzing the same. The disclosure is based, in part, on the incorporation of unique identification and/or ratio determination (IDR) sequences into distinct RNA species of a RNA composition at a similar position on each RNA species (e.g., within a non-coding region). Without wishing to be bound by theory, it is believed that RNAs can be digested to release RNA fragments comprising the IDR sequence, and analytical methods can be used to quantify the types and amounts of RNA fragments containing each IDR, to generate a profile of the types and/or amounts of each RNA species in a RNA composition. Use of IDR sequences for analysis allows characterization of multivalent RNA compositions comprising several distinct RNA species, even if multiple RNA species are difficult to distinguish by length or coding sequence. For example, a multivalent RNA composition comprising eight RNA species, each encoding a different serotype of the same protein, may have similar lengths and coding sequences, but each RNA species may comprise a different IDR sequence in a non-coding region. Because each IDR sequence unambiguously identifies a particular RNA species, the abundance of IDR sequences may be measured to determine the abundance of RNAs encoding each serotype. Furthermore, the coding sequence of one or more RNA species in a multivalent RNA composition may be modified (e.g., to alter the structure of an encoded therapeutic protein or antigen) independently from the IDR sequence, such that the same analytical methods may be used to evaluate a RNA composition in which one or more RNA coding sequences are modified.

Additional aspects of the disclosure relate to methods for producing multivalent RNA compositions. The disclosure is based, in part, on methods for determining the proper amount of input DNA (e.g., plasmid DNA, chemically synthesized DNA, etc.) for in vitro transcription (IVT) reactions that will result in RNA being transcribed from the input DNA in a pre-determined ratio. In some aspects, the disclosure relates to pharmaceutical compositions comprising multivalent RNA compositions produced by methods described by the disclosure.

Accordingly, some aspects of the disclosure relate to a method for analyzing a multivalent RNA composition, the method comprising:
(i) contacting a multivalent RNA composition, comprising a first RNA species and a second RNA species, with two or more RNase H guide oligonucleotides;
(ii) digesting the first RNA species and the second RNA species with an RNase H enzyme to release a plurality of first RNA fragments and second RNA fragments; and
(iii) measuring a presence and/or amount of the released first RNA fragments and second RNA fragments.

In some embodiments, the first RNA species comprises a first identifying sequence and the second RNA species comprises a second identifying sequence, wherein the first and second identifying sequences are different.

In some embodiments, each of the first and second identifying sequences has a nucleotide length that is independently selected from between 1 to 25 nucleotides.

In some embodiments, the first identifying sequence is not a sequence isomer of the second identifying sequence.

In some embodiments, the first and second identifying sequences have different nucleotide lengths.

In some embodiments, the first identifying sequence has a first identifying mass equal to a mass of an RNA consisting of the first identifying sequence, wherein the second identifying sequence has a second identifying mass equal to a mass of an RNA consisting of the second identifying sequence, wherein the first and second identifying masses are different.

In some embodiments, the first and second identifying masses differ by 9 Da or more, 25 Da or more, 50 Da or more, 75 Da or more, or 100 Da or more.

In some embodiments, the measuring comprises detecting the released first RNA fragments and second RNA fragments by LC-MS.

In some embodiments, the measuring comprises detecting the released first RNA fragments and second RNA fragments by LC-UV.

In some embodiments, the method further comprises calculating a ratio between the amounts of the released first RNA fragments and second RNA fragments.

In some embodiments, the first RNA species comprises a first 5′ UTR, wherein the second RNA species comprises a second 5′ UTR, wherein each of the two or more RNase H guide oligonucleotides is capable of hybridizing with a nucleotide sequence in the first 5′ UTR and the second 5′ UTR.

In some embodiments, the method comprises cleaving the first 5′ UTR to release the first RNA fragment, and cleaving the second 5′ UTR to release the second RNA fragment, wherein the first RNA fragment comprises a first cap and the second RNA fragment comprises a second cap.

In some embodiments, the first RNA fragment comprises the first identifying sequence and the second RNA fragment comprises the second identifying sequence.

In some embodiments, the method comprises cleaving the first 5′ UTR to release the first RNA fragment, and cleaving the second 5′ UTR to release the second RNA fragment, wherein the first RNA fragment comprises the first identifying sequence and the second RNA fragment comprises the second identifying sequence.

In some embodiments, the method comprises:
(i) cleaving the first 5′ UTR at a position upstream from the first identifying sequence and at a position downstream of the first identifying sequence to release the first RNA fragment, wherein the first RNA fragment comprises the first identifying sequence; and
(ii) cleaving the second 5′ UTR at a position upstream from the second identifying sequence and at a position downstream from the second identifying sequence to release the second RNA fragment, wherein the second RNA fragment comprises the second identifying sequence.

In some embodiments, the first RNA species comprises a first 3′ UTR, wherein the second RNA species comprises a second 3′ UTR, wherein each of the two or more RNase H guide oligonucleotides is capable of hybridizing with a nucleotide sequence in the first 3′ UTR and the second 3′ UTR.

In some embodiments, the method comprises cleaving the first 3′ UTR to release the first RNA fragment, and cleaving the second 3′ UTR to release the second RNA fragment, wherein the first RNA fragment comprises a first poly(A) tail and the second RNA fragment comprises a second poly(A) tail.

In some embodiments, the first RNA fragment comprises the first identifying sequence and the second RNA fragment comprises the second identifying sequence.

In some embodiments, the method comprises cleaving the first 3′ UTR to release the first RNA fragment, and cleaving the second 3′ UTR to release the second RNA fragment, wherein the first RNA fragment comprises the first identifying sequence and the second RNA fragment comprises the second identifying sequence.

In some embodiments, the method comprises:
(i) cleaving the first 3′ UTR at a position upstream from the first identifying sequence and at a position downstream of the first identifying sequence to release the first RNA fragment, wherein the first RNA fragment comprises the first identifying sequence; and
(ii) cleaving the second 3′ UTR at a position upstream from the second identifying sequence and at a position downstream from the second identifying sequence to release the second RNA fragment, wherein the second RNA fragment comprises the second identifying sequence.

In some embodiments, the method comprises contacting the multivalent RNA composition with a first and second RNase H guide oligonucleotide,
wherein the first RNase H guide oligonucleotide is capable of hybridizing with a sequence upstream from the identifying sequence,
wherein the second RNase H guide oligonucleotide is capable of hybridizing with a sequence downstream from the identifying sequence.

In some embodiments, the nucleotide sequences of the released first and second RNA fragments are identical except for the first identifying sequence in the first RNA fragment and the second identifying sequence in the second RNA fragment.

In some embodiments, the each of the two or more RNase H guide oligonucleotides comprises a nucleotide sequence represented by the formula:

[R]ₚD₁D₂D₃D₄[R]_{q}

wherein each R is an RNA nucleotide, each D is a DNA nucleotide, and each of p and q are independently an integer between 1 and 50.

In some embodiments, one or more RNA nucleotides of the two or more RNase H guide oligonucleotides are modified RNA nucleotides.

In some embodiments, each RNA nucleotide of the two or more RNase H guide oligonucleotides is a modified RNA nucleotide.

In some embodiments, one or more modified RNA nucleotides of the two or more RNase H guide oligonucleotides are 2′-O-methyl RNA nucleotides.

In some embodiments, each RNA nucleotide of the two or more RNase H guide oligonucleotides is a 2′-O-methyl RNA nucleotide.

In some embodiments, one or more modified RNA nucleotides of the two or more RNase H guide oligonucleotides comprises:
(a) a modified nucleobase selected from the group consisting of xanthine, allyaminouracil, allyaminothymidine, hypoxanthine, digoxigeninated adenine, digoxigeninated cytosine, digoxigeninated guanine, digoxigeninated uracil, 6-chloropurineriboside, N6-methyladenine, methylpseudouracil, 2-thiocytosine, 2-thiouracil, 5-methyluracil, 4-thiothymidine, 4-thiouracil, 5,6-dihydro-5-methyluracil, 5,6-dihydrouracil, 5-[(3-Indolyl)propionamide-N-allyl]uracil, 5-aminoallylcytosine, 5-aminoallyluracil, 5-bromouracil, 5-bromocytosine, 5-carboxycytosine, 5-carboxymethylesteruracil, 5-carboxyuracil, 5-fluorouracil, 5-formylcytosine, 5-formyluracil, 5-hydroxycytosine, 5-hydroxymethylcytosine, 5-hydroxymethyluracil, 5-hydroxyuracil, 5-iodocytosine, 5-iodouracil, 5-methoxycytosine, 5-methoxyuracil, 5-methylcytosine, 5-methyluracil, 5-propargylaminocytosine, 5-propargylaminouracil, 5-propynylcytosine, 5-propynyluracil, 6-azacytosine, 6-azauracil, 6-chloropurine, 6-thioguanine, 7-deazaadenine, 7-deazaguanine, 7-deaza-7-propargylaminoadenine, 7-deaza-7-propargylaminoguanine, 8-azaadenine, 8-azidoadenine, 8-chloroadenine, 8-oxoadenine, 8-oxoguanine, araadenine, aracytosine, araguanine, arauracil, biotin-16-7-deaza-7-propargylaminoguanine, biotin-16-aminoallylcytosine, biotin-16-aminoallyluracil, cyanine 3-5-propargylaminocytosine, cyanine 3-6-propargylaminouracil, cyanine 3-aminoallylcytosine, cyanine 3-aminoallyluracil, cyanine 5-6-propargylaminocytosine, cyanine 5-6-propargylaminouracil, cyanine 5-aminoallylcytosine, cyanine 5-aminoallyluracil, cyanine 7-aminoallyluracil, dabcyl-5-3-aminoallyluracil, desthiobiotin-16-aminoallyl-uracil, desthiobiotin-6-aminoallylcytosine, isoguanine, N1-ethylpseudouracil, N1-methoxymethylpseudouracil, N1-methyladenine, N1-methylpseudouracil, N1-propylpseudouracil, N2-methylguanine, N4-biotin-OBEA-cytosine, N4-methylcytosine, N6-methyladenine, O6-methylguanine, pseudoisocytosine, pseudouracil, thienocytosine, thienoguanine, thienouracil, xanthosine, 3-deazaadenine, 2,6-diaminoadenine, 2,6-daminoguanine, 5-carboxamide-uracil, 5-ethynyluracil, N6-isopentenyladenine (i6A), 2-methyl-thio-N6-isopentenyladenine (ms2i6A), 2-methylthio-N6-methyladenine (ms2m6A), N6-(cis-hydroxyisopentenyl)adenine (io6A), 2-methylthio-N6-(cis-hydroxyisopentenyl)adenine (ms2io6A), N6-glycinylcarbamoyladenine (g6A), N6-threonylcarbamoyladenine (t6A), 2-methylthio-N6-threonyl carbamoyladenine (ms2t6A), N6-methyl-N6-threonylcarbamoyladenine (m6t6A), N6-hydroxynorvalylcarbamoyladenine (hn6A), 2-methylthio-N6-hydroxynorvalyl carbamoyladenine (ms2hn6A), N6,N6-dimethyladenine (m62A), and N6-acetyladenine (ac6A);
(b) a modified sugar selected from the group consisting of 2′-thioribose, 2′,3′-dideoxyribose, 2′-amino-2′-deoxyribose, 2′ deoxyribose, 2′-azido-2′-deoxyribose, 2′-fluoro-2′-deoxyribose, 2′-O-methylribose, 2′-O-methyldeoxyribose, 3′-amino-2′,3′-dideoxyribose, 3′-azido-2′,3′-dideoxyribose, 3′-deoxyribose, 3′-O-(2-nitrobenzyl)-2′-deoxyribose, 3′-O-methylribose, 5′-aminoribose, 5′-thioribose, 5-nitro-1-indolyl-2′-deoxyribose, 5′-biotin-ribose, 2′-O,4′-C-methylene-linked, 2′-O,4′-C-amino-linked ribose, and 2′-O,4′-C-thio-linked ribose; and/or
(c) a modified phosphate selected from the group consisting of phosphorothioate (PS), thiophosphate, 5′-O-methylphosphonate, 3′-O-methylphosphonate, 5′-hydroxyphosphonate, hydroxyphosphanate, phosphoroselenoate, selenophosphate, phosphoramidate, carbophosphonate, methylphosphonate, phenylphosphonate, ethylphosphonate, H-phosphonate, guanidinium ring, triazole ring, boranophosphate (BP), methylphosphonate, and guanidinopropyl phosphoramidate.

In some embodiments, one or more DNA nucleotides of the two or more RNase H guide oligonucleotides are modified DNA nucleotides.

In some embodiments, each DNA nucleotide of the two or more RNase H guide oligonucleotides is a modified DNA nucleotide.

In some embodiments, one or more modified DNA nucleotides of the two or more RNase H guide oligonucleotides are 5-nitroindole, Inosine, 4-nitroindole, 6-nitroindole, 3-nitropyrrole, a 2-6-diaminopurine, 2-amino-adenine, or 2-thio-thiamine DNA nucleotides.

In some embodiments, each of the two or more RNase H guide oligonucleotides does not comprise a nucleotide sequence comprising 6 or more, 5 or more, or 4 or more consecutive DNA nucleotides having the same nucleobase.

In some embodiments, one or more of the RNAs is an mRNA.

In some embodiments, each of the RNAs are mRNAs.

In some embodiments, one or more of the RNAs are in vitro transcribed (IVT) mRNAs.

In some embodiments, each of the RNAs are IVT mRNAs.

In some aspects, the disclosure relates to an RNA composition comprising two or more RNA species, wherein the first RNA species comprises a first identifying sequence and the second RNA species comprises a second identifying sequence, wherein the first and second identifying sequences are different.

In some embodiments, each of the first and second identifying sequences has a nucleotide length that is independently selected from between 1 to 25 nucleotides.

In some embodiments, the first identifying sequence is not a sequence isomer of the second identifying sequence.

In some embodiments, the first and second identifying sequences have different nucleotide lengths.

In some embodiments, the first identifying sequence has a first identifying mass equal to a mass of an RNA consisting of the first identifying sequence, wherein the second identifying sequence has a second identifying mass equal to a mass of an RNA consisting of the second identifying sequence, wherein the first and second identifying masses are different.

In some embodiments, the first and second identifying masses differ by 9 Da or more, 25 Da or more, 50 Da or more, 75 Da or more, or 100 Da or more.

In some embodiments, one or more of the RNAs is an mRNA.

In some embodiments, each of the RNAs are mRNAs.

In some embodiments, one or more of the RNAs are in vitro transcribed (IVT) mRNAs.

In some embodiments, each of the RNAs are IVT mRNAs.

In some embodiments, the RNA composition comprises 2, 3, 4, 5, 6, 7, 8, 9, or 10 RNA species.

In some embodiments, each RNA species comprises an open reading frame encoding a therapeutic peptide or therapeutic protein.

In some embodiments, each RNA species comprises an open reading frame encoding an antigenic peptide or antigenic protein.

In some embodiments, at least 80%, at least 85%, at least 90%, at least 95%, at least 96%, at least 97%, at least 98%, at least 99%, or up to 100% of RNAs of the RNA composition comprise a poly(A) tail.

In some embodiments, the amount of each RNA species in the RNA composition is between 0.2 times to 5 times, 0.3 times to 3 times, or 0.5 times to 2 times, 0.75 times to 1.4 times, 0.8 times to 1.25 times, or 0.9 to 1.15 times the amount of each other RNA species in the RNA composition.

In some embodiments, the disclosure relates to a pharmaceutical composition comprising:
(a) an RNA composition described herein; and
(b) one or more pharmaceutically acceptable excipients.

In some embodiments, the RNAs of the RNA composition are packaged in a lipid-based particle.

In some embodiments, the lipid-based particle is a liposome or a lipid nanoparticle.

In some aspects, the disclosure relates to a method for producing a multivalent RNA composition, the method comprising:
(a) combining a linearized first DNA molecule encoding a first RNA and a linearized second DNA molecule encoding a second RNA into a single reaction vessel, wherein the first DNA molecule and the second DNA molecule are obtained from different sources; and
(b) simultaneously in vitro transcribing the linearized first DNA molecule and the linearized second DNA molecule to obtain a multivalent RNA composition.

In some aspects, the disclosure relates to a method for producing a multivalent RNA composition, the method comprising:
(a) producing a first DNA molecule in a first bacterial cell culture,
(b) producing a second DNA molecule in a second bacterial cell culture, wherein the first bacterial cell culture and second bacterial cell culture are not co-cultured,
(c) purifying and linearizing the first DNA molecule and second DNA molecule;
(d) combining the purified and linearized first DNA molecule and the purified and linearized second DNA molecule into a single IVT reaction mixture, and then
(e) simultaneously in vitro transcribing the first and second DNA molecules to obtain a multivalent RNA composition.

In some aspects, the disclosure relates to a method for producing a multivalent RNA composition, the method comprising:
(a) simultaneously in vitro transcribing at least two DNA molecules in a reaction mixture comprising:
   (i) a first population of DNA molecules encoding a first RNA; and
   (ii) a second population of DNA molecules encoding a second RNA that is different than the first RNA,
wherein the amounts of the first and second populations of DNA molecules present in the reaction mixture prior to the start of the IVT are normalized; and
(b) obtaining a multivalent RNA composition.

In some aspects, the disclosure relates to a method for producing a multivalent RNA composition, the method comprising:
(a) simultaneously in vitro transcribing at least two DNA molecules in a reaction mixture comprising:
   (i) a first population of DNA molecules encoding a first RNA; and
   (ii) a second population of DNA molecules encoding a second RNA that is different than the first RNA; and
(b) obtaining a multivalent RNA composition having a pre-defined ratio of the first RNA to the second RNA produced by the IVT of step (a), wherein the multivalent RNA composition comprises >40% polyA-tailed RNAs.

In some aspects, the disclosure relates to a method for producing a multivalent RNA composition, the method comprising:
(a) simultaneously in vitro transcribing at least two DNA molecules in a reaction mixture comprising:
   (i) a first population of DNA molecules encoding a first RNA; and
   (ii) a second population of DNA molecules encoding a second RNA that is different than the first RNA by at least 100 nucleotides in length; and
(b) obtaining a multivalent RNA composition having a pre-defined ratio of the first RNA to the second RNA produced by the IVT of step (a).

In some embodiments, the first and/or second population of DNA molecules comprises plasmid DNA (pDNA), chemically-synthesized DNA, or complementary DNA (cDNA).

In some embodiments, the IVT comprises co-transcriptional capping.

In some embodiments, the first RNA and/or the second RNA comprises a 5′ cap.

In some embodiments, at least 75% of the first RNAs each comprise a polyA tail.

In some embodiments, at least 75% of the second RNAs each comprise a polyA tail.

In some embodiments, the first RNA and/or the second RNA comprises messenger RNA (mRNA).

In some embodiments, the first RNA and/or second RNA encodes a therapeutic peptide or therapeutic protein.

In some embodiments, the first RNA and/or second RNA encodes an antigenic peptide or antigenic protein.

In some embodiments, the normalization is based on molar mass, degradation rate (e.g., of the input DNA and/or output RNA), nucleotide content, purity, and/or polyA-tailing efficiency.

In some embodiments, the molar amounts of the first and second populations of DNA molecules are normalized according to the higher polyA-tailing efficiency between the first DNA population and second DNA population.

In some embodiments, the reaction mixture further comprises 1, 2, 3, 4, 5, 6, 7, 8, 9, or 10 additional DNA populations.

In some embodiments, the multivalent RNA composition comprises 1, 2, 3, 4, 5, 6, 7, 8, 9, or 10 additional RNAs.

In some embodiments, each of the additional RNAs encodes a therapeutic peptide or therapeutic protein.

In some embodiments, the method further comprises a step of purifying the multivalent RNA composition from the reaction mixture.

In some embodiments, the purifying comprises chromatography or gel electrophoresis.

In some embodiments, the purifying comprises column chromatography.

In some embodiments, the first RNAs and/or the second RNAs comprise a 5′ untranslated region (5′ UTR).

In some embodiments, the first RNAs and/or the second RNAs comprise a 3′ untranslated region (3′ UTR).

In some embodiments, the multivalent RNA composition has a pre-defined RNA ratio of the first RNA to the second RNA.

In some aspects, the disclosure relates to a multivalent RNA composition produced by a method described herein.

In some aspects, the disclosure relates to a pharmaceutical composition comprising:
(a) a multivalent RNA composition described herein; and
(b) one or more pharmaceutically acceptable excipients.

In some embodiments, the RNAs of the multivalent RNA composition are packaged in a lipid-based particle.

In some embodiments, the lipid-based particle is a liposome or a lipid nanoparticle.

### BRIEF DESCRIPTION OF DRAWINGS

FIGS. 1A-1B show schematics depicting production of multivalent RNA compositions. FIG. 1A depicts a workflow in which each RNA of a multivalent RNA composition is separately transcribed, purified, mixed, and then formulated into a multivalent composition. FIG. 1B depicts a workflow, according to some embodiments, in which >2 DNAs are in vitro transcribed (IVT) in a single reaction and the resulting multivalent RNA composition is purified and formulated without further mixing of RNAs.

FIG. 2 shows representative data comparing RNA produced by co-transcription and admixing. Data indicate that admixes have linear correlations with the same slope, whereas DNA1 and DNA2 co-in vitro transcriptions (co-IVTs) have linear correlations with different slopes. No obvious length bias over different input amounts of DNA was observed.

FIGS. 3A-3B show representative RNase T1 fingerprinting analysis data. FIG. 3A shows representative RNase T1 fingerprinting data, which indicates that co-IVT RNAs mixed at different ratios (for example 0% RNA1 and 100% RNA2 versus 100% RNA1 and 0% RNA2) produce distinct RNase T1 fingerprints. FIG. 3B shows representative data indicating co-IVT RNAs and admixed RNAs have the same RNase T1 fingerprint.

FIGS. 4A-4D show representative data for normalization of input DNA. FIG. 4A shows a linear graph demonstrating the production of RNA1 in a non-normalized mass ratio input of DNA1 and DNA2 in the IVT reaction producing RNA1 and RNA2. Y=X dotted lines depicts no compositional bias between DNA input and RNA output by mass ratio. Data indicate a compositional bias by mass ratio, as the dots do not fall within or close to the dotted line. FIG. 4B shows a linear graph showing the production of RNA1 in a normalized molar ratio input of DNA1 and DNA2 IVT reaction producing RNA1 and RNA2. Y=X dotted lines depicts no compositional bias between DNA input and RNA output by molar ratio. Data indicate no compositional bias, as the dots of normalized DNA input fall within or close to the dotted line. FIG. 4C shows a linear graph showing the production of RNA3 in a non-normalized molar ratio input of DNA3 and DNA4 in a IVT reaction using a T7 polymerase variant producing RNA1 and RNA2. Y=X dotted lines depicts no compositional bias between DNA input and RNA output by mass ratio. Data indicate a compositional bias by molar ratio, as the dots do not fall within or close to the dotted line. FIG. 4D shows a linear graph showing the production of RNA3 in a normalized molar ratio input of DNA3 and DNA4 IVT reaction using a T7 polymerase variant producing RNA1 and RNA2. Y=X black dotted lines depicts no compositional bias between DNA input and RNA output by molar ratio. Data indicate no compositional bias, as the dots of normalized DNA input fall within or close to the dotted line.

FIGS. 5A-5B show representative data for co-IVT of two monoclonal antibodies (Ab1 and Ab2). FIG. 5A shows representative data indicating that polyA tail percentage is higher in the LC-encoding RNA than the HC-encoding RNA for both antibodies. FIG. 5B shows representative data that there is no compositional bias introduced during polyA purification.

FIG. 6 shows representative data indicating that normalizing the input DNA mass to the highest efficiency polyA tailing RNA resulted in production of multivalent antibody RNA compositions not only having the correct ratio of HC:LC but also that the RNAs in those compositions were polyA-tailed.

FIG. 7 shows a mass spectrum of RNA fragments containing three different IDR sequences.

FIGS. 8A-8L show mass spectra of RNA fragments produced by RNase H-mediated cleavage, in which a DNA guide was hybridized with an RNA, and the RNA:DNA hybrid was cleaved by RNase. FIG. 8A shows a mass spectrum of cleavage using a first IDR sequence. FIG. 8B shows a deconvoluted mass spectrum depicting the average mass of the fragments shown in FIG. 8A. FIG. 8C shows a mass spectrum of cleavage using a second IDR sequence. FIG. 8D shows a deconvoluted mass spectrum depicting the average mass of the fragments shown in FIG. 8C. FIG. 8E shows a mass spectrum of cleavage using a third IDR sequence. FIG. 8F shows a deconvoluted mass spectrum depicting the average mass of the fragments shown in FIG. 8E. FIG. 8G shows a mass spectrum of cleavage using a fourth IDR sequence. FIG. 8H shows a deconvoluted mass spectrum depicting the average mass of the fragments shown in FIG. 8G. FIG. 8I shows a mass spectrum of cleavage using a fifth IDR sequence. FIG. 8J shows a deconvoluted mass spectrum depicting the average mass of the fragments shown in FIG. 8I. FIG. 8K shows a mass spectrum of cleavage using a sixth IDR sequence. FIG. 8L shows a deconvoluted mass spectrum depicting the average mass of the fragments shown in FIG. 8K. Vertical lines in FIGS. 8A, 8C, 8E, 8G, 8I, and 8K represent the expected position of RNase H-mediated cleavage.

FIGS. 9A-9D show antigen-specific IgG titers in sera obtained from mice immunized with quadrivalent mRNA mixtures comprising four mRNAs (RNA5, RNA6, RNA7, RNA8), with each composition comprising mRNAs with no identifying sequences (groups 2-5), or mRNAs with distinct identifying sequences (groups 6-9). Mice were immunized with 0.5 μg, 1 μg, 2 μg, or 4 μg of a given mRNA composition (N=10 per dose group, 5 per PBS control group), and sera were collected on day 21. FIG. 9A shows Ag5-specific IgG titers. FIG. 9B shows Ag6-specific IgG titers. FIG. 9C shows Ag7-specific IgG titers. FIG. 9D shows Ag8-specific IgG titers.

### DETAILED DESCRIPTION

Aspects of the disclosure relate to methods for producing and/or analyzing compositions comprising multivalent different RNAs (e.g., 2 or more different RNAs). In some aspects, the disclosure is based on methods of selecting amounts of input DNA for IVT reactions that result in multivalent RNA compositions having higher purity than RNA compositions produced using previous methods. As described further in the Examples, it was observed that certain characteristics or properties of DNA molecules being co-transcribed (e.g., transcribed simultaneously in vitro), such as differences in length between DNA molecules, polyA-tailing efficiency of DNA molecules, etc., and/or other reagents present in the co-IVT reaction mixture (e.g., RNA polymerase, nucleotide triphosphates (NTPs), etc.) may introduce compositional bias into the resulting multivalent RNA compositions. Surprisingly, methods were discovered that reduce such compositional bias. In some embodiments, modifying input DNA amounts results in production of multivalent RNA compositions having increased purity (e.g., as measured by percentage of RNAs comprising polyA tails) relative to RNA compositions produced by previous methods. It was also surprisingly discovered that co-IVT methods described herein result in high purity multivalent RNA compositions even when there is a large difference (e.g., >100 nucleotides) in the lengths of the input DNAs used in the IVT reaction.

Accordingly, in some aspects, the disclosure relates to a method for producing a multivalent RNA composition, the method comprising simultaneously in vitro transcribing at least two DNA molecules in a reaction mixture comprising: a first population of DNA molecules encoding a first RNA; a second population of DNA molecules encoding a second RNA that is different than the first RNA; and obtaining a multivalent RNA composition having a pre-defined ratio of the first RNA to the second RNA produced by the IVT.

As used herein, the term “multivalent RNA composition” refers to a composition comprising more than two different mRNAs. A multivalent RNA composition may comprise 2 or more different RNAs, for example 2, 3, 4, 5, 6, 7, 8, 9, 10, or more different RNAs. In some embodiments, a multivalent RNA composition comprises more than 10 different RNAs. The term “different RNAs” refers to any RNA that is not the same as another RNA in a multivalent RNA composition. For example, two RNAs are different if they have i) different lengths (whether or not the RNAs are identical over the entirety of the shorter of the two lengths), ii) different nucleotide sequences, iii) different chemical modification patterns, or iv) any combination of the foregoing.

In some embodiments, mRNA present in a multivalent mRNA composition is at a pre-defined mRNA ratio, which may comprise a ratio between 2, 3, 4, 5, 6, 7, 8, 9, 10, or more different RNAs (e.g., depending on the number of different RNAs in a composition). In some embodiments, a pre-defined ratio comprises a ratio between more than 10 RNAs. As used herein, a “pre-defined mRNA ratio” refers to the desired final ratio of RNA molecules in a multivalent RNA composition. The desired final ratio of an RNA composition will depend upon the final peptide(s) or polypeptide product(s) encoded by the RNAs. For example, a multivalent RNA mixture may comprise two RNAs (e.g., a RNA encoding a heavy chain (HC) of an antibody and a light chain (LC) of an antibody); in this instance the desired final ratio of RNA molecules may be 1 HC RNA:1 LC RNA. In another example, a multivalent RNA composition may comprise several (e.g., 3, 4, 5, 6, 7, 8, 9, 10, or more) RNAs encoding different antigenic peptides (e.g., for use as a vaccine); in that instance the desired ratio may comprise between 3 and 10 RNAs (e.g., a:b:c, a:b:c:d, a:b:c:d:e, a:b:c:d:e:f, a:b:c:d:e:f:g, a:b:c:d:e:f:g:h, a:b:c:d:e:f:g:h:i, a:b:c:d:e:f:g:h:i:j, etc., where each of a-h is a number between 1 and 100).

### Nucleic Acids

Aspects of the disclosure relate to compositions comprising nucleic acids. As used herein, the term “nucleic acid” refers to multiple nucleotides (i.e., molecules comprising a sugar (e.g., ribose or deoxyribose) linked to a phosphate group and to an exchangeable organic base, which is either a substituted pyrimidine (e.g., cytosine (C), thymine (T) or uracil (U)) or a substituted purine (e.g., adenine (A) or guanine (G))). As used herein, the term nucleic acid refers to polyribonucleotides as well as polydeoxyribonucleotides. The term nucleic acid shall also include polynucleotides (i.e., a polynucleotide minus the phosphate) and any other organic base containing polymer. Non-limiting examples of nucleic acids include chromosomes, genomic loci, genes or gene segments that encode polynucleotides or polypeptides, coding sequences, non-coding sequences (e.g., intron, 5′-UTR, or 3′-UTR) of a gene, pri-mRNA, pre-mRNA, cDNA, mRNA, etc. A nucleic acid (e.g., mRNA) may include a substitution and/or modification. In some embodiments, the substitution and/or modification is in one or more bases and/or sugars. For example, in some embodiments a nucleic acid (e.g., mRNA) includes nucleic acids having backbone sugars that are covalently attached to low molecular weight organic groups other than a hydroxyl group at the 2′ position and other than a phosphate group or hydroxy group at the 5′ position. Thus, in some embodiments, a substituted or modified nucleic acid (e.g., mRNA) includes a 2′-O-alkylated ribose group. In some embodiments, a modified nucleic acid (e.g., mRNA) includes sugars such as hexose, 2′-F hexose, 2′-amino ribose, constrained ethyl (cEt), locked nucleic acid (LNA), arabinose or 2′-fluoroarabinose instead of ribose. Thus, in some embodiments, a nucleic acid (e.g., mRNA) is heterogeneous in backbone composition thereby containing any possible combination of polymer units linked together such as peptide-nucleic acids (which have an amino acid backbone with nucleic acid bases).

The nucleic acid sequences include nucleic acid sequences that have been removed from their naturally occurring environment, recombinant or cloned DNA isolates, and chemically synthesized analogues or analogues biologically synthesized by heterologous systems.

An “engineered nucleic acid” is a nucleic acid that does not occur in nature. It should be understood, however, that while an engineered nucleic acid as a whole is not naturally-occurring, it may include nucleotide sequences that occur in nature. In some embodiments, an engineered nucleic acid comprises nucleotide sequences from different organisms (e.g., from different species). For example, in some embodiments, an engineered nucleic acid includes a bacterial nucleotide sequence, a human nucleotide sequence, and/or a viral nucleotide sequence. Engineered nucleic acids include recombinant nucleic acids and synthetic nucleic acids. A “recombinant nucleic acid” is a molecule that is constructed by joining nucleic acids (e.g., isolated nucleic acids, synthetic nucleic acids or a combination thereof) and, in some embodiments, can replicate in a living cell. A “synthetic nucleic acid” is a molecule that is amplified or chemically, or by other means, synthesized. A synthetic nucleic acid includes those that are chemically modified, or otherwise modified, but can base pair with naturally-occurring nucleic acid molecules. Recombinant and synthetic nucleic acids also include those molecules that result from the replication of either of the foregoing. A nucleic may comprise naturally occurring nucleotides and/or non-naturally occurring nucleotides such as modified nucleotides.

In some embodiments, a nucleic acid is present in (or on) a vector. Examples of vectors include but are not limited to bacterial plasmids, phage, cosmids, phasmids, fosmids, bacterial artificial chromosomes, yeast artificial chromosomes, viruses and retroviruses (for example vaccinia, adenovirus, adeno-associated virus, lentivirus, herpes-simplex virus, Epstein-Barr virus, fowlpox virus, pseudorabies, baculovirus) and vectors derived therefrom. In some embodiments, a nucleic acid (e.g., DNA) used as an input molecule for in vitro transcription (IVT) is present in a plasmid vector.

When applied to a nucleic acid sequence, the term “isolated” denotes that the polynucleotide sequence has been removed from its natural genetic milieu and is thus free of other extraneous or unwanted coding sequences (but may include naturally occurring 5′ and 3′ untranslated regions such as promoters and terminators), and is in a form suitable for use within genetically engineered protein production systems. Such isolated molecules are those that are separated from their natural environment.

In some embodiments, an input DNA for IVT is a nucleic acid vector. A “nucleic acid vector” is a polynucleotide that carries at least one foreign or heterologous nucleic acid fragment. A nucleic acid vector may function like a “molecular carrier”, delivering fragments of nucleic acids or polynucleotides into a host cell or as a template for IVT. An “in vitro transcription template” (IVT template), or “input DNA” as used herein, refers to deoxyribonucleic acid (DNA) suitable for use in an IVT reaction for the production of messenger RNA (mRNA). In some embodiments, an IVT template encodes a 5′ untranslated region, contains an open reading frame, and encodes a 3′ untranslated region and a polyA tail. The particular nucleotide sequence composition and length of an IVT template will depend on the mRNA of interest encoded by the template.

In some embodiments the nucleic acid vector is a circular nucleic acid such as a plasmid. In other embodiments it is a linearized nucleic acid. According to some embodiments the nucleic acid vector comprises a predefined restriction site, which can be used for linearization. The linearization restriction site determines where the vector nucleic acid is opened/linearized. The restriction enzymes chosen for linearization should preferably not cut within the critical components of the vector.

A nucleic acid vector may include an insert which may be an expression cassette or open reading frame (ORF). An “open reading frame” is a continuous stretch of DNA beginning with a start codon (e.g., methionine (ATG)), and ending with a stop codon (e.g., TAA, TAG or TGA) and encodes a protein or peptide (e.g., a therapeutic protein or therapeutic peptide). In some embodiments, an expression cassette encodes a RNA including at least the following elements: a 5′ untranslated region, an open reading frame region encoding the mRNA, a 3′ untranslated region and a polyA tail. The open reading frame may encode any mRNA sequence, or portion thereof.

In some embodiments, a nucleic acid vector comprises a 5′ untranslated region (UTR). A “5′ untranslated region (UTR)” refers to a region of an mRNA that is directly upstream (i.e., 5′) from the start codon (i.e., the first codon of an mRNA transcript translated by a ribosome) that does not encode a protein or peptide. 5′ UTRs are further described herein, for example in the section entitled “Untranslated Regions”.

In some embodiments, a nucleic acid vector comprises a 3′ untranslated region (UTR). A “3′ untranslated region (UTR)” refers to a region of an mRNA that is directly downstream (i.e., 3′) from the stop codon (i.e., the codon of an mRNA transcript that signals a termination of translation) that does not encode a protein or peptide. 3′ UTRs are further described herein, for example in the section entitled “Untranslated Regions”.

The terms 5′ and 3′ are used herein to describe features of a nucleic acid sequence related to either the position of genetic elements and/or the direction of events (5′ to 3′), such as e.g. transcription by RNA polymerase or translation by the ribosome which proceeds in 5′ to 3′ direction. Synonyms are upstream (5′) and downstream (3′). Conventionally, DNA sequences, gene maps, vector cards and RNA sequences are drawn with 5′ to 3′ from left to right or the 5′ to 3′ direction is indicated with arrows, wherein the arrowhead points in the 3′ direction. Accordingly, 5′ (upstream) indicates genetic elements positioned towards the left-hand side, and 3′ (downstream) indicates genetic elements positioned towards the right-hand side, when following this convention.

Aspects of the disclosure relate to populations of molecules. As used herein, a “population” of molecules (e.g., DNA molecules) generally refers to a preparation (e.g., a plasmid preparation) comprising a plurality of copies of the molecule (e.g., DNA) of interest, for example a cell extract preparation comprising a plurality of expression vectors encoding a molecule of interest (e.g., a DNA encoding a RNA of interest).

A nucleic acid (e.g., mRNA) typically comprises a plurality of nucleotides. A nucleotide includes a nitrogenous base, a five-carbon sugar (ribose or deoxyribose), and at least one phosphate group. Nucleotides include nucleoside monophosphates, nucleoside diphosphates, and nucleoside triphosphates. A nucleoside monophosphate (NMP) includes a nucleobase linked to a ribose and a single phosphate; a nucleoside diphosphate (NDP) includes a nucleobase linked to a ribose and two phosphates; and a nucleoside triphosphate (NTP) includes a nucleobase linked to a ribose and three phosphates. Nucleotide analogs are compounds that have the general structure of a nucleotide or are structurally similar to a nucleotide. Nucleotide analogs, for example, include an analog of the nucleobase, an analog of the sugar and/or an analog of the phosphate group(s) of a nucleotide.

A nucleoside includes a nitrogenous base and a 5-carbon sugar. Thus, a nucleoside plus a phosphate group yields a nucleotide. Nucleoside analogs are compounds that have the general structure of a nucleoside or are structurally similar to a nucleoside. Nucleoside analogs, for example, include an analog of the nucleobase and/or an analog of the sugar of a nucleoside.

It should be understood that the term “nucleotide” includes naturally-occurring nucleotides, synthetic nucleotides and modified nucleotides, unless indicated otherwise. Examples of naturally-occurring nucleotides used for the production of RNA, e.g., in an IVT reaction, as described herein include adenosine triphosphate (ATP), guanosine triphosphate (GTP), cytidine triphosphate (CTP), uridine triphosphate (UTP), and 5-methyluridine triphosphate (m⁵UTP). In some embodiments, adenosine diphosphate (ADP), guanosine diphosphate (GDP), cytidine diphosphate (CDP), and/or uridine diphosphate (UDP) are used.

Examples of nucleotide analogs include, but are not limited to, antiviral nucleotide analogs, phosphate analogs (soluble or immobilized, hydrolyzable or non-hydrolyzable), dinucleotide, trinucleotide, tetranucleotide, e.g., a cap analog, or a precursor/substrate for enzymatic capping (vaccinia or ligase), a nucleotide labeled with a functional group to facilitate ligation/conjugation of cap or 5′ moiety (IRES), a nucleotide labeled with a 5′ PO₄ to facilitate ligation of cap or 5′ moiety, or a nucleotide labeled with a functional group/protecting group that can be chemically or enzymatically cleaved. Examples of antiviral nucleotide/nucleoside analogs include, but are not limited, to Ganciclovir, Entecavir, Telbivudine, Vidarabine and Cidofovir.

Modified nucleotides may include modified nucleobases. For example, a RNA transcript (e.g., mRNA transcript) may include a modified nucleobase selected from pseudouridine (W), 1-methylpseudouridine (m1ψ), 1-ethylpseudouridine, 2-thiouridine, 4′-thiouridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine (mo5U) and 2′-O-methyl uridine. In some embodiments, a RNA transcript (e.g., mRNA transcript) includes a combination of at least two (e.g., 2, 3, 4 or more) of the foregoing modified nucleobases.

### In Vitro Transcription (IVI)

Aspects of the present disclosure relate to methods of producing (e.g., synthesizing) a RNA transcript (e.g., mRNA transcript) comprising contacting a DNA template (e.g., a first input DNA and a second input DNA) with a RNA polymerase (e.g., a T7 RNA polymerase, a T7 RNA polymerase variant, etc.) under conditions that result in the production of the RNA transcript. This process is referred to as “in vitro transcription” or “IVT”. IVT conditions typically require a purified linear DNA template containing a promoter, nucleoside triphosphates, a buffer system that includes dithiothreitol (DTT) and magnesium ions, and a RNA polymerase. The exact conditions used in the transcription reaction depend on the amount of RNA needed for a specific application. Typical IVT reactions are performed by incubating a DNA template with a RNA polymerase and nucleoside triphosphates, including GTP, ATP, CTP, and UTP (or nucleotide analogs) in a transcription buffer. A RNA transcript having a 5′ terminal guanosine triphosphate is produced from this reaction.

In some embodiments, a wild-type T7 polymerase is used in an IVT reaction. In some embodiments, a modified or mutant T7 polymerase is used in an IVT reaction. In some embodiments, a T7 RNA polymerase variant comprises an amino acid sequences that shares at least 50%, 60%, 70%, 80%, 90%, 95%, or 99% identity with a wild-type T7 (WT T7) polymerase. In some embodiments, the T7 polymerase variant is a T7 polymerase variant described by International Application Publication Number WO2019/036682 or WO2020/172239, the entire contents of each of which are incorporated herein by reference. In some embodiments, the RNA polymerase (e.g., T7 RNA polymerase or T7 RNA polymerase variant) is present in a reaction (e.g., an IVT reaction) at a concentration of 0.01 mg/ml to 1 mg/ml. For example, the RNA polymerase may be present in a reaction at a concentration of 0.01 mg/mL, 0.05 mg/ml, 0.1 mg/ml, 0.5 mg/ml or 1.0 mg/ml.

The “percent identity,” “sequence identity,” “% identity,” or “% sequence identity” (as they may be interchangeably used herein) of two sequences (e.g., nucleic acid or amino acid) refers to a quantitative measurement of the similarity between two sequences (e.g., nucleic acid or amino acid). Percent identity can be determined using the algorithms of Karlin and Altschul, Proc. Natl. Acad. Sci. USA 87:2264-68, 1990, modified as in Karlin and Altschul, Proc. Natl. Acad. Sci. USA 90:5873-77, 1993. Such algorithms are incorporated into the NBLAST and XBLAST programs (version 2.0) of Altschul et al., J. Mol. Biol. 215:403-10, 1990. BLAST protein searches can be performed with the XBLAST program, score=50, word length=3, to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used. When a percent identity is stated, or a range thereof (e.g., at least, more than, etc.), unless otherwise specified, the endpoints shall be inclusive and the range (e.g., at least 70% identity) shall include all ranges within the cited range.

The input deoxyribonucleic acid (DNA) serves as a nucleic acid template for RNA polymerase. A DNA template may include a polynucleotide encoding a polypeptide of interest (e.g., an antigenic polypeptide). A DNA template, in some embodiments, includes a RNA polymerase promoter (e.g., a T7 RNA polymerase promoter) located 5′ from and operably linked to polynucleotide encoding a polypeptide of interest. A DNA template may also include a nucleotide sequence encoding a polyadenylation (polyA) tail located at the 3′ end of the gene of interest. In some embodiments, an input DNA comprises plasmid DNA (pDNA). As used herein, “plasmid DNA” or “pDNA” refers to an extrachromosomal DNA molecule that is physically separated from chromosomal DNA in a cell and can replicate independently. In some embodiments, plasmid DNA is isolated from a cell (e.g., as a plasmid DNA preparation). In some embodiments, plasmid DNA comprises an origin of replication, which may contain one or more heterologous nucleic acids, for example nucleic acids encoding therapeutic proteins that may serve as a template for RNA polymerase. Plasmid DNA may be circularized or linear (e.g., plasmid DNA that has been linearized by a restriction enzyme digest).

In some embodiments, each input DNA (e.g., population of input DNA molecules) in a co-IVT reaction is obtained from a different source (e.g., synthesized separately, for example in different cells or populations of cells). In some embodiments, each input DNA (e.g., population of input DNA) is obtained from a different bacterial cell or population of bacterial cells. For example, in a co-IVT reaction having three populations of input DNAs, the first input DNA is produced in bacterial cell population A, the second input DNA is produced in bacterial cell population B, and the third input DNA is produced in bacterial population C, where each of A, B, and C are not the same bacterial culture (e.g., co-cultured in the same container or plate). In another example, two input DNAs obtained from different sources are i) chemically synthesized in separate synthesis reactions, or ii) produced by separate amplification (e.g., polymerase chain reactions (PCR reactions)). Methods of obtaining populations of input DNAs (e.g., plasmid DNAs) are known, for example as described by Sambrook, Joseph. Molecular Cloning: a Laboratory Manual. Cold Spring Harbor, N.Y.: Cold Spring Harbor Laboratory Press, 2001.

Some aspects comprise normalizing the amount of DNA used in the multivalent co-IVT reaction. In some embodiments, the normalization is based on the molar mass of the input DNAs. In some embodiments, the normalization is based on the degradation rate of the input DNAs. In some embodiments, the normalization is based on the degradation rate of the resultant mRNAs (e.g., measured based upon polyA variants present in the reaction mixture, or T7 polymerase abortive transcripts or truncated transcripts). In some embodiments, the normalization is based on the nucleotide content (e.g., amount of A, G, C, U, or any combination thereof) of the input DNAs. In some embodiments, the normalization is based on the purity of the input DNAs. In some embodiments the normalization is based on the polyA-tailing efficiency of the input DNAs. In some embodiments, the normalization is based on the lengths of the input DNAs.

In some embodiments, the normalization is based on the lowest level present in the input DNAs (e.g., lowest molar mass, degradation rate (e.g., of the input DNA and/or output RNA), nucleotide content, purity, and/or polyA-tailing efficiency). In some embodiments, the normalization is based on the highest level present in the input DNAs (e.g., highest molar mass, degradation rate (e.g., of the input DNA and/or output RNA), nucleotide context, purity, and/or polyA-tailing efficiency). In some embodiments, the normalization is based on the rate of RNA production of the input DNAs (e.g., the highest rate of RNA production of an input DNA or the lowest rate of RNA production of an input DNA in a reaction mixture).

In some aspects, the disclosure relates to IVT methods in which the amount of input DNA (e.g., a first DNA or second DNA) is adjusted or normalized in order to improve production of multivalent RNA compositions having a pre-defined mRNA ratio of components. The disclosure is based, in part, on the discovery that certain factors affecting multivalent RNA composition purity, such as large differences in size between input DNAs (e.g., a difference of more than 100, 200, 500, 1000, or more nucleotides in length) and/or polyA-tailing efficiency of a given DNA during IVT, may be addressed prior to the IVT by normalizing the amount of input DNA based upon one or more of those factors. For example, in some embodiments, the amount of two input DNAs is calculated based upon the desired molar ratio of the first RNA to the second RNA that are transcribed from the input DNAs. In some embodiments, the calculating comprises determining a plasmid mass ratio based upon the desired molar ratio of the input DNAs. In some embodiments, the amount of input DNAs is normalized based upon the highest polyA-tailing efficiency of the input DNAs during IVT.

The number of input DNAs (e.g., populations of input DNA molecules) used in an IVT reaction may vary, depending upon the number of different RNA molecules desired to be included in the multivalent RNA composition. In some embodiments, an IVT reaction mixture comprises 2 or more different input DNAs, for example 2, 3, 4, 5, 6, 7, 8, 9, 10, or more different input DNAs. In some embodiments, the IVT reaction comprises more than 10 different input DNAs. The term “different input DNAs” encompasses input DNAs that encode different RNAs, e.g., that have i) different lengths (whether or not the RNAs are identical over the entirety of the shorter of the two lengths), ii) different nucleotide sequences, iii) different chemical modification patterns, or iv) any combination of the foregoing.

The concentration of each of the populations of DNA molecules may also vary. In some embodiments, the concentration of each population of DNA molecules in an IVT reaction ranges from about 0.005 mg/mL to about 0.5 mg/ml. In some embodiments, the concentration of each population of DNA molecules in an IVT reaction ranges from about 0.02 mg/ml to about 0.05 mg/ml, 0.02 to about 0.15 mg/ml, about 0.05 mg/ml to about 0.20 mg/ml, about 0.175 to about 0.3 mg/ml, about 0.2 mg/ml to about 0.5 mg/ml, about 0.3 mg/ml to about 0.6 mg/ml, about 0.5 mg/ml to about 0.75 mg/ml, about 0.5 mg/ml to about 1.0 mg/ml, about 0.75 mg/ml to about 0.9 mg/ml, about 0.75 mg/ml to about 1.5 mg/ml, about 0.8 mg/ml to about 1.2 mg/ml, about 1.0 mg/ml to about 1.5 mg/ml, about 1.0 mg/ml to about 2.5 mg/ml, about 1.5 mg/ml to about 3.0 mg/ml, about 2.0 mg/ml to about 4.0 mg/ml, or about 2.5 mg/ml to about 5.0 mg/ml.

In some embodiments, the input DNAs are added to an IVT reaction are a predefined DNA ratio, which may comprise a ratio between 2, 3, 4, 5, 6, 7, 8, 9, 10, or more different input DNAs (e.g., depending on the number of different RNAs in a composition). In some embodiments, a pre-defined input DNA ratio comprises a ratio between more than 10 input DNAs. As used herein, a “pre-defined input DNA ratio” refers to the desired final ratio of DNA molecules in an IVT reaction. The desired final ratio of input DNAs can depend upon the final peptide(s) or polypeptide product(s) encoded by RNAs encoded by the input DNAs. In some embodiments, the input DNAs can have a desired ratio that may comprise between 2 and 8 input DNAs (e.g., a:b, a:b:c, a:b:c:d, a:b:c:d:e, a:b:c:d:e:f, a:b:c:d:e:f:g, a:b:c:d:e:f:g:h, etc., where each of a-h is a number between 1 and 10). In some embodiments, the pre-defined input DNA ratio is different form the pre-defined mRNA ratio.

The size of two or more input DNAs (e.g., DNAs in two or more different populations of input DNAs) may vary. In some embodiments, an input DNA includes from about 15 to about 8,000 base pairs (e.g., from 15 to 50, 15 to 100, 15 to 200, 15 to 300, 15 to 400, 15 to 500, 15 to 600, 15 to 700, 15 to 800, 15 to 900, 15 to 1000, 15 to 1200, 15 to 1400, 15 to 1500, 15 to 1800, 15 to 2000, 15 to 2500, 15 to 3000, 50 to 100, 50 to 200, 50 to 300, 50 to 400, 50 to 500, 50 to 600, 50 to 700, 50 to 800, 50 to 900, 50 to 1000, 50 to 1200, 50 to 1400, 50 to 1500, 50 to 1800, 50 to 2000, 50 to 2500, 50 to 3000, 100 to 200, 100 to 300, 100 to 400, 100 to 500, 100 to 600, 100 to 700, 100 to 800, 100 to 900, 100 to 1000, 100 to 1200, 100 to 1400, 100 to 1500, 100 to 1800, 100 to 2000, 100 to 2500, 100 to 3000, 200 to 300, 200 to 400, 200 to 500, 200 to 600, 200 to 700, 200, to 800, 200 to 900, 200 to 1000, 200 to 1500, 200 to 3000, 500 to 1000, 500 to 1500, 500 to 2000, 500 to 2500, 500 to 3000, 1000 to 1500, 1000 to 2000, 1000 to 2500, 1000 to 3000, 1500 to 3000, 2500 to 3000, 2000 to 3000, 2500 to 4000, 3000 to 5000, 3500 to 6500, 5000 to 7500, or 6500 to 8000 base pairs.

The mass of each population of input DNA molecules in an IVT reaction may vary. In some embodiments, the mass of each population of input DNA ranges based upon the total volume of the IVT reaction mixture. In some embodiments, the mass of each population of each input DNA molecule in an IVT mixture individually varies from about 0.5% to about 99.9% of the total input DNA present in the IVT reaction mixture. In some embodiments, the molar ratio of each population of input DNA molecules in an IVT reaction may vary.

In some embodiments, two or more of the input DNA molecules used in an IVT reaction have a different length (e.g., comprises a different number of nucleotides). In some embodiments, the difference in length between two or more (e.g., 3, 4, 5, 6, 7, 8, 9, 10, or more) of the different input DNA molecules in an IVT reaction mixture is greater than 70 base pairs, 80 base pairs, 90 base pairs, or 100 base pairs (e.g., two input DNAs in a composition are not within 70, 80, 90, or 100 base pairs in length of one another). In some embodiments, the difference in length between two or more (e.g., 3, 4, 5, 6, 7, 8, 9, 10, or more) of the different input DNA molecules is more than 100 base pairs, for example 500 base pairs, 1000 base pairs, 1500 base pairs, 2000 base pairs, 3000 base pairs, 4000 base pairs, 5000 base pairs, 6000 base pairs, 7000 base pairs, 8000 base pairs, or more.

In some embodiments, two or more of the input DNA molecules used in an IVT reaction encode mRNA molecules that have a different length (e.g., comprises a different number of nucleotides). In some embodiments, the difference in length between two or more of the mRNA molecules encoded by different input DNA molecules in an VT reaction mixture is greater than 70 nucleotides, 80 nucleotides, 90 nucleotides, or 100 nucleotides (e.g., two input DNAs in a composition encode mRNA molecules that are not are within 70, 80, 90, or 100 nucleotides in length of one another). In some embodiments, the difference in length between two or more of the mRNA molecules encoded by different input DNA molecules is more than 100 nucleotides, for example 500 nucleotides, 1000 nucleotides, 1500 nucleotides, 2000 nucleotides, 3000 nucleotides, 4000 nucleotides, or more.

In some embodiments, the multivalent IVT comprises co-transcription of at least 2 different input DNAs (e.g., at least 2 of DNA A, B, C, D, E, F, F, H, 1, J, etc.) at a ratio of A:B:C:D:E:F:G:H:I:J, wherein if DNA A is normalized to 1, one or more of DNA B, C, D, E, F, G, H, I, J, etc. can each independently be present at an amount (e.g., a concentration) that is from 0.01 to 100 times the amount (e.g., a concentration) of A, such as from 0.05 times to 20 times the amount of A, 0.1 times to 10 times the amount of A, 0.2 times to 5 times the amount of A, 0.3 times to 3 times the amounts of A, 0.5 times to 2 times the amounts of A, 0.75 times to 1.4 times the amount of A, 0.8 times to 1.25 times the amount of A, or 0.9 times to 1.15 times the amount of A. One or more of DNA B, C, D, E, F, G, H, I, or J may also be absent.

In some embodiments, a multivalent RNA composition is produced by combining RNA transcripts (e.g., mRNAs) from separate sources. In some embodiments, a multivalent RNA composition is produced by separately transcribing two or more DNA templates in separate IVT reactions, and combining the transcribed RNAs. In some embodiments, an RNA transcript is produced by IVT, then added to one or more other RNAs. RNAs may be combined in any desired amount to produce a multivalent RNA composition comprising two or more RNAs in a specific ratio.

A RNA transcript, in some embodiments, is the product of an IVT reaction. A RNA transcript, in some embodiments, is a messenger RNA (mRNA) that includes a nucleotide sequence encoding a polypeptide of interest (e.g., a therapeutic protein or therapeutic peptide) linked to a polyA tail. In some embodiments, the mRNA is modified mRNA (mmRNA), which includes at least one modified nucleotide.

The nucleoside triphosphates (NTPs) as described herein may comprise unmodified or modified ATP, modified or unmodified UTP, modified or unmodified GTP, and/or modified or unmodified CTP. In some embodiments, NTPs of an IVT reaction comprise unmodified ATP. In some embodiments, NTPs of an IVT reaction comprise modified ATP. In some embodiments, NTPs of an IVT reaction comprise unmodified UTP. In some embodiments, NTPs of an IVT reaction comprise modified UTP. In some embodiments, NTPs of an IVT reaction comprise unmodified GTP. In some embodiments, NTPs of an IVT reaction comprise modified GTP. In some embodiments, NTPs of an IVT reaction comprise unmodified CTP. In some embodiments, NTPs of an IVT reaction comprise modified CTP.

The composition of NTPs in an IVT reaction may also vary. In some embodiments, each NTP in an IVT reaction is present in an equimolar amount. In some embodiments, each NTP in an IVT reaction is present in non-equimolar amounts. For example, ATP may be used in excess of GTP, CTP and UTP. As a non-limiting example, an IVT reaction may include 7.5 millimolar GTP, 7.5 millimolar CTP, 7.5 millimolar UTP, and 3.75 millimolar ATP. In some embodiments, the molar ratio of G:C:U:A is 2:1:0.5:1. In some embodiments, the molar ratio of G:C:U:A is 1:1:0.7:1. In some embodiments, the molar ratio of G:C:A:U is 1:1:1:1. The same IVT reaction may include 3.75 millimolar cap analog (e.g., trinucleotide cap or tetranucleotide cap). In some embodiments, the molar ratio of G:C:U:A:cap is 1:1:1:0.5:0.5. In some embodiments, the molar ratio of G:C:U:A:cap is 1:1:0.5:1:0.5. In some embodiments, the molar ratio of G:C:U:A:cap is 1:0.5:1:1:0.5. In some embodiments, the molar ratio of G:C:U:A:cap is 0.5:1:1:1:0.5. In some embodiments, the amount of NTPs in a co-IVT reaction is calculated empirically. For example, the rate of consumption for each NTP in an IVT reaction may be empirically determined for each individual input DNA, and then balanced ratios of NTPs based on those individual NTP consumption rates may be added to a co-IVT comprising multiple of the input DNAs.

In some embodiments, an IVT reaction mixture further comprises cap analog (e.g., as further described herein in the section entitled “RNA Capping”). The concentration of nucleoside triphosphates and cap analog present in an IVT reaction may vary. In some embodiments, NTPs and cap analog are present in the reaction at equimolar concentrations. In some embodiments, the molar ratio of cap analog (e.g., trinucleotide cap or tetranucleotide cap) to nucleoside triphosphates in the reaction is greater than 1:1. For example, the molar ratio of cap analog to nucleoside triphosphates in the reaction may be 2:1, 3:1, 4:1, 5:1, 6:1, 7:1, 8:1, 9:1, 10:1, 15:1, 20:1, 25:1, 50:1, or 100:1. In some embodiments, the molar ratio of cap analog (e.g., trinucleotide cap or tetranucleotide cap) to nucleoside triphosphates in the reaction is less than 1:1. For example, the molar ratio of cap analog (e.g., trinucleotide cap or tetranucleotide cap) to nucleoside triphosphates in the reaction may be 1:2, 1:3, 1:4, 1:5, 1:6, 1:7, 1:8, 1:9, 1:10, 1:15, 1:20, 1:25, 1:50, or 1:100.

In some embodiments, a RNA transcript (e.g., mRNA transcript) includes a modified nucleobase selected from pseudouridine (ψ), 1-methylpseudouridine (m¹ψ), 5-methoxyuridine (mo⁵U), 5-methylcytidine (m⁵C), α-thio-guanosine and α-thio-adenosine. In some embodiments, a RNA transcript (e.g., mRNA transcript) includes a combination of at least two (e.g., 2, 3, 4 or more) of the foregoing modified nucleobases.

In some embodiments, a RNA transcript (e.g., mRNA transcript) includes pseudouridine (ψ). In some embodiments, a RNA transcript (e.g., mRNA transcript) includes 1-methylpseudouridine (m¹ψ). In some embodiments, a RNA transcript (e.g., mRNA transcript) includes 5-methoxyuridine (mo⁵U). In some embodiments, a RNA transcript (e.g., mRNA transcript) includes 5-methylcytidine (m⁵C). In some embodiments, a RNA transcript (e.g., mRNA transcript) includes α-thio-guanosine. In some embodiments, a RNA transcript (e.g., mRNA transcript) includes α-thio-adenosine.

In some embodiments, the polynucleotide (e.g., RNA polynucleotide, such as mRNA polynucleotide) is uniformly modified (e.g., fully modified, modified throughout the entire sequence) for a particular modification. For example, a polynucleotide can be uniformly modified with 1-methylpseudouridine (m¹ψ), meaning that all uridine residues in the mRNA sequence are replaced with 1-methylpseudouridine (m¹ψ). Similarly, a polynucleotide can be uniformly modified for any type of nucleoside residue present in the sequence by replacement with a modified residue such as any of those set forth above. Alternatively, the polynucleotide (e.g., RNA polynucleotide, such as mRNA polynucleotide) may not be uniformly modified (e.g., partially modified, part of the sequence is modified). Each possibility represents a separate embodiment.

The buffer system of an IVT reaction mixture may vary. In some embodiments, the buffer system contains tris. The concentration of tris used in an IVT reaction, for example, may be at least 10 mM, at least 20 mM, at least 30 mM, at least 40 mM, at least 50 mM, at least 60 mM, at least 70 mM, at least 80 mM, at least 90 mM, at least 100 mM or at least 110 mM phosphate. In some embodiments, the concentration of phosphate is 20-60 mM or 10-100 mM.

In some embodiments, the buffer system contains dithiothreitol (DTT). The concentration of DTT used in an IVT reaction, for example, may be at least 1 mM, at least 5 mM, or at least 50 mM. In some embodiments, the concentration of DTT used in an IVT reaction is 1-50 mM or 5-50 mM. In some embodiments, the concentration of DTT used in an IVT reaction is 5 mM.

In some embodiments, the buffer system contains magnesium. In some embodiments, the molar ratio of NTP to magnesium ions (Mg²⁺; e.g., MgCl₂) present in an IVT reaction is 1:1 to 1:5. For example, the molar ratio of NTP to magnesium ions may be 1:0.25, 1:0.5, 1:1, 1:2, 1:3, 1:4 or 1:5.

In some embodiments, the molar ratio of NTP plus cap analog (e.g., trinucleotide cap, such as GAG) to magnesium ions (Mg²⁺; e.g., MgCl₂) present in an IVT reaction is 1:1 to 1:5. For example, the molar ratio of NTP+trinucleotide cap (e.g., GAG) to magnesium ions may be 1:1, 1:2, 1:3, 1:4 or 1:5.

In some embodiments, the buffer system contains Tris-HCl, spermidine (e.g., at a concentration of 1-30 mM), TRITON® X-100 (polyethylene glycol p-(1,1,3,3-tetramethylbutyl)-phenyl ether) and/or polyethylene glycol (PEG).

In some embodiments, IVT methods further comprise a step of separating (e.g., purifying) in vitro transcription products (e.g., mRNA) from other reaction components. In some embodiments, the separating comprises performing chromatography on the IVT reaction mixture. In some embodiments, the chromatography comprises size-based (e.g., length-based) chromatography. In some embodiments, the chromatography comprises oligo-dT chromatography.

### RNA Capping

The multivalent RNA compositions described herein may comprise one or more mRNAs having open reading frames that encode proteins or peptides. Each of these mRNAs may have a 5′ Cap. The 5′ Cap may be added during the co-IVT reaction (e.g., transcriptional co-capping) or after the IVT reaction.

The disclosure also includes a polynucleotide that comprises both a 5′ Cap and a polynucleotide (e.g., a polynucleotide comprising a nucleotide sequence encoding a polypeptide to be expressed).

The 5′ cap structure of a natural mRNA is involved in nuclear export, increasing mRNA stability and binds the mRNA Cap Binding Protein (CBP), for example eIF4E, which is responsible for mRNA stability in the cell and translation competency through the association of CBP with poly(A) binding protein to form the mature cyclic mRNA species. The cap further assists the removal of 5′ proximal introns during mRNA splicing.

Endogenous mRNA molecules can be 5′-end capped generating a 5′-ppp-5′-triphosphate linkage between a terminal guanosine cap residue and the 5′-terminal transcribed sense nucleotide of the mRNA molecule. This 5′-guanylate cap can then be methylated to generate an N7-methyl-guanylate residue. The ribose sugars of the terminal and/or anteterminal transcribed nucleotides of the 5′ end of the mRNA can optionally also be 2′-O-methylated. 5′-decapping through hydrolysis and cleavage of the guanylate cap structure can target a nucleic acid molecule, such as an mRNA molecule, for degradation.

In some embodiments, the polynucleotides (e.g., a polynucleotide comprising a nucleotide sequence encoding a polypeptide) incorporate a cap moiety.

In some embodiments, polynucleotides comprise a non-hydrolyzable cap structure preventing decapping and thus increasing mRNA half-life. Because cap structure hydrolysis requires cleavage of 5′-ppp-5′ phosphodiester linkages, modified nucleotides can be used during the capping reaction. For example, a Vaccinia Capping Enzyme from New England Biolabs (Ipswich, MA) can be used with α-thio-guanosine nucleotides according to the manufacturer's instructions to create a phosphothioate linkage in the 5′-ppp-5′ cap. Additional modified guanosine nucleotides can be used such as α-methyl-phosphonate and seleno-phosphate nucleotides.

Additional modifications include, but are not limited to, 2′-O-methylation of the ribose sugars of 5′-terminal and/or 5′-anteterminal nucleotides of the polynucleotide (as mentioned above) on the 2′-hydroxyl group of the sugar ring. Multiple distinct 5′-cap structures can be used to generate the 5′-cap of a nucleic acid molecule, such as a polynucleotide that functions as an mRNA molecule. Cap analogs, which herein are also referred to as synthetic cap analogs, chemical caps, chemical cap analogs, or structural or functional cap analogs, differ from natural (i.e., endogenous, wild-type or physiological) 5′-caps in their chemical structure, while retaining cap function. Cap analogs can be chemically (i.e., non-enzymatically) or enzymatically synthesized and/or linked to the polynucleotides.

For example, the Anti-Reverse Cap Analog (ARCA) cap contains two guanines linked by a 5′-5′-triphosphate group, wherein one guanine contains an N7 methyl group as well as a 3′-O-methyl group (i.e., N7,3′-O-dimethyl-guanosine-5′-triphosphate-5′-guanosine (m⁷G-3′mppp-G; which can equivalently be designated 3′ O-Me-m⁷G(5′)ppp(5′)G). The 3′-O atom of the other, unmodified, guanine becomes linked to the 5′-terminal nucleotide of the capped polynucleotide. The N7- and 3′-O-methylated guanine provides the terminal moiety of the capped polynucleotide.

Another exemplary cap is mCAP, which is similar to ARCA but has a 2′-O-methyl group on guanosine (i.e., N7,2′-O-dimethyl-guanosine-5′-triphosphate-5′-guanosine, m⁷Gm-ppp-G).

Another exemplary cap is m⁷G-ppp-Gm-AG (i.e., N7,guanosine-5′-triphosphate-2′-O-dimethyl-guanosine-adenosine-guanosine).

In some embodiments, the cap is a dinucleotide cap analog. As a non-limiting example, the dinucleotide cap analog can be modified at different phosphate positions with a boranophosphate group or a phosphoroselenoate group such as the dinucleotide cap analogs described in U.S. Pat. No. 8,519,110, the contents of which are herein incorporated by reference in its entirety.

In another embodiment, the cap is a cap analog is a N7-(4-chlorophenoxyethyl) substituted dinucleotide form of a cap analog known in the art and/or described herein. Non-limiting examples of a N7-(4-chlorophenoxyethyl) substituted dinucleotide form of a cap analog include a N7-(4-chlorophenoxyethyl)-G(5′)ppp(5′)G and a N7-(4-chlorophenoxyethyl)-m^{3′-O}G(5′)ppp(5′)G cap analog (See, e.g., the various cap analogs and the methods of synthesizing cap analogs described in Kore et al. Bioorganic & Medicinal Chemistry 2013 21:4570-4574; the contents of which are herein incorporated by reference in its entirety). In another embodiment, a cap analog is a 4-chloro/bromophenoxyethyl analog.

Polynucleotides can also be capped post-manufacture (whether IVT or chemical synthesis), using enzymes, in order to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function and/or structure as compared to synthetic features or analogs, etc., of the prior art, or which outperforms the corresponding endogenous, wild-type, natural or physiological feature in one or more respects. Non-limiting examples of more authentic 5′cap structures are those that, among other things, have enhanced binding of cap binding proteins, increased half-life, reduced susceptibility to 5′ endonucleases and/or reduced 5′decapping, as compared to synthetic 5′cap structures known in the art (or to a wild-type, natural or physiological 5′cap structure). For example, recombinant Vaccinia Virus Capping Enzyme and recombinant 2′-O-methyltransferase enzyme can create a canonical 5′-5′-triphosphate linkage between the 5′-terminal nucleotide of a polynucleotide and a guanine cap nucleotide wherein the cap guanine contains an N7 methylation and the 5′-terminal nucleotide of the mRNA contains a 2′-O-methyl. Such a structure is termed the Cap1 structure. This cap results in a higher translational-competency and cellular stability and a reduced activation of cellular pro-inflammatory cytokines, as compared, e.g., to other 5′cap analog structures known in the art. Cap structures include, but are not limited to, 7mG(5′)ppp(5′)N,pN2p (cap 0), 7mG(5′)ppp(5′)NlmpNp (cap 1), and 7mG(5′)-ppp(5′)NlmpN2mp (cap 2).

As a non-limiting example, capping chimeric polynucleotides post-manufacture can be more efficient as nearly 100% of the chimeric polynucleotides can be capped. This is in contrast to ˜80% when a cap analog is linked to a chimeric polynucleotide in the course of an in vitro transcription reaction.

In some embodiments, 5′ terminal caps can include endogenous caps or cap analogs. In some embodiments, a 5′ terminal cap can comprise a guanine analog. Useful guanine analogs include, but are not limited to, inosine, N1-methyl-guanosine, 2′fluoro-guanosine, 7-deaza-guanosine, 8-oxo-guanosine, 2-amino-guanosine, LNA-guanosine, and 2-azido-guanosine.

Also described herein are exemplary caps including those that can be used in co-transcriptional capping methods for ribonucleic acid (RNA) synthesis, using RNA polymerase, e.g., wild type RNA polymerase or variants thereof, e.g., such as those variants described herein. In one embodiment, caps can be added when RNA is produced in a “one-pot” reaction, without the need for a separate capping reaction. Thus, the methods, in some embodiments, comprise reacting a polynucleotide template with a RNA polymerase variant, nucleoside triphosphates, and a cap analog under in vitro transcription reaction conditions to produce RNA transcript.

In some embodiments, the cap analog binds to a polynucleotide template that comprises a promoter region comprising a transcriptional start site having a first nucleotide at nucleotide position +1, a second nucleotide at nucleotide position +2, and a third nucleotide at nucleotide position +3. In some embodiments, the cap analog hybridizes to the polynucleotide template at least at nucleotide position +1, such as at the +1 and +2 positions, or at the +1, +2, and +3 positions.

A cap analog may be, for example, a dinucleotide cap, a trinucleotide cap, or a tetranucleotide cap. In some embodiments, a cap analog is a dinucleotide cap. In some embodiments, a cap analog is a trinucleotide cap. In some embodiments, a cap analog is a tetranucleotide cap. As used here the term “cap” includes the inverted G nucleotide and can comprise additional nucleotides 3′ of the inverted G, e.g., 1, 2, or more nucleotides 3′ of the inverted G and 5′ to the 5′ UTR.

Exemplary caps comprise a sequence GG, GA, or GGA wherein the underlined, italicized G is an inverted G.

A trinucleotide cap, in some embodiments, comprises a compound of formula (I)

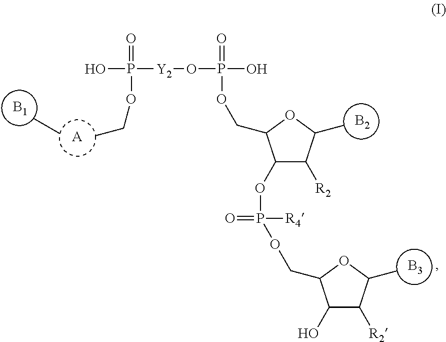

or a stereoisomer, tautomer or salt thereof, wherein

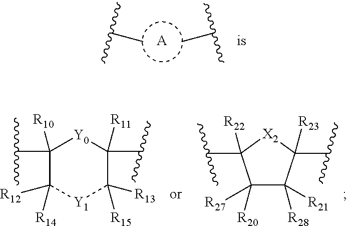

ring B₁ is a modified or unmodified Guanine;
ring B₂ and ring B₃ each independently is a nucleobase or a modified nucleobase;
X₂ is O, S(O)ₚ, NR₂₄ or CR₂₅R₂₆ in which p is 0, 1, or 2;
Y₀ is O or CR₆R₇;
Y₁ is O, S(O)ₙ, CR₆R₇, or NR₈, in which n is 0, 1, or 2;
each [symbol] is a single bond or absent, wherein when each [symbol] is a single bond, Yi is O, S(O)ₙ, CR₆R₇, or NR₈; and when each [symbol] is absent, Y₁ is void;
Y₂ is (OP(O)R₄)ₘ in which m is 0, 1, or 2, or —O—(CR₄₀R₄₁)u-Q₀-(CR₄₂R₄₃)v-, in which Q₀ is a bond, O, S(O)ᵣ, NR₄₄, or CR₄₅R₄₆, r is 0, 1, or 2, and each of u and v independently is 1, 2, 3 or 4;
each R₂ and R₂′ independently is halo, LNA, or OR₃;
each R₃ independently is H, C₁-C₆ alkyl, C₂-C₆ alkenyl, or C₂-C₆ alkynyl and R₃, when being C₁-C₆ alkyl, C₂-C6 alkenyl, or C₂-C₆ alkynyl, is optionally substituted with one or more of halo, OH and C₁-C₆ alkoxyl that is optionally substituted with one or more OH or OC(O)—C₁-C₆ alkyl;
each R₄ and R₄′ independently is H, halo, C₁-C₆ alkyl, OH, SH, SeH, or BH₃⁻;
each of R₆, R₇, and R₈, independently, is -Q₁-T₁, in which Q₁ is a bond or C₁-C₃ alkyl linker optionally substituted with one or more of halo, cyano, OH and C₁-C₆ alkoxy, and T₁ is H, halo, OH, COOH, cyano, or Rₛ₁, in which Rₛ₁ is C₁-C₃ alkyl, C₂-C₆ alkenyl, C₂-C₆ alkynyl, C₁-C₆ alkoxyl, C(O)O—C₁-C₆ alkyl, C₃-C₈ cycloalkyl, C₆-C₁₀ aryl, NR₃₁R₃₂, (NR₃₁R₃₂R₃₃)⁺, 4 to 12-membered heterocycloalkyl, or 5- or 6-membered heteroaryl, and Rₛ₁ is optionally substituted with one or more substituents selected from the group consisting of halo, OH, oxo, C₁-C₆ alkyl, COOH, C(O)O—C₁-C₆ alkyl, cyano, C₁-C₆ alkoxyl, NR₃₁R₃₂, (NR₃₁R₃₂R₃₃)⁺, C₃-C₈ cycloalkyl, C₆-C₁₀ aryl, 4 to 12-membered heterocycloalkyl, and 5- or 6-membered heteroaryl;
each of R₁₀, R₁₁, R₁₂, R₁₃ R₁₄, and R₁₅, independently, is -Q₂-T₂, in which Q₂ is a bond or C₁-C₃ alkyl linker optionally substituted with one or more of halo, cyano, OH and C₁-C₆ alkoxy, and T₂ is H, halo, OH, NH₂, cyano, NO₂, N₃, Rₛ₂, or ORₛ₂, in which Rₛ₂ is C₁-C₆ alkyl, C₂-C₆ alkenyl, C₂-C₆ alkynyl, C₃-C₈ cycloalkyl, C₆-C₁₀ aryl, NHC(O)—C₁-C₆ alkyl, NR₃₁R₃₂, (NR₃₁R₃₂R₃₃)⁺, 4 to 12-membered heterocycloalkyl, or 5- or 6-membered heteroaryl, and Rₛ₂ is optionally substituted with one or more substituents selected from the group consisting of halo, OH, oxo, C₁-C₆ alkyl, COOH, C(O)O—C₁-C₆ alkyl, cyano, C₁-C₆ alkoxyl, NR₃₁R₃₂, (NR₃₁R₃₂R₃₃)⁺, C₃-C₈ cycloalkyl, C₆-C₁₀ aryl, 4 to 12-membered heterocycloalkyl, and 5- or 6-membered heteroaryl; or alternatively R₁₂ together with R₁₄ is oxo, or R₁₃ together with R₁₅ is oxo,
each of R₂₀, R₂₁, R₂₂, and R₂₃ independently is -Q₃-T₃, in which Q₃ is a bond or C₁-C₃ alkyl linker optionally substituted with one or more of halo, cyano, OH and C₁-C₆ alkoxy, and T₃ is H, halo, OH, NH₂, cyano, NO₂, N₃, R_{S3}, or OR_{S3}, in which R_{S3} is C₁-C₆ alkyl, C₂-C₆ alkenyl, C₂-C₆ alkynyl, C₃-C₈ cycloalkyl, C₆-C₁₀ aryl, NHC(O)—C₁-C₆ alkyl, mono-C₁-C₆ alkylamino, di-C₁-C₆ alkylamino, 4 to 12-membered heterocycloalkyl, or 5- or 6-membered heteroaryl, and Rs₃ is optionally substituted with one or more substituents selected from the group consisting of halo, OH, oxo, C₁-C₆ alkyl, COOH, C(O)O—C₁-C₆ alkyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl;
   each of R₂₄, R₂₅, and R₂₆ independently is H or C₁-C₆ alkyl;
each of R₂₇ and R₂₈ independently is H or OR₂₉; or R₂₇ and R₂₈ together form O—R₃₀—O;
   each R₂₉ independently is H, C₁-C₆ alkyl, C₂-C₆ alkenyl, or C₂-C₆ alkynyl and R₂₉, when being C₁-C₆ alkyl, C₂-C₆ alkenyl, or C₂-C₆ alkynyl, is optionally substituted with one or more of halo, OH and C₁-C₆ alkoxyl that is optionally substituted with one or more OH or OC(O)—C₁-C₆ alkyl;
R₃₀ is C₁-C₆ alkylene optionally substituted with one or more of halo, OH and C₁-C₆ alkoxyl;
each of R₃₁, R₃₂, and R₃₃, independently is H, C₁-C₆ alkyl, C₃-C₈ cycloalkyl, C₆-C₁₀ aryl, 4 to 12-membered heterocycloalkyl, or 5- or 6-membered heteroaryl;
each of R₄₀, R₄₁, R₄₂, and R₄₃ independently is H, halo, OH, cyano, N₃, OP(O)R₄₇R₄₈, or C₁-C₆ alkyl optionally substituted with one or more OP(O)R₄₇R₄₈, or one R₄₁ and one R₄₃, together with the carbon atoms to which they are attached and Q₀, form C₄-C₁₀ cycloalkyl, 4- to 14-membered heterocycloalkyl, C₆-C₁₀ aryl, or 5- to 14-membered heteroaryl, and each of the cycloalkyl, heterocycloalkyl, phenyl, or 5- to 6-membered heteroaryl is optionally substituted with one or more of OH, halo, cyano, N₃, oxo, OP(O)R₄₇R₄₈, C₁-C₆ alkyl, C₁-C₆ haloalkyl, COOH, C(O)O—C₁-C₆ alkyl, C₁-C₆ alkoxyl, C₁-C₆ haloalkoxyl, amino, mono-C₁-C₆ alkylamino, and di-C₁-C₆ alkylamino;
R₄₄ is H, C₁-C₆ alkyl, or an amine protecting group;
each of R₄₅ and R₄₆ independently is H, OP(O)R₄₇R₄₈, or C₁-C₆ alkyl optionally substituted with one or more OP(O)R₄₇R₄₈, and
each of R₄₇ and R₄₈, independently is H, halo, C₁-C₆ alkyl, OH, SH, SeH, or BH₃⁻.

It should be understood that a cap analog, as described herein, may include any of the cap analogs described in international publication WO 2017/066797, published on 20 Apr. 2017, incorporated by reference herein in its entirety.

In some embodiments, the B₂ middle position can be a non-ribose molecule, such as arabinose.

In some embodiments R₂ is ethyl-based.

Thus, in some embodiments, a trinucleotide cap comprises the following structure:

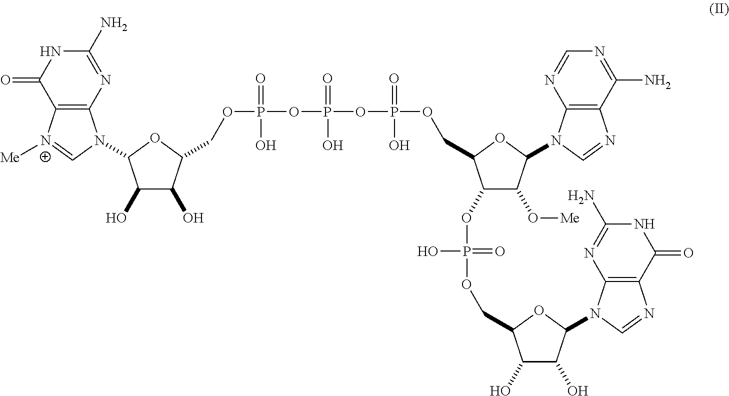

In yet other embodiments, a trinucleotide cap comprises the following structure:

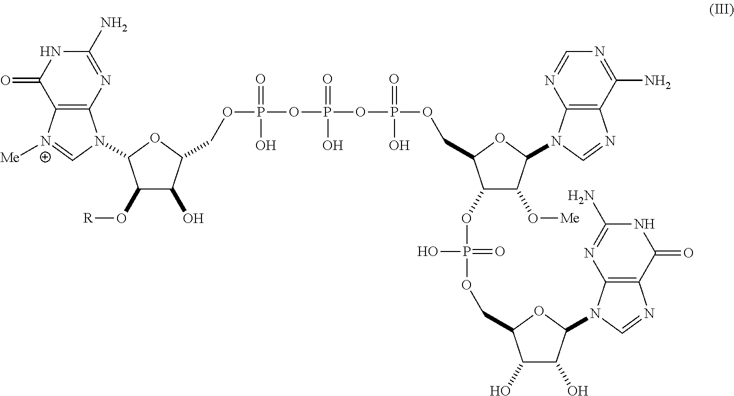

In still other embodiments, a trinucleotide cap comprises the following structure:

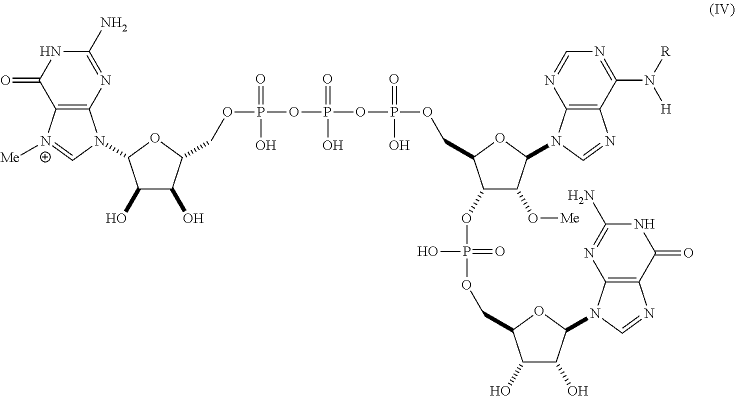

In some embodiments, R is an alkyl (e.g., C₁-C₆ alkyl). In some embodiments, R is a methyl group (e.g., C₁ alkyl). In some embodiments, R is an ethyl group (e.g., C₂ alkyl).

A trinucleotide cap, in some embodiments, comprises a sequence selected from the following sequences: GAA, GAC, GAG, GAU, GCA, GCC, GCG, GCU, GGA, GGC, GGG, GGU, GUA, GUC, GUG, and GUU. In some embodiments, a trinucleotide cap comprises GAA. In some embodiments, a trinucleotide cap comprises GAC. In some embodiments, a trinucleotide cap comprises GAG. In some embodiments, a trinucleotide cap comprises GAU. In some embodiments, a trinucleotide cap comprises GCA. In some embodiments, a trinucleotide cap comprises GCC. In some embodiments, a trinucleotide cap comprises GCG. In some embodiments, a trinucleotide cap comprises GCU. In some embodiments, a trinucleotide cap comprises GGA. In some embodiments, a trinucleotide cap comprises GGC. In some embodiments, a trinucleotide cap comprises GGG. In some embodiments, a trinucleotide cap comprises GGU. In some embodiments, a trinucleotide cap comprises GUA. In some embodiments, a trinucleotide cap comprises GUC. In some embodiments, a trinucleotide cap comprises GUG. In some embodiments, a trinucleotide cap comprises GUU.

In some embodiments, a trinucleotide cap comprises a sequence selected from the following sequences: m⁷GpppApA, m⁷GpppApC, m⁷GpppApG, m⁷GpppApU, m⁷GpppCpA, m⁷GpppCpC, m⁷GpppCpG, m⁷GpppCpU, m⁷GpppGpA, m⁷GpppGpC, m⁷GpppGpG, m⁷GpppGpU, m⁷GpppUpA, m⁷GpppUpC, m⁷GpppUpG, and m⁷GpppUpU.

In some embodiments, a trinucleotide cap comprises m⁷GpppApA. In some embodiments, a trinucleotide cap comprises m⁷GpppApC. In some embodiments, a trinucleotide cap comprises m⁷GpppApG. In some embodiments, a trinucleotide cap comprises m⁷GpppApU. In some embodiments, a trinucleotide cap comprises m⁷GpppCpA. In some embodiments, a trinucleotide cap comprises m⁷GpppCpC. In some embodiments, a trinucleotide cap comprises m⁷GpppCpG. In some embodiments, a trinucleotide cap comprises m⁷GpppCpU. In some embodiments, a trinucleotide cap comprises m⁷GpppGpA. In some embodiments, a trinucleotide cap comprises m⁷GpppGpC. In some embodiments, a trinucleotide cap comprises m⁷GpppGpG. In some embodiments, a trinucleotide cap comprises m⁷GpppGpU. In some embodiments, a trinucleotide cap comprises m⁷GpppUpA. In some embodiments, a trinucleotide cap comprises m⁷GpppUpC. In some embodiments, a trinucleotide cap comprises m⁷GpppUpG. In some embodiments, a trinucleotide cap comprises m⁷GpppUpU.

A trinucleotide cap, in some embodiments, comprises a sequence selected from the following sequences: m⁷G_{3′OMe}pppApA, m⁷G_{3′OMe}pppApC, m⁷G_{3′OMe}pppApG, m⁷G_{3′OMe}pppApU, m⁷G_{3′OMe}pppCpA, m⁷G_{3′OMe}pppCpC, m⁷G_{3′OMe}pppCpG, m⁷G_{3′OMe}pppCpU, m⁷G_{3′OMe}pppGpA, m⁷G_{3′OMe}pppGpC, m⁷G_{3′OMe}pppGpG, m⁷G_{3′OMe}pppGpU, m⁷G_{3′OMe}pppUpA, m⁷G_{3′OMe}pppUpC, m⁷G_{3′OMe}pppUpG, and m⁷G_{3′OMe}pppUpU.

In some embodiments, a trinucleotide cap comprises m⁷G_{3′OMe}pppApA. In some embodiments, a trinucleotide cap comprises m⁷G_{3′OMe}pppApC. In some embodiments, a trinucleotide cap comprises m⁷G_{3′OMe}pppApG. In some embodiments, a trinucleotide cap comprises m⁷G_{3′OMe}pppApU. In some embodiments, a trinucleotide cap comprises m⁷G_{3′OMe}pppCpA. In some embodiments, a trinucleotide cap comprises m⁷G_{3′OMe}pppCpC. In some embodiments, a trinucleotide cap comprises m⁷G_{3′OMe}pppCpG. In some embodiments, a trinucleotide cap comprises m⁷G_{3′OMe}pppCpU. In some embodiments, a trinucleotide cap comprises m⁷G_{3′OMe}pppGpA. In some embodiments, a trinucleotide cap comprises m⁷G_{3′OMe}pppGpC. In some embodiments, a trinucleotide cap comprises m⁷G_{3′OMe}pppGpG. In some embodiments, a trinucleotide cap comprises m⁷G_{3′OMe}pppGpU. In some embodiments, a trinucleotide cap comprises m⁷G_{3′OMe}pppUpA. In some embodiments, a trinucleotide cap comprises m⁷G_{3′OMe}pppUpC. In some embodiments, a trinucleotide cap comprises m⁷G_{3′OMe}pppUpG. In some embodiments, a trinucleotide cap comprises m⁷G_{3′OMe}pppUpU.

A trinucleotide cap, in other embodiments, comprises a sequence selected from the following sequences: m⁷G_{3′OMe}pppA_{2′OMe}pA, m⁷G_{3′OMe}pppA_{2′OMe}pC, m⁷G_{3′OMe}pppA_{2′OMe}pG, m⁷G_{3′OMe}pppA_{2′OMe}pU, m⁷G_{3′OMe}pppC_{2′OMe}pA, m⁷G_{3′OMe}pppC_{2′OMe}pC, m⁷G_{3′OMe}pppC_{2′OMe}pG, m⁷G_{3′OMe}pppC_{2′OMe}pU, m⁷G_{3′OMe}pppG_{2′OMe}pA, m⁷G_{3′OMe}pppG_{2′OMe}pC, m⁷G_{3′OMe}pppG_{2′OMe}pG, m⁷G_{3′OMe}pppG_{2′OMe}pU, m⁷G_{3′OMe}pppU_{2′OMe}pA, m⁷G_{3′OMe}pppU_{2′OMe}pC, m⁷G_{3′OMe}pppU_{2′OMe}pG, and m⁷G_{3′OMe}pppU_{2′OMe}pU.

In some embodiments, a trinucleotide cap comprises m⁷G_{3′OMe}pppA_{2′OMe}pA. In some embodiments, a trinucleotide cap comprises m⁷G_{3′OMe}pppA_{2′OMe}pC. In some embodiments, a trinucleotide cap comprises m⁷G_{3′OMe}pppA_{2′OMe}pG. In some embodiments, a trinucleotide cap comprises m⁷G_{3′OMe}pppA_{2′OMe}pU. In some embodiments, a trinucleotide cap comprises m⁷G_{3′OMe}pppC_{2′OMe}pA. In some embodiments, a trinucleotide cap comprises m⁷G_{3′OMe}pppC_{2′OMe}pC. In some embodiments, a trinucleotide cap comprises m⁷G_{3′OMe}pppC_{2′OMe}pG. In some embodiments, a trinucleotide cap comprises m⁷G_{3′OMe}pppC_{2′OMe}pU. In some embodiments, a trinucleotide cap comprises m⁷G_{3′OMe}pppG_{2′OMe}pA. In some embodiments, a trinucleotide cap comprises m⁷G_{3′OMe}pppG_{2′OMe}pC. In some embodiments, a trinucleotide cap comprises m⁷G_{3′OMe}pppG_{2′OMe}pG. In some embodiments, a trinucleotide cap comprises m⁷G_{3′OMe}pppG_{2′OMe}pU. In some embodiments, a trinucleotide cap comprises m⁷G_{3′OMe}pppU_{2′OMe}pA. In some embodiments, a trinucleotide cap comprises m⁷G_{3′OMe}pppU_{2′OMe}pC. In some embodiments, a trinucleotide cap comprises m⁷G_{3′OMe}pppU_{2′OMe}pG. In some embodiments, a trinucleotide cap comprises m⁷G_{3′OMe}pppU_{2′OMe}pU.

A trinucleotide cap, in still other embodiments, comprises a sequence selected from the following sequences: m⁷GpppA_{2′OMe}pA, m⁷GpppA_{2′OMe}pC, m⁷GpppA_{2′OMe}pG, m⁷GpppA_{2′OMe}pU, m⁷GpppC_{2′OMe}pA, m⁷GpppC_{2′OMe}pC, m⁷GpppC_{2′OMe}pG, m⁷GpppC_{2′OMe}pU, m⁷GpppG_{2′OMe}pA, m⁷GpppG_{2′OMe}pC, m⁷GpppG_{2′OMe}pG, m⁷GpppG_{2′OMe}pU, m⁷GpppU_{2′OMe}pA, m⁷GpppU_{2′OMe}pC, m⁷GpppU_{2′OMe}pG, and m⁷GpppU_{2′OMe}pU.

In some embodiments, a trinucleotide cap comprises m⁷GpppA_{2′OMe}pA. In some embodiments, a trinucleotide cap comprises m⁷GpppA_{2′OMe}pC. In some embodiments, a trinucleotide cap comprises m⁷GpppA_{2′OMe}pG. In some embodiments, a trinucleotide cap comprises m⁷GpppA_{2′OMe}pU. In some embodiments, a trinucleotide cap comprises m⁷GpppC_{2′OMe}pA. In some embodiments, a trinucleotide cap comprises m⁷GpppC_{2′OMe}pC. In some embodiments, a trinucleotide cap comprises m⁷GpppC_{2′OMe}pG. In some embodiments, a trinucleotide cap comprises m⁷GpppC_{2′OMe}pU. In some embodiments, a trinucleotide cap comprises m⁷GpppG_{2′OMe}pA. In some embodiments, a trinucleotide cap comprises m⁷GpppG_{2′OMe}pC. In some embodiments, a trinucleotide cap comprises m⁷GpppG_{2′OMe}pG. In some embodiments, a trinucleotide cap comprises m⁷GpppG_{2′OMe}pU. In some embodiments, a trinucleotide cap comprises m⁷GpppU_{2′OMe}pA. In some embodiments, a trinucleotide cap comprises m⁷GpppU_{2′OMe}pC. In some embodiments, a trinucleotide cap comprises m⁷GpppU_{2′OMe}pG. In some embodiments, a trinucleotide cap comprises m⁷GpppU_{2′OMe}pU.

In some embodiments, a trinucleotide cap comprises m⁷Gpppm⁶A_{2′OMe}pG. In some embodiments, a trinucleotide cap comprises m⁷Gpppe⁶A_{2′OMe}pG.

In some embodiments, a trinucleotide cap comprises GAG. In some embodiments, a trinucleotide cap comprises GCG. In some embodiments, a trinucleotide cap comprises GUG. In some embodiments, a trinucleotide cap comprises GGG.

In some embodiments, a trinucleotide cap comprises any one of the following structures:

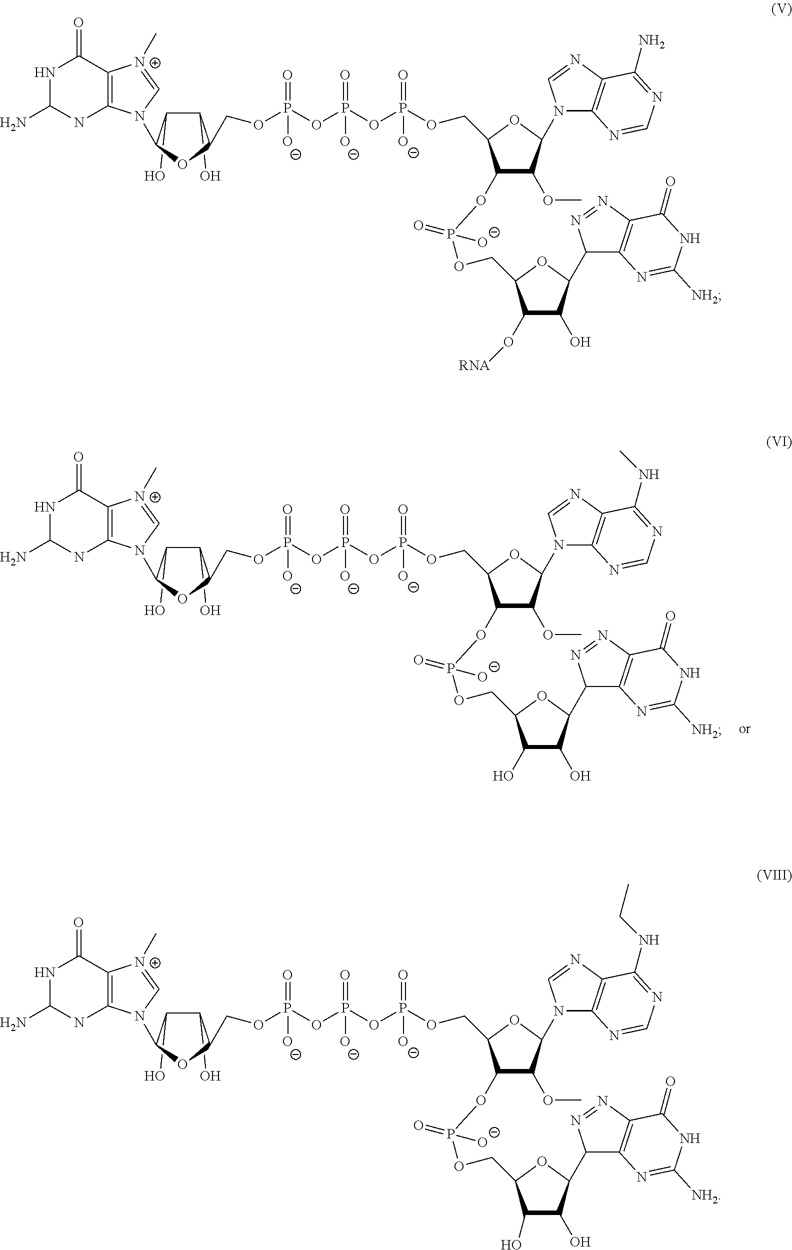

In some embodiments, the cap analog comprises a tetranucleotide cap. In some embodiments, the tetranucleotide cap comprises a trinucleotide as set forth above. In some embodiments, the tetranucleotide cap comprises m^{m7}GpppN₁N₂N₃, where N₁, N₂, and N₃ are optional (i.e., can be absent or one or more can be present) and are independently a natural, a modified, or an unnatural nucleoside base. In some embodiments, ^{m7}G is further methylated, e.g., at the 3′ position. In some embodiments, the ^{m7}G comprises an O-methyl at the 3′ position. In some embodiments N₁, N₂, and N₃ if present, optionally, are independently an adenine, a uracil, a guanidine, a thymine, or a cytosine. In some embodiments, one or more (or all) of N₁, N₂, and N₃, if present, are methylated, e.g., at the 2′ position. In some embodiments, one or more (or all) of N₁, N₂, and N₃, if present have an O-methyl at the 2′ position.

In some embodiments, the tetranucleotide cap comprises the following structure:

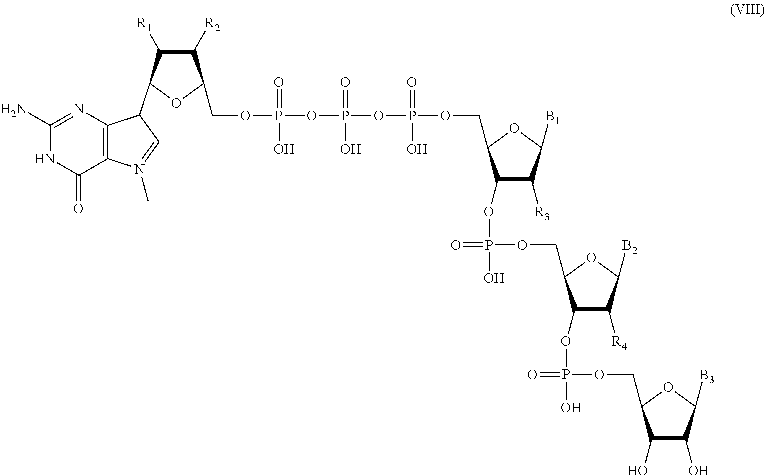

wherein B₁, B₂, and B₃ are independently a natural, a modified, or an unnatural nucleoside based, and R₁, R₂, R₃, and R₄ are independently OH or O-methyl. In some embodiments, R₃ is O-methyl and R₄ is OH. In some embodiments, R₃ and R₄ are O-methyl. In some embodiments, R₄ is O-methyl. In some embodiments, R₁ is OH, R₂ is OH, R₃ is O-methyl, and R₄ is OH In some embodiments, R₁ is OH, R₂ is OH, R₃ is O-methyl, and R₄ is O-methyl. In some embodiments, at least one of R₁ and R₂ is O-methyl, R₃ is O-methyl, and R₄ is OH. In some embodiments, at least one of R₁ and R₂ is O-methyl, R₃ is O-methyl, and R₄ is O-methyl.

In some embodiments, B₁, B₃, and B₃ are natural nucleoside bases. In some embodiments, at least one of B₁, B₂, and B₃ is a modified or unnatural base. In some embodiments, at least one of B₁, B₂, and B₃ is N6-methyladenine. In some embodiments, B₁ is adenine, cytosine, thymine, or uracil. In some embodiments, B₁ is adenine, B₂ is uracil, and B₃ is adenine. In some embodiments, R₁ and R₂ are OH, R₃ and R₄ are O-methyl, B₁ is adenine, B₂ is uracil, and B₃ is adenine.

In some embodiments the tetranucleotide cap comprises a sequence selected from the following sequences: GAAA, GACA, GAGA, GAUA, GCAA, GCCA, GCGA, GCUA, GGAA, GGCA, GGGA, GGUA, GUCA, and GUUA. In some embodiments the tetranucleotide cap comprises a sequence selected from the following sequences: GAAG, GACG, GAGG, GAUG, GCAG, GCCG, GCGG, GCUG, GGAG, GGCG, GGGG, GGUG, GUCG, GUGG, and GUUG. In some embodiments the tetranucleotide cap comprises a sequence selected from the following sequences: GAAU, GACU, GAGU, GAUU, GCAU, GCCU, GCGU, GCUU, GGAU, GGCU, GGGU, GGUU, GUAU, GUCU, GUGU, and GUUU. In some embodiments the tetranucleotide cap comprises a sequence selected from the following sequences: GAAC, GACC, GAGC, GAUC, GCAC, GCCC, GCGC, GCUC, GGAC, GGCC, GGGC, GGUC, GUAC, GUCC, GUGC, and GUUC.

A tetranucleotide cap, in some embodiments, comprises a sequence selected from the following sequences: m⁷G_{3′OMe}pppApApN, m⁷G_{3′OMe}pppApCpN, m⁷G_{3′OMe}pppApGpN, m⁷G_{3′OMe}pppApUpN, m⁷G_{3′OMe}pppCpApN, m⁷G_{3′OMe}pppCpCpN, m⁷G_{3′OMe}pppCpGpN, m⁷G_{3′OMe}pppCpUpN, m⁷G_{3′OMe}pppGpApN, m⁷G_{3′OMe}pppGpCpN, m⁷G_{3′OMe}pppGpGpN, m⁷G_{3′OMe}pppGpUpN, m⁷G_{3′OMe}pppUpApN, m⁷G_{3′OMe}pppUpCpN, m⁷G_{3′OMe}pppUpGpN, and m⁷G_{3′OMe}pppUpUpN, where N is a natural, a modified, or an unnatural nucleoside base.

A tetranucleotide cap, in other embodiments, comprises a sequence selected from the following sequences: m⁷G_{3′OMe}pppA_{2′OMe}pApN, m⁷G_{3′OMe}pppA_{2′OMe}pCpN, m⁷G_{3′OMe}pppA_{2′OMe}pGpN, m⁷G_{3′OMe}pppA_{2′OMe}pUpN, m⁷G_{3′OMe}pppC_{2′OMe}pApN, m⁷G_{3′OMe}pppC_{2′OMe}pCpN, m⁷G_{3′OMe}pppC_{2′OMe}pGpN, m¹G_{3′OMe}pppC_{2′OMe}pUpN, m⁷G_{3′OMe}pppG_{2′OMe}pApN, m⁷G_{3′OMe}pppG_{2′OMe}pCpN, m⁷G_{3′OMe}pppG_{2′OMe}pGpN, m⁷G_{3′OMe}pppG_{2′OMe}pUpN, m⁷G_{3′OMe}pppU_{2′OMe}pApN, m⁷G_{3′OMe}pppU_{2′OMe}pCpN, m⁷G_{3′OMe}pppU_{2′OMe}pGpN, and m⁷G_{3′OMe}pppU_{2′OMe}pUpN, where N is a natural, a modified, or an unnatural nucleoside base.

A tetranucleotide cap, in still other embodiments, comprises a sequence selected from the following sequences: m⁷GpppA_{2′OMe}pApN, m⁷GpppA_{2′OMe}pCpN, m⁷GpppA_{2′OMe}pGpN, m⁷GpppA_{2′OMe}pUpN, m⁷GpppC_{2′OMe}pApN, m⁷GpppC_{2′OMe}pCpN, m⁷GpppC_{2′OMe}pGpN, m⁷GpppC_{2′OMe}pUpN, m⁷GpppG_{2′OMe}pApN, m⁷GpppG_{2′OMe}pCpN, m⁷GpppG_{2′OMe}pGpN, m⁷GpppG_{2′OMe}pUpN, m⁷GpppU_{2′OMe}pApN, m⁷GpppU_{2′OMe}pCpN, m⁷GpppU_{2′OMe}pGpN, and m⁷GpppU_{2′OMe}pUpN, where N is a natural, a modified, or an unnatural nucleoside base.

A tetranucleotide cap, in other embodiments, comprises a sequence selected from the following sequences: m⁷G_{3′OMe}pppA_{2′OMe}A_{2′OMe}pN, m⁷G_{3′OMe}pppA_{2′OMe}pC_{2′OMe}pN, m⁷G_{3′OMe}pppA_{2′OMe}pG_{2′OMe}pN, m⁷G_{3′OMe}pppA_{2′OMe}pU_{2′OMe}pN, m⁷G_{3′OMe}pppC_{2′OMe}pA_{2′OMe}pN, m⁷G_{3′OMe}pppC_{2′OMe}pC_{2′OMe}pN, m⁷G_{3′OMe}pppC_{2′OMe}pG_{2′OMe}pN, m⁷G_{3′OMe}pppC_{2′OMe}pU_{2′OMe}pN, m⁷G_{3′OMe}pppG_{2′OMe}pA_{2′OMe}pN, m⁷G_{3′OMe}pppG_{2′OMe}pC_{2′OMe}pN, m⁷G_{3′OMe}pppG_{2′OMe}pG_{2′OMe}pN, m⁷G_{3′OMe}pppG_{2′OMe}pU_{2′OMe}pN, m⁷G_{3′OMe}pppU_{2′OMe}pA_{2′OMe}pN, m⁷G_{3′OMe}pppU_{2′OMe}pC_{2′OMe}pN, m⁷G_{3′OMe}pppU_{2′OMe}pG_{2′OMe}pN, and m⁷G_{3′OMe}pppU_{2′OMe}pU_{2′OMe}pN, where N is a natural, a modified, or an unnatural nucleoside base.

A tetranucleotide cap, in still other embodiments, comprises a sequence selected from the following sequences: m⁷GpppA_{2′OMe}pA_{2′OMe}pN, m⁷GpppA_{2′OMe}pC_{2′OMe}pN, m⁷GpppA_{2′OMe}pG_{2′OMe}pN, m⁷GpppA_{2′OMe}pU_{2′OMe}pN, m⁷GpppC_{2′OMe}pA_{2′OMe}pN, m⁷GpppC_{2′OMe}pC_{2′OMe}pN, m⁷GpppC_{2′OMe}pG_{2′OMe}pN, m⁷GpppC_{2′OMe}pU_{2′OMe}pN, m⁷GpppG_{2′OMe}pA_{2′OMe}pN, m⁷GpppG_{2′OMe}pC_{2′OMe}pN, m⁷GpppG_{2′OMe}pG_{2′OMe}pN, m⁷GpppG_{2′OMe}pU_{2′OMe}pN, m⁷GpppU_{2′OMe}pA_{2′OMe}pN, m⁷GpppU_{2′OMe}pC_{2′OMe}pN, m⁷GpppU_{2′OMe}pG_{2′OMe}pN, and m⁷GpppU_{2′OMe}pU_{2′OMe}pN, where N is a natural, a modified, or an unnatural nucleoside base.

In some embodiments, a tetranucleotide cap comprises GGAG. In some embodiments, a tetranucleotide cap comprises the following structure:

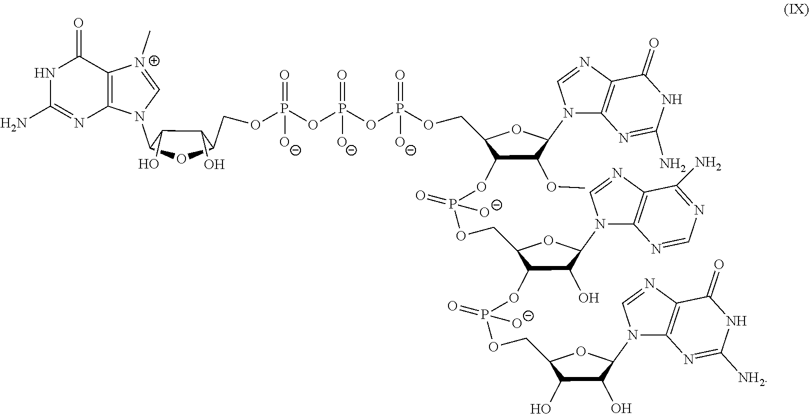

The capping efficiency of a post-transcriptional or co-transcriptional capping reaction may vary. As used herein “capping efficiency” refers to the amount (e.g., expressed as a percentage) of mRNAs comprising a cap structure relative to the total mRNAs in a mixture (e.g., a post-translational capping reaction or a co-transcriptional calling reaction). In some embodiments, the capping efficiency of a capping reaction is at least 60%, 70%, 80%, 90%, 95%, 99%, or 99.9% (e.g., after the capping reaction at least 60%, 70%, 80%, 90%, 95%, 99%, or 99.9% of the input mRNAs comprise a cap). In some embodiments, multivalent co-IVT reactions described herein do not affect the capping efficiency of the mRNAs resulting from the IVT reaction.

### Untranslated Regions (UTRs)

Untranslated regions (UTRs) are sections of a nucleic acid before a start codon (5′ UTR) and after a stop codon (3′ UTR) that are not translated. In some embodiments, a nucleic acid (e.g., a ribonucleic acid (RNA), e.g., a messenger RNA (mRNA)) of the disclosure comprising an open reading frame (ORF) encoding one or more peptide epitopes further comprises one or more UTRs (e.g., a 5′ UTR or functional fragment thereof, a 3′ UTR or functional fragment thereof, or a combination thereof).

A UTR can be homologous or heterologous to the coding region in a nucleic acid. In some embodiments, the UTR is homologous to the ORF encoding the one or more peptide epitopes. In some embodiments, the UTR is heterologous to the ORF encoding the one or more peptide epitopes. In some embodiments, the nucleic acid comprises two or more 5′ UTRs or functional fragments thereof, each of which have the same or different nucleotide sequences. In some embodiments, the nucleic acid comprises two or more 3′ UTRs or functional fragments thereof, each of which have the same or different nucleotide sequences.

In some embodiments, the 5′ UTR or functional fragment thereof, 3′ UTR or functional fragment thereof, or any combination thereof is sequence optimized.

In some embodiments, the 5′ UTR or functional fragment thereof, 3′ UTR or functional fragment thereof, or any combination thereof comprises at least one chemically modified nucleobase, e.g., 5-methoxyuracil.

UTRs can have features that provide a regulatory role, e.g., increased or decreased stability, localization, and/or translation efficiency. A nucleic acid comprising a UTR can be administered to a cell, tissue, or organism, and one or more regulatory features can be measured using routine methods. In some embodiments, a functional fragment of a 5′ UTR or 3′ UTR comprises one or more regulatory features of a full length 5′ or 3′ UTR, respectively.

Natural 5′ UTRs bear features that play roles in translation initiation. They harbor signatures like Kozak sequences that are commonly known to be involved in the process by which the ribosome initiates translation of many genes. 5′ UTRs also have been known to form secondary structures that are involved in elongation factor binding.

By engineering the features typically found in abundantly expressed genes of specific target organs, one can enhance the stability and protein production of a nucleic acid. For example, introduction of 5′ UTR of liver-expressed mRNA, such as albumin, serum amyloid A, Apolipoprotein A/B/E, transferrin, alpha fetoprotein, erythropoietin, or Factor VIII, can enhance expression of nucleic acids in hepatic cell lines or liver. Likewise, use of 5′ UTRs from other tissue-specific mRNA to improve expression in that tissue is possible for muscle (e.g., MyoD, Myosin, Myoglobin, Myogenin, Herculin), for endothelial cells (e.g., Tie-1, CD36), for myeloid cells (e.g., C/EBP, AML1, G-CSF, GM-CSF, CD11b, MSR, Fr-1, i-NOS), for leukocytes (e.g., CD45, CD18), for adipose tissue (e.g., CD36, GLUT4, ACRP30, adiponectin), and for lung epithelial cells (e.g., SP-A/B/C/D).

In some embodiments, UTRs are selected from a family of transcripts whose proteins share a common function, structure, feature, or property. For example, an encoded polypeptide can belong to a family of proteins (i.e., that share at least one function, structure, feature, localization, origin, or expression pattern), which are expressed in a particular cell, tissue or at some time during development. The UTRs from any of the genes or mRNA can be swapped for any other UTR of the same or different family of proteins to create a new nucleic acid.

In some embodiments, the 5′ UTR and the 3′ UTR can be heterologous. In some embodiments, the 5′ UTR can be derived from a different species than the 3′ UTR. In some embodiments, the 3′ UTR can be derived from a different species than the 5′ UTR.

International Patent Application No. PCT/US2014/021522 (Publ. No. WO/2014/164253) provides a listing of exemplary UTRs that may be utilized in the nucleic acids as flanking regions to an ORF. This publication is incorporated by reference herein for this purpose.

Additional exemplary UTRs that may be utilized in the nucleic acids include, but are not limited to, one or more 5′ UTRs and/or 3′ UTRs derived from the nucleic acid sequence of: a globin, such as an α- or β-globin (e.g., a Xenopus, mouse, rabbit, or human globin); a strong Kozak translational initiation signal; a CYBA (e.g., human cytochrome b-245 α polypeptide); an albumin (e.g., human albumin7); a HSD17B4 (hydroxysteroid (17-β) dehydrogenase); a virus (e.g., a tobacco etch virus (TEV), a Venezuelan equine encephalitis virus (VEEV), a Dengue virus, a cytomegalovirus (CMV; e.g., CMV immediate early 1 (IE1)), a hepatitis virus (e.g., hepatitis B virus), a sindbis virus, or a PAV barley yellow dwarf virus); a heat shock protein (e.g., hsp70); a translation initiation factor (e.g., eIF4G); a glucose transporter (e.g., hGLUT1 (human glucose transporter 1)); an actin (e.g., human α or β actin); a GAPDH; a tubulin; a histone; a citric acid cycle enzyme; a topoisomerase (e.g., a 5′ UTR of a TOP gene lacking the 5′ TOP motif (the oligopyrimidine tract)); a ribosomal protein Large 32 (L32); a ribosomal protein (e.g., human or mouse ribosomal protein, such as, for example, rps9); an ATP synthase (e.g., ATP5A1 or the β subunit of mitochondrial H⁺-ATP synthase); a growth hormone (e.g., bovine (bGH) or human (hGH)); an elongation factor (e.g., elongation factor 1 α1 (EEF1A1)); a manganese superoxide dismutase (MnSOD); a myocyte enhancer factor 2A (MEF2A); a β-F1-ATPase, a creatine kinase, a myoglobin, a granulocyte-colony stimulating factor (G-CSF); a collagen (e.g., collagen type I, alpha 2 (Col12), collagen type I, alpha 1 (Col11), collagen type VI, alpha 2 (Col6A2), collagen type VI, alpha 1 (Col6A1)); a ribophorin (e.g., ribophorin I (RPNI)); a low density lipoprotein receptor-related protein (e.g., LRP1); a cardiotrophin-like cytokine factor (e.g., Nnt1); calreticulin (Calr); a procollagen-lysine, 2-oxoglutarate 5-dioxygenase 1 (Plod1); and a nucleobindin (e.g., Nucb1).

In some embodiments, the 5′ UTR is selected from the group consisting of a β-globin 5′ UTR; a 5′ UTR containing a strong Kozak translational initiation signal; a cytochrome b-245 α polypeptide (CYBA) 5′ UTR; a hydroxysteroid (17-β) dehydrogenase (HSD17B4) 5′ UTR; a Tobacco etch virus (TEV) 5′ UTR; a Venezuelen equine encephalitis virus (TEEV) 5′ UTR; a 5′ proximal open reading frame of rubella virus (RV) RNA encoding nonstructural proteins; a Dengue virus (DEN) 5′ UTR; a heat shock protein 70 (Hsp70) 5′ UTR; a eIF4G 5′ UTR; a GLUT1 5′ UTR; functional fragments thereof and any combination thereof.

In some embodiments, the 3′ UTR is selected from the group consisting of a β-globin 3′ UTR; a CYBA 3′ UTR; an albumin 3′ UTR; a growth hormone (GH) 3′ UTR; a VEEV 3′ UTR; a hepatitis B virus (HBV) 3′ UTR; α-globin 3′ UTR; a DEN 3′ UTR; a PAV barley yellow dwarf virus (BYDV-PAV) 3′ UTR; an elongation factor 1 α1 (EEF1A1) 3′ UTR; a manganese superoxide dismutase (MnSOD) 3′ UTR; a P subunit of mitochondrial H(+)-ATP synthase (β-mRNA) 3′ UTR; a GLUT1 3′ UTR; a MEF2A 3′ UTR; a β-F1-ATPase 3′ UTR; functional fragments thereof and combinations thereof.

Wild-type UTRs derived from any gene or mRNA can be incorporated into the nucleic acids of the disclosure. In some embodiments, a UTR can be altered relative to a wild type or native UTR to produce a variant UTR, e.g., by changing the orientation or location of the UTR relative to the ORF; or by inclusion of additional nucleotides, deletion of nucleotides, swapping or transposition of nucleotides. In some embodiments, variants of 5′ or 3′ UTRs can be utilized, for example, mutants of wild type UTRs, or variants wherein one or more nucleotides are added to or removed from a terminus of the UTR.

Additionally, one or more synthetic UTRs can be used in combination with one or more non-synthetic UTRs. See, e.g., Mandal and Rossi, Nat. Protoc. 2013 8(3):568-82, and sequences available at www.addgene.org/Derrick_Rossi/, the contents of each are incorporated herein by reference in their entirety. UTRs or portions thereof can be placed in the same orientation as in the transcript from which they were selected or can be altered in orientation or location. Hence, a 5′ and/or 3′ UTR can be inverted, shortened, lengthened, or combined with one or more other 5′ UTRs or 3′ UTRs.

In some embodiments, the nucleic acid may comprise multiple UTRs, e.g., a double, a triple or a quadruple 5′ UTR or 3′ UTR. For example, a double UTR comprises two copies of the same UTR either in series or substantially in series. For example, a double beta-globin 3′ UTR can be used (see, for example, US2010/0129877, the contents of which are incorporated herein by reference for this purpose).

The nucleic acids of the disclosure can comprise combinations of features. For example, the ORF can be flanked by a 5′ UTR that comprises a strong Kozak translational initiation signal and/or a 3′ UTR comprising an oligo(dT) sequence for templated addition of a polyA tail. A 5′ UTR can comprise a first nucleic acid fragment and a second nucleic acid fragment from the same and/or different UTRs (see, e.g., US2010/0293625, herein incorporated by reference in its entirety for this purpose). In some embodiments, a UTR comprises one or more IDR sequences. In some embodiments, a 5′ UTR comprises one or more IDR sequences. In some embodiments, a 3′ UTR comprises one or more IDR sequences.

Other non-UTR sequences can be used as regions or subregions within the nucleic acids of the disclosure. For example, introns or portions of intron sequences can be incorporated into the nucleic acids of the disclosure. Incorporation of intronic sequences can increase protein production as well as nucleic acid expression levels. In some embodiments, the nucleic acid of the disclosure comprises an internal ribosome entry site (IRES) instead of or in addition to a UTR (see, e.g., Yakubov et al., Biochem. Biophys. Res. Commun. 2010 394(1):189-193, the contents of which are incorporated herein by reference in their entirety). In some embodiments, the nucleic acid comprises an IRES instead of a 5′ UTR sequence. In some embodiments, the nucleic acid comprises an ORF and a viral capsid sequence. In some embodiments, the nucleic acid comprises a synthetic 5′ UTR in combination with a non-synthetic 3′ UTR.

In some embodiments, the UTR can also include at least one translation enhancer nucleic acid, translation enhancer element, or translational enhancer elements (collectively, “TEE,” which refers to nucleic acid sequences that increase the amount of polypeptide or protein produced from a polynucleotide. As a non-limiting example, the TEE can include those described in US2009/0226470, incorporated herein by reference in its entirety for this purpose, and others known in the art. As a non-limiting example, the TEE can be located between the transcription promoter and the start codon. In some embodiments, the 5′ UTR comprises a TEE. In one aspect, a TEE is a conserved element in a UTR that can promote translational activity of a nucleic acid such as, but not limited to, cap-dependent or cap-independent translation. In one non-limiting example, the TEE comprises the TEE sequence in the 5′-leader of the Gtx homeodomain protein. See Chappell et al., PNAS 2004 101:9590-9594, incorporated herein by reference in its entirety for this purpose.

Identification and/or Ratio Determination (IDR) Sequences Aspects of the disclosure relate to RNA compositions (e.g., multivalent RNA compositions) which comprise mRNAs having one or more (e.g., 1, 2, 3, 4, or more) unique sequences or sequences for identification and/or ratio determination (IDR), and methods of analyzing such RNA compositions. As used herein, an “IDR sequence” (as well as the terms “barcode sequence,” “identifier sequence,” and “identifying sequence”) refers to a sequence of a biological molecule (e.g., nucleic acid, protein, etc.) that serves to identify the other biological molecule. Typically, an IDR sequence is a heterologous sequence that is incorporated within or appended to a sequence of a target biological molecule and utilized as a reference in order to identify a target molecule of interest. In some embodiments, an IDR sequence is a sequence of a nucleic acid (e.g., a heterologous or synthetic nucleic acid) that is incorporated within or appended to a target nucleic acid and utilized as a reference in order to identify the target nucleic acid. Such use of an IDR sequence to identify a reference nucleic acid can allow evaluation of an RNA composition containing a single RNA species, to determine the presence and/or amount of an RNA species of interest which has the IDR sequence. Additionally, the incorporation of different IDR sequences onto different RNA species, allows the presence and/or abundance of different IDR sequences to be measured to identify the RNA species present in a composition comprising multiple RNA species. In some embodiments, an IDR sequence is of the formula (N)ₙ. In some embodiments, n is an integer in the range of 3 to 20, 3 to 10, 5 to 20, 5 to 10, 10 to 20, 7 to 20, or 7 to 30. In some embodiments, n is 2, 3, 4, 5, 6, 7, 8, 9, 10, 11, 12, 13, 14, 15, 16, 17, 18, 19, 20, 21, 22, 23, 24, 25, 26, 27, 28, 29, 30 or more. In some embodiments, N are each nucleotides that are independently selected from A, G, T, U, and C, or analogues thereof. Examples of nucleotide analogues include, but are not limited to, antiviral nucleotide analogs, phosphate analogs (soluble or immobilized, hydrolyzable or non-hydrolyzable), dinucleotide, trinucleotide, tetranucleotide, e.g., a cap analog, or a precursor/substrate for enzymatic capping (vaccinia or ligase), a nucleotide labeled with a functional group to facilitate ligation/conjugation of cap or 5′ moiety (IRES), a nucleotide labeled with a 5′ PO₄ to facilitate ligation of cap or 5′ moiety, or a nucleotide labeled with a functional group/protecting group that can be chemically or enzymatically cleaved. Examples of antiviral nucleotide/nucleoside analogs include, but are not limited, to Ganciclovir, Entecavir, Telbivudine, Vidarabine and Cidofovir. Modified nucleotides may comprise a modified nucleobase. In some embodiments, a nucleotide analogue comprises a modified nucleobase selected from the group consisting of xanthine, allyaminouracil, allyaminothymidine, hypoxanthine, digoxigeninated adenine, digoxigeninated cytosine, digoxigeninated guanine, digoxigeninated uracil, 6-chloropurineriboside, N6-methyladenine, methylpseudouracil, 2-thiocytosine, 2-thiouracil, 5-methyluracil, 4-thiothymidine, 4-thiouracil, 5,6-dihydro-5-methyluracil, 5,6-dihydrouracil, 5-[(3-Indolyl)propionamide-N-allyl]uracil, 5-aminoallylcytosine, 5-aminoallyluracil, 5-bromouracil, 5-bromocytosine, 5-carboxycytosine, 5-carboxymethylesteruracil, 5-carboxyuracil, 5-fluorouracil, 5-formylcytosine, 5-formyluracil, 5-hydroxycytosine, 5-hydroxymethylcytosine, 5-hydroxymethyluracil, 5-hydroxyuracil, 5-iodocytosine, 5-iodouracil, 5-methoxycytosine, 5-methoxyuracil, 5-methylcytosine, 5-methyluracil, 5-propargylaminocytosine, 5-propargylaminouracil, 5-propynylcytosine, 5-propynyluracil, 6-azacytosine, 6-azauracil, 6-chloropurine, 6-thioguanine, 7-deazaadenine, 7-deazaguanine, 7-deaza-7-propargylaminoadenine, 7-deaza-7-propargylaminoguanine, 8-azaadenine, 8-azidoadenine, 8-chloroadenine, 8-oxoadenine, 8-oxoguanine, araadenine, aracytosine, araguanine, arauracil, biotin-16-7-deaza-7-propargylaminoguanine, biotin-16-aminoallylcytosine, biotin-16-aminoallyluracil, cyanine 3-5-propargylaminocytosine, cyanine 3-6-propargylaminouracil, cyanine 3-aminoallylcytosine, cyanine 3-aminoallyluracil, cyanine 5-6-propargylaminocytosine, cyanine 5-6-propargylaminouracil, cyanine 5-aminoallylcytosine, cyanine 5-aminoallyluracil, cyanine 7-aminoallyluracil, dabcyl-5-3-aminoallyluracil, desthiobiotin-16-aminoallyl-uracil, desthiobiotin-6-aminoallylcytosine, isoguanine, N1-ethylpseudouracil, N1-methoxymethylpseudouracil, N1-methyladenine, N1-methylpseudouracil, N1-propylpseudouracil, N2-methylguanine, N4-biotin-OBEA-cytosine, N4-methylcytosine, N6-methyladenine, O6-methylguanine, pseudoisocytosine, pseudouracil, thienocytosine, thienoguanine, thienouracil, xanthosine, 3-deazaadenine, 2,6-diaminoadenine, 2,6-daminoguanine, 5-carboxamide-uracil, 5-ethynyluracil, N6-isopentenyladenine (i6A), 2-methyl-thio-N6-isopentenyladenine (ms2i6A), 2-methylthio-N6-methyladenine (ms2m6A), N6-(cis-hydroxyisopentenyl)adenine (io6A), 2-methylthio-N6-(cis-hydroxyisopentenyl)adenine (ms2io6A), N6-glycinylcarbamoyladenine (g6A), N6-threonylcarbamoyladenine (t6A), 2-methylthio-N6-threonyl carbamoyladenine (ms2t6A), N6-methyl-N6-threonylcarbamoyladenine (m6t6A), N6-hydroxynorvalylcarbamoyladenine (hn6A), 2-methylthio-N6-hydroxynorvalyl carbamoyladenine (ms2hn6A), N6,N6-dimethyladenine (m62A), and N6-acetyladenine (ac6A). Modified nucleotides may comprise a modified sugar. In some embodiments, a nucleotide analogue comprises a modified sugar selected from the group consisting of 2′-thioribose, 2′,3′-dideoxyribose, 2′-amino-2′-deoxyribose, 2′ deoxyribose, 2′-azido-2′-deoxyribose, 2′-fluoro-2′-deoxyribose, 2′-O-methylribose, 2′-O-methyldeoxyribose, 3′-amino-2′,3′-dideoxyribose, 3′-azido-2′,3′-dideoxyribose, 3′-deoxyribose, 3′-O-(2-nitrobenzyl)-2′-deoxyribose, 3′-O-methylribose, 5′-aminoribose, 5′-thioribose, 5-nitro-1-indolyl-2′-deoxyribose, 5′-biotin-ribose, 2′-O,4′-C-methylene-linked, 2′-O,4′-C-amino-linked ribose, and 2′-O,4′-C-thio-linked ribose. Modified nucleotides may comprise a modified phosphate. In some embodiments, a nucleotide analogue comprises a modified phosphate selected from the group consisting of phosphorothioate (PS), thiophosphate, 5′-O-methylphosphonate, 3′-O-methylphosphonate, 5′-hydroxyphosphonate, hydroxyphosphanate, phosphoroselenoate, selenophosphate, phosphoramidate, carbophosphonate, methylphosphonate, phenylphosphonate, ethylphosphonate, H-phosphonate, guanidinium ring, triazole ring, boranophosphate (BP), methylphosphonate, and guanidinopropyl phosphoramidate. In some embodiments, a nucleotide analogue comprises two or more of a modified nucleobase, modified sugar, and modified phosphate. In some embodiments, an IDR sequence comprises multiple nucleotide analogues. In some embodiments, an IDR sequence comprises multiple different nucleotide analogues. Thus, some embodiments comprise nucleic acids (e.g., mRNAs) that (i) have a target sequence of interest (e.g., a coding sequence (e.g., that encodes therapeutic peptide or therapeutic protein)); and (ii) comprise a unique IDR sequence.

In some embodiments, an IDR sequence is of the formula A-Nₙ-A, A-Nₙ—C, A-Nₙ-G, A-Nₙ—U, C—Nₙ-A, C—Nₙ—C, C—Nₙ-G, C—Nₙ—U, G-Nₙ-A, G-Nₙ—C, G-Nₙ-G, G-Nₙ—U, U—Nₙ-A, U—Nₙ-A, U—Nₙ-G, or U—Nₙ—U, where n is an integer in the range of 3-20 inclusive, and each N is independently selected from A, C, G, or U or an analogue of A, C, G, or U. In some embodiments, an IDR sequence is of the formula A-Nₘ-A-Nₙ, Nₘ-A-Nₙ-A, A-Nₘ—C—Nₙ, Nₘ-A-Nₙ—C, A-Nₘ-G-Nₙ, Nₘ-A-Nₙ-G, A-Nₘ—U—Nₙ, Nₘ-A-Nₙ—U, C—Nₘ-A-Nₙ, Nₘ—C—Nₙ-A, C—Nₘ—C—Nₙ, Nₘ—C—Nₙ—C, C—Nₘ-G-Nₙ, Nₘ—C—Nₙ-G, C—Nₘ—U—Nₙ, Nₘ—C—Nₙ—U, G-Nₘ-A-Nₙ, Nₘ-G-Nₙ-A, G-Nₘ—C—Nₙ, Nₘ-G-Nₙ—C, G-Nₘ-G-Nₙ, Nₘ-G-Nₙ-G, G-Nₘ—U—Nₙ, Nₘ-G-Nₙ—U, U—Nₘ-A-Nₙ, Nₘ—U—Nₙ-A, U—Nₘ—C—Nₙ, Nₘ—U—Nₙ—C, U—Nₘ-G-Nₙ, Nₘ—U—Nₙ-G, U—Nₘ—U—Nₙ, Nₘ—U—Nₙ—U, Nₘ-AA-Nₙ, Nₘ-AC-Nₙ, Nₘ-AG-Nₙ, Nₘ-AU-Nₙ, Nₘ-CA-Nₙ, Nₘ-CG-Nₙ, Nₘ-CG-Nₙ, Nₘ—CU—Nₙ, Nₘ-GA-Nₙ, Nₘ-GC-Nₙ, Nₘ-GG-Nₙ, Nₘ-GU-Nₙ, Nₘ—UA-Nₙ, Nₘ—UC—Nₙ, Nₘ—UG-Nₙ, Nₘ—UU—Nₙ, where m and n are each integers independently selected from the range of 3-20 inclusive, and each N is independently selected from A, C, G, or U or an analogue of A, C, G, or U. Examples of nucleotide analogues include, but are not limited to, antiviral nucleotide analogs, phosphate analogs (soluble or immobilized, hydrolyzable or non-hydrolyzable), dinucleotide, trinucleotide, tetranucleotide, e.g., a cap analog, or a precursor/substrate for enzymatic capping (vaccinia or ligase), a nucleotide labeled with a functional group to facilitate ligation/conjugation of cap or 5′ moiety (IRES), a nucleotide labeled with a 5′ PO₄ to facilitate ligation of cap or 5′ moiety, or a nucleotide labeled with a functional group/protecting group that can be chemically or enzymatically cleaved. Examples of antiviral nucleotide/nucleoside analogs include, but are not limited, to Ganciclovir, Entecavir, Telbivudine, Vidarabine and Cidofovir. Modified nucleotides may comprise a modified nucleobase. In some embodiments, a nucleotide analogue comprises a modified nucleobase selected from the group consisting of xanthine, allyaminouracil, allyaminothymidine, hypoxanthine, digoxigeninated adenine, digoxigeninated cytosine, digoxigeninated guanine, digoxigeninated uracil, 6-chloropurineriboside, N6-methyladenine, methylpseudouracil, 2-thiocytosine, 2-thiouracil, 5-methyluracil, 4-thiothymidine, 4-thiouracil, 5,6-dihydro-5-methyluracil, 5,6-dihydrouracil, 5-[(3-Indolyl)propionamide-N-allyl]uracil, 5-aminoallylcytosine, 5-aminoallyluracil, 5-bromouracil, 5-bromocytosine, 5-carboxycytosine, 5-carboxymethylesteruracil, 5-carboxyuracil, 5-fluorouracil, 5-formylcytosine, 5-formyluracil, 5-hydroxycytosine, 5-hydroxymethylcytosine, 5-hydroxymethyluracil, 5-hydroxyuracil, 5-iodocytosine, 5-iodouracil, 5-methoxycytosine, 5-methoxyuracil, 5-methylcytosine, 5-methyluracil, 5-propargylaminocytosine, 5-propargylaminouracil, 5-propynylcytosine, 5-propynyluracil, 6-azacytosine, 6-azauracil, 6-chloropurine, 6-thioguanine, 7-deazaadenine, 7-deazaguanine, 7-deaza-7-propargylaminoadenine, 7-deaza-7-propargylaminoguanine, 8-azaadenine, 8-azidoadenine, 8-chloroadenine, 8-oxoadenine, 8-oxoguanine, araadenine, aracytosine, araguanine, arauracil, biotin-16-7-deaza-7-propargylaminoguanine, biotin-16-aminoallylcytosine, biotin-16-aminoallyluracil, cyanine 3-5-propargylaminocytosine, cyanine 3-6-propargylaminouracil, cyanine 3-aminoallylcytosine, cyanine 3-aminoallyluracil, cyanine 5-6-propargylaminocytosine, cyanine 5-6-propargylaminouracil, cyanine 5-aminoallylcytosine, cyanine 5-aminoallyluracil, cyanine 7-aminoallyluracil, dabcyl-5-3-aminoallyluracil, desthiobiotin-16-aminoallyl-uracil, desthiobiotin-6-aminoallylcytosine, isoguanine, N1-ethylpseudouracil, N1-methoxymethylpseudouracil, N1-methyladenine, N1-methylpseudouracil, N1-propylpseudouracil, N2-methylguanine, N4-biotin-OBEA-cytosine, N4-methylcytosine, N6-methyladenine, O6-methylguanine, pseudoisocytosine, pseudouracil, thienocytosine, thienoguanine, thienouracil, xanthosine, 3-deazaadenine, 2,6-diaminoadenine, 2,6-daminoguanine, 5-carboxamide-uracil, 5-ethynyluracil, N6-isopentenyladenine (i6A), 2-methyl-thio-N6-isopentenyladenine (ms2i6A), 2-methylthio-N6-methyladenine (ms2m6A), N6-(cis-hydroxyisopentenyl)adenine (io6A), 2-methylthio-N6-(cis-hydroxyisopentenyl)adenine (ms2io6A), N6-glycinylcarbamoyladenine (g6A), N6-threonylcarbamoyladenine (t6A), 2-methylthio-N6-threonyl carbamoyladenine (ms2t6A), N6-methyl-N6-threonylcarbamoyladenine (m6t6A), N6-hydroxynorvalylcarbamoyladenine (hn6A), 2-methylthio-N6-hydroxynorvalyl carbamoyladenine (ms2hn6A), N6,N6-dimethyladenine (m62A), and N6-acetyladenine (ac6A). Modified nucleotides may comprise a modified sugar. In some embodiments, a nucleotide analogue comprises a modified sugar selected from the group consisting of 2′-thioribose, 2′,3′-dideoxyribose, 2′-amino-2′-deoxyribose, 2′ deoxyribose, 2′-azido-2′-deoxyribose, 2′-fluoro-2′-deoxyribose, 2′-O-methylribose, 2′-O-methyldeoxyribose, 3′-amino-2′,3′-dideoxyribose, 3′-azido-2′,3′-dideoxyribose, 3′-deoxyribose, 3′-O-(2-nitrobenzyl)-2′-deoxyribose, 3′-O-methylribose, 5′-aminoribose, 5′-thioribose, 5-nitro-1-indolyl-2′-deoxyribose, 5′-biotin-ribose, 2′-O,4′-C-methylene-linked, 2′-O,4′-C-amino-linked ribose, and 2′-O,4′-C-thio-linked ribose. Modified nucleotides may comprise a modified phosphate. In some embodiments, a nucleotide analogue comprises a modified phosphate selected from the group consisting of phosphorothioate (PS), thiophosphate, 5′-O-methylphosphonate, 3′-O-methylphosphonate, 5′-hydroxyphosphonate, hydroxyphosphanate, phosphoroselenoate, selenophosphate, phosphoramidate, carbophosphonate, methylphosphonate, phenylphosphonate, ethylphosphonate, H-phosphonate, guanidinium ring, triazole ring, boranophosphate (BP), methylphosphonate, and guanidinopropyl phosphoramidate. In some embodiments, a nucleotide analogue comprises two or more of a modified nucleobase, modified sugar, and modified phosphate. In some embodiments, an IDR sequence comprises multiple nucleotide analogues. In some embodiments, use of IDR sequences corresponding to a particular formula allows variable sequences, such as the internal Nₘ and/or Nₙ nucleotides, to be varied between RNA species, while the presence of conserved first, last, and/or internal nucleotides allows identification of contaminating RNAs or RNA fragments that do not contain the correct conserved nucleotide(s) of the formula. Additionally, the presence of one or more conserved nucleotides at the beginning or end of an IDR sequence allows the conserved nucleotides to be utilized in sequence-dependent cloning methods (e.g., restriction enzyme digestion and ligation), such that IDR sequences containing diverse internal sequences can be inserted into DNA templates using the same cloning method. In some embodiments, an IDR sequence is of the formula A-Nₙ-A, where n is 3-20. In some embodiments, an IDR sequence is of the formula A-Nₙ—C, where n is 3-20. In some embodiments, an IDR sequence is of the formula A-Nₙ-G, where n is 3-20. In some embodiments, an IDR sequence is of the formula A-Nₙ—U, where n is 3-20. In some embodiments, an IDR sequence is of the formula C—Nₙ-A, where n is 3-20. In some embodiments, an IDR sequence is of the formula C—Nₙ—C, where n is 3-20. In some embodiments, an IDR sequence is of the formula C—Nₙ-G, where n is 3-20. In some embodiments, an IDR sequence is of the formula C—Nₙ—U, where n is 3-20. In some embodiments, an IDR sequence is of the formula G-Nₙ-A, where n is 3-20. In some embodiments, an IDR sequence is of the formula G-Nₙ—C, where n is 3-20. In some embodiments, an IDR sequence is of the formula G-Nₙ-G, where n is 3-20. In some embodiments, an IDR sequence is of the formula G-Nₙ—U, where n is 3-20. In some embodiments, an IDR sequence is of the formula U—Nₙ-A, where n is 3-20. In some embodiments, an IDR sequence is of the formula U—Nₙ—C, where n is 3-20. In some embodiments, an IDR sequence is of the formula U—Nₙ-G, where n is 3-20. In some embodiments, an IDR sequence is of the formula U—Nₙ—U, where n is 3-20. In some embodiments, an IDR sequence is of the formula A-Nₘ-A-Nₙ, where m is 3-20 and n is 3-20. In some embodiments, an IDR sequence is of the formula Nₘ-A-Nₙ-A, where m is 3-20 and n is 3-20. In some embodiments, an IDR sequence is of the formula A-Nₘ—C—Nₙ, where m is 3-20 and n is 3-20. In some embodiments, an IDR sequence is of the formula Nₘ-A-Nₙ—C, where m is 3-20 and n is 3-20. In some embodiments, an IDR sequence is of the formula A-Nₘ-G-Nₙ, where m is 3-20 and n is 3-20. In some embodiments, an IDR sequence is of the formula Nₘ-A-Nₙ-G, where m is 3-20 and n is 3-20. In some embodiments, an IDR sequence is of the formula A-Nₘ—U—Nₙ, where m is 3-20 and n is 3-20. In some embodiments, an IDR sequence is of the formula Nₘ-A-Nₙ—U, where m is 3-20 and n is 3-20. In some embodiments, an IDR sequence is of the formula C—Nₘ-A-Nₙ, where m is 3-20 and n is 3-20. In some embodiments, an IDR sequence is of the formula Nₘ—C—Nₙ-A, where m is 3-20 and n is 3-20. In some embodiments, an IDR sequence is of the formula C—Nₘ—C—Nₙ, where m is 3-20 and n is 3-20. In some embodiments, an IDR sequence is of the formula Nₘ—C—Nₙ—C, where m is 3-20 and n is 3-20. In some embodiments, an IDR sequence is of the formula C—Nₘ-G-Nₙ, where m is 3-20 and n is 3-20. In some embodiments, an IDR sequence is of the formula Nₘ—C—Nₙ-G, where m is 3-20 and n is 3-20. In some embodiments, an IDR sequence is of the formula C—Nₘ—U—Nₙ, where m is 3-20 and n is 3-20. In some embodiments, an IDR sequence is of the formula Nₘ—C—Nₙ—U, where m is 3-20 and n is 3-20. In some embodiments, an IDR sequence is of the formula G-Nₘ-A-Nₙ, where m is 3-20 and n is 3-20. In some embodiments, an IDR sequence is of the formula Nₘ-G-Nₙ-A, where m is 3-20 and n is 3-20. In some embodiments, an IDR sequence is of the formula G-Nₘ—C—Nₙ, where m is 3-20 and n is 3-20. In some embodiments, an IDR sequence is of the formula Nₘ-G-Nₙ—C, where m is 3-20 and n is 3-20. In some embodiments, an IDR sequence is of the formula G-Nₘ-G-Nₙ, where m is 3-20 and n is 3-20. In some embodiments, an IDR sequence is of the formula Nₘ-G-Nₙ-G, where m is 3-20 and n is 3-20. In some embodiments, an IDR sequence is of the formula G-Nₘ—U—Nₙ, where m is 3-20 and n is 3-20. In some embodiments, an IDR sequence is of the formula Nₘ-G-Nₙ—U, where m is 3-20 and n is 3-20. In some embodiments, an IDR sequence is of the formula U—Nₘ-A-Nₙ, where m is 3-20 and n is 3-20. In some embodiments, an IDR sequence is of the formula Nₘ—U—Nₙ-A, where m is 3-20 and n is 3-20. In some embodiments, an IDR sequence is of the formula U—Nₘ—C—Nₙ, where m is 3-20 and n is 3-20. In some embodiments, an IDR sequence is of the formula Nₘ—U—Nₙ—C, where m is 3-20 and n is 3-20. In some embodiments, an IDR sequence is of the formula U—Nₘ-G-Nₙ, where m is 3-20 and n is 3-20. In some embodiments, an IDR sequence is of the formula Nₘ—U—Nₙ-G, where m is 3-20 and n is 3-20. In some embodiments, an IDR sequence is of the formula U—Nₘ—U—Nₙ, where m is 3-20 and n is 3-20. In some embodiments, an IDR sequence is of the formula Nₘ—U—Nₙ—U, where m is 3-20 and n is 3-20. In some embodiments, an IDR sequence is of the formula Nₘ-AA-Nₙ, where m is 3-20 and n is 3-20. In some embodiments, an IDR sequence is of the formula Nₘ-AC-Nₙ, where m is 3-20 and n is 3-20. In some embodiments, an IDR sequence is of the formula Nₘ-AG-Nₙ, where m is 3-20 and n is 3-20. In some embodiments, an IDR sequence is of the formula Nₘ-AU-Nₙ, where m is 3-20 and n is 3-20. In some embodiments, an IDR sequence is of the formula Nₘ-CA-Nₙ, where m is 3-20 and n is 3-20. In some embodiments, an IDR sequence is of the formula Nₘ—CC—Nₙ, where m is 3-20 and n is 3-20. In some embodiments, an IDR sequence is of the formula Nₘ-CG-Nₙ, where m is 3-20 and n is 3-20. In some embodiments, an IDR sequence is of the formula Nₘ—CU—Nₙ, where m is 3-20 and n is 3-20. In some embodiments, an IDR sequence is of the formula Nₘ-GA-Nₙ, where m is 3-20 and n is 3-20. In some embodiments, an IDR sequence is of the formula Nₘ-GC-Nₙ, where m is 3-20 and n is 3-20. In some embodiments, an IDR sequence is of the formula Nₘ-GG-Nₙ, where m is 3-20 and n is 3-20. In some embodiments, an IDR sequence is of the formula Nₘ-GU-Nₙ, where m is 3-20 and n is 3-20. In some embodiments, an IDR sequence is of the formula Nₘ—UA-Nₙ, where m is 3-20 and n is 3-20. In some embodiments, an IDR sequence is of the formula Nₘ—UC—Nₙ, where m is 3-20 and n is 3-20. In some embodiments, an IDR sequence is of the formula Nₘ—UG-Nₙ, where m is 3-20 and n is 3-20. In some embodiments, an IDR sequence is of the formula Nₘ—UU—Nₙ, where m is 3-20 and n is 3-20. In some embodiments, an IDR sequence is of the formula Nₘ—UG-Nₙ, where m is 3-20 and n is 21 or more. In some embodiments, an IDR sequence is of the formula Nₘ—UG-Nₙ, where m is 21 or more, and n is 3-20 or more. In some embodiments, an IDR sequence is of the formula Nₘ—UG-Nₙ, where each of m and n are 21 or more. In some embodiments, m is 21 or more, 30 or more, 40 or more, 50 or more, 60 or more, 70 or more, 80 or more, 90 or more, 100 or more, or more. In some embodiments, n is 21 or more, 30 or more, 40 or more, 50 or more, 60 or more, 70 or more, 80 or more, 90 or more, 100 or more, or more.

In some embodiments, one or more RNA species (e.g., RNA of a given sequence) of a RNA composition (e.g., a multivalent RNA composition) comprises an IDR sequence with a distinct mass. IDR sequences may differ in mass due to differences in sequence length, base composition, or sequence length and base composition. In some embodiments, each RNA species in a multivalent RNA composition comprises an IDR sequence that differs from the mass of every other IDR sequence (i.e., associated with other RNA species) in the multivalent RNA composition by about 9 to about 8000 Da or more, such as 50-2000 Da, 100-1500 Da, 200-1000 Da, or 400-800 Da. In some embodiments, each RNA species in a multivalent RNA composition comprises an IDR sequence that differs from the mass of every other IDR sequence in the multivalent RNA composition by at least 50 Da, at least 100 Da, at least 200 Da, at least 300 Da, at least 400 Da, at least 500 Da, at least 600 Da, at least 700 Da, at least 800 Da, at least 900 Da, at least 1000 Da, at least 1100 Da, at least 1200 Da, at least 1300 Da, at least 1400 Da, at least 1500 Da, at least 1600 Da, at least 1700 Da, at least 1800 Da, at least 1900 Da, at least 1900 Da, at least 2000 Da, at least 3000 Da, at least 4000 Da, at least 5000 Da, at least 6000 Da, at least 7000 Da, or at least 8000 Da or more. In some embodiments, an RNA species in an RNA composition has an IDR sequence with a mass of about 9 DA, or more, such as about 50 Da, about 100 Da, about 200 Da, about 300 Da, about 400 Da, about Da, about 600 Da, about 700 Da, about 800 Da, about 900 Da, about 1000 Da, about 1100 Da, about 1200 Da, about 1300 Da, about 1400 Da, about 1500 Da, about 1600 Da, about 1700 Da, about 1800 Da, about 1900 Da, about 1900 Da, about 2000 Da, about 3000 Da, about 4000 Da, about 5000 Da, about 6000 Da, about 7000 Da, or about 8000 Da or more. In some embodiments, an RNA species in an RNA composition has an IDR sequence with a mass of about 50 Da or less.

In some embodiments, each RNA species in a multivalent RNA composition comprises an IDR sequence with a different length. In some embodiments, one RNA species in a multivalent RNA composition comprises an IDR sequence of length 0. An IDR sequence of length 0 refers to the absence of nucleotides in the position where other RNA species in a multivalent RNA composition comprise IDR sequences. RNA species with IDR sequences of length 0 can be distinguished from other RNA species due to their lack of nucleotides in the position where other RNA species have IDR sequences, which reduces their mass relative to other RNA species. In some embodiments, each RNA species in a multivalent RNA composition comprises an IDR sequence with length between 0 and 100, 0 and 50, 0 and 30, 0 and 20, 0 and 10, or 0 and 5 nucleotides. In some embodiments, each RNA species in a multivalent RNA composition comprises an IDR sequence with length between 1 and 100, 1 and 50, 1 and 30, 1 and 20, 1 and 10, or 1 and 5 nucleotides.

In some embodiments, two or more RNA species in a multivalent RNA composition comprise IDR sequences of identical lengths but different masses. In some embodiments, no RNA species in a multivalent RNA composition comprise an IDR sequence that is a sequence isomer of an IDR sequence that is comprised on a different RNA species. As used herein, a “sequence isomer” refers to a nucleic acid sequence that comprises the same number of each base as a reference sequence, wherein the order of bases in a sequence isomer differs from that of the reference sequence. For example, each of the RNA sequences AGUU, GUUA, and UUGA is a sequence isomer of the reference sequence UGUA. Methods of determining the mass of a nucleic acid sequence, such as an IDR sequence, are known in the art, and include methods such as mass spectrometry. In some embodiments, cleavage of each distinct RNA species by RNase H produces an RNA fragment with a distinct mass. In some embodiments, the mass of each RNA or RNA fragment is determined by mass spectrometry. In some embodiments, cleavage of at least 90%, at least 91%, at least 92%, at least 93%, at least 94%, at least 95%, at least 96%, at least 97%, at least 98%, at least 99%, or up to 100% of the RNAs of a given RNA species produces RNA fragments with about the same mass. Two RNA fragments are said to have “about the same mass” if the mass of one RNA fragment is at least 90%, and no more than 110%, the mass of the other RNA fragment. In some embodiments cleavage of each RNA species in a multivalent RNA composition produces an RNA fragment with a mass that differs from the mass of RNA fragments produced by cleavage of every other RNA species in the composition by at least 9 Da, such as at least 50 Da, at least 100 Da, at least 200 Da, at least 300 Da, at least 400 Da, at least 500 Da, at least 600 Da, at least 700 Da, at least 800 Da, at least 900 Da, at least 1000 Da, at least 1100 Da, at least 1200 Da, at least 1300 Da, at least 1400 Da, at least 1500 Da, at least 1600 Da, at least 1700 Da, at least 1800 Da, at least 1900 Da, at least 1900 Da, at least 2000 Da, at least 3000 Da, at least 4000 Da, at least 5000 Da, at least 6000 Da, at least 7000 Da, or at least 8000 Da or more. In some embodiments cleavage of each RNA species in a multivalent RNA composition produces an RNA fragment with a mass that differs from the mass of RNA fragments produced by cleavage of every other RNA species in the composition by 9-8000 Da, 50-2000 Da, 100-1500 Da, 200-1000 Da, or 400-800 Da. Exemplary IDR sequences with distinct masses include: —(0 length IDR sequence), A, GG, CC, GGA, CCA, GGAA, GGUUA, GACCA, GGACCA, GGCCAAA, GGCCAAGA, GGCCAAGGA, and CCCGUACCCCC (SEQ ID NO: 1).

In some embodiments, two or more RNA species in a multivalent RNA composition comprise IDR sequences with identical lengths but different masses, where each RNA species comprises an IDR sequence with the same first and last nucleotide. In some embodiments, the IDR sequence of each RNA species in a multivalent RNA composition is of a formula, wherein the formula is selected from the group consisting of A-Nₙ-A, A-Nₙ—C, A-Nₙ-G, A-Nₙ—U, C—Nₙ-A, C—Nₙ—C, C—Nₙ-G, C—Nₙ—U, G-Nₙ-A, G-Nₙ—C, G-Nₙ-G, G-Nₙ—U, U—Nₙ-A, U—Nₙ-A, U—Nₙ-G, or U—Nₙ—U, where n is an integer in the range of 3-20 inclusive, and each N is independently selected from A, C, G, or U or an analogue of A, C, G, or U. In some embodiments, each RNA comprises an IDR sequence having a formula that is independently selected from the group consisting of A-Nₙ-A, A-Nₙ—C, A-Nₙ-G, A-Nₙ—U, C—Nₙ-A, C—Nₙ—C, C—Nₙ-G, C—Nₙ—U, G-Nₙ-A, G-Nₙ—C, G-Nₙ-G, G-Nₙ—U, U—Nₙ-A, U—Nₙ-A, U—Nₙ-G, U—Nₙ—U, A-Nₘ-A-Nₙ, Nₘ-A-Nₙ-A, A-Nₘ—C—Nₙ, Nₘ-A-Nₙ—C, A-Nₘ-G-Nₙ, Nₘ-A-Nₙ-G, A-Nₘ—U—Nₙ, Nₘ-A-Nₙ—U, C—Nₘ-A-Nₙ, Nₘ—C—Nₙ-A, C—Nₘ—C—Nₙ, Nₘ—C—Nₙ—C, C—Nₘ-G-Nₙ, Nₘ—C—Nₙ-G, C—Nₘ—U—Nₙ, Nₘ—C—Nₙ—U, G-Nₘ-A-Nₙ, Nₘ-G-Nₙ-A, G-Nₘ—C—Nₙ, Nₘ-G-Nₙ—C, G-Nₘ-G-Nₙ, Nₘ-G-Nₙ-G, G-Nₙ—U—Nₙ, Nₘ-G-Nₙ—U, U—Nₘ-A-Nₙ, Nₘ—U—Nₙ-A, U—Nₘ—C—Nₙ, Nₘ—U—Nₙ—C, U—Nₘ-G-Nₙ, Nₘ—U—Nₙ-G, U—Nₘ—U—Nₙ, and Nₘ—U—Nₙ—U, where m and n are each integers independently selected from the range of 3-20 inclusive, and each N is independently selected from A, C, G, or U or an analogue of A, C, G, or U. In some embodiments, each RNA comprises an IDR sequence having the same formula that is selected from the group consisting of A-Nₙ-A, A-Nₙ—C, A-Nₙ-G, A-Nₙ—U, C—Nₙ-A, C—Nₙ—C, C—Nₙ-G, C—Nₙ—U, G-Nₙ-A, G-Nₙ—C, G-Nₙ-G, G-Nₙ—U, U—Nₙ-A, U—Nₙ-A, U—Nₙ-G, U—Nₙ—U, A-Nₘ-A-Nₙ, Nₘ-A-Nₙ-A, A-Nₘ—C—Nₙ, Nₘ-A-Nₙ—C, A-Nₘ-G-Nₙ, Nₘ-A-Nₙ-G, A-Nₘ—U—Nₙ, Nₘ-A-Nₙ—U, C—Nₘ-A-Nₙ, Nₘ—C—Nₙ-A, C—Nₘ—C—Nₙ, Nₘ—C—Nₙ—C, C—Nₘ-G-Nₙ, Nₘ—C—Nₙ-G, C—Nₘ—U—Nₙ, Nₘ—C—Nₙ—U, G-Nₘ-A-Nₙ, Nₘ-G-Nₙ-A, G-Nₘ—C—Nₙ, Nₘ-G-Nₘ—C, G-Nₘ-G-Nₙ, Nₘ-G-Nₙ-G, G-Nₘ—U—Nₙ, Nₘ-G-Nₙ—U, U—Nₘ-A-Nₙ, Nₘ—U—Nₙ-A, U—Nₘ—C—Nₙ, Nₘ—U—Nₙ—C, U—Nₘ-G-Nₙ, Nₘ—U—Nₙ-G, U—Nₘ—U—Nₙ, and Nₘ—U—Nₙ—U, where m and n are each integers independently selected from the range of 3-20 inclusive, and each N is independently selected from A, C, G, or U or an analogue of A, C, G, or U. In some embodiments, each RNA species comprises an IDR sequence having the formula A-Nₙ-A, where n is 3-20. In some embodiments, each RNA species comprises an IDR sequence having the formula A-Nₙ—C, where n is 3-20. In some embodiments, each RNA species comprises an IDR sequence having the formula A-Nₙ-G, where n is 3-20. In some embodiments, each RNA species comprises an IDR sequence having the formula A-Nₙ—U, where n is 3-20. In some embodiments, each RNA species comprises an IDR sequence having the formula C—Nₙ-A, where n is 3-20. In some embodiments, each RNA species comprises an IDR sequence having the formula C—Nₙ—C, where n is 3-20. In some embodiments, each RNA species comprises an IDR sequence having the formula C—Nₙ-G, where n is 3-20. In some embodiments, each RNA species comprises an IDR sequence having the formula U—Nₙ—U, where n is 3-20. In some embodiments, each RNA species comprises an IDR sequence having the formula G-Nₙ-A, where n is 3-20. In some embodiments, each RNA species comprises an IDR sequence having the formula G-Nₙ—C, where n is 3-20. In some embodiments, each RNA species comprises an IDR sequence having the formula G-Nₙ-G, where n is 3-20. In some embodiments, each RNA species comprises an IDR sequence having the formula G-Nₙ—U, where n is 3-20. In some embodiments, each RNA species comprises an IDR sequence having the formula U—Nₙ-A, where n is 3-20. In some embodiments, each RNA species comprises an IDR sequence having the formula U—Nₙ—C, where n is 3-20. In some embodiments, each RNA species comprises an IDR sequence having the formula U—Nₙ-G, where n is 3-20. In some embodiments, each RNA species comprises an IDR sequence having the formula U—Nₙ—U, where n is 3-20. In some embodiments, each RNA species comprises an IDR sequence having the formula A-Nₘ-A-Nₙ, where m is 3-20 and n is 3-20. In some embodiments, each RNA species comprises an IDR sequence having the formula Nₘ-A-Nₙ-A, where m is 3-20 and n is 3-20. In some embodiments, each RNA species comprises an IDR sequence having the formula A-Nₘ—C—Nₙ, where m is 3-20 and n is 3-20. In some embodiments, each RNA species comprises an IDR sequence having the formula Nₘ-A-Nₙ—C, where m is 3-20 and n is 3-20. In some embodiments, each RNA species comprises an IDR sequence having the formula A-Nₘ-G-Nₙ, where m is 3-20 and n is 3-20. In some embodiments, each RNA species comprises an IDR sequence having the formula Nₘ-A-Nₙ-G, where m is 3-20 and n is 3-20. In some embodiments, each RNA species comprises an IDR sequence having the formula A-Nₘ—U—Nₙ, where m is 3-20 and n is 3-20. In some embodiments, each RNA species comprises an IDR sequence having the formula Nₘ-A-Nₙ—U, where m is 3-20 and n is 3-20. In some embodiments, each RNA species comprises an IDR sequence having the formula C—Nₘ-A-Nₙ, where m is 3-20 and n is 3-20. In some embodiments, each RNA species comprises an IDR sequence having the formula Nₘ—C—Nₙ-A, where m is 3-20 and n is 3-20. In some embodiments, each RNA species comprises an IDR sequence having the formula C—Nₘ—C—Nₙ, where m is 3-20 and n is 3-20. In some embodiments, each RNA species comprises an IDR sequence having the formula Nₘ—C—Nₙ—C, where m is 3-20 and n is 3-20. In some embodiments, each RNA species comprises an IDR sequence having the formula C—Nₘ-G-Nₙ, where m is 3-20 and n is 3-20. In some embodiments, each RNA species comprises an IDR sequence having the formula Nₘ—C—Nₙ-G, where m is 3-20 and n is 3-20. In some embodiments, each RNA species comprises an IDR sequence having the formula C—Nₘ—U—Nₙ, where m is 3-20 and n is 3-20. In some embodiments, each RNA species comprises an IDR sequence having the formula Nₘ—C—Nₙ—U, where m is 3-20 and n is 3-20. In some embodiments, each RNA species comprises an IDR sequence having the formula G-Nₘ-A-Nₙ, where m is 3-20 and n is 3-20. In some embodiments, each RNA species comprises an IDR sequence having the formula Nₘ-G-Nₙ-A, where m is 3-20 and n is 3-20. In some embodiments, each RNA species comprises an IDR sequence having the formula G-Nₘ—C—Nₙ, where m is 3-20 and n is 3-20. In some embodiments, each RNA species comprises an IDR sequence having the formula Nₘ-G-Nₙ—C, where m is 3-20 and n is 3-20. In some embodiments, each RNA species comprises an IDR sequence having the formula G-Nₘ-G-Nₙ, where m is 3-20 and n is 3-20. In some embodiments, each RNA species comprises an IDR sequence having the formula Nₘ-G-Nₙ-G, where m is 3-20 and n is 3-20. In some embodiments, each RNA species comprises an IDR sequence having the formula G-Nₘ—U—Nₙ, where m is 3-20 and n is 3-20. In some embodiments, each RNA species comprises an IDR sequence having the formula Nₘ-G-Nₙ—U, where m is 3-20 and n is 3-20. In some embodiments, each RNA species comprises an IDR sequence having the formula U—Nₘ-A-Nₙ, where m is 3-20 and n is 3-20. In some embodiments, each RNA species comprises an IDR sequence having the formula Nₘ—U—Nₙ-A, where m is 3-20 and n is 3-20. In some embodiments, each RNA species comprises an IDR sequence having the formula U—Nₘ—C—Nₙ, where m is 3-20 and n is 3-20. In some embodiments, each RNA species comprises an IDR sequence having the formula Nₘ—U—Nₙ—C, where m is 3-20 and n is 3-20. In some embodiments, each RNA species comprises an IDR sequence having the formula U—Nₘ-G-Nₙ, where m is 3-20 and n is 3-20. In some embodiments, each RNA species comprises an IDR sequence having the formula Nₘ—U—Nₙ-G, where m is 3-20 and n is 3-20. In some embodiments, each RNA species comprises an IDR sequence having the formula U—Nₘ—U—Nₙ, where m is 3-20 and n is 3-20. In some embodiments, each RNA species comprises an IDR sequence having the formula Nₘ—U—Nₙ—U, where m is 3-20 and n is 3-20. In some embodiments, each RNA species comprises an IDR sequence having the formula Nₘ-AA-Nₙ, where m is 3-20 and n is 3-20. In some embodiments, each RNA species comprises an IDR sequence having the formula Nₘ-AC-Nₙ, where m is 3-20 and n is 3-20. In some embodiments, each RNA species comprises an IDR sequence having the formula Nₘ-AG-Nₙ, where m is 3-20 and n is 3-20. In some embodiments, each RNA species comprises an IDR sequence having the formula Nₘ-AU-Nₙ, where m is 3-20 and n is 3-20. In some embodiments, each RNA species comprises an IDR sequence having the formula Nₘ-CA-Nₙ, where m is 3-20 and n is 3-20. In some embodiments, each RNA species comprises an IDR sequence having the formula Nₘ—CC—Nₙ, where m is 3-20 and n is 3-20. In some embodiments, each RNA species comprises an IDR sequence having the formula Nₘ-CG-Nₙ, where m is 3-20 and n is 3-20. In some embodiments, each RNA species comprises an IDR sequence having the formula Nₘ—CU—Nₙ, where m is 3-20 and n is 3-20. In some embodiments, each RNA species comprises an IDR sequence having the formula Nₘ-GA-Nₙ, where m is 3-20 and n is 3-20. In some embodiments, each RNA species comprises an IDR sequence having the formula Nₘ-GC-Nₙ, where m is 3-20 and n is 3-20. In some embodiments, each RNA species comprises an IDR sequence having the formula Nₘ-GG-Nₙ, where m is 3-20 and n is 3-20. In some embodiments, each RNA species comprises an IDR sequence having the formula Nₘ-GU-Nₙ, where m is 3-20 and n is 3-20. In some embodiments, each RNA species comprises an IDR sequence having the formula Nₘ—UA-Nₙ, where m is 3-20 and n is 3-20. In some embodiments, each RNA species comprises an IDR sequence having the formula Nₘ—UC—Nₙ, where m is 3-20 and n is 3-20. In some embodiments, each RNA species comprises an IDR sequence having the formula Nₘ—UG-Nₙ, where m is 3-20 and n is 3-20. In some embodiments, each RNA species comprises an IDR sequence having the formula Nₘ—UU—Nₙ, where m is 3-20 and n is 3-20.

In some embodiments, one or more in vitro transcribed mRNAs comprise one or more IDR sequences in an untranslated region (UTR), such as a 5′ UTR or 3′ UTR. Inclusion of an IDR sequence in the UTR of an mRNA prevents the IDR sequence from being translated into a peptide. In some embodiments, inclusion of an IDR sequence in a UTR does not negatively affect the translation of (e.g., reduce translation of) the mRNA into a protein. In some embodiments, an IDR sequence is positioned in a 3′ UTR of an mRNA. In some embodiments, the IDR sequence is positioned upstream of the polyA tail of the mRNA. In some embodiments, the IDR sequence is positioned downstream of (e.g., after) the polyA tail of the mRNA. In some embodiments, the IDR sequence is positioned between the last codon of the ORF of the mRNA and the first “A” of the polyA tail of the mRNA. In some embodiments, a polynucleotide IDR sequence positioned in a UTR comprises between 1 and 30 nucleotides (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides).

In some embodiments, the UTR comprising a polynucleotide IDR sequence further comprises one or more (e.g., 1, 2, 3, or more) RNase cleavage sites, such as RNase H cleavage sites.

In some embodiments, each different RNA of a multivalent RNA composition comprises a different (e.g., unique) IDR sequence. In some embodiments, each IDR sequence has a length that is independently selected from between 0 to 25 nucleotides. An IDR sequence of length 0 refers to a lack of nucleotides where other RNA species contain an IDR sequence having length 1 or more. An RNA species or mRNA fragment comprising an IDR sequence with length 0 may be distinguished from RNA species or mRNA fragments having IDR sequences having 1 or more nucleotides on the basis of mass (due to the lower mass of RNA having an IDR sequence of length 0) and/or sequence (due to the absence of nucleotides corresponding to an IDR sequence in the nucleotide sequence of the RNA). In some embodiments, each IDR sequence has a length that is independently selected from between 1 to 25 nucleotides. In some embodiments, each RNA species comprises an IDR sequence with a different length. In some embodiments, the UTR comprises a recognition sequence that is complementary to an RNase H guide.

An RNase H guide refers to a polynucleotide comprising one or more DNA nucleotides, and is capable of hybridizing to an RNA to form an RNA:DNA hybrid, thereby facilitating cleavage of the RNA of the RNA:DNA hybrid by RNase H. In some embodiments, an RNase H guide is a chimeric polynucleotide comprising one or more DNA nucleotides, and one or more RNA nucleotides. In some embodiments, an RNase H guide is represented by the formula [R]_{q}D₁D₂D₃D₄[R]ₚ or [R]_{q}D₁D₂D₃[R]ₚ, where each R is an RNA nucleotide, each D is a DNA nucleotide, and each of p and q are independently selected integers between 0 and 50. In some embodiments, the method comprises hybridizing one or more oligonucleotides having a nucleotide sequence represented by the formula [R]ₚD₁D₂D₃D₄[R]_{q},wherein each R is an RNA nucleotide, each D is a DNA nucleotide, and each of p and q are independently an integer between 1 and 50. In some embodiments, the method comprises cleaving an RNA fragment comprising an IDR sequence from the RNA by hybridizing one or more oligonucleotides to the RNA (e.g., hybridizing in the presence of an RNase H enzyme), where the one or more oligonucleotides have a nucleotide sequence represented by the formula [R]ₚD₁D₂D₃D₄[R]_{q}, wherein each R is an RNA nucleotide, each D is a DNA nucleotide, and each of p and q are independently an integer between 1 and 50.

In some embodiments, a method comprises contacting an RNA composition with two or more RNase H guide oligonucleotides. In some embodiments, the two or more RNase H guide oligonucleotides are oligonucleotides that hybridize to different nucleotide sequences present on different RNAs in an RNA composition. Binding of two or more RNase H guide oligonucleotides to distinct sequences of different RNAs in a composition allows targeted cleavage of RNAs having different sequences. In some embodiments, a first RNase H guide oligonucleotides is capable of hybridizing to a first nucleotide sequence of an RNA, and a second RNase H guide oligonucleotide is capable of hybridizing to a second nucleotide sequence of the RNA. Binding of two or more RNase guide oligonucleotides to the same RNA can direct RNase H to cleave the RNA at multiple sites, allowing release of an RNA fragment comprising a nucleotide sequence that is located between the sites of RNase H-mediated cleavage.

In some embodiments, a method comprises hybridizing one or more RNase H guide oligonucleotides to a sequence in the 5′ UTR of the RNA. In some embodiments, the method comprises cleaving the 5′ UTR of the RNA by hybridizing one or more oligonucleotides to a sequence in the 5′ UTR of the RNA (e.g., in the presence of an RNase H enzyme) to release an RNA fragment comprising an IDR sequence. In some embodiments, the released RNA fragment comprising an IDR sequence further comprises a cap. In some embodiments, the method comprises cleaving the 5′ UTR of the RNA at a position upstream of the IDR sequence, and at a position downstream of the IDR sequence, such that cleaving the 5′ UTR upstream and downstream from the IDR sequence releases an RNA fragment comprising the IDR sequence, but not a 5′ cap or portion of the open reading frame. In some embodiments, the method comprises contacting the RNA with a first RNase H guide oligonucleotide that hybridizes with a nucleotide sequence in the 5′ UTR that is upstream from the IDR sequence, and a second RNase H guide oligonucleotide that hybridizes with a nucleotide sequence in the 5′ UTR that is downstream from the IDR sequence. Hybridization of the first (or front) RNase H guide oligonucleotide upstream from the IDR sequence allows RNase H to recognize and cleave a portion of the RNA upstream from the IDR sequence, thereby releasing the 5′ cap from the RNA fragment comprising the IDR sequence. Hybridization of the second (or rear) RNase H guide oligonucleotide downstream from the IDR sequence allows RNase H to recognize and cleave a portion of the RNA downstream from the IDR sequence, thereby releasing the open reading frame and downstream elements from the RNA fragment comprising the IDR sequence. Combining the use of a first (front) and second (rear) RNase H guide oligonucleotides to cleave at positions in the 5′ UTR upstream and downstream from the IDR sequence, respectively, allows the release of an RNA fragment comprising the IDR sequence, but not a 5′ cap or open reading frame and downstream elements (e.g., 3′ UTR and polyA tail).

In some embodiments, a method comprises hybridizing one or more RNase H guide oligonucleotides to a sequence in the 3′ UTR of the RNA. In some embodiments, the method comprises cleaving the 3′ UTR of the RNA by hybridizing one or more oligonucleotides to a sequence in the 3′ UTR of the RNA (e.g., in the presence of an RNase H enzyme) to release an RNA fragment comprising an IDR sequence. In some embodiments, the released RNA fragment comprising an IDR sequence further comprises a poly(A) tail. In some embodiments, the method comprises cleaving the 3′ UTR of the RNA at a position upstream of the IDR sequence, and at a position downstream of the IDR sequence, such that cleaving the 3′ UTR upstream and downstream from the IDR sequence releases an RNA fragment comprising the IDR sequence, but not a poly(A) tail or portion of the open reading frame. In some embodiments, the method comprises contacting the RNA with a first RNase H guide oligonucleotide that hybridizes with a nucleotide sequence in the 3′ UTR that is upstream from the IDR sequence, and a second RNase H guide oligonucleotide that hybridizes with a nucleotide sequence in the 3′ UTR that is downstream from the IDR sequence. Hybridization of the first (or front) RNase H guide oligonucleotide upstream from the IDR sequence allows RNase H to recognize and cleave a portion of the RNA upstream from the IDR sequence, thereby releasing the open reading frame and upstream elements (e.g., 5′ cap and 5′ UTR) from the RNA fragment comprising the IDR sequence. Hybridization of the second (or rear) RNase H guide oligonucleotide downstream from the IDR sequence allows RNase H to recognize and cleave a portion of the RNA downstream from the IDR sequence, thereby releasing the polyA tail. Combining the use of a first (front) and second (rear) RNase H guide oligonucleotides to cleave at positions in the 3′ UTR upstream and downstream from the IDR sequence, respectively, allows the release of an RNA fragment comprising the IDR sequence, but not the open reading frame and upstream elements (e.g., 5′ cap and 5′ UTR) or polyA tail.

In some embodiments, cleaving at two positions within a 5′ UTR or two positions within a 3′ UTR of an RNA in a multivalent RNA composition comprises contacting the multivalent RNA composition with a first and second RNase H guide oligonucleotide, where the first RNase H guide oligonucleotide is capable of hybridizing with a sequence upstream from the IDR sequence, and the second RNase H guide oligonucleotide is capable of hybridizing with a sequence downstream from the IDR sequence. Hybridization of both the first and second RNase H guide oligonucleotides to the RNA allow RNase H to cleave the RNA at positions upstream and downstream from the IDR sequence, causing release of an RNA fragment comprising the IDR sequence from each RNA. In some embodiments, such as when the IDR sequence is located within the 3′ UTR of the RNA, cleaving upstream from the IDR sequence releases an RNA fragment from the upstream coding sequence of the RNA, and cleaving downstream from the IDR sequence releases the RNA fragment from the polyA tail. Releasing the poly(A) tail in this manner prevents the generation of RNA fragments having identical IDR sequences but different poly(A) tail lengths, which may differ in mass.

In some embodiments, a front RNase H guide oligonucleotide is capable of binding to a nucleotide sequence that is present in each RNA of an RNA composition. In some embodiments, a rear RNase H guide oligonucleotide is capable of binding to a nucleotide sequence that is present in each RNA of an RNA composition. Contacting an RNA composition comprising multiple RNAs with a front and/or rear RNase H guide oligonucleotide that is capable of binding to each RNA in the composition allows the same RNase H guide oligonucleotide to direct RNase H-mediated cleavage of each RNA in the composition.

In some embodiments, the method comprises contacting an RNA composition with a first front RNase H guide oligonucleotide and a second front RNase H guide oligonucleotide. In some embodiments, the method comprises contacting an RNA composition with a first rear RNase H guide oligonucleotide and a second rear RNase H guide oligonucleotide. In some embodiments, the first and/or second RNase H guide oligonucleotides are not capable of binding to each RNA of the RNA composition. Thus, in some embodiments, RNase H guide oligonucleotides are used to direct cleavage of different RNAs in an RNA composition.

In some embodiments, at least one R is a modified RNA nucleotide, for example a 2′-O-methyl modified RNA nucleotide. In some embodiments, each R is a modified RNA nucleotide. In some embodiments, at least one R is a 2′-O-methyl modified RNA nucleotide. In some embodiments, each R is a 2′-O-methyl modified RNA nucleotide.

In some embodiments, at least one D is a modified DNA nucleotide. Non-limiting examples of modified deoxyribonucleotides that from which modified DNA nucleotides of an RNase H guide oligonucleotide may be selected include 5-nitroindole, Inosine, 4-nitroindole, 6-nitroindole, 3-nitropyrrole, a 2-6-diaminopurine, 2-amino-adenine, or 2-thio-thiamine DNA nucleotides. In some embodiments, each D is a modified DNA nucleotide. In some embodiments, each of D₁ and D₂ are unmodified (e.g., natural) deoxyribonucleotide bases. As used herein, “unmodified deoxyribonucleotide base” refers to a natural DNA base, such as adenosine, guanosine, cytosine, thymine, or uracil. In some embodiments, D₃, D₄, or D₃ and D₄ are unnatural (e.g., modified) deoxyribonucleotide bases.

The length of each of [R]_{q} and [R]ₚ can independently vary in length. For example, in some embodiments, q is an integer between 0 and 50 (e.g., 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) and p is an integer between 0 and 50 (e.g., 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50).

In some embodiments, q is an integer between 0 and 30 (e.g., 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30) and p is an integer between 0 and 50 (e.g., 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30).

In some embodiments, q is an integer between 0 and 15 (e.g., 0, 1, 2, 3, 4, 5, 6, 7, 8, 9, 10, 11, 12, 13, 14, or 15) and p is an integer between 0 and 15 (e.g., 0, 1, 2, 3, 4, 5, 6, 7, 8, 9, 10, 11, 12, 13, 14, or 15).

In some embodiments, q is an integer between 0 and 6 (e.g., 0, 1, 2, 3, 4, 5, or 6) and p is an integer between 1 and 10 (e.g., 1, 2, 3, 4, 5, 6, 7, 8, 9, or 10). In some embodiments, p is an integer between 0 and 6 (e.g., 0, 1, 2, 3, 4, 5, or 6) and q is an integer between 1 and 10 (e.g., 1, 2, 3, 4, 5, 6, 7, 8, 9, or 10).

In some embodiments, one or more RNAs of an RNA composition comprises one or more recognition sequences for one or more RNase H guide oligonucleotides. In some embodiments, an RNA comprises one or more recognition sequences upstream from an IDR sequence on the RNA, and one or more recognition sequences downstream from the IDR sequence. A recognition sequence upstream from the IDR sequence may be referred to as a “front” recognition sequence, and an RNase H guide oligonucleotide that is capable of hybridizing to a front recognition sequence may be referred to as a “front” RNase H guide oligonucleotide. In some embodiments, a front RNase H guide binds to a nucleotide sequence that is 5-10, 10-20, 20-30, 30-50, 50-75, 75-100, 100-150, 150-200, 200-300, 300-400, or 400-500 nucleotides upstream from the IDR sequence. A recognition sequence downstream from the IDR sequence may be referred to as a “rear” recognition sequence, and an RNase H guide oligonucleotide that is capable of hybridizing to a rear recognition sequence may be referred to as a “rear” RNase H guide oligonucleotide. In some embodiments, a rear RNase H guide binds to a nucleotide sequence that is 5-10, 10-20, 20-30, 30-50, 50-75, 75-100, 100-150, 150-200, 200-300, 300-400, or 400-500 nucleotides downstream from the IDR sequence. In some embodiments, hybridizing a front RNase H guide oligonucleotide to a front recognition sequence and a rear RNase H guide oligonucleotide to a rear recognition sequence, and cleaving the RNA at positions upstream and downstream from the IDR sequence, thereby releasing an RNA fragment comprising the IDR sequence. In some embodiments, the released RNA fragment does not comprise a 5′ cap, open reading frame, or polyA tail.

In some embodiments, a recognition sequence comprises every nucleotide of the RNA that is bound by the RNase H guide. In some embodiments, a recognition sequence comprises the nucleotides that are bound by DNA nucleotides of the RNase H guide. In some embodiments, an RNase H guide comprises one or more RNA nucleotides, and the recognition sequence comprises the RNA nucleotides of the mRNA that are bound by DNA nucleotides of the RNase H guide. In some embodiments, a nucleotide sequence of an RNA that is bound by an RNase H guide oligonucleotide is referred to as an RNase H cleavage sequence.

In some embodiments, a recognition sequence and/or RNase H cleavage sequence of an RNA does not comprise a homopolymeric repeat. In some embodiments, a front RNase H guide oligonucleotide does not comprise a homopolymeric repeat. In some embodiments, a front RNase H guide oligonucleotide does not comprise a homopolymeric repeat of DNA nucleotides. In some embodiments, a rear RNase H guide oligonucleotide does not comprise a homopolymeric repeat. In some embodiments, a rear RNase H guide oligonucleotide does not comprise a homopolymeric repeat of DNA nucleotides. A homopolymeric repeat refers to a sequence of consecutive nucleotides comprising the same nucleobase. For example, the nucleotide sequence CCCC is a homopolymeric repeat of cytidine bases of length 4. The presence of homopolymeric repeats in a recognition sequence and/or RNase H cleavage sequence of an mRNA, or a corresponding homopolymeric repeat in an RNase H guide nucleotide sequence, can reduce the specificity of binding by the RNase H guide, as portions of the RNase H guide may bind at multiple different positions within the recognition sequence of the mRNA. This reduced binding specificity can result in cleavage of the same mRNA into different RNA fragments, depending on where the RNase H guide or DNA portion of the RNase H guide binds, and thus interferes with analysis of RNA fragments, as multiple RNA fragments could correspond to the same mRNA, or the same RNA fragment could correspond to multiple different mRNAs. Reducing the number and length of homopolymeric repeats in a recognition sequence and/or RNase H cleavage sequence of an mRNA, and thus reducing the number and length of homopolymeric repeats in the complementary RNase H guides or DNA portions of RNase H guides, can improve the specificity of RNase H guide binding and subsequent RNase H-mediated cleavage.

In some embodiments, the recognition sequence and/or RNase H cleavage sequence comprises no homopolymeric repeats that are 2 or more, 3 or more, 4 or more, 5 or more, 6 or more, 7 or more, 8 or more, 9 or more, or 10 or more identical bases in length. In some embodiments, a front RNase H guide oligonucleotide sequence comprises no homopolymeric repeats that are 2 or more, 3 or more, 4 or more, 5 or more, 6 or more, 7 or more, 8 or more, 9 or more, or 10 or more identical bases in length. In some embodiments, a front RNase H guide oligonucleotide sequence comprises no homopolymeric repeats of DNA nucleotides that are 2 or more, 3 or more, 4 or more, 5 or more, 6 or more, 7 or more, 8 or more, 9 or more, or 10 or more identical bases in length. In some embodiments, a rear RNase H guide oligonucleotide sequence comprises no homopolymeric repeats that are 2 or more, 3 or more, 4 or more, 5 or more, 6 or more, 7 or more, 8 or more, 9 or more, or 10 or more identical bases in length. In some embodiments, a rear RNase H guide oligonucleotide sequence comprises no homopolymeric repeats of DNA nucleotides that are 2 or more, 3 or more, 4 or more, 5 or more, 6 or more, 7 or more, 8 or more, 9 or more, or 10 or more identical bases in length.

In some embodiments, the recognition sequence and/or RNase H cleavage sequence does not comprise any homopolymeric repeats that are 3 or more bases in length. In some embodiments, a front RNase H guide oligonucleotide does not comprise any homopolymeric repeats that are 3 or more bases in length. In some embodiments, a front RNase H guide oligonucleotide does not comprise any homopolymeric repeats of DNA nucleotides that are 3 or more bases in length. In some embodiments, a rear RNase H guide oligonucleotide does not comprise any homopolymeric repeats that are 3 or more bases in length. In some embodiments, a rear RNase H guide oligonucleotide does not comprise any homopolymeric repeats of DNA nucleotides that are 3 or more bases in length. In some embodiments, the recognition sequence and/or RNase H cleavage sequence does not comprise any homopolymeric repeats that are 3 bases in length (homotrimers). In some embodiments, a front RNase H guide oligonucleotide sequence does not comprise any homopolymeric repeats that are 3 bases in length (homotrimers). In some embodiments, a front RNase H guide oligonucleotide sequence does not comprise any homopolymeric repeats of DNA nucleotides that are 3 bases in length (homotrimers). In some embodiments, a rear RNase H guide oligonucleotide sequence does not comprise any homopolymeric repeats that are 3 bases in length (homotrimers). In some embodiments, a rear RNase H guide oligonucleotide sequence does not comprise any homopolymeric repeats of DNA nucleotides that are 3 bases in length (homotrimers).

In some embodiments, the recognition sequence, identifying sequence, 5′ UTR, or 3′ UTR does not comprise a start codon. In some embodiments, the recognition sequence, identifying sequence, 5′ UTR, or 3′ UTR does not comprise the sequence ‘AUG’. Lack of start codons in the 5′ UTR or 3′ UTR prevents sequences in the 3′ UTR of the mRNA from being translated into undesired proteins. In some embodiments, the recognition sequence, identifying sequence, 5′ UTR, or 3′ UTR does not comprise an XbaI recognition site. In some embodiments, the recognition sequence, identifying sequence, 5′ UTR, or 3′ UTR does not comprise the nucleic acid sequence ‘UCUAG’. Lack of an XbaI recognition site in the 5′ UTR or 3′ UTR sequence allows the restriction enzyme XbaI to be used in generation and modification of a DNA template for in vitro transcription, without affecting the 5′ UTR or 3′ UTR sequence of the transcribed mRNA. In some embodiments, the recognition sequence, identifying sequence, 5′ UTR, or 3′ UTR does not comprise a palindromic sequence. With respect to a nucleic acid sequence, a palindromic sequence comprises the same bases in 5′-to-3′ order as in 3′-to-5′ order. For example, the nucleic acid sequence 5′-TACACAT-3′ is a palindromic sequence. In some embodiments, the recognition sequence, identifying sequence, or 3′ UTR does not comprise a nucleic acid sequence that is 5 or more, 6 or more, 7 or more, 8 or more, 9 or more, or 10 or more nucleotides in length, and is complementary to a sequence on the mRNA. An mRNA comprising a nucleic acid sequence that is complementary to a nucleic acid sequence on the mRNA can hybridize with other identical mRNA molecules to form a double-stranded RNA (dsRNA). dsRNA in cells triggers an innate immune response with multiple undesired effects, such as hydrolysis of the RNA and changes in cell physiology, including cell death. In some embodiments, the recognition sequence, identifying sequence, 5′ UTR, or 3′ UTR does not comprise a microRNA (miRNA) binding site. Information about the sequences, origins, and functions of known microRNAs maybe found in publicly available databases (e.g., mirbase.org, all versions, as described in Kozomara et al., Nucleic Acids Res 2014 42:D68-D73; Kozomara et al., Nucleic Acids Res 2011 39:D152-D157; Griffiths-Jones et al., Nucleic Acids Res 2008 36:D154-D158; Griffiths-Jones et al., Nucleic Acids Res 2006 34:D140-D144; and Griffiths-Jones et al., Nucleic Acids Res 2004 32:D109-D111, including the most recently released version miRBase 21, which contains “high confidence” microRNAs).

In some embodiments, each RNA species in a multivalent RNA composition comprises an IDR sequence with a different length. In some embodiments, the RNase H guide directs cleavage of distinct RNA species in a multivalent RNA composition into RNA fragments with distinct masses. In some embodiments, the RNase H guide directs cleavage of distinct RNA species in a multivalent RNA composition into RNA fragments with distinct lengths. In some embodiments, two or more RNA species in a multivalent RNA composition comprise IDR sequences of identical lengths but different masses. In some embodiments, the RNase H guide directs cleavage of distinct RNA species in a multivalent RNA composition into RNA fragments with identical lengths but distinct masses. In some embodiments, no RNA species in a multivalent RNA composition comprises an IDR sequence that is a sequence isomer of an IDR sequence that is comprised on a different RNA species. In some embodiments, the RNase H guide directs cleavage of distinct RNA species in a multivalent RNA composition into RNA fragments comprising IDR sequences that are not sequence isomers of each other.

In some embodiments, RNAs of a multivalent RNA composition are detected and/or purified according to the polynucleotide IDR sequences of the RNAs. In some embodiments, the mRNA IDR sequences are used to identify the presence of mRNA or determine a relative ratio of different mRNAs in a sample (e.g., a reaction product or a drug product). In some embodiments, the mRNA IDR sequences are detected using one or more of deep sequencing, PCR, and Sanger sequencing. In some embodiments, the mRNA IDR sequences are detected via liquid chromatography-ultraviolet (UV) detection (LC-UV). LC-UV is a technique combining liquid chromatography for separating molecules in a composition based on properties including size and surface charge, and using ultraviolet spectroscopy to detect the amounts of different molecules. See, e.g., Russell and Limbach. J Chromatogr B Analyt Technol Biomed Life Sci. 2013. 923-924:74-82. In some embodiments, detecting the amounts of different molecules (e.g., RNA species of a multivalent RNA composition) is used to calculate the concentration of each molecule, and the amounts of two molecules is divided to calculate the ratio between different molecules. For example, in some embodiments, LC-UV is used to detect the amounts of RNA fragments comprising IDR sequences corresponding to distinct RNA species in a multivalent RNA composition, and the amounts of detected RNA fragments comprising different IDR sequences to calculate one or more ratios of corresponding RNA species in the multivalent RNA composition.

In some embodiments, the mRNA IDR sequences are detected via HPLC. In some embodiments, mRNAs with distinct IDR sequences are detected using mass spectrometry of RNA fragments produced by RNase cleavage. Mass spectrometry determines the mass-to-charge ratios of analytes (e.g., nucleic acids) in a composition by ionizing the analytes, accelerating them through a magnetic field, which deflects the ions based on their size, with lighter and more strongly charged ions being deflected more strongly. In some embodiments, RNA fragments are analyzed by liquid chromatography-mass spectrometry (LC-MS). LC-MS is a technique that combines use of liquid chromatography (e.g., HPLC) to separate molecules in a composition based on properties including size and charge, followed by mass spectrometry to determine the mass-to-charge ratio of separated molecules. See, e.g., Russell and Limbach. J Chromatogr B Analyt Technol Biomed Life Sci. 2013. 923-924:74-82. In some embodiments, the amounts of RNA fragments detected by LC-MS corresponding to different mRNA species are used to calculate a ratio between the different mRNA species in the multivalent RNA composition. For example, in some embodiments, LC-MS is used to detect the amounts of RNA fragments comprising IDR sequences corresponding to distinct RNA species in a multivalent RNA composition, and the amounts of detected RNA fragments comprising different IDR sequences to calculate one or more ratios of corresponding RNA species in the multivalent RNA composition.

In some embodiments, analysis of a multivalent RNA composition comprises detecting the amounts of RNA fragments comprising IDR sequences corresponding to RNA species in the multivalent RNA composition, and using the detected amounts of RNA fragments to calculate a ratio between two RNA species in the multivalent RNA composition. For example, in some embodiments, the amount of a first RNA fragment corresponding to a first RNA species is divided by the amount of a second RNA fragment corresponding to a second RNA species, and the resulting quotient represents the ratio of the first RNA species to the second RNA species. In some embodiments, amounts of each RNA species are determined by measuring amounts of RNA fragments corresponding to each RNA species, and ratios between each pair of RNA species are calculated. In some embodiments, the ratios between each pair of 3 RNA species are calculated. In some embodiments, the ratios between each pair of 4 RNA species are calculated. In some embodiments, the ratios between each pair of 5 RNA species are calculated. In some embodiments, the ratios between each pair of 6 RNA species are calculated. In some embodiments, the ratios between each pair of 7 RNA species are calculated. In some embodiments, the ratios between each pair of 8 RNA species are calculated. In some embodiments, the ratios between each pair of 9 RNA species are calculated. In some embodiments, the ratios between each pair of 10 RNA species are calculated. In some embodiments, the ratios between each pair of 11 RNA species are calculated. In some embodiments, the ratios between each pair of 12 RNA species are calculated. In some embodiments, the ratios between each pair of 13 RNA species are calculated. In some embodiments, the ratios between each pair of 14 RNA species are calculated. In some embodiments, the ratios between each pair of 15 RNA species are calculated. In some embodiments, the ratios between each pair of 16 RNA species are calculated. In some embodiments, the ratios between each pair of 17 RNA species are calculated. In some embodiments, the ratios between each pair of 18 RNA species are calculated. In some embodiments, the ratios between each pair of 19 RNA species are calculated. In some embodiments, the ratios between each pair of 20 RNA species are calculated. In some embodiments, the ratios between each pair of 21 RNA species are calculated. In some embodiments, the ratios between each pair of 22 RNA species are calculated. In some embodiments, the ratios between each pair of 23 RNA species are calculated. In some embodiments, the ratios between each pair of 24 RNA species are calculated. In some embodiments, the ratios between each pair of 25 RNA species are calculated.

In some embodiments, a multivalent RNA composition comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more 24 or more, or 25 or more RNA species. In some embodiments, a multivalent RNA composition comprises 2 RNA species. In some embodiments, a multivalent RNA composition comprises 3 RNA species. In some embodiments, a multivalent RNA composition comprises 4 RNA species. In some embodiments, a multivalent RNA composition comprises 5 RNA species. In some embodiments, a multivalent RNA composition comprises 6 RNA species. In some embodiments, a multivalent RNA composition comprises 7 RNA species. In some embodiments, a multivalent RNA composition comprises 8 RNA species. In some embodiments, a multivalent RNA composition comprises 9 RNA species. In some embodiments, a multivalent RNA composition comprises 10 RNA species. In some embodiments, a multivalent RNA composition comprises 11 RNA species. In some embodiments, a multivalent RNA composition comprises 12 RNA species. In some embodiments, a multivalent RNA composition comprises 13 RNA species. In some embodiments, a multivalent RNA composition comprises 14 RNA species. In some embodiments, a multivalent RNA composition comprises 15 RNA species. In some embodiments, a multivalent RNA composition comprises 16 RNA species. In some embodiments, a multivalent RNA composition comprises 17 RNA species. In some embodiments, a multivalent RNA composition comprises 18 RNA species. In some embodiments, a multivalent RNA composition comprises 19 RNA species. In some embodiments, a multivalent RNA composition comprises 20 RNA species. In some embodiments, a multivalent RNA composition comprises 21 RNA species. In some embodiments, a multivalent RNA composition comprises 22 RNA species. In some embodiments, a multivalent RNA composition comprises 23 RNA species. In some embodiments, a multivalent RNA composition comprises 24 RNA species. In some embodiments, a multivalent RNA composition comprises 25 RNA species. In some embodiments, a multivalent RNA composition consists of 2 RNA species. In some embodiments, a multivalent RNA composition consists of 3 RNA species. In some embodiments, a multivalent RNA composition consists of 4 RNA species. In some embodiments, a multivalent RNA composition consists of 5 RNA species. In some embodiments, a multivalent RNA composition consists of 6 RNA species. In some embodiments, a multivalent RNA composition consists of 7 RNA species. In some embodiments, a multivalent RNA composition consists of 8 RNA species. In some embodiments, a multivalent RNA composition consists of 9 RNA species. In some embodiments, a multivalent RNA composition consists of 10 RNA species. In some embodiments, a multivalent RNA composition consists of 11 RNA species. In some embodiments, a multivalent RNA composition consists of 12 RNA species. In some embodiments, a multivalent RNA composition consists of 13 RNA species. In some embodiments, a multivalent RNA composition consists of 14 RNA species. In some embodiments, a multivalent RNA composition consists of 15 RNA species. In some embodiments, a multivalent RNA composition consists of 16 RNA species. In some embodiments, a multivalent RNA composition consists of 17 RNA species. In some embodiments, a multivalent RNA composition consists of 18 RNA species. In some embodiments, a multivalent RNA composition consists of 19 RNA species. In some embodiments, a multivalent RNA composition consists of 20 RNA species. In some embodiments, a multivalent RNA composition consists of 21 RNA species. In some embodiments, a multivalent RNA composition consists of 22 RNA species. In some embodiments, a multivalent RNA composition consists of 23 RNA species. In some embodiments, a multivalent RNA composition consists of 24 RNA species. In some embodiments, a multivalent RNA composition consists of 25 RNA species.

In some embodiments, the mass of the RNA fragment produced by RNase cleavage of a given RNA species in a multivalent RNA composition differs from the mass of the RNA fragments produced by RNase cleavage of every other RNA species in the composition by at least 9 Da, at least 50 Da, at least 100 Da, at least 200 Da, at least 300 Da, at least 400 Da, at least 500 Da, at least 600 Da, at least 700 Da, at least 800 Da, at least 900 Da, at least 1000 Da, at least 1100 Da, at least 1200 Da, at least 1300 Da, at least 1400 Da, at least 1500 Da, at least 1600 Da, at least 1700 Da, at least 1800 Da, at least 1900 Da, at least 1900 Da, at least 2000 Da, at least 3000 Da, at least 4000 Da, at least 5000 Da, at least 6000 Da, at least 7000 Da, or at least 8000 Da, or more. In some embodiments, the mass of the RNA fragment produced by RNase cleavage of a given RNA species in a multivalent RNA composition differs from the mass of the RNA fragments produced by RNase cleavage of every other RNA species in the composition by 50-2000 Da, 100-1500 Da, 200-1000 Da, or 400-800 Da. Exemplary IDR sequences with distinct masses include: —(0 length IDR sequence), A, GG, CC, GGA, CCA, GGAA, GGUUA, GACCA, GGACCA, GGCCAAA, GGCCAAGA, GGCCAAGGA, and CCCGUACCCCC (SEQ ID NO: 1). Other exemplary IDR sequences include: AACGUGAU; AAACAUCG; AUGCCUAA; AGUGGUCA; ACCACUGU; ACAUUGGC; CAGAUCUG; CAUCAAGU; CGCUGAUC; ACAAGCUA; CUGUAGCC; AGUACAAG; AACAACCA; AACCGAGA; AACGCUUA; AAGACGGA; AAGGUACA; ACACAGAA; ACAGCAGA; ACCUCCAA; ACGCUCGA; ACGUAUCA; ACUAUGCA; AGAGUCAA; AGAUCGCA; AGCAGGAA; AGUCACUA; AUCCUGUA; AUUGAGGA; CAACCACA; GACUAGUA; GUCCAUCA; CAAUGGAA; CACUUCGA; CAGCGUUA; CAUACCAA; CCAGUUCA; CCGAAGUA; ACAGUG; CGAUGU; UUAGGC; AUCACG; UGACCA; GACCUACGA; CCAA; GUUA; CCUUA; AGACC; UUACCA; GGAGGA; GUACGGA; GCACACA; GUUCAUU; GGCUUCUGACCA (SEQ ID NO: 2); GGCCACUCGUUAAGA (SEQ ID NO: 3); GGCCACUGAAGCCAUUGAAG (SEQ ID NO: 4); GGCCACUGAAGCCAUUGUCAAGGA (SEQ ID NO: 5); GGCCACUGAAGCCAUUGUCACCGAA (SEQ ID NO: 6); GGCGAAGCACUCGUGGCCAUUCGCA (SEQ ID NO: 7); GGCCAAGGA; GGCCAAGGAA (SEQ ID NO: 8); GGCCAAGGAAA (SEQ ID NO: 9); GGCCACUGAAGA (SEQ ID NO: 10); GGCCACUGAAGCCAUU (SEQ ID NO: 11); GGCCACUGAAGGAAG (SEQ ID NO: 12); CCGGACUAGAGA (SEQ ID NO: 13); GAAUAGAGAGGAA (SEQ ID NO: 14); GGCCACUGAAGGAAGA (SEQ ID NO: 15); GAACACUGAUCGUAGAA (SEQ ID NO: 16); GGCCACUGAAGGAAGAGA (SEQ ID NO: 17), GGAUAGAUAGCGAA (SEQ ID NO: 18), GGAGUGAGAGAGAA (SEQ ID NO: 19), or GGCCACAUAGCGAA (SEQ ID NO: 20). In some embodiments, each RNA species in a multivalent RNA composition comprises different IDR sequence with a distinct mass from each IDR sequence of each other RNA species in the composition, and each RNA species comprises an IDR sequence selected from the group consisting of—(0 length IDR sequence), A, GG, CC, GGA, CCA, GGAA, GGUUA, GACCA, GGACCA, GGCCAAA, GGCCAAGA, GGCCAAGGA, CCCGUACCCCC, AACGUGAU, AAACAUCG, AUGCCUAA, AGUGGUCA, ACCACUGU, ACAUUGGC, CAGAUCUG, CAUCAAGU, CGCUGAUC, ACAAGCUA, CUGUAGCC, AGUACAAG, AACAACCA, AACCGAGA, AACGCUUA, AAGACGGA, AAGGUACA, ACACAGAA, ACAGCAGA, ACCUCCAA, ACGCUCGA, ACGUAUCA, ACUAUGCA, AGAGUCAA, AGAUCGCA, AGCAGGAA, AGUCACUA, AUCCUGUA, AUUGAGGA, CAACCACA, GACUAGUA, GUCCAUCA, CAAUGGAA, CACUUCGA, CAGCGUUA, CAUACCAA, CCAGUUCA, CCGAAGUA, ACAGUG, CGAUGU, UUAGGC, AUCACG, UGACCA, GACCUACGA, CCAA, GUUA, CCUUA, AGACC, UUACCA, GGAGGA, GUACGGA, GCACACA, GUUCAUU, GGCUUCUGACCA (SEQ ID NO: 2), GGCCACUCGUUAAGA (SEQ ID NO: 3), GGCCACUGAAGCCAUUGAAG (SEQ ID NO:4), GGCCACUGAAGCCAUUGUCAAGGA (SEQ ID NO: 5), GGCCACUGAAGCCAUUGUCACCGAA (SEQ ID NO: 6), GGCGAAGCACUCGUGGCCAUUCGCA (SEQ ID NO: 7), GGCCAAGGA, GGCCAAGGAA (SEQ ID NO: 8), GGCCAAGGAAA (SEQ ID NO: 9), GGCCACUGAAGA (SEQ ID NO: 10), GGCCACUGAAGCCAUU (SEQ ID NO: 11), GGCCACUGAAGGAAG (SEQ ID NO: 12), CCGGACUAGAGA (SEQ ID NO: 13), GAAUAGAGAGGAA (SEQ ID NO: 14), GGCCACUGAAGGAAGA (SEQ ID NO: 15), GAACACUGAUCGUAGAA (SEQ ID NO: 16), GGCCACUGAAGGAAGAGA (SEQ ID NO: 17), GGAUAGAUAGCGAA (SEQ ID NO: 18), GGAGUGAGAGAGAA (SEQ ID NO: 19), or GGCCACAUAGCGAA (SEQ ID NO: 20). In some embodiments, each RNA species in a multivalent RNA composition comprises a different IDR sequence with a distinct mass from each IDR sequence of each other RNA species in the composition, and the IDR sequence of each RNA in the multivalent RNA composition comprises a nucleotide sequence with at least 80%, at least 85%, at least 90%, at least 95%, or at least 97% sequence identity to an IDR sequence selected from the group consisting of A, GG, CC, GGA, CCA, GGAA, GGUUA, GACCA, GGACCA, GGCCAAA, GGCCAAGA, GGCCAAGGA, CCCGUACCCCC, AACGUGAU, AAACAUCG, AUGCCUAA, AGUGGUCA, ACCACUGU, ACAUUGGC, CAGAUCUG, CAUCAAGU, CGCUGAUC, ACAAGCUA, CUGUAGCC, AGUACAAG, AACAACCA, AACCGAGA, AACGCUUA, AAGACGGA, AAGGUACA, ACACAGAA, ACAGCAGA, ACCUCCAA, ACGCUCGA, ACGUAUCA, ACUAUGCA, AGAGUCAA, AGAUCGCA, AGCAGGAA, AGUCACUA, AUCCUGUA, AUUGAGGA, CAACCACA, GACUAGUA, GUCCAUCA, CAAUGGAA, CACUUCGA, CAGCGUUA, CAUACCAA, CCAGUUCA, CCGAAGUA, ACAGUG, CGAUGU, UUAGGC, AUCACG, UGACCA, GACCUACGA, CCAA, GUUA, CCUUA, AGACC, UUACCA, GGAGGA, GUACGGA, GCACACA, GUUCAUU, GGCUUCUGACCA (SEQ ID NO: 2), GGCCACUCGUUAAGA (SEQ ID NO: 3), GGCCACUGAAGCCAUUGAAG (SEQ ID NO:4), GGCCACUGAAGCCAUUGUCAAGGA (SEQ ID NO: 5), GGCCACUGAAGCCAUUGUCACCGAA (SEQ ID NO: 6), GGCGAAGCACUCGUGGCCAUUCGCA (SEQ ID NO: 7), GGCCAAGGA, GGCCAAGGAA (SEQ ID NO: 8), GGCCAAGGAAA (SEQ ID NO: 9), GGCCACUGAAGA (SEQ ID NO: 10), GGCCACUGAAGCCAUU (SEQ ID NO: 11), GGCCACUGAAGGAAG (SEQ ID NO: 12), CCGGACUAGAGA (SEQ ID NO: 13), GAAUAGAGAGGAA (SEQ ID NO: 14), GGCCACUGAAGGAAGA (SEQ ID NO: 15), GAACACUGAUCGUAGAA (SEQ ID NO: 16), GGCCACUGAAGGAAGAGA (SEQ ID NO: 17), GGAUAGAUAGCGAA (SEQ ID NO: 18), GGAGUGAGAGAGAA (SEQ ID NO: 19), or GGCCACAUAGCGAA (SEQ ID NO: 20).

In some embodiments, each RNA species in a multivalent RNA composition comprises an IDR sequence with a distinct mass, such that RNA fragments produced by RNase cleavage of a given RNA species differ from the mass of RNA fragments produced by RNase cleavage of each other RNA species in the composition; no RNA species comprises an IDR sequence having a homopolymeric repeat of length 4 or more; and each RNA species comprises an IDR sequence with at least 80%, at least 85%, at least 90%, at least 95%, at least 97%, or up to 100% sequence identity to a nucleotide sequence selected from the group consisting of—(0 length IDR sequence), A, GG, CC, GGA, CCA, GGAA, GGUUA, GACCA, GGACCA, GGCCAAA, GGCCAAGA, GGCCAAGGA, CCCGUACCCCC, AACGUGAU, AAACAUCG, AUGCCUAA, AGUGGUCA, ACCACUGU, ACAUUGGC, CAGAUCUG, CAUCAAGU, CGCUGAUC, ACAAGCUA, CUGUAGCC, AGUACAAG, AACAACCA, AACCGAGA, AACGCUUA, AAGACGGA, AAGGUACA, ACACAGAA, ACAGCAGA, ACCUCCAA, ACGCUCGA, ACGUAUCA, ACUAUGCA, AGAGUCAA, AGAUCGCA, AGCAGGAA, AGUCACUA, AUCCUGUA, AUUGAGGA, CAACCACA, GACUAGUA, GUCCAUCA, CAAUGGAA, CACUUCGA, CAGCGUUA, CAUACCAA, CCAGUUCA, CCGAAGUA, ACAGUG, CGAUGU, UUAGGC, AUCACG, UGACCA, GACCUACGA, CCAA, GUUA, CCUUA, AGACC, UUACCA, GGAGGA, GUACGGA, GCACACA, GUUCAUU, GGCUUCUGACCA (SEQ ID NO: 2), GGCCACUCGUUAAGA (SEQ ID NO: 3), GGCCACUGAAGCCAUUGAAG (SEQ ID NO:4), GGCCACUGAAGCCAUUGUCAAGGA (SEQ ID NO: 5), GGCCACUGAAGCCAUUGUCACCGAA (SEQ ID NO: 6), GGCGAAGCACUCGUGGCCAUUCGCA (SEQ ID NO: 7), GGCCAAGGA, GGCCAAGGAA (SEQ ID NO: 8), GGCCAAGGAAA (SEQ ID NO: 9), GGCCACUGAAGA (SEQ ID NO: 10), GGCCACUGAAGCCAUU (SEQ ID NO: 11), GGCCACUGAAGGAAG (SEQ ID NO: 12), CCGGACUAGAGA (SEQ ID NO: 13), GAAUAGAGAGGAA (SEQ ID NO: 14), GGCCACUGAAGGAAGA (SEQ ID NO: 15), GAACACUGAUCGUAGAA (SEQ ID NO: 16), GGCCACUGAAGGAAGAGA (SEQ ID NO: 17), GGAUAGAUAGCGAA (SEQ ID NO: 18), GGAGUGAGAGAGAA (SEQ ID NO: 19), or GGCCACAUAGCGAA (SEQ ID NO: 20).

### PolyA Tails

Aspects of the disclosure relate to methods of producing multivalent RNA compositions that have increased purity (e.g., as measured by the percentage of mRNAs comprising polyA tails) than previous co-IVT methods. A “polyA tail” is a region of mRNA that is downstream, e.g., directly downstream (i.e., 3′), from the 3′ UTR that contains multiple, consecutive adenosine monophosphates. A polyA tail may contain 10 to 300 adenosine monophosphates. For example, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a polyA tail contains 50 to 250 adenosine monophosphates. In a relevant biological setting (e.g., in cells, in vivo, etc.) the poly(A) tail functions to protect mRNA from enzymatic degradation, e.g., in the cytoplasm, and aids in transcription termination, export of the mRNA from the nucleus, and translation.

Some embodiments comprise normalizing the molar amounts of the first and second populations of DNA molecules present in an IVT reaction mixture prior to the start of the IVT according to the polyA-tailing efficiency of the first or second population of DNA molecules results in multivalent RNA compositions where at least 85% of the RNAs in the composition comprise a polyA tail. As used herein, “polyA-tailing efficiency” refers to the amount (e.g., expressed as a percentage) of mRNAs having polyA tails that are produced by an IVT reaction using an input DNA relative to the total amount of mRNAs produced in the IVT reaction using the input DNA. The polyA-tailing efficiency of an IVT reaction may vary, for example depending upon the RNA polymerase used, amount or purity of input DNA used, etc. In some embodiments, the polyA-tailing efficiency of an IVT reaction is greater than 85%, 90%, 95%, or 99.9%. Methods of calculating polyA-tailing efficiency are known, for example by determining the amount of polyA tail-containing mRNA relative to total mRNA produced in an IVT reaction by column chromatography (e.g., oligo-dT chromatography). In some embodiments, the normalizing comprises dividing the final molar percentage of desired RNA by the polyA-tailing efficiency of the highest efficiency polyA RNA in the composition. In some embodiments, normalizing further comprises determining the mass amount of each input DNA to add based upon calculating the desired molar amount of input DNA, or RNA in the pre-determined ratio. In some embodiments, normalizing comprises dividing the final molar percentage of desired RNA (e.g., a pre-determined ratio of RNAs) by the polyA-tailing efficiency of the final processed (e.g., purified) polyA RNA efficiency of each different RNA in the multivalent RNA composition. In some embodiments, normalizing further comprises determining the rate of the RNA production ratio of each different RNA to determine the input DNA ratio for the different RNAs to achieve a pre-determined ratio.

In some embodiments, at least 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or 99.9% of RNAs in a multivalent RNA composition produced by a method described herein comprise a polyA tail. In some embodiments, at least 85%, 86%, 87%, 88%, 89%, 90%, 91%, 92%, 93%, 94%, 95%, 96%, 97%, 98%, 99%, or 99.9% of each RNA in a multivalent RNA composition produced by a method described herein comprise a polyA tail. The amount (e.g., percentage of polyA-tailed RNAs in a multivalent RNA composition may be measured i) after the IVT reaction and before purification, or ii) after the multivalent RNA composition has been purified (e.g., by chromatography, such as oligo-dT chromatography).

In some embodiments, terminal groups on the poly A tail can be incorporated for stabilization. Polynucleotides can include des-3′ hydroxyl tails. They can also include structural moieties or 2′-Omethyl modifications as taught by Junjie Li, et al. (Current Biology, Vol. 15, 1501-1507, Aug. 23, 2005, the contents of which are incorporated herein by reference in its entirety for this purpose).

Unique polyA tail lengths provide certain advantages to nucleic acids. Generally, the length of a polyA tail, when present, is greater than 30 nucleotides in length. In another embodiment, the polyA tail is greater than 35 nucleotides in length (e.g., at least or greater than about 15, 20, 25, 30, 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, or 3,000 nucleotides).

In some embodiments, the polyA tail is designed relative to the length of the overall nucleic acid or the length of a particular region of the nucleic acid. This design can be based on the length of a coding region, the length of a particular feature or region or based on the length of the ultimate product expressed from the nucleic acids.

In this context, the polyA tail can be 10, 20, 30, 40, 50, 60, 70, 80, 90, or 100% greater in length than the nucleic acid or feature thereof. The polyA tail can also be designed as a fraction of the nucleic acid to which it belongs. In this context, the polyA tail can be 10, 20, 30, 40, 50, 60, 70, 80, or 90% or more of the total length of the construct, a construct region or the total length of the construct minus the polyA tail. Further, engineered binding sites and conjugation of nucleic acids for PolyA binding protein can enhance expression.

### Lipid Compositions

In some embodiments, the nucleic acids are formulated as a lipid composition, such as a composition comprising a lipid nanoparticle, a liposome, and/or a lipoplex. In some embodiments, nucleic acids are formulated as lipid nanoparticle (LNP) compositions. Lipid nanoparticles typically comprise amino lipid, non-cationic lipid, structural lipid, and PEG lipid components along with the nucleic acid cargo of interest. The lipid nanoparticles can be generated using components, compositions, and methods as are generally known in the art, see for example PCT/US2016/052352; PCT/US2016/068300; PCT/US2017/037551; PCT/US2015/027400; PCT/US2016/047406; PCT/US2016/000129; PCT/US2016/014280; PCT/US2017/038426; PCT/US2014/027077; PCT/US2014/055394; PCT/US2016/052117; PCT/US2012/069610; PCT/US2017/027492; PCT/US2016/059575; PCT/US2016/069491; PCT/US2016/069493; and PCT/US2014/66242, all of which are incorporated by reference herein in their entirety.

In some embodiments, the lipid nanoparticle comprises at least one ionizable amino lipid, at least one non-cationic lipid, at least one sterol, and/or at least one polyethylene glycol (PEG)-modified lipid.

In some embodiments, the lipid nanoparticle comprises a molar ratio of 20-60% ionizable amino lipid, 5-25% non-cationic lipid, 25-55% structural lipid, and 0.5-15% PEG-modified lipid.

In some embodiments, the lipid nanoparticle comprises a molar ratio of 20-60% ionizable amino lipid, 5-30% non-cationic lipid, 10-55% structural lipid, and 0.5-15% PEG-modified lipid.

In some embodiments, the lipid nanoparticle comprises 40-50 mol % ionizable lipid, optionally 45-50 mol %, for example, 45-46 mol %, 46-47 mol %, 47-48 mol %, 48-49 mol %, or 49-50 mol % for example about 45 mol %, 45.5 mol %, 46 mol %, 46.5 mol %, 47 mol %, 47.5 mol %, 48 mol %, 48.5 mol %, 49 mol %, or 49.5 mol %.

In some embodiments, the lipid nanoparticle comprises 20-60 mol % ionizable amino lipid. For example, the lipid nanoparticle may comprise 20-50 mol %, 20-40 mol %, 20-30 mol %, 30-60 mol %, 30-50 mol %, 30-40 mol %, 40-60 mol %, 40-50 mol %, or 50-60 mol % ionizable amino lipid. In some embodiments, the lipid nanoparticle comprises 20 mol %, 30 mol %, 40 mol %, 50 mol %, or 60 mol % ionizable amino lipid. In some embodiments, the lipid nanoparticle comprises 35 mol %, 36 mol %, 37 mol %, 38 mol %, 39 mol %, 40 mol %, 41 mol %, 42 mol %, 43 mol %, 44 mol %, 45 mol %, 46 mol %, 47 mol %, 48 mol %, 49 mol %, 50 mol %, 51 mol %, 52 mol %, 53 mol %, 54 mol %, or 55 mol % ionizable amino lipid.

In some embodiments, the lipid nanoparticle comprises 45-55 mole percent (mol %) ionizable amino lipid. For example, lipid nanoparticle may comprise 45, 46, 47, 48, 49, 50, 51, 52, 53, 54, or 55 mol % ionizable amino lipid.

### Ionizable Amino Lipids

In some embodiments, the ionizable amino lipid of the present disclosure is a compound of Formula (AI):

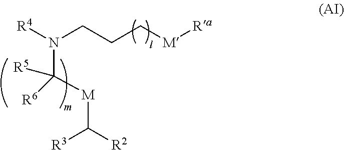

or its N-oxide, or a salt or isomer thereof,
wherein R′^{a} is R′^{branched}; wherein
R′^{branched} is:

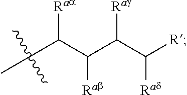

wherein

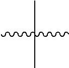

denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH, wherein n is selected from the group consisting of 1, 2, 3, 4, and 5, and

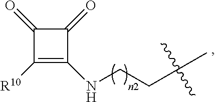

wherein

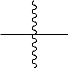

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments of the compounds of Formula (A), R′^{a} is R′^{branched}; R′^{branched} is

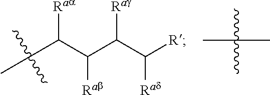

denotes a point of attachment; R^{aα}, R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some embodiments of the compounds of Formula (AI), R′^{a} is R′^{branched}; R′^{branched} is

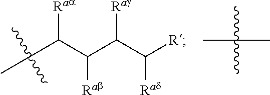

denotes a point of attachment; R^{aα}, R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; l is 3; and m is 7.

In some embodiments of the compounds of Formula (AI), R′^{a} is R′^{branched}; R′^{branched} is

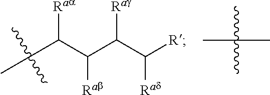

denotes a point of attachment, R^{aα} is C₂₋₁₂ alkyl; R^{aβ}, R^{aγ}, and R^{aδ} are each H; R² and R³ are each C₁₋₁₄ alkyl; R⁴ is

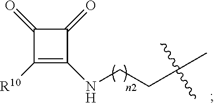

R¹⁰ NH(C₁₋₆ alkyl); n2 is 2; R⁵ is H; each R⁶ is H; M and M′ are each —C(O)O—; R′ is a C₁₋₁₂ alkyl; l is 5; and m is 7.

In some embodiments of the compounds of Formula (I), R′^{a} is R′^{branched}; R′^{branched} is

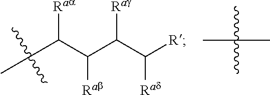

denotes a point of attachment; R^{aα}, R^{aβ}, and R^{aδ} are each H; R^{aγ} is C₂₋₁₂ alkyl; R² and R³ are each C₁₋₁₄ alkyl; R⁴ is —(CH₂)ₙOH; n is 2; each R⁵ is H, each R⁶ is H; M and M′ are each —C(O)O—; R′ is a C₁₋₁₂ alkyl; 1 is 5; and m is 7.

In some embodiments, the compound of Formula (I) is selected from:

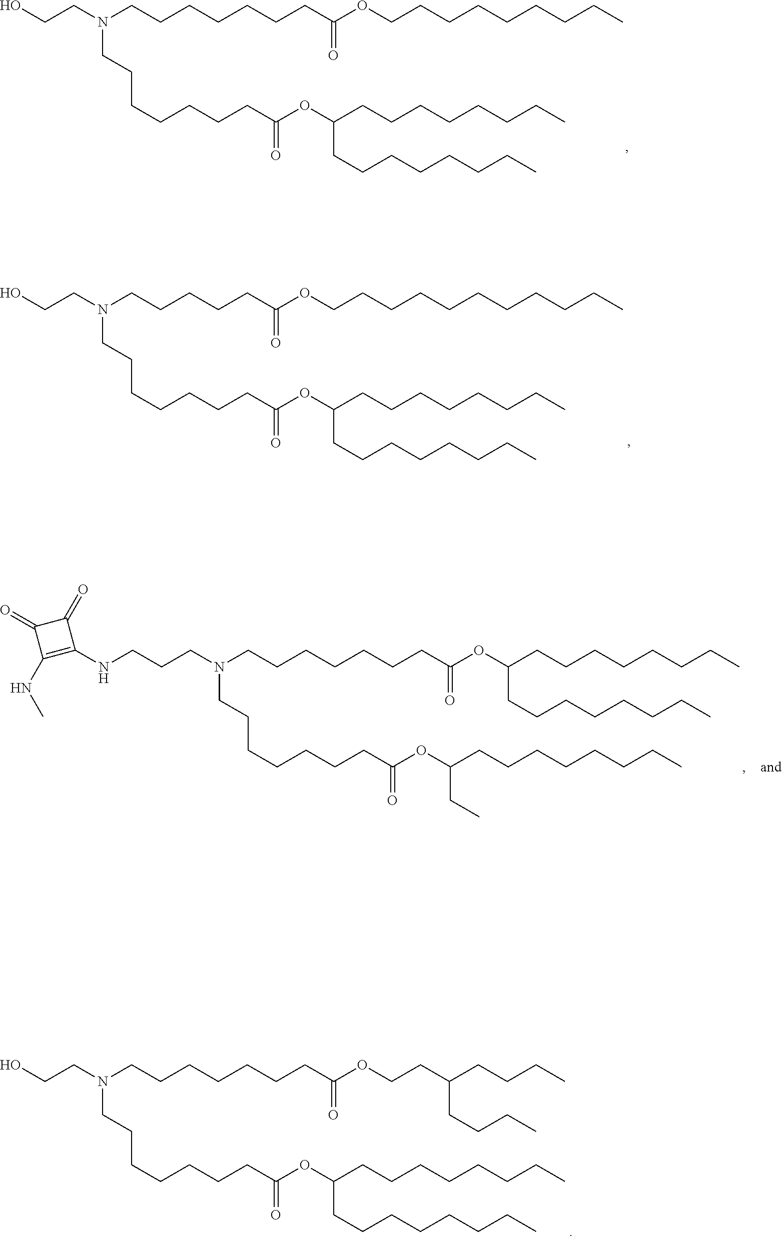 [SMILES: CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC ; CCCCCCCCC(CC)OC(=O)CCCCCCCN(CCCCCCCC(=O)OC(CCCCCCCC)CCCCCCCC)CCCNc1c(NC)c(=O)c1=O ; CCCCCCCCC(CCCCCCCC)OC(=O)CCCCCCCN(CCO)CCCCCCCC(=O)OCCC(CCCC)CCCC]

In some embodiments, the ionizable amino lipid is a compound of Formula (Ala):

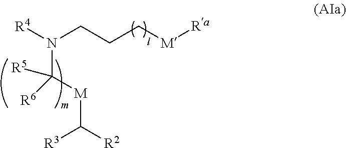

or its N-oxide, or a salt or isomer thereof,
wherein R′^{a} is R′^{branched}; wherein
R′^{branched} is:

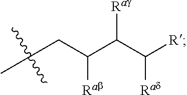

wherein

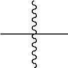

denotes a point of attachment;
wherein R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

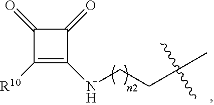

wherein

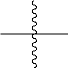

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments, the ionizable amino lipid is a compound of Formula (AIb):

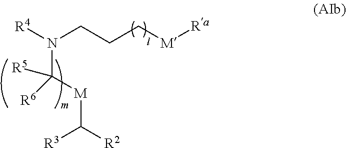

or its N-oxide, or a salt or isomer thereof,
wherein R′^{a} is R′^{branched}; wherein
R′^{branched} is:

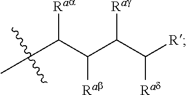

wherein

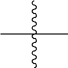

denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is —(CH₂)ₙOH, wherein n is selected from the group consisting of 1, 2, 3, 4, and 5;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments of Formula (AI) or (AIb), R′^{a} is R′^{branched}; R′^{branched} is

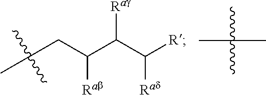

denotes a point of attachment; R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; l is 5; and m is 7.

In some embodiments of Formula (AI) or (AIb), R′^{a} is R′^{branched}; R′^{branched} is

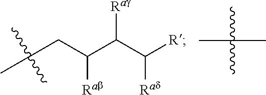

denotes a point of attachment; R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 3, and m is 7.

In some embodiments of Formula (AI) or (AIb), R′^{a} is R′^{branched}; R′^{branched} is

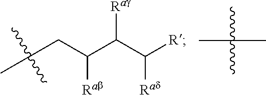

denotes a point of attachment; R^{aβ} and R^{aδ} are each H; R^{aγ} is C₂₋₁₂ alkyl; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; l is 5; and m is 7.

In some embodiments, the ionizable amino lipid is a compound of Formula (AIc):

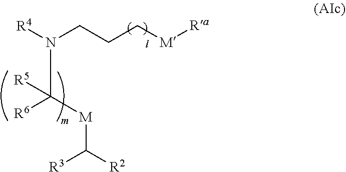

or its N-oxide, or a salt or isomer thereof,
wherein R′^{a} is R′^{branched}; wherein
   R′^{branched} is:

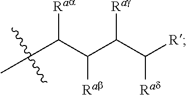

wherein

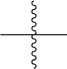

denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is

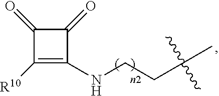

wherein

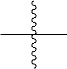

denotes a point of attachment; wherein R¹⁰ is N(R)₂; each R is independently selected from the group consisting of C₁₋₆ alkyl, C₂₋₃ alkenyl, and H; n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments, R′^{a} is R′^{branched}; R′^{branched} is

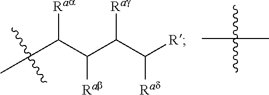

denotes a point of attachment; R^{aβ}, R^{aγ}, and R^{aδ} are each H; R is C₂₋₁₂ alkyl; R² and R³ are each C₁₋₁₄ alkyl; R⁴ is

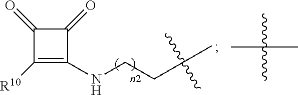

denotes a point of attachment; R¹⁰ is NH(C₁₋₆ alkyl); n2 is 2; each R⁵ is H; each R⁶ is H; M and M′ are each —C(O)O—; R′ is a C₁₋₁₂ alkyl; l is 5; and m is 7.

In some embodiments, the compound of Formula (AIc) is:

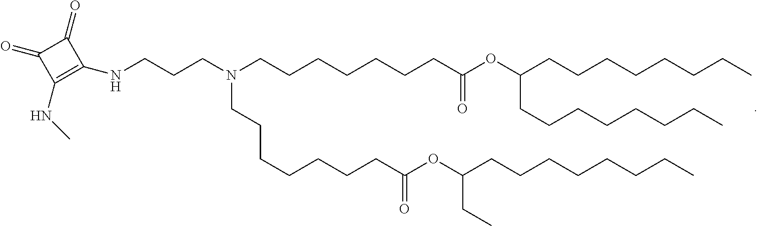 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

In some embodiments, the ionizable amino lipid is a compound of Formula (AII):

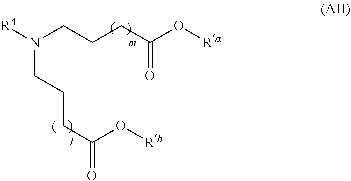

or its N-oxide, or a salt or isomer thereof,
wherein R′^{a} is R′^{branched} or R′^{cyclic}; wherein
R′^{branched} is:

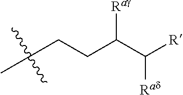

and R′^{cyclic} is:

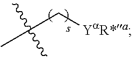

and
R′^{b} is:

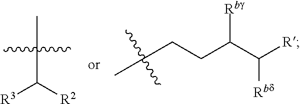

wherein

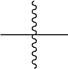

denotes a point of attachment;
R^{aγ} and R^{aδ} are each independently selected from the group consisting of H, C₁₋₁₂ alkyl, and C₂₋₁₂ alkenyl, wherein at least one of R^{aγ} and R^{aδ} is selected from the group consisting of C₁₋₁₂ alkyl and C₂₋₁₂ alkenyl;
R^{bγ} and R^{bδ} are each independently selected from the group consisting of H, C₁₋₁₂ alkyl, and C₂₋₂ alkenyl, wherein at least one of R^{bγ} and R^{bδ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

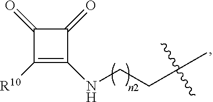

wherein

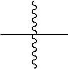

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
Y^{a} is a C₃₋₆ carbocycle;
R*″^{a} is selected from the group consisting of C₁₋₁₅ alkyl and C₂₋₁₅ alkenyl; and
s is 2 or 3;
m is selected from 1, 2, 3, 4, 5, 6, 7, 8, and 9;
l is selected from 1, 2, 3, 4, 5, 6, 7, 8, and 9.

In some embodiments, the ionizable amino lipid is a compound of Formula (AII-a):

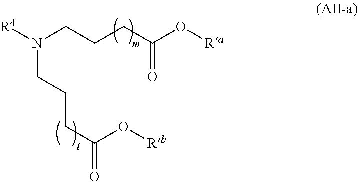

or its N-oxide, or a salt or isomer thereof,
wherein R′^{a} is R′^{branched} or R′^{cyclic}; wherein
R′^{branched} is:

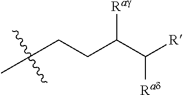

and R′^{b} is

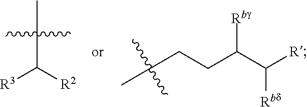

wherein

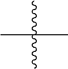

denotes a point of attachment;
R^{aγ} and R^{aδ} are each independently selected from the group consisting of H, C₁₋₁₂ alkyl, and C₂₋₁₂ alkenyl, wherein at least one of R^{aγ} and R^{aδ} is selected from the group consisting of C₁₋₁₂ alkyl and C₂₋₁₂ alkenyl;
R^{bγ} and R^{bδ} are each independently selected from the group consisting of H, C₁₋₁₂ alkyl, and C₂₋₁₂ alkenyl, wherein at least one of R^{bγ} and R^{bδ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

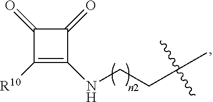

wherein

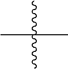

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid is a compound of Formula (AII-b):

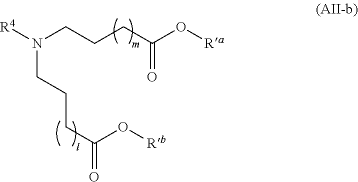

or its N-oxide, or a salt or isomer thereof,
wherein R′^{a} is R′^{branched} or R′^{cyclic}; wherein
R′^{branched} is:

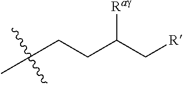

and R′^{b} is:

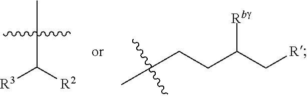

wherein

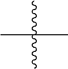

denotes a point of attachment;
R^{aγ} and R^{bγ} are each independently selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

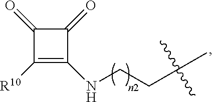

wherein

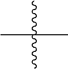

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid is a compound of Formula (AII-c):

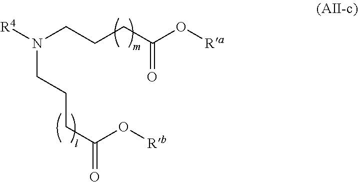

or its N-oxide, or a salt or isomer thereof,
wherein R′^{a} is R′^{branched} or R′^{cyclic}; wherein
R′^{branched} is:

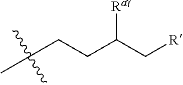

and R′^{b} is:

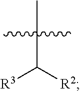

wherein

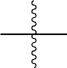

denotes a point of attachment;
wherein R^{aγ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

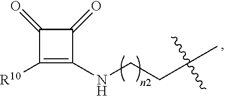

wherein

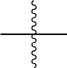

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
R′ is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid is a compound of Formula (AII-d):

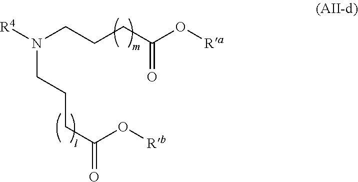

or its N-oxide, or a salt or isomer thereof,
wherein R′^{a} is R′^{branched} or R′^{cyclic}; wherein
R′^{branched} is:

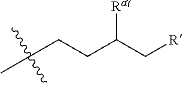

R and R′^{b} is:

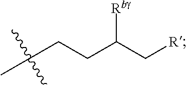

wherein

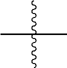

denotes a point of attachment;
wherein R^{aγ} and R^{bγ} are each independently selected from the group consisting of C₁₋₁₂ alkyl and C₂₋₁₂ alkenyl;
R⁴ is selected from the group consisting of —(CH₂)ₙOH wherein n is selected from the group consisting of 1, 2, 3, 4, and 5, and

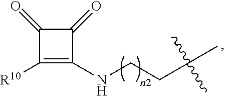

wherein

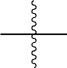

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid is a compound of Formula (AII-e):

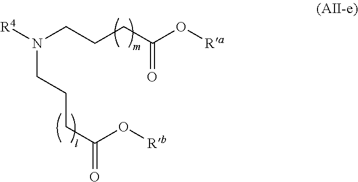

or its N-oxide, or a salt or isomer thereof,
wherein R′^{a} is R′^{branched} or R′^{cyclic}; wherein
R′^{branched} is:

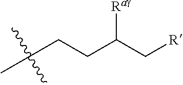

and R′^{b} is:

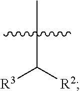

wherein

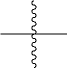

denotes a point of attachment;
wherein R^{aγ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is —(CH₂)ₙOH wherein n is selected from the group consisting of 1, 2, 3, 4, and 5;
R′ is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments of the compound of Formula (AII), (AII-a), (AII-b), (AII-c), (AII-d), or (AII-e), m and l are each independently selected from 4, 5, and 6. In some embodiments of the compound of Formula (AII), (AII-a), (AII-b), (AII-c), (AII-d), or (AII-e), m and 1 are each 5.

In some embodiments of the compound of Formula (AII), (AII-a), (AII-b), (AII-c), (AII-d), or (AII-e), each R′ independently is a C₁₋₁₂ alkyl. In some embodiments of the compound of Formula (AII), (AII-a), (AII-b), (AII-c), (AII-d), or (AII-e), each R′ independently is a C₂₋₅ alkyl.

In some embodiments of the compound of Formula (AII), (AII-a), (AII-b), (AII-c), (AII-d), or (AII-e), R′^{b} is:

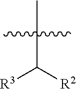

and R² and R³ are each independently a C₁₋₁₄ alkyl. In some embodiments of the compound of Formula (AII), (AII-a), (AII-b), (AII-c), (AII-d), or (AII-e), R′^{b} is:

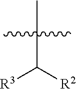

and R² and R³ are each independently a C₆₋₁₀ alkyl. In some embodiments of the compound of Formula (AII), (AII-a), (AII-b), (AII-c), (AII-d), or (AII-e), R′^{b} is:

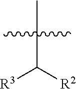

and R² and R³ are each a C₈ alkyl.

In some embodiments of the compound of Formula (AII), (AII-a), (AII-b), (AII-c), (AII-d), or (AII-e), R′^{branched} is:

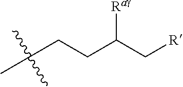

### and R′^{b} is:

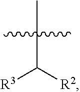

R^{aγ} is a C₁₋₁₂ alkyl and R² and R³ are each independently a C₆₋₁₀ alkyl. In some embodiments of the compound of Formula (AII), (AII-a), (AII-b), (AII-c), (AII-d), or (AII-e), R′^{branched} is:

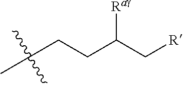

### and R′^{b} is:

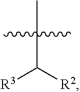

R^{aγ} is a C₂₋₆ alkyl and R² and R³ are each independently a C₆₋₁₀ alkyl. In some embodiments of the compound of Formula (AII), (AII-a), (AII-b), (AII-c), (AII-d), or (AII-e), R′^{branched} is:

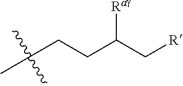

### R′^{b} is:

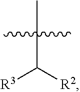

R^{aγ} is a C₂₋₆ alkyl, and R² and R³ are each a C₈ alkyl.

In some embodiments of the compound of Formula (AII), (AII-a), (AII-b), (AII-c), (AII-d), or (AII-e), R′^{branched} is:

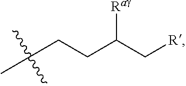

### R′^{b} is:

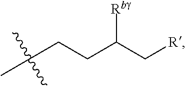

and R^{aγ} and R^{bγ} are each a C₁₋₁₂ alkyl. In some embodiments of the compound of Formula (AII), (AII-a), (AII-b), (AII-c), (AII-d), or (AII-e), R′^{branched} is:

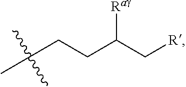

### R′^{b} is:

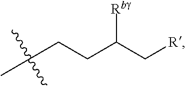

and R^{aγ} and R^{bγ} are each a C₂₋₆ alkyl.

In some embodiments of the compound of Formula (AII), (AII-a), (AII-b), (AII-c), (AII-d), or (AII-e), m and 1 are each independently selected from 4, 5, and 6 and each R′ independently is a C₁₋₁₂ alkyl. In some embodiments of the compound of Formula (AII), (AII-a), (AII-b), (AII-c), (AII-d), or (AII-e), m and 1 are each 5 and each R′ independently is a C₂₋₅ alkyl.

In some embodiments of the compound of (AII), (AII-a), (AII-b), (AII-c), (AII-d), or (AII-e), R′^{branched} is:

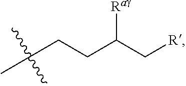

### R′^{b} is:

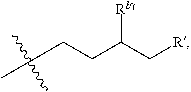

m and l are each independently selected from 4, 5, and 6, each R′ independently is a C₁₋₁₂ alkyl, and R^{aγ} and R^{bγ} are each a C₁₋₁₂ alkyl. In some embodiments of the compound of Formula (AII), (AII-a), (AII-b), (AII-c), (AII-d), or (AII-e), R′^{branched} is:

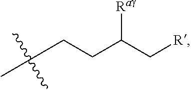

### R′^{b} is:

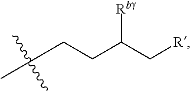

m and l are each 5, each R′ independently is a C₂₋₅ alkyl, and R^{aγ} and R^{bγ} are each a C₂₋₆ alkyl.

In some embodiments of the compound of Formula (AII), (AII-a), (AII-b), (AII-c), (AII-d), or (AII-e), R′^{branched} is:

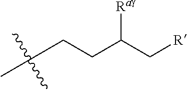

### and R′^{b} is:

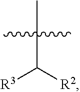

m and l are each independently selected from 4, 5, and 6, R′ is a C₁₋₁₂ alkyl, R^{aγ} is a C₁₋₁₂ alkyl and R² and R³ are each independently a C₆₋₁₀ alkyl.

In some embodiments of the compound of Formula (AII), (AII-a), (AII-b), (AII-c), (AII-d), or (AII-e), R′^{branched} is:

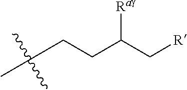

### and R′^{b} is:

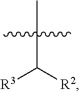

m and l are each 5, R′ is a C₂₋₅ alkyl, R^{aγ} is a C₂₋₆ alkyl, and R² and R³ are each a C₈ alkyl.

In some embodiments of the compound of (AII), (AII-a), (AII-b), (AII-c), (AII-d), or (AII-e), R⁴ is

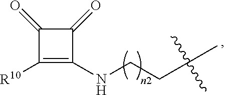

wherein R¹⁰ is NH(C₁₋₆ alkyl) and n2 is 2. In some embodiments of the compound of Formula (AII), (AII-a), (AII-b), (AII-c), (AII-d), or (AII-e), R⁴ is

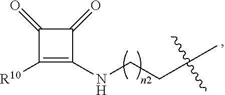

wherein R¹⁰ is NH(CH₃) and n2 is 2.

In some embodiments of the compound of Formula (AII), (AII-a), (AII-b), (AII-c), (AII-d), or (AII-e), R′^{branched} is:

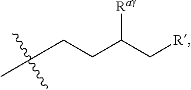

### R′^{b} is:

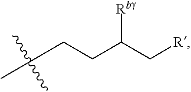

m and l are each independently selected from 4, 5, and 6, each R′ independently is a C₁₋₁₂ alkyl, R^{aγ} and R^{bγ} are each a C₁₋₁₂ alkyl, and R⁴ is

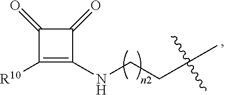

wherein R¹⁰ is NH(C₁₋₆ alkyl), and n2 is 2. In some embodiments of the compound of Formula (AII), (AII-a), (AII-b), (AII-c), (AII-d), or (AII-e), R′^{branched} is:

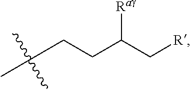

### R′^{b} is:

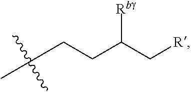

m and l are each 5, each R′ independently is a C₂₋₅ alkyl, R^{aγ} and R^{bγ} are each a C₂₋₆ alkyl, and R⁴ is

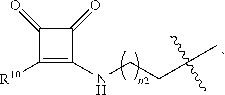

wherein R¹⁰ is NH(CH₃) and n2 is 2.

In some embodiments of the compound of Formula (AII), (AII-a), (AII-b), (AII-c), (AII-d), or (AII-e), R′^{branched} is:

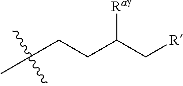

### and R′^{b} is:

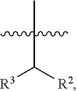

m and l are each independently selected from 4, 5, and 6, R′ is a C₁₋₁₂ alkyl, R² and R³ are each independently a C₆₋₁₀ alkyl, R^{aγ} is a C₁₋₁₂ alkyl, and R⁴ is

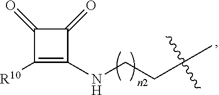

wherein R¹⁰ is NH(C₁₋₆ alkyl) and n2 is 2. In some embodiments of the compound of Formula (AII), (AII-a), (AII-b), (AII-c), (AII-d), or (AII-e), R′^{branched} is:

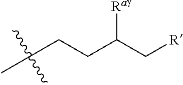

### and R′^{b} is:

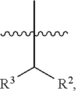

m and l are each 5, R′ is a C₂₋₅ alkyl, R^{aγ} is a C₂₋₆ alkyl, R² and R³ are each a C₈ alkyl, and R⁴ is

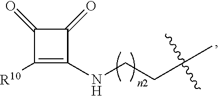

wherein R¹⁰ is NH(CH₃) and n2 is 2.

In some embodiments of the compound of Formula (AII), (AII-a), (AII-b), (AII-c), (AII-d), or (AII-e), R⁴ is —(CH₂)ₙOH and n is 2, 3, or 4. In some embodiments of the compound of Formula (AII), (AII-a), (AII-b), (AII-c), (AII-d), or (AII-e), R⁴ is —(CH₂)ₙOH and n is 2.

In some embodiments of the compound of Formula (AII), (AII-a), (AII-b), (AII-c), (AII-d), or (AII-e), R′^{branched} is:

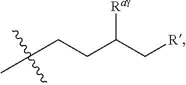

### R′^{b} is:

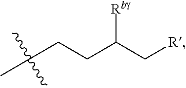

m and l are each independently selected from 4, 5, and 6, each R′ independently is a C₁₋₁₂ alkyl, R^{aγ} and R^{bγ} are each a C₁₋₁₂ alkyl, R⁴ is —(CH₂)ₙOH, and n is 2, 3, or 4. In some embodiments of the compound of Formula (AII), (AII-a), (AII-b), (AII-c), (AII-d), or (AII-e), R′^{branched} is:

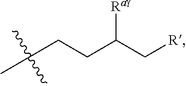

### R′^{b} is:

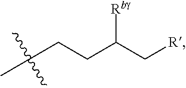

m and l are each 5, each R′ independently is a C₂₋₅ alkyl, R^{aγ} and R^{bγ} are each a C₂₋₆ alkyl, R⁴ is —(CH₂)ₙOH, and n is 2.

In some embodiments, the ionizable amino lipid is a compound of Formula (AII-f):

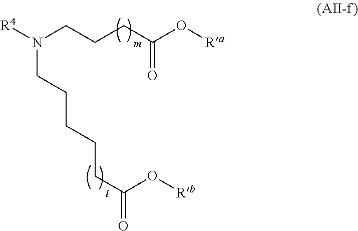

or its N-oxide, or a salt or isomer thereof,
wherein R′^{a} is R′^{branched} or R′^{cyclic}; wherein
R′^{branched} is:

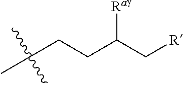

and R′^{b} is:

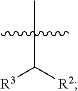

wherein

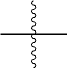

denotes a point of attachment;
R^{aγ} is a C₁₋₁₂ alkyl;
R² and R³ are each independently a C₁₋₁₄ alkyl;
R⁴ is —(CH₂)ₙOH wherein n is selected from the group consisting of 1, 2, 3, 4, and 5;
R′ is a C₁₋₁₂ alkyl;
m is selected from 4, 5, and 6; and
l is selected from 4, 5, and 6.

In some embodiments of the compound of Formula (AII-f), m and l are each 5, and n is 2, 3, or 4.

In some embodiments of the compound of Formula (AII-f) R′ is a C₂₋₅ alkyl, R^{aγ} is a C₂₋₆ alkyl, and R² and R³ are each a C₆₋₁₀ alkyl.

In some embodiments of the compound of Formula (AII-f), m and l are each 5, n is 2, 3, or 4, R′ is a C₂₋₅ alkyl, R^{aγ} is a C₂₋₆ alkyl, and R² and R³ are each a C₆₋₁₀ alkyl.

In some embodiments, the ionizable amino lipid is a compound of Formula (AII-g):

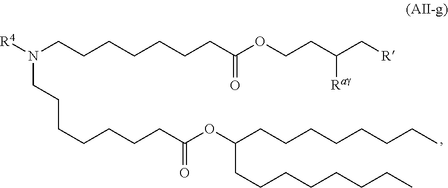

wherein
R^{aγ} is a C₂₋₆ alkyl;
R′ is a C₂₋₅ alkyl; and
R⁴ is selected from the group consisting of —(CH₂)ₙOH wherein n is selected from the group consisting of 3, 4, and 5, and

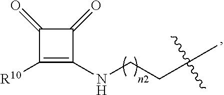

wherein

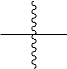

denotes a point of attachment, R¹⁰ is NH(C₁₋₆ alkyl), and n2 is selected from the group consisting of 1, 2, and 3.

In some embodiments, the ionizable amino lipid is a compound of Formula (AII-h):

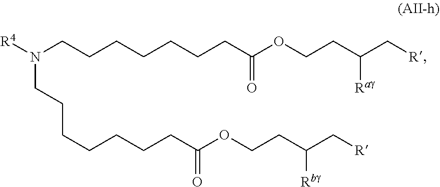

wherein
R^{aγ} and R^{bγ} are each independently a C₂₋₆ alkyl;
each R′ independently is a C₂₋₃ alkyl; and
R⁴ is selected from the group consisting of —(CH₂)ₙOH wherein n is selected from the group consisting of 3, 4, and 5, and

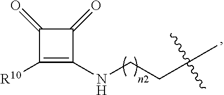

wherein

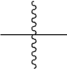

denotes a point of attachment, R¹⁰ is NH(C₁₋₆ alkyl), and n2 is selected from the group consisting of 1, 2, and 3.

In some embodiments of the compound of Formula (AII-g) or (AII-h), R⁴ is

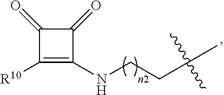

wherein
R¹⁰ is NH(CH₃) and n2 is 2.

In some embodiments of the compound of Formula (AII-g) or (AII-h), R⁴ is —(CH₂)₂OH.

In some embodiments, the ionizable amino lipids of the present disclosure may be one or more of compounds of Formula (VI):

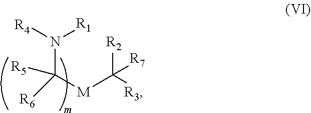

or their N-oxides, or salts or isomers thereof, wherein:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of hydrogen, a C₃₋₆ carbocycle, —(CH₂)ₙQ, —(CH₂)ₙCHQR, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R₈, —N(R)S(O)₂R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and —C(R)N(R)₂C(O)OR, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —OC(O)-M″-C(O)O—, —C(O)N(R′)′, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C₁₋₁₃ alkyl or C₂₋₁₃ alkenyl;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₅ alkyl and C₃₋₁₅ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13; and wherein when R₄ is —(CH₂)˜Q, —(CH₂)ₙCHQR, —CHQR, or -CQ(R)₂, then (i) Q is not —N(R)₂ when n is 1, 2, 3, 4 or 5, or (ii) Q is not 5, 6, or 7-membered heterocycloalkyl when n is 1 or 2.

In some embodiments, another subset of compounds of Formula (VI) includes those in which:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and a 5- to 14-membered heterocycloalkyl having one or more heteroatoms selected from N, O, and S which is substituted with one or more substituents selected from oxo (═O), OH, amino, mono- or di-alkylamino, and C₁₋₃ alkyl, and each n is independently selected from 1, 2, 3, 4, and 5;
each R₅ is independently selected from the group consisting of C₁₋₃ alkyl, C₂₋₃ alkenyl, and H;
each R_{b} is independently selected from the group consisting of C₁₋₃ alkyl, C₂₋₃ alkenyl, and H;
M and M′ are independently selected from —C(O)O—, —OC(O)—, —C(O)N(R′)′, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (VI) includes those in which:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heterocycle having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5; and when Q is a 5- to 14-membered heterocycle and (i) R₄ is —(CH₂)ₙQ in which n is 1 or 2, or (ii) R₄ is —(CH₂)ₙCHQR in which n is 1, or (iii) R₄ is —CHQR, and —CQ(R)₂, then Q is either a 5- to 14-membered heteroaryl or 8- to 14-membered heterocycloalkyl;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)′, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (VI) includes those in which:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)′, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (VI) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₂₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is —(CH₂)ₙQ or —(CH₂)ₙCHQR, where Q is —N(R)₂, and n is selected from 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)′, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (VI) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of C₁₋₁₄ alkyl, C₂₋₁₄ alkenyl, —R*YR″, —YR″, and —R*OR″, or R₂ and R₃, together with the atom to which they are attached, form a heterocycle or carbocycle,
R₄ is selected from the group consisting of —(CH₂)ₙQ, —(CH₂)ₙCHQR, —CHQR, and —CQ(R)₂, where Q is —N(R)₂, and n is selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)′, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In certain embodiments, a subset of compounds of Formula (VI) includes those of Formula (VI-A):

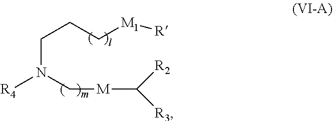

or its N-oxide, or a salt or isomer thereof, wherein 1 is selected from 1, 2, 3, 4, and 5; m is selected from 5, 6, 7, 8, and 9; M₁ is a bond or M′; R₄ is hydrogen, unsubstituted C₁₋₃ alkyl, or —(CH₂).Q, in which Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)-M″-C(O)O—, —C(O)N(R′)′, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. For example, m is 5, 7, or 9. For example, Q is OH, —NHC(S)N(R)₂, or —NHC(O)N(R)₂. For example, Q is —N(R)C(O)R, or —N(R)S(O)₂R.

In certain embodiments, a subset of compounds of Formula (VI) includes those of Formula (VI-B):

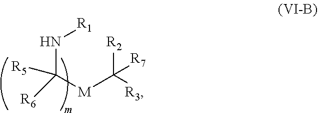

or its N-oxide, or a salt or isomer thereof in which all variables are as defined herein. For example, m is selected from 5, 6, 7, 8, and 9; R₄ is hydrogen, unsubstituted C₁₋₃ alkyl, or —(CH₂)ₙQ, in which Q is H, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)-M″-C(O)O—, —C(O)N(R′)′, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. For example, m is 5, 7, or 9. For example, Q is OH, —NHC(S)N(R)₂, or —NHC(O)N(R)₂. For example, Q is —N(R)C(O)R, or —N(R)S(O)₂R.

In certain embodiments, a subset of compounds of Formula (VI) includes those of Formula (VII):

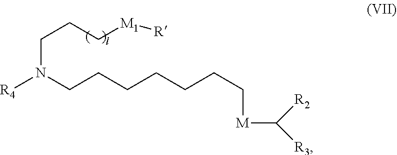

or its N-oxide, or a salt or isomer thereof, wherein 1 is selected from 1, 2, 3, 4, and 5; M₁ is a bond or M′; R₄ is hydrogen, unsubstituted C₁₋₃ alkyl, or —(CH₂)ₙQ, in which n is 2, 3, or 4, and Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)-M″-C(O)O—, —C(O)N(R′)′, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In some embodiments, the compounds of Formula (VI) are of Formula (VIIa),

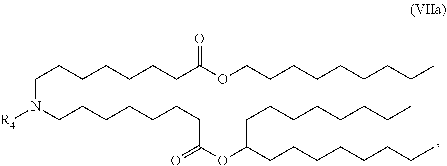

or their N-oxides, or salts or isomers thereof, wherein R₄ is as described herein.

In another embodiment, the compounds of Formula (VI) are of Formula (VIIb),

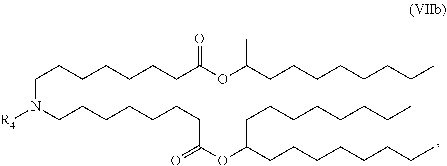

or their N-oxides, or salts or isomers thereof, wherein R₄ is as described herein.

In another embodiment, the compounds of Formula (VI) are of Formula (VIIc) or (VIIe):

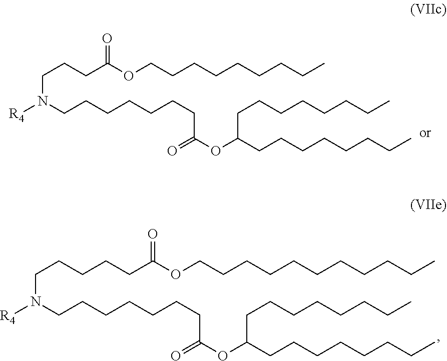

or their N-oxides, or salts or isomers thereof, wherein R₄ is as described herein.

In another embodiment, the compounds of Formula (VI) are of Formula (VIIf):

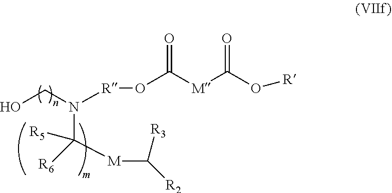

or their N-oxides, or salts or isomers thereof,

wherein M is —C(O)O— or —OC(O)—, M″ is C₁₋₆ alkyl or C₂₋₆ alkenyl, R₂ and R₃ are independently selected from the group consisting of C₅₋₁₄ alkyl and C₅₋₁₄ alkenyl, and n is selected from 2, 3, and 4.

In a further embodiment, the compounds of Formula (VI) are of Formula (VIId),

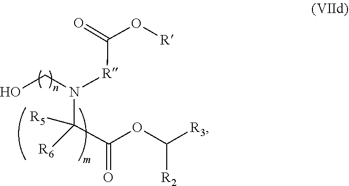

or their N-oxides, or salts or isomers thereof, wherein n is 2, 3, or 4; and m, R′, R″, and R₂ through R₆ are as described herein. For example, each of R₂ and R₃ may be independently selected from the group consisting of C₅₋₁₄ alkyl and C₅₋₁₄ alkenyl.

In some embodiments, an ionizable amino lipid of the disclosure comprises a compound having structure:

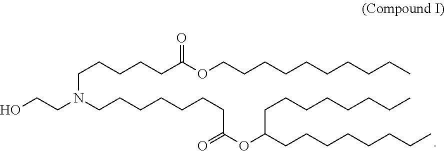 [SMILES: CCCCCCCCCCOC(=O)CCCCCN(CCO)CCCCCCCC(=O)OC(CCCCCCCC)CCCCCCCC]

In some embodiments, an ionizable amino lipid of the disclosure comprises a compound having structure:

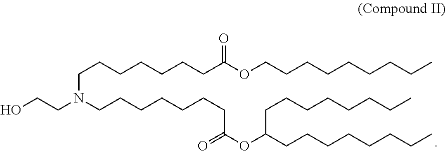 [SMILES: CCCCCCCCCOC(=O)CCCCCCCN(CCO)CCCCCCCC(=O)OC(CCCCCCCC)CCCCCCCC]

In a further embodiment, the compounds of Formula (VI) are of Formula (VIIg),

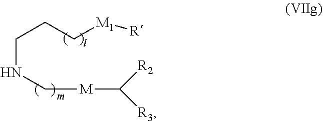

or their N-oxides, or salts or isomers thereof, wherein 1 is selected from 1, 2, 3, 4, and 5; m is selected from 5, 6, 7, 8, and 9; M₁ is a bond or M′; M and M′ are independently selected from —C(O)O—, —OC(O)—, —OC(O)-M″-C(O)O—, —C(O)N(R′)′, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. For example, M″ is C₁₋₆ alkyl (e.g., C₁₋₄ alkyl) or C₂₋₆ alkenyl (e.g. C₂₋₄ alkenyl). For example, R₂ and R₃ are independently selected from the group consisting of C₅₋₁₄ alkyl and C₅₋₁₄ alkenyl.

In some embodiments, the ionizable amino lipids are one or more of the compounds described in U.S. Application Nos. 62/220,091, 62/252,316, 62/253,433, 62/266,460, 62/333,557, 62/382,740, 62/393,940, 62/471,937, 62/471,949, 62/475,140, and 62/475,166, and PCT Application No. PCT/US2016/052352.

The central amine moiety of a lipid according to Formula (VI), (VI-A), (VI-B), (VII), (VIIa), (VIIb), (VIIc), (VIId), (VIIe), (VIIf), or (VIIg) may be protonated at a physiological pH. Thus, a lipid may have a positive or partial positive charge at physiological pH. Such amino lipids may be referred to as cationic lipids, ionizable lipids, cationic amino lipids, or ionizable amino lipids. Amino lipids may also be zwitterionic, i.e., neutral molecules having both a positive and a negative charge.

In some embodiments, the ionizable amino lipids of the present disclosure may be one or more of compounds of formula (VIII),

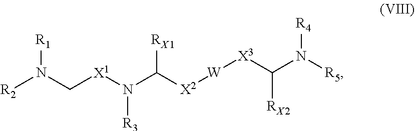

or salts or isomers thereof, wherein
W is

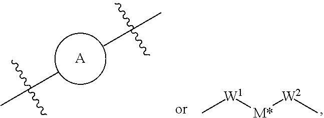

ring A is

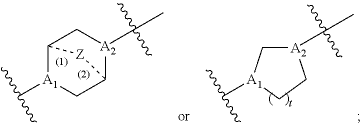

t is 1 or 2;
A₁ and A₂ are each independently selected from CH or N;
Z is CH₂ or absent wherein when Z is CH₂, the dashed lines (1) and (2) each represent a single bond; and when Z is absent, the dashed lines (1) and (2) are both absent;
R₁, R₂, R₃, R₄, and R₅ are independently selected from the group consisting of C₅₋₂₀ alkyl, C₅₋₂₀ alkenyl, —R″MR′, —R*YR″, —YR″, and —R*OR″;
R_{X1} and R_{X2} are each independently H or C₁₋₃ alkyl;
each M is independently selected from the group consisting of —C(O)O—, —OC(O)—, —OC(O)O—, —C(O)N(R′)′, —N(R′)C(O)—, —C(O)—, —C(S)—, —C(S)S—, —SC(S)—, —CH(OH)—, —P(O)(OR′)O—, —S(O)₂—, —C(O)S—, —SC(O)—, an aryl group, and a heteroaryl group;
M* is C₁-C₆ alkyl,
W¹ and W² are each independently selected from the group consisting of —O— and —N(R₆)—;
each R₆ is independently selected from the group consisting of H and C₁₋₅ alkyl;
X¹, X², and X³ are independently selected from the group consisting of a bond, —CH₂—, —(CH₂)₂—, —CHR—, —CHY—, —C(O)—, —C(O)O—, —OC(O)—, —(CH₂)ₙ—C(O)—, —C(O)—(CH₂)ₙ—, —(CH₂)ₙ—C(O)O—, —OC(O)—(CH₂)ₙ—, —(CH₂)ₙ—OC(O)—, —C(O)O—(CH₂)ₙ—, —CH(OH)—, —C(S)—, and —CH(SH)—;
each Y is independently a C₃₋₆ carbocycle;
each R* is independently selected from the group consisting of C₁₋₁₂ alkyl and C₂₋₁₂ alkenyl;
each R is independently selected from the group consisting of C₁₋₃ alkyl and a C₃₋₆ carbocycle;
each R′ is independently selected from the group consisting of C₁₋₁₂ alkyl, C₂₋₁₂ alkenyl, and H;
each R″ is independently selected from the group consisting of C₃₋₁₂ alkyl, C₃₋₁₂ alkenyl and —R*MR′; and
n is an integer from 1-6,
wherein when ring A is

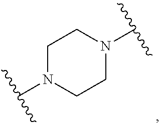 [SMILES: *N1CCN(*)CC1]

then
i) at least one of X¹, X², and X³ is not —CH₂—; and/or
ii) at least one of R₁, R₂, R₃, R₄, and R₅ is —R″MR′.

In some embodiments, the compound is of an of formulae (VIIIa1)-(VIIIa8):

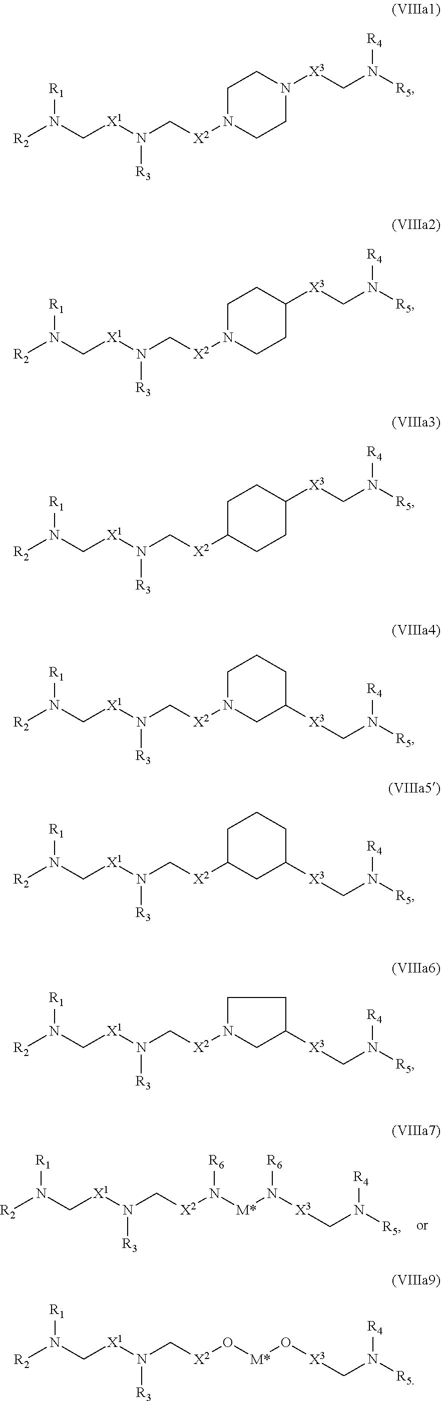

In some embodiments, the ionizable amino lipid is

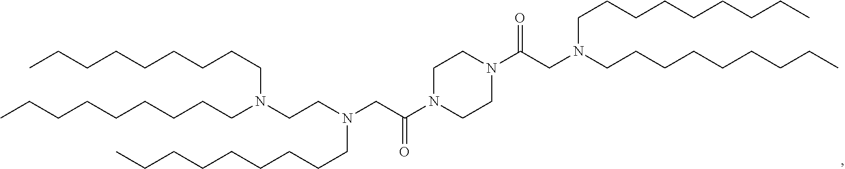 [SMILES: CCCCCCCCCN(CCCCCCCCC)CCN(CCCCCCCCC)CC(=O)N1CCN(C(=O)CN(CCCCCCCCC)CCCCCCCCC)CC1]

or a salt thereof.

The central amine moiety of a lipid according to Formula (VIII), (VIIIa1), (VIIIa2), (VIIIa3), (VIIIa4), (VIIIa5), (VIIIa6), (VIIIa7), or (VIIIa8) may be protonated at a physiological pH. Thus, a lipid may have a positive or partial positive charge at physiological pH.

In some embodiments, the lipid nanoparticle comprises a lipid having the structure:

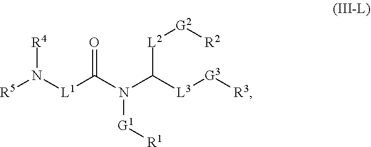

or a pharmaceutically acceptable salt, tautomer, or stereoisomer thereof, wherein:
R¹ is optionally substituted C₁-C₂₄ alkyl or optionally substituted C₂-C₂₄ alkenyl;
R² and R³ are each independently optionally substituted C₁-C₃₆ alkyl;
R⁴ and R⁵ are each independently optionally substituted C₁-C₆ alkyl, or R⁴ and R⁵ join, along with the N to which they are attached, to form a heterocyclyl or heteroaryl;
L¹, L², and L³ are each independently optionally substituted C₁-C₁₈ alkylene;
G¹ is a direct bond, —(CH₂)ₙO(C═O)—, —(CH₂)ₙ(C═O)O—, or —(C═O)—;
G² and G³ are each independently —(C═O)O— or —O(C═O)—; and n is an integer greater than 0.

In some embodiments, the lipid nanoparticle comprises a lipid having the structure:

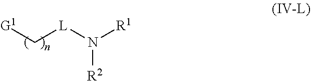

or a pharmaceutically acceptable salt, tautomer, or stereoisomer thereof, wherein:
G¹ is —N(R³)R⁴ or —OR⁵—;
R¹ is optionally substituted branched, saturated or unsaturated C₁₂-C₃₆ alkyl;
R² is optionally substituted branched or unbranched, saturated or unsaturated C₁₂-C₃₆ alkyl when L is —C(═O)—; or R² is optionally substituted branched or unbranched, saturated or unsaturated C₄-C₃₆ alkyl when L is C₆-C₁₂ alkylene, C₆-C₁₂ alkenylene, or C₂-C₆ alkynylene;
R³ and R⁴ are each independently H, optionally substituted branched or unbranched, saturated or unsaturated C₁-C₆ alkyl; or R³ and R⁴ are each independently optionally substituted branched or unbranched, saturated or unsaturated C₁-C₆ alkyl when L is C₆-C₁₂ alkylene, C₆-C₁₂ alkenylene, or C₂-C₆ alkynylene; or R³ and R⁴, together with the nitrogen to which they are attached, join to form a heterocyclyl;
R⁵ is H or optionally substituted C₁-C₆ alkyl;
L is —C(═O)—, C₆-C₁₂ alkylene, C₆-C₁₂ alkenylene, or C₂-C₆ alkynylene; and
n is an integer from 1 to 12.

In some embodiments, the lipid nanoparticle comprises a lipid having the structure:

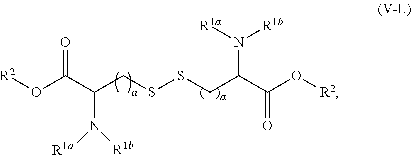

or a pharmaceutically acceptable salt thereof, wherein:
each R^{1a} is independently hydrogen, R^{1c}, or R^{1d};
each R^{1b} is independently R^{1c} or R^{1d};
each R^{1c} is independently —[CH₂]₂C(O)X¹R³;
each R^{1d} Is independently —C(O)R⁴;
each R² is independently —[C(R^{2a})₂]_{c}R^{2b};
each R^{2a} is independently hydrogen or C₁-C₆ alkyl;
R^{2b} is —N(L₁-B)₂; —(OCH₂CH₂)₆OH, or —(OCH₂CH₂)_{b}OCH₃;
each R³ and R⁴ is independently C₆-C₃₀ aliphatic;
each L₃ is independently C₁-C₁₀ alkylene;
each B is independently hydrogen or an ionizable nitrogen-containing group;
each X¹ is independently a covalent bond or O;
each a is independently an integer of 1-10;
each b is independently an integer of 1-10; and
each c is independently an integer of 1-10.

In some embodiments, the lipid nanoparticle comprises a lipid having the structure:

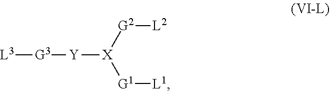

or a pharmaceutically acceptable salt, prodrug or stereoisomer thereof, wherein:
X is N, and Y is absent; or X is CR, and Y is NR;
L¹ is —O(C—O)R¹, —(C═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c}, or —NR^{a}C(═O)OR¹;
L² is —O(C═O)R², —(C═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R²;
L³ is —O(C═O)R³ or —(C═O)OR³;
G¹ and G² are each independently C₂-C₁₂ alkylene or C₂-C₁₂ alkenylene;
G³ is C₁-C₂₄ alkylene, C₂-C₂₄ alkenylene, C₁-C₂₄ heteroalkylene or C₂-C₂₄ heteroalkenylene when X is CR, and Y is NR; and G³ is C₁-C₂₄ heteroalkylene or C₂-C₂₄ heteroalkenylene when X is N, and Y is absent;
R^{a}, R^{b}, R^{d} and R^{e} are each independently H or C₁-C₁₂ alkyl or C₁-C₁₂ alkenyl;
R^{c} and R^{f} are each independently C₁-C₁₂ alkyl or C₂-C₁₂ alkenyl;
each R is independently H or Ci-Cu alkyl;
R¹, R² and R³ are each independently C₁-C₂₄ alkyl or C₂-C₂₄ alkenyl; and x is 0, 1 or 2, and
wherein each alkyl, alkenyl, alkylene, alkenylene, heteroalkylene and heteroalkenylene is independently substituted or unsubstituted unless otherwise specified.

In some embodiments, the lipid nanoparticle comprises a lipid having the structure:

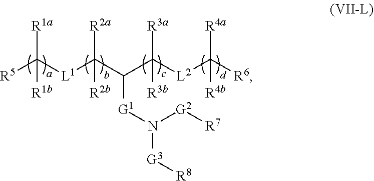

or a pharmaceutically acceptable salt, tautomer, prodrug or stereoisomer thereof, wherein:
L¹ and L² are each independently —O(C═O)—, —(C═O)O—, —C(═O)—, —O—, —S(O)x-ₛ-S—S—, —C(═O)S—, —SC(═O)—, —NR^{a}C(═O)—, —C(═O)NR^{a}—, —NR^{a}C(═O)NR^{a}—, —OC(═O)NR^{a}—, —NR^{a}C(═O)O— or a direct bond;
G¹ is C, —C₂ alkylene, —(C═O)—, —O(C═O)—, —SC(═O)—, —NR^{a}C(═O)— or a direct bond;
G² is —C(O)—, —(CO)O—, —C(═O)S—, —C(═O)NR^{a}— or a direct bond;
G³ is C₁-C₆ alkylene;
R^{a} is H or C₁-C₁₂ alkyl;
R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a): H or C₁-C₁₂ alkyl; or (b) R^{3a} is H or C₁-C₁₂ alkyl, and R^{3b} together with the carbon atom to which it is bound is taken together with an adjacent R and the carbon atom to which it is bound to form a carbon-carbon double bond;
R^{4A} and R^{4B} are, at each occurrence, independently either: (a) H or C₁-C₁₂ alkyl; or (b) R^{4A} is H or C₁-C₁₂ alkyl, and R^{4B} together with the carbon atom to which it is bound is taken together with an adjacent R^{4B} and the carbon atom to which it is bound to form a carbon-carbon double bond;
R⁵ and R⁶ are each independently H or methyl;
R⁷ is H or C, —C₂₀ alkyl;
R⁸ is OH, —N(R⁹)(C═O)R¹⁰, —(C═O)NR⁹R¹⁰, —NR⁹R¹⁰, —(C═O)OR″¹ or —O(C═O)R″, provided that G³ is C₄-C₆ alkylene when R⁸ is —NR⁹R¹⁰,
R⁹ and R¹⁰ are each independently H or C₁-C₁₂ alkyl;
R″ is aralkyl;
a, b, c and d are each independently an integer from 1 to 24; and x is 0, 1 or 2,
wherein each alkyl, alkylene and aralkyl is optionally substituted.

In some embodiments, the lipid nanoparticle comprises a lipid having the structure:

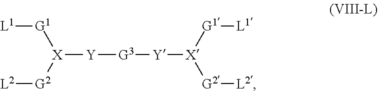

or a pharmaceutically acceptable salt, prodrug or stereoisomer thereof, wherein:
X and X′ are each independently N or CR;
Y and Y′ are each independently absent, —O(C═O)—, —(C═O)O— or NR, provided that:
   a) Y is absent when X is N;
   b) Y′ is absent when X is N;
   c) Y is —O(C═O)—, —(C═O)O— or NR when X is CR; and
   d) Y′ is —O(C═O)—, —(C═O)O— or NR when X is CR,
L¹ and L^{1′} are each independently —O(C═O)R′, —(C═O)OR′, —C(═O)R′, —ORI, —S(O)_{z}R′, —S—SR¹, —C(═O)SR′, —SC(═O)R′, —NR^{a}C(═O)R′, —C(═O)NR^{b}R^{c}, —NR^{a}C(═O)NR^{b}R^{c}, —OC(═O)NR^{b}R^{c} or —NR^{a}C(═O)OR′;
L² and L^{2′} are each independently —O(C═O)R², —(C═O)OR², —C(═O)R², —OR², —S(O)_{z}R², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R²;
G¹, G^{1′}, G² and G^{2′} are each independently C₂-C₁₂ alkylene or C₂-C₁₂ alkenylene; G is C₂-C₂₄ heteroalkylene or C₂-C₂₄ heteroalkenylene;
R^{a}, R^{b}, R^{d} and R^{e} are, at each occurrence, independently H, C₁-C₁₂ alkyl or C₂-C₁₂alkenyl;
R^{e} and R^{f} are, at each occurrence, independently C₁-C₁₂ alkyl or C₂-C₁₂ alkenyl;
R is, at each occurrence, independently H or C₁-C₁₂ alkyl;
R¹ and R² are, at each occurrence, independently branched C₆-C₂₄ alkyl or branched C₆-C₂₄ alkenyl;
z is 0, 1 or 2, and wherein each alkyl, alkenyl, alkylene, alkenylene, heteroalkylene and heteroalkenylene is independently substituted or unsubstituted unless otherwise specified.

In some embodiments, the lipid nanoparticle comprises a lipid having the structure:

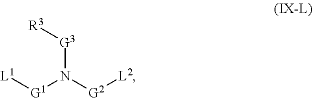

or a pharmaceutically acceptable salt, prodrug or stereoisomer thereof, wherein:
L¹ is —O(C═O)R¹, —(C═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c} or —NR^{a}C(═O)OR¹;
L² is —O(C═O)R², —(C═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R²;
G¹ and G² are each independently C₂-C₁₂ alkylene or C₂-C₁₂ alkenylene;
G³ is C₁-C₂₄ alkylene, C₂-C₂₄ alkenylene, C₃-C₈ cycloalkylene or C₃-C₈ cycloalkenylene;
R^{a}, R^{b}, R^{d} and R^{e} are each independently H or C₁-C₁₂ alkyl or C₁-C₁₂ alkenyl;
R^{e} and R^{f} are each independently C₁-C₁₂ alkyl or C₂-C₁₂ alkenyl;
R¹ and R² are each independently branched C₆-C₂₄ alkyl or branched C₆-C₂₄ alkenyl;
R³ is —N(R⁴)R⁵;
R⁴ is C₁-C₁₂ alkyl;
R⁵ is substituted C₁-C₁₂ alkyl; and
x is 0, 1 or 2, and
wherein each alkyl, alkenyl, alkylene, alkenylene, cycloalkylene, cycloalkenylene, aryl and aralkyl is independently substituted or unsubstituted unless otherwise specified.

In some embodiments, the lipid nanoparticle comprises a lipid having the structure:

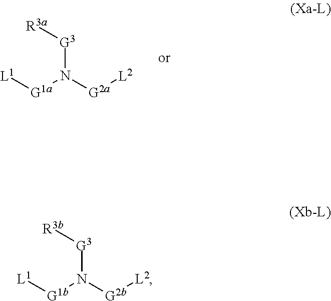

or a pharmaceutically acceptable salt, prodrug or stereoisomer thereof, wherein:
L¹ is —O(C═O)R¹, —(C═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c} or —NR^{a}C(═O)OR¹;
L² is —O(C═O)R², —(C═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R²;
G^{1a} and G^{2b} are each independently C₂-C₁₂ alkylene or C₂-C₁₂ alkenylene;
G^{1b} and G^{2b} are each independently C₁-C₁₂ alkylene or C₂-C₁₂ alkenylene;
G³ is C₁-C₂₄ alkylene, C₂-C₂₄ alkenylene, C₃-C₈ cycloalkylene or C₃-C₈ cycloalkenylene;
R^{a}, R^{b}, R^{d} and R^{e} are each independently H or C₁-C₁₂ alkyl or C₂-C₁₂ alkenyl;
R^{c} and R^{f} are each independently C₁-C₁₂ alkyl or C₂-C₁₂ alkenyl;
R¹ and R² are each independently branched C₆-C₂₄ alkyl or branched C₆-C₂₄ alkenyl;
R^{3a} is —C(═O)N(R^{4a})R^{5a} or —C(═O)OR⁶;
R^{3b} is —NR^{4b}C(═O)R^{5b};
R^{4a} is C₁-C12 alkyl;
R^{4b} is H, C₁-C₁₂ alkyl or C₂-C₁₂ alkenyl;
R^{5a} is H, C₁-C₈ alkyl or C₂-C₈ alkenyl;
R^{5b} is C₂-C₁₂ alkyl or C₂-C₁₂ alkenyl when R^{4b} is H; or R^{5b} is C₁-C₁₂ alkyl or C₂-C₁₂ alkenyl when R^{4b} is C₁-C₁₂ alkyl or C₂-C₁₂ alkenyl;
R⁶ is H, aryl or aralkyl; and
x is 0, 1 or 2, and
wherein each alkyl, alkenyl, alkylene, alkenylene, cycloalkylene, cycloalkenylene, aryl and aralkyl is independently substituted or unsubstituted.

In some embodiments, the lipid nanoparticle comprises a lipid having the structure:

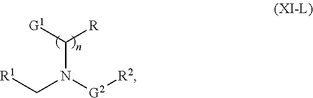

or a pharmaceutically acceptable salt, prodrug or stereoisomer thereof, wherein:
G¹ is —OH, —R³R⁴, —(C═O)R⁵ or —R³(C═O)R⁵;
G² is —CH₂— or —(C═O)—;
R is, at each occurrence, independently H or OH;
R¹ and R² are each independently optionally substituted branched, saturated or unsaturated C₁₂-C₃₆ alkyl;
R³ and R⁴ are each independently H or optionally substituted straight or branched, saturated or unsaturated C₁-C₆ alkyl;
R⁵ is optionally substituted straight or branched, saturated or unsaturated C₁-C₆ alkyl; and
n is an integer from 2 to 6.

In some embodiments, the lipid nanoparticle comprises a lipid having the structure:

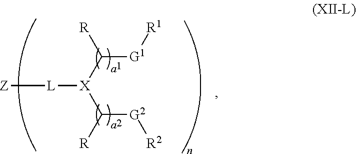

or a pharmaceutically acceptable salt, prodrug or stereoisomer thereof, wherein:
one of G¹ or G² is, at each occurrence, —O(C═O)—, —(C═O)O—, —C(═O)—, —O—, —S(O), —S—S—, —C(═O)S—, SC(═O)—, —N(R^{a})C(═O)—, —C(═O)N(R³)—, —N(R³)C(═O)N(R′)′, —OC(═O)N(R^{a})— or —N(R^{a})C(═O)O—, and the other of G¹ or G² is, at each occurrence, —O(C═O)—, —(C═O)O—, —C(═O)—, —O—, —S(O), —S—S—, —C(═O)S—, —SC(═O)—, —N(R^{a})C(═O)—, —C(═O)N(R^{a})—, —N(R)C(═O)N(R^{a})—, —OC(═O)N(R^{a})— or —N(R^{a})C(═O)O— or a direct bond;
L is, at each occurrence, ˜O(C═O)—, wherein ˜ represents a covalent bond to X;

### X is CR^{a};

Z is alkyl, cycloalkyl or a monovalent moiety comprising at least one polar functional group when n is 1; or Z is alkylene, cycloalkylene or a polyvalent moiety comprising at least one polar functional group when n is greater than 1;
R^{a} is, at each occurrence, independently H, C₁-C₁₂ alkyl, C₁-C₁₂ hydroxylalkyl, C₁-C₁₂ aminoalkyl, C₁-C₁₂ alkylaminylalkyl, C₁-C₁₂ alkoxyalkyl, C₁-C₁₂ alkoxycarbonyl, C₁-C₁₂ alkylcarbonyloxy, C₁-C₁₂ alkylcarbonyloxyalkyl or C₁-C₁₂ alkylcarbonyl;
R is, at each occurrence, independently either: (a) H or C₁-C₁₂ alkyl; or (b) R together with the carbon atom to which it is bound is taken together with an adjacent R and the carbon atom to which it is bound to form a carbon-carbon double bond;
R¹ and R² have, at each occurrence, the following structure, respectively:

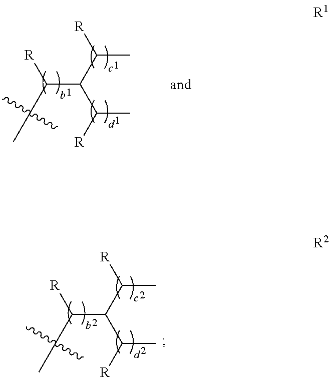

a¹ and a² are, at each occurrence, independently an integer from 3 to 12; b¹ and b² are, at each occurrence, independently 0 or 1;
c¹ and c² are, at each occurrence, independently an integer from 5 to 10; d¹ and d² are, at each occurrence, independently an integer from 5 to 10; y is, at each occurrence, independently an integer from 0 to 2; and n is an integer from 1 to 6,
wherein each alkyl, alkylene, hydroxylalkyl, aminoalkyl, alkylaminylalkyl, alkoxyalkyl, alkoxycarbonyl, alkylcarbonyloxy, alkylcarbonyloxyalkyl and alkylcarbonyl is optionally substituted with one or more substituent.

In some embodiments, the lipid nanoparticle comprises a lipid having the structure:

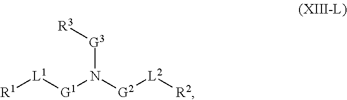

or a pharmaceutically acceptable salt, prodrug or stereoisomer thereof, wherein:
one of L¹ or L² is —O(C═O)—, —(C═O)O—, —C(═O)—, —O—, —S(O)ₓ—, —S—S—, —C(═O)S—, —SC(═O)—, —R^{a}C(═O)—, —C(═O)R^{a}—, R^{a}C(═O)R^{a}—, —OC(═O)R^{a}— or —R^{a}C(═O)O—, and the other of L¹ or L² is —O(C═O)—, —(C═O)O—, —C(═O)—, —O—, —S(O)ₓ—, —S—S—, —C(═O)S—, SC(═O)—, —R^{a}C(═O)—, —C(═O)R^{a}—, R^{a}C(═O)R^{a}—, —OC(═O)R^{a}— or —NR^{a}C(═O)O— or a direct bond;
G¹ and G² are each independently unsubstituted C₁-C₁₂ alkylene or C₁-C₂ alkenylene;
G³ is C₁-C₂₄ alkylene, C₁-C₂₄ alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR⁵, CN, —C(═O)OR⁴, —OC(═O)R⁴ or —R⁵C(═O)R⁴;
R⁴ is C₁-C₁₂ alkyl;
R⁵ is H or C₁-C₆ alkyl; and
x is 0, 1 or 2.

In some embodiments, the lipid nanoparticle comprises a lipid having the structure:

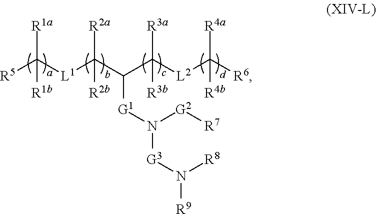

or a pharmaceutically acceptable salt, tautomer, prodrug or stereoisomer thereof, wherein:
L¹ and L² are each independently —O(C═O)—, —(C═O)O—, —C(═O)—, —O—, —S(O)ₓ—, —S—S—, —C(═O)S—, —SC(═O)—, —R^{a}C(═O)—, —C(═O)R^{a}—, —R^{a}C(═O)R^{a}—, —OC(═O)R^{a}—, —R^{a}C(═O)O— or a direct bond;
G¹ is C₁-C₂ alkylene, —(C═O)—, —O(C═O)—, —SC(═O)—, —R^{a}C(═O)— or a direct bond;
G² is —C(═O)—, —(C═O)O—, —C(═O)S—, —C(═O)NR^{a}— or a direct bond;
G³ is C₁-C₆ alkylene;
R^{a} is H or C₁-C₂ alkyl;
R^{1a} and R^{1b} are, at each occurrence, independently either: (a) H or C₁-C₁₂ alkyl; or (b) R^{1a} is H or C₁-C₁₂ alkyl, and Rib together with the carbon atom to which it is bound is taken together with an adjacent R^{1b} and the carbon atom to which it is bound to form a carbon-carbon double bond;
R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a): H or C₁-C₁₂ alkyl; or (b) R^{3a} is H or C₁-C₁₂ alkyl, and R^{3b} together with the carbon atom to which it is bound is taken together with an adjacent R and the carbon atom to which it is bound to form a carbon-carbon double bond;
R^{4a} and R^{4b} are, at each occurrence, independently either: (a) H or C₁-C₁₂ alkyl; or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently H or methyl;
R⁷ is C₄-C₂₀ alkyl;
R⁸ and R⁹ are each independently C₁-C₁₂ alkyl; or R⁸ and R⁹, together with the nitrogen atom to which they are attached, form a 5, 6 or 7-membered heterocyclic ring;
a, b, c and d are each independently an integer from 1 to 24; and x is 0, 1 or 2.

In some embodiments, the lipid nanoparticle comprises a lipid having the stricture:

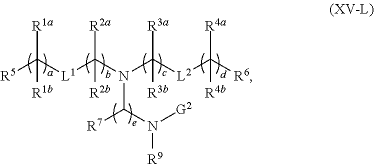

or a pharmaceutically acceptable salt, tautomer, prodrug or stereoisomer thereof, wherein:
L¹ and L² are each independently —O(C═O)—, —(C═O)O— or a carbon-carbon double bond;
R^{1a} and R^{1b} are, at each occurrence, independently either (a) H or C₁-C₁₂ alkyl, or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either (a) H or C₁-C₁₂ alkyl, or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a) H or C₁-C₁₂ alkyl, or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;
R^{4a} and R^{4b} are, at each occurrence, independently either (a) H or C₁-C₁₂ alkyl, or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently methyl or cycloalkyl;
R⁷ is, at each occurrence, independently H or C₁-C₁₂ alkyl; R⁸ and R⁹ are each independently unsubstituted C₁-C₁₂ alkyl; or R⁸ and R⁹, together with the nitrogen atom to which they are attached, form a 5, 6 or 7-membered heterocyclic ring comprising one nitrogen atom;
a and d are each independently an integer from 0 to 24; b and c are each independently an integer from 1 to 24; and e is 1 or 2,
provided that:
at least one of R^{1a}, R^{2a}, R^{3a} or R^{4a} is C₁-C₁₂ alkyl, or at least one of L¹ or L² is —O(C═O)— or —(C═O)O—; and
R^{1a} and R^{1b} are not isopropyl when a is 6 or n-butyl when a is 8.

In some embodiments, the lipid nanoparticle comprises a lipid having the structure:

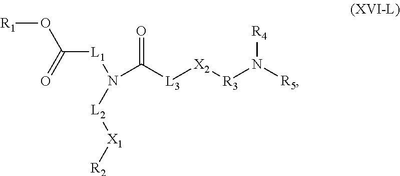

or a pharmaceutically acceptable salt thereof, wherein
R₁ and R₂ are the same or different, each a linear or branched alkyl with 1-9 carbons, or as alkenyl or alkynyl with 2 to 11 carbon atoms,
L₁ and L₂ are the same or different, each a linear alkyl having 5 to 18 carbon atoms, or form a heterocycle with N,
X₁ is a bond, or is -CG-G- whereby L2-CO—O—R₂ is formed,
X₂ is S or O,
L₃ is a bond or a lower alkyl, or form a heterocycle with N,
R₃ is a lower alkyl, and
R₄ and R₅ are the same or different, each a lower alkyl.

In some embodiments, the lipid nanoparticle comprises an ionizable lipid having the structure:

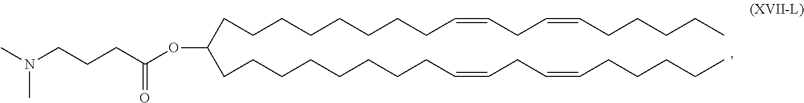 [SMILES: CCCCC/C=C\C/C=C\CCCCCCCCC(CCCCCCCC/C=C\C/C=C\CCCCC)OC(=O)CCCN(C)C]

or a pharmaceutically acceptable salt thereof.

In some embodiments, the lipid nanoparticle comprises a lipid having the structure:

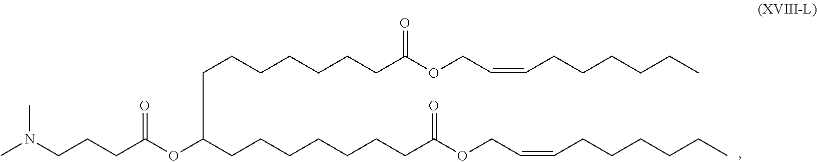 [SMILES: CCCCCC/C=C\COC(=O)CCCCCCCC(CCCCCCCC(=O)OC/C=C\CCCCCC)OC(=O)CCCN(C)C]

acceptable salt thereof.

In some embodiments, the lipid nanoparticle comprises a lipid having the structure:

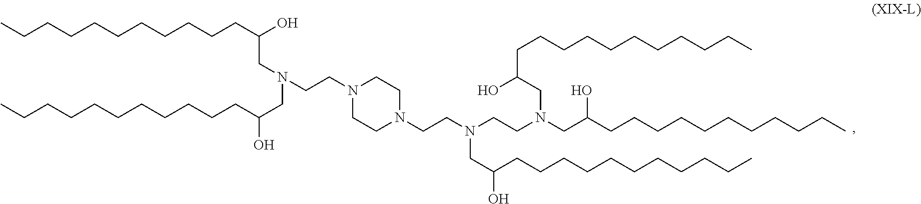 [SMILES: CCCCCCCCCCCC(O)CN(CCN1CCN(CCN(CC(O)CCCCCCCCCCC)CC(O)CCCCCCCCCCC)CC1)CCN(CC(O)CCCCCCCCCCC)CC(O)CCCCCCCCCCC]

or a pharmaceutically acceptable salt thereof.

In some embodiments, the lipid nanoparticle comprises a lipid having the structure:

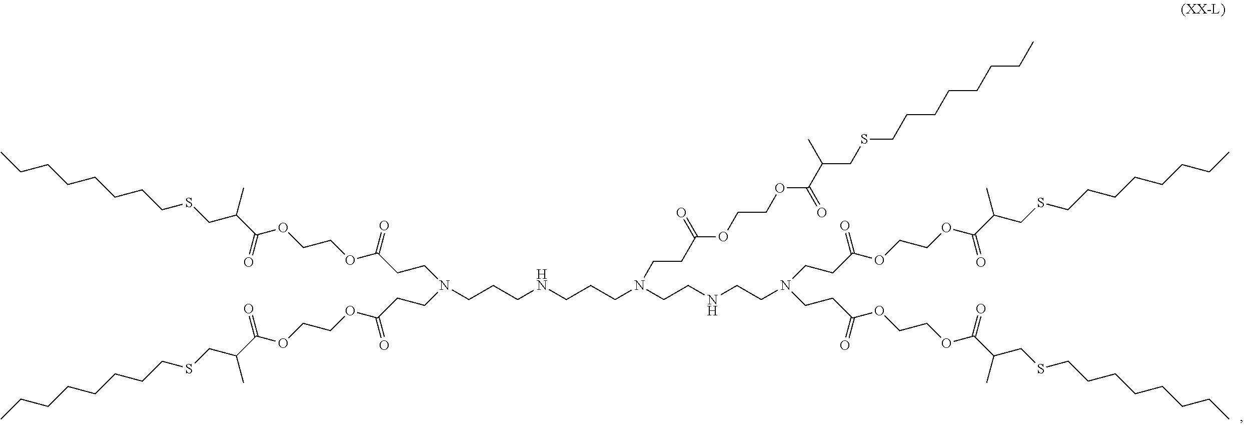 [SMILES: CCCCCCCCSCC(C)C(=O)OCCOC(=O)CCN(CCCNCCCN(CCC(=O)OCCOC(=O)C(C)CSCCCCCCCC)CCC(=O)OCCOC(=O)C(C)CSCCCCCCCC)CCNCCN(CCC(=O)OCCOC(=O)C(C)CSCCCCCCCC)CCC(=O)OCCOC(=O)C(C)CSCCCCCCCC]

L), or a pharmaceutically acceptable salt thereof.

In some embodiments, the lipid nanoparticle comprises a lipid having the structure:

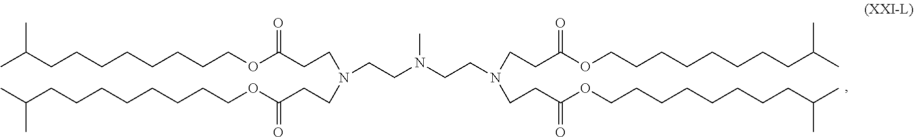 [SMILES: CC(C)CCCCCCCCOC(=O)CCN(CCC(=O)OCCCCCCCCC(C)C)CCN(C)CCN(CCC(=O)OCCCCCCCCC(C)C)CCC(=O)OCCCCCCCCC(C)C]

pharmaceutically acceptable salt thereof.

In some embodiments, the lipid nanoparticle comprises a lipid having the structure:

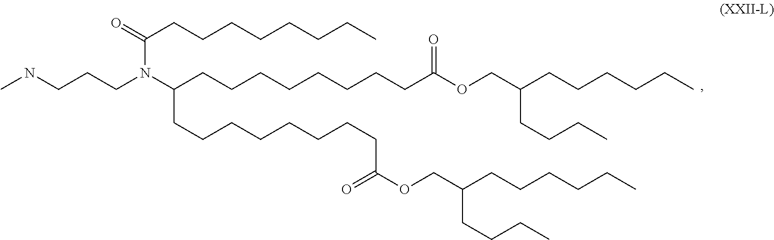 [SMILES: CCCCCCCCC(=O)N(CCCNC)C(CCCCCCCCC(=O)OCC(CCCC)CCCCCC)CCCCCCCCC(=O)OCC(CCCC)CCCCCC]

or a pharmaceutically acceptable salt thereof.

In some embodiments, the lipid nanoparticle comprises a lipid having the structure:

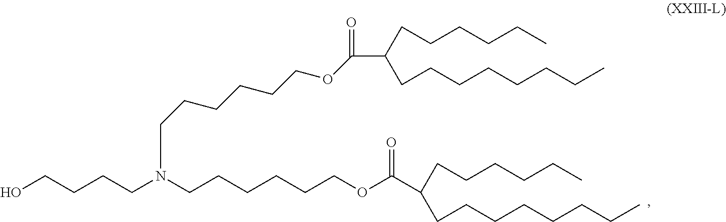 [SMILES: CCCCCCCCC(CCCCCC)C(=O)OCCCCCCN(CCCCO)CCCCCCOC(=O)C(CCCCCC)CCCCCCCC]

or a pharmaceutically acceptable salt thereof.

In some embodiments, the lipid nanoparticle comprises a lipid having the structure:

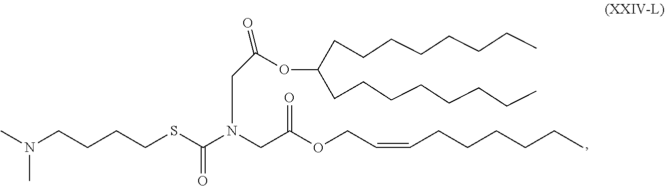 [SMILES: CCCCCC/C=C\COC(=O)CN(CC(=O)OC(CCCCCCCC)CCCCCCCC)C(=O)SCCCCN(C)C]

or a pharmaceutically acceptable salt thereof.

In some embodiments, the lipid nanoparticle comprises a lipid having the structure:

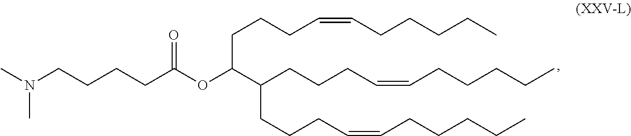 [SMILES: CCCCC/C=C\CCCC(CCC/C=C\CCCCC)C(CCC/C=C\CCCCC)OC(=O)CCCCN(C)C]

or a pharmaceutically acceptable salt thereof.

In some embodiments, the lipid nanoparticle comprises a lipid having the structure:

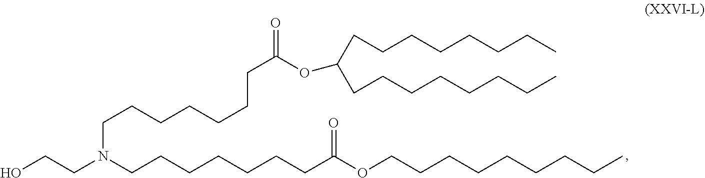 [SMILES: CCCCCCCCCOC(=O)CCCCCCCN(CCO)CCCCCCCC(=O)OC(CCCCCCCC)CCCCCCCC]

or a pharmaceutically acceptable salt thereof.

In some embodiments, the lipid nanoparticle comprises a lipid having the structure:

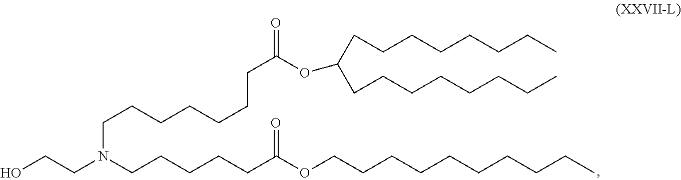 [SMILES: CCCCCCCCCCOC(=O)CCCCCN(CCO)CCCCCCCC(=O)OC(CCCCCCCC)CCCCCCCC]

or a pharmaceutically acceptable salt thereof

### Non-Cationic Lipids

In certain embodiments, the lipid nanoparticles described herein comprise one or more non-cationic lipids. Non-cationic lipids may be phospholipids.

In some embodiments, the lipid nanoparticle comprises 5-25 mol % non-cationic lipid. For example, the lipid nanoparticle may comprise 5-20 mol %, 5-15 mol %, 5-10 mol %, 10-25 mol %, 10-20 mol %, 10-25 mol %, 15-25 mol %, 15-20 mol %, or 20-25 mol % non-cationic lipid. In some embodiments, the lipid nanoparticle comprises 5 mol %, 10 mol %, 15 mol %, 20 mol %, or 25 mol % non-cationic lipid.

In some embodiments, a non-cationic lipid of the disclosure comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, or mixtures thereof.

In some embodiments, the lipid nanoparticle comprises 5-15 mol %, 5-10 mol %, or 10-15 mol % DSPC. For example, the lipid nanoparticle may comprise 5, 6, 7, 8, 9, 10, 11, 12, 13, 14, or 15 mol % DSPC.

In certain embodiments, the lipid composition of the lipid nanoparticle composition disclosed herein can comprise one or more phospholipids, for example, one or more saturated or (poly)unsaturated phospholipids or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

Non-natural phospholipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidyl glycerols, and phosphatidic acids. Phospholipids also include phosphosphingolipid, such as sphingomyelin.

In some embodiments, a phospholipid comprises 1,2-distearoyl-sn-glycero-3-phosphocholine (DSPC), 1,2-Distearoyl-sn-glycero-3-phosphoethanolamine (DSPE), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, or mixtures thereof.

In certain embodiments, a phospholipid is an analog or variant of DSPC. In certain embodiments, a phospholipid is a compound of Formula (IX):

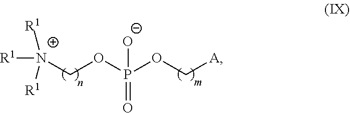

or a salt thereof, wherein:
each R¹ is independently optionally substituted alkyl; or optionally two R¹ are joined together with the intervening atoms to form optionally substituted monocyclic carbocyclyl or optionally substituted monocyclic heterocyclyl; or optionally three R¹ are joined together with the intervening atoms to form optionally substituted bicyclic carbocyclyl or optionally substitute bicyclic heterocyclyl;
n is 1, 2, 3, 4, 5, 6, 7, 8, 9, or 10;
m is 0, 1, 2, 3, 4, 5, 6, 7, 8, 9, or 10;
A is of the formula: or

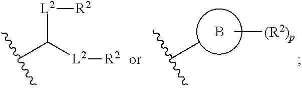

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), —NR^{N}C(O)O, or NR^{N}C(O)N(R^{N});
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), —OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), S(O)O, —OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or —N(R^{N})S(O)₂O;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2;
provided that the compound is not of the formula:

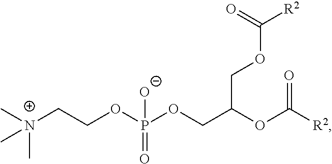

wherein each instance of R² is independently unsubstituted alkyl, unsubstituted alkenyl, or unsubstituted alkynyl.

In some embodiments, the phospholipids may be one or more of the phospholipids described in PCT Application No. PCT/US2018/037922.

In some embodiments, the lipid nanoparticle comprises a molar ratio of 5-25% non-cationic lipid relative to the other lipid components. For example, the lipid nanoparticle may comprise a molar ratio of 5-30%, 5-15%, 5-10%, 10-25%, 10-20%, 10-25%, 15-25%, 15-20%, 20-25%, or 25-30% non-cationic lipid. In some embodiments, the lipid nanoparticle comprises a molar ratio of 5%, 10%, 15%, 20%, 25%, or 30% non-cationic lipid.

In some embodiments, the lipid nanoparticle comprises a molar ratio of 5-25% phospholipid relative to the other lipid components. For example, the lipid nanoparticle may comprise a molar ratio of 5-30%, 5-15%, 5-10%, 10-25%, 10-20%, 10-25%, 15-25%, 15-20%, 20-25%, or 25-30% phospholipid. In some embodiments, the lipid nanoparticle comprises a molar ratio of 5%, 10%, 15%, 20%, 25%, or 30% phospholipid lipid.

### Structural Lipids

The lipid composition of a pharmaceutical composition disclosed herein can comprise one or more structural lipids. As used herein, the term “structural lipid” includes sterols and also to lipids containing sterol moieties.

Incorporation of structural lipids in the lipid nanoparticle may help mitigate aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In some embodiments, the structural lipids may be one or more of the structural lipids described in U.S. application Ser. No. 16/493,814.

In some embodiments, the lipid nanoparticle comprises a molar ratio of 25-55% structural lipid relative to the other lipid components. For example, the lipid nanoparticle may comprise a molar ratio of 10-55%, 25-50%, 25-45%, 25-40%, 25-35%, 25-30%, 30-55%, 30-50%, 30-45%, 30-40%, 30-35%, 35-55%, 35-50%, 35-45%, 35-40%, 40-55%, 40-50%, 40-45%, 45-55%, 45-50%, or 50-55% structural lipid. In some embodiments, the lipid nanoparticle comprises a molar ratio of 10%, 15%, 20%, 25%, 30%, 35%, 40%, 45%, 50%, or 55% structural lipid.

In some embodiments, the lipid nanoparticle comprises 30-45 mol % sterol, optionally 35-40 mol %, for example, 30-31 mol %, 31-32 mol %, 32-33 mol %, 33-34 mol %, 35-35 mol %, 35-36 mol %, 36-37 mol %, 38-38 mol %, 38-39 mol %, or 39-40 mol %. In some embodiments, the lipid nanoparticle comprises 25-55 mol % sterol. For example, the lipid nanoparticle may comprise 25-50 mol %, 25-45 mol %, 25-40 mol %, 25-35 mol %, 25-30 mol %, 30-55 mol %, 30-50 mol %, 30-45 mol %, 30-40 mol %, 30-35 mol %, 35-55 mol %, 35-50 mol %, 35-45 mol %, 35-40 mol %, 40-55 mol %, 40-50 mol %, 40-45 mol %, 45-55 mol %, 45-50 mol %, or 50-55 mol % sterol. In some embodiments, the lipid nanoparticle comprises 25 mol %, 30 mol %, 35 mol %, 40 mol %, 45 mol %, 50 mol %, or 55 mol % sterol.

In some embodiments, the lipid nanoparticle comprises 35-40 mol % cholesterol. For example, the lipid nanoparticle may comprise 35, 35.5, 36, 36.5, 37, 37.5, 38, 38.5, 39, 39.5, or 40 mol % cholesterol.

### Polyethylene Glycol (PEG)-Lipids

The lipid composition of a pharmaceutical composition disclosed herein can comprise one or more polyethylene glycol (PEG) lipids.

As used herein, the term “PEG-lipid” or “PEG-modified lipid” refers to polyethylene glycol (PEG)-modified lipids. Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines, and PEG-modified 1,2-diacyloxypropan-3-amines. Such lipids are also referred to as PEGylated lipids. For example, a PEG lipid can be PEG-c-DOMG, PEG-DMG, PEG-DLPE, PEG-DMPE, PEG-DPPC, or a PEG-DSPE lipid.

In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)] (PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In some embodiments, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is PEG-DMG, PEG-c-DOMG (also referred to as PEG-DOMG), PEG-DSG, and/or PEG-DPG.

In some embodiments, the lipid moiety of the PEG-lipids includes those having lengths of from about C₁₄ to about C₂₂, preferably from about C₁₄ to about C₁₆. In some embodiments, a PEG moiety, for example an mPEG-NH₂, has a size of about 1000, 2000, 5000, 10,000, 15,000 or 20,000 daltons. In some embodiments, the PEG-lipid is PEG₂ₖ-DMG.

In some embodiments, the lipid nanoparticles described herein can comprise a PEG lipid which is a non-diffusible PEG. Non-limiting examples of non-diffusible PEGs include PEG-DSG and PEG-DSPE.

PEG-lipids are known in the art, such as those described in U.S. Pat. No. 8,158,601 and International Publ. No. WO 2015/130584 A2, which are incorporated herein by reference in their entirety.

In general, some of the other lipid components (e.g., PEG lipids) of various formulae described herein may be synthesized as described International Patent Application No. PCT/US2016/000129, filed Dec. 10, 2016, entitled “Compositions and Methods for Delivery of Therapeutic Agents,” which is incorporated by reference in its entirety.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments the PEG-modified lipids are a modified form of PEG DMG. PEG-DMG has the following structure:

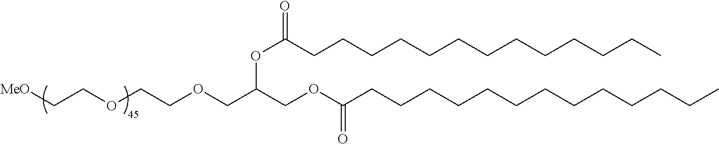

In some embodiments, PEG lipids can be PEGylated lipids described in International Publication No. WO2012099755, the contents of which are herein incorporated by reference in their entirety. Any of these exemplary PEG lipids described herein may be modified to comprise a hydroxyl group on the PEG chain. In certain embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment.

In certain embodiments, a PEG lipid is a compound of Formula (X):

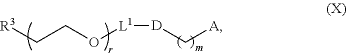

or salts thereof, wherein:
R³ is —OR^{O};
R^{O} is hydrogen, optionally substituted alkyl, or an oxygen protecting group,
r is an integer between 1 and 100, inclusive;
L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, O, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N})
D is a moiety obtained by click chemistry or a moiety cleavable under physiological conditions;
m is 0, 1, 2, 3, 4, 5, 6, 7, 8, 9, or 10;
A is of the formula:

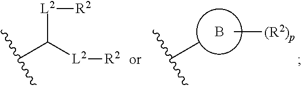

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), —NR^{N}C(O)O, or NR^{N}C(O)N(R^{N});
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), —OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), S(O)O, —OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or —N(R^{N})S(O)₂O;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2.

In certain embodiments, the compound of Formula (X) is a PEG-OH lipid (i.e., R³ is —OR^{O}, and R^{O} is hydrogen). In certain embodiments, the compound of Formula (X) is of Formula (X-OH):

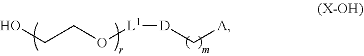

or a salt thereof.

In certain embodiments, a PEG lipid is a PEGylated fatty acid. In certain embodiments, a PEG lipid is a compound of Formula (XI). Provided herein are compounds of Formula (XI):

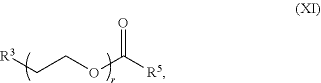

or a salts thereof, wherein:
R³ is —OR^{O};
R^{O} is hydrogen, optionally substituted alkyl or an oxygen protecting group;
r is an integer between 1 and 100, inclusive;
R⁵ is optionally substituted C₁₀₋₄₀ alkyl, optionally substituted C₁₀₋₄₀ alkenyl, or optionally substituted C₁₀₋₄₀ alkynyl; and optionally one or more methylene groups of R⁵ are replaced with optionally substituted carbocyclylene, optionally substituted heterocyclylene, optionally substituted arylene, optionally substituted heteroarylene, N(R^{N}), O, S, C(O), —C(O)N(R^{N}), NR^{N}C(O), NR^{N}C(O)N(R^{N}), C(O)O, OC(O), OC(O)O, OC(O)N(R^{N}), NR^{N}C(O)O, C(O)S, SC(O), C(═NR^{N}), C(═NR^{N})N(R^{N}), NR^{N}C(═NR^{N}) NR^{N}C(═NR^{N})N(R^{N}) C(S), —C(S)N(R^{N}), NR^{N}C(S), NR^{N}C(S)N(R^{N}), S(O), OS(O), S(O)O, OS(O)O, OS(O)₂, S(O)₂O, —OS(O)₂O, N(R^{N})S(O), S(O)N(R^{N}), N(R^{N})S(O)N(R^{N}), OS(O)N(R^{N}), N(R^{N})S(O)O, S(O)₂, —N(R^{N})S(O)₂, S(O)₂N(R^{N}), N(R^{N})S(O)₂N(R^{N}), OS(O)₂N(R^{N}), or N(R^{N})S(O)₂O; and
each instance of R^{N} is independently hydrogen, optionally substituted alkyl, or a nitrogen protecting group.

In certain embodiments, the compound of Formula (XI) is of Formula (XI-OH):

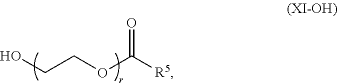

or a salt thereof. In some embodiments, r is 40-50.

In yet other embodiments the compound of Formula (XI) is:

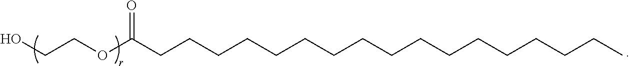

or a salt thereof.

In some embodiments, the compound of Formula (XI) is:

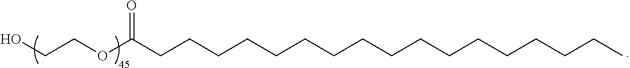

In some embodiments, the lipid composition of the pharmaceutical compositions disclosed herein does not comprise a PEG-lipid.

In some embodiments, the PEG-lipids may be one or more of the PEG lipids described in U.S. Application No. U.S. Ser. No. 15/674,872.

In some embodiments, the lipid nanoparticle comprises a molar ratio of 0.5-15% PEG lipid relative to the other lipid components. For example, the lipid nanoparticle may comprise a molar ratio of 0.5-10%, 0.5-5%, 1-15%, 1-10%, 1-5%, 2-15%, 2-10%, 2-5%, 5-15%, 5-10%, or 10-15% PEG lipid. In some embodiments, the lipid nanoparticle comprises a molar ratio of 0.5%, 1%, 2%, 3%, 4%, 5%, 6%, 7%, 8%, 9%, 10%, 11%, 12%, 13%, 14%, or 15% PEG-lipid.

In some embodiments, the lipid nanoparticle comprises 1-5% PEG-modified lipid, optionally 1-3 mol %, for example 1.5 to 2.5 mol %, 1-2 mol %, 2-3 mol %, 3-4 mol %, or 4-5 mol %. In some embodiments, the lipid nanoparticle comprises 0.5-15 mol % PEG-modified lipid. For example, the lipid nanoparticle may comprise 0.5-10 mol %, 0.5-5 mol %, 1-15 mol %, 1-10 mol %, 1-5 mol %, 2-15 mol %, 2-10 mol %, 2-5 mol %, 5-15 mol %, 5-10 mol %, or 10-15 mol %. In some embodiments, the lipid nanoparticle comprises 0.5 mol %, I mol %, 2 mol %, 3 mol %, 4 mol %, 5 mol %, 6 mol %, 7 mol %, 8 mol %, 9 mol %, 10 mol %, 11 mol %, 12 mol %, 13 mol %, 14 mol %, or 15 mol % PEG-modified lipid.

Some embodiments comprise adding PEG to a composition comprising an LNP encapsulating a nucleic acid (e.g., which already includes PEG in the amounts listed above). Without being bound by theory, it is believed that spiking a LNP composition with additional PEG can provide benefits during lyophilization. Thus, some embodiments, comprise adding additional PEG as compared to an amount used for a non-lyophilized LNP composition. In embodiments comprise adding about 0.5 mol % or more PEG to an LNP composition, such as about 1 mol %, about 1.5 mol %, about 2 mol %, about 2.5 mol %, about 3 mol %, about 3.5 mol %, about 4 mol %, about 5 mol %, or more after formation of an LNP composition (e.g., which already contains PEG in amount listed elsewhere herein).

In some embodiments, the lipid nanoparticle comprises 20-60 mol % ionizable amino lipid, 5-25 mol % non-cationic lipid, 25-55 mol % sterol, and 0.5-15 mol % PEG-modified lipid.

In some embodiments, a LNP of the disclosure comprises an ionizable amino lipid of Compound 1, wherein the non-cationic lipid is DSPC, the structural lipid that is cholesterol, and the PEG lipid is DMG-PEG.

In some embodiments, a LNP comprises an ionizable amino lipid of any of Formula VI, VII or VIIII, a phospholipid comprising DSPC, a structural lipid, and a PEG lipid comprising PEG-DMG.

In some embodiments, a LNP comprises an ionizable amino lipid of any of Formula VI, VII or VIII, a phospholipid comprising DSPC, a structural lipid, and a PEG lipid comprising a compound having Formula XI.

In some embodiments, a LNP comprises an ionizable amino lipid of Formula VI, VII or VIII, a phospholipid comprising a compound having Formula VIII, a structural lipid, and the PEG lipid comprising a compound having Formula X or XI.

In some embodiments, a LNP comprises an ionizable amino lipid of Formula VI, VII or VIII, a phospholipid comprising a compound having Formula IX, a structural lipid, and the PEG lipid comprising a compound having Formula X or XI.

In some embodiments, a LNP comprises an ionizable amino lipid of Formula VI, VII or VIII, a phospholipid having Formula IX, a structural lipid, and a PEG lipid comprising a compound having Formula XI.

In some embodiments, the lipid nanoparticle comprises 49 mol % ionizable amino lipid, 10 mol % DSPC, 38.5 mol % cholesterol, and 2.5 mol % DMG-PEG.

In some embodiments, the lipid nanoparticle comprises 49 mol % ionizable amino lipid, 11 mol % DSPC, 38.5 mol % cholesterol, and 1.5 mol % DMG-PEG.

In some embodiments, the lipid nanoparticle comprises 48 mol % ionizable amino lipid, 11 mol % DSPC, 38.5 mol % cholesterol, and 2.5 mol % DMG-PEG.

In some embodiments, a LNP comprises an N:P ratio of from about 2:1 to about 30:1.

In some embodiments, a LNP comprises an N:P ratio of about 6:1.

In some embodiments, a LNP comprises an N:P ratio of about 3:1, 4:1, or 5:1.

In some embodiments, a LNP comprises a wt/wt ratio of the ionizable amino lipid component to the RNA of from about 10:1 to about 100:1.

In some embodiments, a LNP comprises a wt/wt ratio of the ionizable amino lipid component to the RNA of about 20:1.

In some embodiments, a LNP comprises a wt/wt ratio of the ionizable amino lipid component to the RNA of about 10:1.

Some embodiments comprise a composition having one or more LNPs having a diameter of about 150 nm or less, such as about 140 nm, 130 nm, 120 nm, 110 nm, 100 nm, 90 nm, 80 nm, 70 nm, 60 nm, 50 nm, 40 nm, 30 nm, or 20 nm or less. Some embodiments comprise a composition having a mean LNP diameter of about 150 nm or less, such as about 140 nm, 130 nm, 120 nm, 110 nm, 100 nm, 90 nm, 80 nm, 70 nm, 60 nm, 50 nm, 40 nm, 30 nm, or 20 nm or less. In some embodiments, the composition has a mean LNP diameter from about 30 nm to about 150 nm, or a mean diameter from about 60 nm to about 120 nm.

A LNP may comprise or one or more types of lipids, including but not limited to amino lipids (e.g., ionizable amino lipids), neutral lipids, non-cationic lipids, charged lipids, PEG-modified lipids, phospholipids, structural lipids and sterols. In some embodiments, a LNP may further comprise one or more cargo molecules, including but not limited to nucleic acids (e.g., mRNA, plasmid DNA, DNA or RNA oligonucleotides, siRNA, shRNA, snRNA, snoRNA, lncRNA, etc.), small molecules, proteins and peptides.

In some embodiments, the composition comprises a liposome. A liposome is a lipid particle comprising lipids arranged into one or more concentric lipid bilayers around a central region. The central region of a liposome may comprises an aqueous solution, suspension, or other aqueous composition.

In some embodiments, a lipid nanoparticle may comprise two or more components (e.g., amino lipid and nucleic acid, PEG-lipid, phospholipid, structural lipid). For instance, a lipid nanoparticle may comprise an amino lipid and a nucleic acid. Compositions comprising the lipid nanoparticles, such as those described herein, may be used for a wide variety of applications, including the stealth delivery of therapeutic payloads with minimal adverse innate immune response.

Effective in vivo delivery of nucleic acids represents a continuing medical challenge. Exogenous nucleic acids (i.e., originating from outside of a cell or organism) are readily degraded in the body, e.g., by the immune system. Accordingly, effective delivery of nucleic acids to cells often requires the use of a particulate carrier (e.g., lipid nanoparticles). The particulate carrier should be formulated to have minimal particle aggregation, be relatively stable prior to intracellular delivery, effectively deliver nucleic acids intracellularly, and illicit no or minimal immune response. To achieve minimal particle aggregation and pre-delivery stability, many conventional particulate carriers have relied on the presence and/or concentration of certain components (e.g., PEG-lipid). However, it has been discovered that certain components may decrease the stability of encapsulated nucleic acids (e.g., mRNA molecules). The reduced stability may limit the broad applicability of the particulate carriers. As such, there remains a need for methods by which to improve the stability of nucleic acid (e.g., mRNA) encapsulated within lipid nanoparticles.

In some embodiments, the lipid nanoparticles comprise one or more of ionizable molecules, polynucleotides, and optional components, such as structural lipids, sterols, neutral lipids, phospholipids and a molecule capable of reducing particle aggregation (e.g., polyethylene glycol (PEG), PEG-modified lipid), such as those described above.

In some embodiments, a LNP described herein may include one or more ionizable molecules (e.g., amino lipids or ionizable lipids). The ionizable molecule may comprise a charged group and may have a certain pKa. In certain embodiments, the pKa of the ionizable molecule may be greater than or equal to about 6, greater than or equal to about 6.2, greater than or equal to about 6.5, greater than or equal to about 6.8, greater than or equal to about 7, greater than or equal to about 7.2, greater than or equal to about 7.5, greater than or equal to about 7.8, greater than or equal to about 8. In some embodiments, the pKa of the ionizable molecule may be less than or equal to about 10, less than or equal to about 9.8, less than or equal to about 9.5, less than or equal to about 9.2, less than or equal to about 9.0, less than or equal to about 8.8, or less than or equal to about 8.5. Combinations of the above referenced ranges are also possible (e.g., greater than or equal to 6 and less than or equal to about 8.5). Other ranges are also possible. In embodiments in which more than one type of ionizable molecule are present in a particle, each type of ionizable molecule may independently have a pKa in one or more of the ranges described above.

In general, an ionizable molecule comprises one or more charged groups. In some embodiments, an ionizable molecule may be positively charged or negatively charged. For instance, an ionizable molecule may be positively charged. For example, an ionizable molecule may comprise an amine group. As used herein, the term “ionizable molecule” has its ordinary meaning in the art and may refer to a molecule or matrix comprising one or more charged moiety. As used herein, a “charged moiety” is a chemical moiety that carries a formal electronic charge, e.g., monovalent (+1, or −1), divalent (+2, or −2), trivalent (+3, or −3), etc. The charged moiety may be anionic (i.e., negatively charged) or cationic (i.e., positively charged). Examples of positively-charged moieties include amine groups (e.g., primary, secondary, and/or tertiary amines), ammonium groups, pyridinium group, guanidine groups, and imidazolium groups. In a particular embodiment, the charged moieties comprise amine groups. Examples of negatively-charged groups or precursors thereof, include carboxylate groups, sulfonate groups, sulfate groups, phosphonate groups, phosphate groups, hydroxyl groups, and the like. The charge of the charged moiety may vary, in some cases, with the environmental conditions, for example, changes in pH may alter the charge of the moiety, and/or cause the moiety to become charged or uncharged. In general, the charge density of the molecule and/or matrix may be selected as desired.

In some cases, an ionizable molecule (e.g., an amino lipid or ionizable lipid) may include one or more precursor moieties that can be converted to charged moieties. For instance, the ionizable molecule may include a neutral moiety that can be hydrolyzed to form a charged moiety, such as those described above. As a non-limiting specific example, the molecule or matrix may include an amide, which can be hydrolyzed to form an amine, respectively. Those of ordinary skill in the art will be able to determine whether a given chemical moiety carries a formal electronic charge (for example, by inspection, pH titration, ionic conductivity measurements, etc.), and/or whether a given chemical moiety can be reacted (e.g., hydrolyzed) to form a chemical moiety that carries a formal electronic charge.

The ionizable molecule (e.g., amino lipid or ionizable lipid) may have any suitable molecular weight. In certain embodiments, the molecular weight of an ionizable molecule is less than or equal to about 2,500 g/mol, less than or equal to about 2,000 g/mol, less than or equal to about 1,500 g/mol, less than or equal to about 1,250 g/mol, less than or equal to about 1,000 g/mol, less than or equal to about 900 g/mol, less than or equal to about 800 g/mol, less than or equal to about 700 g/mol, less than or equal to about 600 g/mol, less than or equal to about 500 g/mol, less than or equal to about 400 g/mol, less than or equal to about 300 g/mol, less than or equal to about 200 g/mol, or less than or equal to about 100 g/mol. In some instances, the molecular weight of an ionizable molecule is greater than or equal to about 100 g/mol, greater than or equal to about 200 g/mol, greater than or equal to about 300 g/mol, greater than or equal to about 400 g/mol, greater than or equal to about 500 g/mol, greater than or equal to about 600 g/mol, greater than or equal to about 700 g/mol, greater than or equal to about 1000 g/mol, greater than or equal to about 1,250 g/mol, greater than or equal to about 1,500 g/mol, greater than or equal to about 1,750 g/mol, greater than or equal to about 2,000 g/mol, or greater than or equal to about 2,250 g/mol. Combinations of the above ranges (e.g., at least about 200 g/mol and less than or equal to about 2,500 g/mol) are also possible. In embodiments in which more than one type of ionizable molecules are present in a particle, each type of ionizable molecule may independently have a molecular weight in one or more of the ranges described above.

In some embodiments, the percentage (e.g., by weight, or by mole) of a single type of ionizable molecule (e.g., amino lipid or ionizable lipid) and/or of all the ionizable molecules within a particle may be greater than or equal to about 15%, greater than or equal to about 16%, greater than or equal to about 17%, greater than or equal to about 18%, greater than or equal to about 19%, greater than or equal to about 20%, greater than or equal to about 21%, greater than or equal to about 22%, greater than or equal to about 23%, greater than or equal to about 24%, greater than or equal to about 25%, greater than or equal to about 30%, greater than or equal to about 35%, greater than or equal to about 40%, greater than or equal to about 42%, greater than or equal to about 45%, greater than or equal to about 48%, greater than or equal to about 50%, greater than or equal to about 52%, greater than or equal to about 55%, greater than or equal to about 58%, greater than or equal to about 60%, greater than or equal to about 62%, greater than or equal to about 65%, or greater than or equal to about 68%. In some instances, the percentage (e.g., by weight, or by mole) may be less than or equal to about 70%, less than or equal to about 68%, less than or equal to about 65%, less than or equal to about 62%, less than or equal to about 60%, less than or equal to about 58%, less than or equal to about 55%, less than or equal to about 52%, less than or equal to about 50%, or less than or equal to about 48%. Combinations of the above referenced ranges are also possible (e.g., greater than or equal to 20% and less than or equal to about 60%, greater than or equal to 40% and less than or equal to about 55%, etc.). In embodiments in which more than one type of ionizable molecule is present in a particle, each type of ionizable molecule may independently have a percentage (e.g., by weight, or by mole) in one or more of the ranges described above. The percentage (e.g., by weight, or by mole) may be determined by extracting the ionizable molecule(s) from the dried particles using, e.g., organic solvents, and measuring the quantity of the agent using high pressure liquid chromatography (i.e., HPLC), liquid chromatography-mass spectrometry (LC-MS), nuclear magnetic resonance (NMR), or mass spectrometry (MS). Those of ordinary skill in the art would be knowledgeable of techniques to determine the quantity of a component using the above-referenced techniques. For example, HPLC may be used to quantify the amount of a component, by, e.g., comparing the area under the curve of a HPLC chromatogram to a standard curve.

It should be understood that the terms “charged” or “charged moiety” does not refer to a “partial negative charge” or “partial positive charge” on a molecule. The terms “partial negative charge” and “partial positive charge” are given their ordinary meaning in the art. A “partial negative charge” may result when a functional group comprises a bond that becomes polarized such that electron density is pulled toward one atom of the bond, creating a partial negative charge on the atom. Those of ordinary skill in the art will, in general, recognize bonds that can become polarized in this way.

According to the disclosures herein, a lipid composition may comprise one or more lipids as described herein. Such lipids may include those useful in the preparation of lipid nanoparticle formulations as described above or as known in the art.

In some embodiments, a subject to which a composition comprising a nucleic acid and a lipid, is administered is a subject that suffers from or is at risk of suffering from a disease, disorder or condition, including a communicable or non-communicable disease, disorder or condition. As used herein, “treating” a subject can include either therapeutic use or prophylactic use relating to a disease, disorder or condition, and may be used to describe uses for the alleviation of symptoms of a disease, disorder or condition, uses for vaccination against a disease, disorder or condition, and uses for decreasing the contagiousness of a disease, disorder or condition, among other uses.

In some embodiments the nucleic acid is an mRNA vaccine designed to achieve particular biologic effects. Exemplary vaccines feature mRNAs encoding a particular antigen of interest (or an mRNA or mRNAs encoding antigens of interest). In exemplary aspects, the vaccines feature an mRNA or mRNAs encoding antigen(s) derived from infectious diseases or cancers.

Diseases or conditions, in some embodiments include those caused by or associated with infectious agents, such as bacteria, viruses, fungi and parasites. Non-limiting examples of such infectious agents include Gram-negative bacteria, Gram-positive bacteria, RNA viruses (including (+)ssRNA viruses, (−)ssRNA viruses, dsRNA viruses), DNA viruses (including dsDNA viruses and ssDNA viruses), reverse transcriptase viruses (including ssRNA-RT viruses and dsDNA-RT viruses), protozoa, helminths, and ectoparasites.

Thus, some embodiments encompass infectious disease vaccines. The antigen of the infectious disease vaccine is a viral or bacterial antigen.

In some embodiments, a disease, disorder, or condition is caused by or associated with a virus.

In some embodiments, the lipid compositions are also useful for treating or preventing a symptom of diseases characterized by missing or aberrant protein activity, by replacing the missing protein activity or overcoming the aberrant protein activity. Because of the rapid initiation of protein production following introduction of mRNAs, as compared to viral DNA vectors, the compounds of the present disclosure are particularly advantageous in treating acute diseases such as sepsis, stroke, and myocardial infarction. Moreover, the lack of transcriptional regulation of the alternative mRNAs of the present disclosure is advantageous in that accurate titration of protein production is achievable. Multiple diseases are characterized by missing (or substantially diminished such that proper protein function does not occur) protein activity. Such proteins may not be present, are present in very low quantities or are essentially non-functional. The present disclosure provides a method for treating such conditions or diseases in a subject by introducing polynucleotide or cell-based therapeutics containing the alternative polynucleotides provided herein, wherein the alternative polynucleotides encode for a protein that replaces the protein activity missing from the target cells of the subject.

Diseases characterized by dysfunctional or aberrant protein activity include, but are not limited to, cancer and other proliferative diseases, genetic diseases (e.g., cystic fibrosis), autoimmune diseases, diabetes, neurodegenerative diseases, cardiovascular diseases, and metabolic diseases. The present disclosure provides a method for treating such conditions or diseases in a subject by introducing polynucleotide or cell-based therapeutics containing the polynucleotides provided herein, wherein the polynucleotides encode for a protein that antagonizes or otherwise overcomes the aberrant protein activity present in the cell of the subject.

In some embodiments, a composition disclosed herein does not comprise a pharmaceutical preservative. In other embodiments, a composition disclosed herein does comprise a pharmaceutical preservative. Non-limiting examples of pharmaceutical preservatives include methyl paragen, ethyl paraben, propyl paraben, butyl paraben, benzyl alcohol, chlorobutanol, phenol, meta cresol (m-cresol), chloro cresol, benzoic acid, sorbic acid, thiomersal, phenylmercuric nitrate, bronopol, propylene glycol, benzalkonium chloride, and benzethonium chloride. In some embodiments, a composition disclosed herein does not comprise phenol, m-cresol, or benzyl alcohol. Compositions in which microbial growth is inhibited may be useful in the preparation of injectable formulations, including those intended for dispensing from multi-dose vials. Multi-dose vials refer to containers of pharmaceutical compositions from which multiple doses can be taken repeatedly from the same container. Compositions intended for dispensing from multi-dose vials typically must meet USP requirements for antimicrobial effectiveness.

In some embodiments, “administering” or “administration” means providing a material to a subject in a manner that is pharmacologically useful. In some embodiments, a composition disclosed herein is administered to a subject enterally. In some embodiments, an enteral administration of the composition is oral. In some embodiments, a composition disclosed herein is administered to the subject parenterally. In some embodiments, a composition disclosed herein is administered to a subject subcutaneously, intraocularly, intravitreally, subretinally, intravenously (IV), intracerebro-ventricularly, intramuscularly, intrathecally (IT), intracisternally, intraperitoneally, via inhalation, topically, or by direct injection to one or more cells, tissues, or organs.

To “treat” a disease as the term is used herein, means to reduce the frequency or severity of at least one sign or symptom of a disease, disorder or condition experienced by a subject. The compositions described above or elsewhere herein are typically administered to a subject in an effective amount, that is, an amount capable of producing a desirable result. The desirable result will depend upon the active agent being administered. For example, an effective amount of a composition comprising a nucleic acid and a lipid may be an amount of the composition that is capable of increasing expression of a protein in the subject. A therapeutically acceptable amount may be an amount that is capable of treating a disease or condition, e.g., a disease or condition that that can be relieved by increasing expression of a protein in a subject. As is well known in the medical and veterinary arts, dosage for any one subject depends on many factors, including the subject's size, body surface area, age, the particular composition to be administered, the active ingredient(s) in the composition, the intended outcome of the administration, time and route of administration, general health, and other drugs being administered concurrently.

In some embodiments, a subject is administered a composition comprising a nucleic acid and a lipid I in an amount sufficient to increase expression of a protein in the subject.

In certain embodiments, LNP preparations (e.g., populations or formulations) are analyzed for polydispersity in size (e.g., particle diameter) and/or composition (e.g., amino lipid amount or concentration, phospholipid amount or concentration, structural lipid amount or concentration, PEG-lipid amount or concentration, mRNA amount (e.g., mass) or concentration) and, optionally, further assayed for in vitro and/or in vivo activity. Fractions or pools thereof can also be analyzed for accessible mRNA and/or purity (e.g., purity as determined by reverse-phase (RP) chromatography).

Particle size (e.g., particle diameter) can be determined by Dynamic Light Scattering (DLS). DLS measures a hydrodynamic diameter. Smaller particles diffuse more quickly, leading to faster fluctuations in the scattering intensity and shorter decay times for the autocorrelation function. Larger particles diffuse more slowly, leading to slower fluctuations in the scattering intensity and longer decay times in the autocorrelation function.

mRNA purity can be determined by reverse phase high-performance liquid chromatography (RP-HPLC) size based separation. This method can be used to assess mRNA integrity by a length-based gradient RP separation and UV detection of RNA at 260 nm. As used herein “main peak” or “main peak purity” refers to the RP-HPLC signal detected from mRNA that corresponds to the full size mRNA molecule loaded within a given LNP formulation. mRNA purity can also be assessed by fragmentation analysis. Fragmentation analysis (FA) is a method by which nucleic acid (e.g., mRNA) fragments can be analyzed by capillary electrophoresis. Fragmentation analysis involves sizing and quantifying nucleic acids (e.g., mRNA), for example by using an intercalating dye coupled with an LED light source. Such analysis may be completed, for example, with a Fragment Analyzer from Advanced Analytical Technologies, Inc.

Compositions formed via the methods described herein may be particularly useful for administering an agent to a subject in need thereof. In some embodiments, the compositions are used to deliver a pharmaceutically active agent. In some instances, the compositions are used to deliver a prophylactic agent. The compositions may be administered in any way known in the art of drug delivery, for example, orally, parenterally, intravenously, intramuscularly, subcutaneously, intradermally, transdermally, intrathecally, submucosally, etc.

Once the compositions have been prepared, they may be combined with pharmaceutically acceptable excipients to form a pharmaceutical composition. As would be appreciated by one of skill in this art, the excipients may be chosen based on the route of administration as described below, the agent being delivered, and the time course of delivery of the agent.

Pharmaceutical compositions described herein and for use in accordance with the embodiments described herein may include a pharmaceutically acceptable excipient. As used herein, the term “pharmaceutically acceptable excipient” means a non-toxic, inert solid, semi-solid or liquid filler, diluent, encapsulating material or formulation auxiliary of any type. Some examples of materials which can serve as pharmaceutically acceptable excipients are sugars such as lactose, glucose, and sucrose; starches such as corn starch and potato starch; cellulose and its derivatives such as sodium carboxymethyl cellulose, methylcellulose, hydroxypropylmethylcellulose, ethyl cellulose, and cellulose acetate; powdered tragacanth; malt; gelatin; talc; excipients such as cocoa butter and suppository waxes; oils such as peanut oil, cottonseed oil, safflower oil, sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen free water; isotonic saline; citric acid, acetate salts, Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The pharmaceutical compositions can be administered to humans and/or to animals, orally, parenterally, intracisternally, intranasally, intraperitoneally, topically (as by powders, creams, ointments, or drops), bucally, or as an oral or nasal spray.

Liquid dosage forms for oral administration include pharmaceutically acceptable emulsions, microemulsions, solutions, suspensions, syrups, and elixirs. In addition to the active ingredients (i.e., the particles), the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3 butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, ethanol, U.S.P., and isotonic sodium chloride solution. In addition, sterile, fixed oils are conventionally employed as a solvent or suspending medium. For this purpose any bland fixed oil can be employed including synthetic mono or diglycerides. In addition, fatty acids such as oleic acid are used in the preparation of injectables.

The injectable formulations can be sterilized, for example, by filtration through a bacteria retaining filter, or by incorporating sterilizing agents in the form of sterile solid compositions which can be dissolved or dispersed in sterile water or other sterile injectable medium prior to use.

Solid dosage forms for oral administration include capsules, tablets, pills, powders, and granules. In such solid dosage forms, the particles are mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents.

Solid compositions of a similar type may also be employed as fillers in soft and hard filled gelatin capsules using such excipients as lactose or milk sugar as well as high molecular weight polyethylene glycols and the like.

The solid dosage forms of tablets, dragées, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Dosage forms for topical or transdermal administration of a pharmaceutical composition include ointments, pastes, creams, lotions, gels, powders, solutions, sprays, inhalants, or patches. The particles are admixed under sterile conditions with a pharmaceutically acceptable carrier and any needed preservatives or buffers as may be required. Ophthalmic formulation, ear drops, and eye drops are also possible.

The ointments, pastes, creams, and gels may contain, in addition to the composition, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the compositions, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compositions in a proper medium. Absorption enhancers can also be used to increase the flux of the compound across the skin. The rate can be controlled by either providing a rate controlling membrane or by dispersing the compositions in a polymer matrix or gel.

In other embodiments, the compositions are loaded and stored in prefilled syringes and cartridges for patient-friendly autoinjector and infusion pump devices.

Kits for use in preparing or administering the compositions are also provided. A kit for forming compositions may include any solvents, solutions, buffer agents, acids, bases, salts, targeting agent, etc. needed in the composition formation process. Different kits may be available for different targeting agents. In certain embodiments, the kit includes materials or reagents for purifying, sizing, and/or characterizing the resulting compositions. The kit may also include instructions on how to use the materials in the kit. The one or more agents (e.g., pharmaceutically active agent) to be contained within the composition are typically provided by the user of the kit.

Kits are also provided for using or administering the compositions. The compositions may be provided in convenient dosage units for administration to a subject. The kit may include multiple dosage units. For example, the kit may include 1-100 dosage units. In certain embodiments, the kit includes a week supply of dosage units, or a month supply of dosage units. In certain embodiments, the kit includes an even longer supply of dosage units. The kits may also include devices for administering the compositions. Exemplary devices include syringes, spoons, measuring devices, etc. The kit may optionally include instructions for administering the compositions (e.g., prescribing information).

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As disclosed herein, the terms “composition” and “formulation” are used interchangeably.

### Pharmaceutical Compositions

Some aspects relate to pharmaceutical compositions comprising multivalent RNA compositions produced by methods described by the disclosure. Multivalent RNA compositions described herein may be formulated or administered in combination with one or more pharmaceutically-acceptable excipients. As a non-limiting set of examples, multivalent RNA compositions can be formulated using one or more excipients to: (1) increase stability; (2) increase cell transfection; (3) permit the sustained or delayed release (e.g., from a depot formulation); (4) alter the biodistribution (e.g., target to specific tissues or cell types); (5) increase the translation of encoded protein in vivo; and/or (6) alter the release profile of encoded protein (antigen) in vivo. In addition to traditional excipients such as any and all solvents, dispersion media, diluents, or other liquid vehicles, dispersion or suspension aids, surface active agents, isotonic agents, thickening or emulsifying agents, preservatives, excipients can include, without limitation, lipidoids, liposomes, lipid nanoparticles, polymers, lipoplexes, core-shell nanoparticles, peptides, proteins, cells transfected with multivalent RNA compositions (e.g., for transplantation into a subject), hyaluronidase, nanoparticle mimics and combinations thereof.

In some embodiments, multivalent RNA compositions comprise at least one additional active substance, such as, for example, a therapeutically-active substance, a prophylactically-active substance, or a combination of both. multivalent RNA compositions may be sterile, pyrogen-free or both sterile and pyrogen-free. General considerations in the formulation and/or manufacture of pharmaceutical agents, such as vaccine compositions, may be found, for example, in Remington: The Science and Practice of Pharmacy 21st ed., Lippincott Williams & Wilkins, 2005 (incorporated herein by reference in its entirety for this purpose).

Formulations of the multivalent RNA compositions described herein may be prepared by any method known or hereafter developed in the art of pharmacology. In general, such preparatory methods include the step of bringing the active ingredient(s) (e.g., mRNAs of the multivalent composition) into association with an excipient and/or one or more other accessory ingredients, and then, if necessary and/or desirable, dividing, shaping and/or packaging the product into a desired single- or multi-dose unit.

The formulation of any of the compositions disclosed herein can include one or more components in addition to those described above. For example, the lipid composition can include one or more permeability enhancer molecules, carbohydrates, polymers, surface altering agents (e.g., surfactants), or other components. For example, a permeability enhancer molecule can be a molecule described by U.S. Patent Application Publication No. 2005/0222064. Carbohydrates can include simple sugars (e.g., glucose) and polysaccharides (e.g., glycogen and derivatives and analogs thereof).

A polymer can be included in and/or used to encapsulate or partially encapsulate a pharmaceutical composition disclosed herein (e.g., a pharmaceutical composition in lipid nanoparticle form). A polymer can be biodegradable and/or biocompatible. A polymer can be selected from, but is not limited to, polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates.

In some embodiments, the compositions described herein may be formulated as lipid nanoparticles (LNPs). Accordingly, the present disclosure also relates to nanoparticle compositions comprising (i) a lipid composition comprising a delivery agent, and (ii) a multivalent RNA composition comprising two or more therapeutic peptides or proteins. In such nanoparticle composition, the lipid composition disclosed herein can encapsulate the nucleic acid encoding one or more peptide epitopes.

Nanoparticle compositions are typically sized on the order of micrometers or smaller and can include a lipid bilayer. Nanoparticle compositions encompass lipid nanoparticles (LNPs), liposomes (e.g., lipid vesicles), and lipoplexes. For example, a nanoparticle composition can be a liposome having a lipid bilayer with a diameter of 500 nm or less.

Nanoparticle compositions include, for example, lipid nanoparticles (LNPs), liposomes, and lipoplexes. In some embodiments, nanoparticle compositions are vesicles including one or more lipid bilayers. In certain embodiments, a nanoparticle composition includes two or more concentric bilayers separated by aqueous compartments. Lipid bilayers can be functionalized and/or crosslinked to one another. Lipid bilayers can include one or more ligands, proteins, or channels.

In some embodiments, a lipid nanoparticle comprises an ionizable lipid, a structural lipid, a phospholipid, and mRNA. In some embodiments, the LNP comprises an ionizable lipid, a PEG-modified lipid, a phospholipid and a structural lipid.

As generally defined herein, the term “lipid” refers to a small molecule that has hydrophobic or amphiphilic properties. Lipids may be naturally occurring or synthetic. Examples of classes of lipids include, but are not limited to, fats, waxes, sterol-containing metabolites, vitamins, fatty acids, glycerolipids, glycerophospholipids, sphingolipids, saccharolipids, and polyketides, and prenol lipids. In some instances, the amphiphilic properties of some lipids lead them to form liposomes, vesicles, or membranes in aqueous media.

In some embodiments, a lipid nanoparticle (LNP) may comprise an ionizable lipid. As used herein, the term “ionizable lipid” has its ordinary meaning in the art and may refer to a lipid comprising one or more charged moieties. In some embodiments, an ionizable lipid may be positively charged or negatively charged. An ionizable lipid may be positively charged, in which case it can be referred to as “cationic lipid”. In certain embodiments, an ionizable lipid molecule may comprise an amine group, and can be referred to as an ionizable amino lipids. As used herein, a “charged moiety” is a chemical moiety that carries a formal electronic charge, e.g., monovalent (+1, or −1), divalent (+2, or −2), trivalent (+3, or −3), etc. The charged moiety may be anionic (i.e., negatively charged) or cationic (i.e., positively charged). Examples of positively-charged moieties include amine groups (e.g., primary, secondary, and/or tertiary amines), ammonium groups, pyridinium group, guanidine groups, and imidazolium groups. In a particular embodiment, the charged moieties comprise amine groups. Examples of negatively-charged groups or precursors thereof, include carboxylate groups, sulfonate groups, sulfate groups, phosphonate groups, phosphate groups, hydroxyl groups, and the like. The charge of the charged moiety may vary, in some cases, with the environmental conditions, for example, changes in pH may alter the charge of the moiety, and/or cause the moiety to become charged or uncharged. In general, the charge density of the molecule may be selected as desired. Ionizable lipids can also be the compounds disclosed in International Publication Nos.: WO2017075531, WO2015199952, WO2013086354, or WO2013116126, or selected from formulae CLI-CLXXXXII of U.S. Pat. No. 7,404,969; each of which is hereby incorporated by reference in its entirety for this purpose.

It should be understood that the terms “charged” or “charged moiety” does not refer to a “partial negative charge” or “partial positive charge” on a molecule. The terms “partial negative charge” and “partial positive charge” are given their ordinary meaning in the art. A “partial negative charge” may result when a functional group comprises a bond that becomes polarized such that electron density is pulled toward one atom of the bond, creating a partial negative charge on the atom. Those of ordinary skill in the art will, in general, recognize bonds that can become polarized in this way.

In some embodiments, the ionizable lipid is an ionizable amino lipid, sometimes referred to in the art as an “ionizable cationic lipid”. In some embodiments, the ionizable amino lipid may have a positively charged hydrophilic head and a hydrophobic tail that are connected via a linker structure. In addition to these, an ionizable lipid may also be a lipid including a cyclic amine group.

Multivalent RNA compositions can be formulated into lipid nanoparticles. In some embodiments, the lipid nanoparticle comprises at least one ionizable amino lipid, at least one non-cationic lipid, at least one sterol, and/or at least one polyethylene glycol (PEG)-modified lipid.

Nanoparticle compositions can be characterized by a variety of methods. For example, microscopy (e.g., transmission electron microscopy or scanning electron microscopy) can be used to examine the morphology and size distribution of a nanoparticle composition. Dynamic light scattering or potentiometry (e.g., potentiometric titrations) can be used to measure zeta potentials. Dynamic light scattering can also be utilized to determine particle sizes. Instruments such as the Zetasizer Nano ZS (Malvern Instruments Ltd, Malvern, Worcestershire, UK) can also be used to measure multiple characteristics of a nanoparticle composition, such as particle size, polydispersity index, and zeta potential.

Nanoparticle compositions can be characterized by a variety of methods. For example, microscopy (e.g., transmission electron microscopy or scanning electron microscopy) can be used to examine the morphology and size distribution of a nanoparticle composition. Dynamic light scattering or potentiometry (e.g., potentiometric titrations) can be used to measure zeta potentials. Dynamic light scattering can also be utilized to determine particle sizes. Instruments such as the Zetasizer Nano ZS (Malvern Instruments Ltd, Malvern, Worcestershire, UK) can also be used to measure multiple characteristics of a nanoparticle composition, such as particle size, polydispersity index, and zeta potential.

The size of the nanoparticles can help counter biological reactions such as, but not limited to, inflammation, or can increase the biological effect of the polynucleotide. As used herein, “size” or “mean size” in the context of nanoparticle compositions refers to the mean diameter of a nanoparticle composition.

### Applications

Multivalent RNA compositions described here comprise two or more different RNA molecules that may include but are not limited to mRNA (including modified mRNA and/or unmodified RNA), lncRNA, self-replicating RNA, circular RNA, CRISPR guide RNA, and the like. In embodiments, the RNA is RNA (e.g., mRNA or self-replicating RNA) that encodes a peptide or polypeptide (e.g., a therapeutic peptide or therapeutic polypeptide). Thus, the RNA transcripts produced using RNA polymerase variants may be used in a myriad of applications.

For example, the different RNA transcripts in a multivalent RNA composition may be used to produce polypeptides of interest, e.g., therapeutic proteins, vaccine antigens, and the like. In some embodiments, the RNA transcripts are therapeutic RNAs. A therapeutic mRNA is an mRNA that encodes a therapeutic protein (the term ‘protein’ encompasses peptides). In some embodiments, multivalent RNA compositions described herein comprise one or more RNAs that encode peptides or proteins that interact or complex in a cell or subject to form a multi-subunit protein (e.g., an antibody comprising a heavy chain and a light chain, a multi-subunit receptor protein, a multi-subunit signaling protein, a multi-subunit antigen, etc.) or a multivalent vaccine.

Therapeutic proteins mediate a variety of effects in a host cell or in a subject to treat a disease or ameliorate the signs and symptoms of a disease. For example, a therapeutic protein can replace a protein that is deficient or abnormal, augment the function of an endogenous protein, provide a novel function to a cell (e.g., inhibit or activate an endogenous cellular activity, or act as a delivery agent for another therapeutic compound (e.g., an antibody-drug conjugate). Therapeutic mRNA may be useful for the treatment of the following diseases and conditions: bacterial infections, viral infections, parasitic infections, cell proliferation disorders, genetic disorders, and autoimmune disorders. Other diseases and conditions are encompassed herein.

A protein or proteins of interest encoded by a multivalent RNA composition as described herein can be essentially any multivalent protein or pool of peptides (e.g., peptide antigens).

In some embodiments, a therapeutic peptide or therapeutic protein is a biologic. A biologic is a polypeptide-based molecule that may be used to treat, cure, mitigate, prevent, or diagnose a serious or life-threatening disease or medical condition. Biologics include, but are not limited to, allergenic extracts (e.g. for allergy shots and tests), blood components, gene therapy products, human tissue or cellular products used in transplantation, vaccines, monoclonal antibodies, cytokines, growth factors, enzymes, thrombolytics, and immunomodulators, among others.

In some embodiments, the therapeutic protein is a cytokine, a growth factor, an antibody (e.g., monoclonal antibody), a fusion protein, or a multivalent vaccine (e.g., a collection of RNAs encoding peptide antigens designed to elicit an immune response in a subject). Non-limiting examples of therapeutic proteins include blood factors (such as Factor VIII and Factor VII), complement factors, Low Density Lipoprotein Receptor (LDLR) and MUT1. Non-limiting examples of cytokines include interleukins, interferons, chemokines, lymphokines and the like. Non-limiting examples of growth factors include erythropoietin, EGFs, PDGFs, FGFs, TGFs, IGFs, TNFs, CSFs, MCSFs, GMCSFs and the like. Non-limiting examples of antibodies include adalimumab, infliximab, rituximab, ipilimumab, tocilizumab, canakinumab, itolizumab, tralokinumab, anti-influenza virus monoclonal antibody, anti-Chikungunya virus monoclonal antibody, anti-Zika virus monoclonal antibody, anti-SARS-CoV-2 monoclonal antibody. Non-limiting examples of fusion proteins include, for example, etanercept, abatacept and belatacept. Non-limiting examples of multivalent vaccines include, for example, multivalent Cytomegalovirus (CMV) vaccine, and personalized cancer vaccines.

One or more biologics currently being marketed or in development may be encoded by the RNA. While not wishing to be bound by theory, it is believed that incorporation of the encoding polynucleotides of a known biologic into the RNA described herein will result in improved therapeutic efficacy due at least in part to the specificity, purity and/or selectivity of the construct designs.

A multivalent RNA composition as disclosed herein may encode one or more antibodies (e.g., may comprise a first mRNA encoding an antibody heavy chain and a second RNA encoding an antibody light chain). The term “antibody” includes monoclonal antibodies (including full length antibodies which have an immunoglobulin Fc region), antibody compositions with polyepitopic specificity, multispecific antibodies (e.g., bispecific antibodies, diabodies, and single-chain molecules), as well as antibody fragments. The term “immunoglobulin” (Ig) is used interchangeably with “antibody” herein. A monoclonal antibody is an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic site.

Monoclonal antibodies specifically include chimeric antibodies (immunoglobulins) in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity. Chimeric antibodies include, but are not limited to, “primatized” antibodies comprising variable domain antigen-binding sequences derived from a non-human primate (e.g., Old World Monkey, Ape etc.) and human constant region sequences.

Antibodies encoded in the multivalent RNA compositions may be utilized to treat conditions or diseases in many therapeutic areas such as, but not limited to, blood, cardiovascular, CNS, poisoning (including antivenoms), dermatology, endocrinology, gastrointestinal, medical imaging, musculoskeletal, oncology, immunology, respiratory, sensory and anti-infective.

A multivalent RNA composition as disclosed herein may encode one or more vaccine antigens. A vaccine antigen is a biological preparation that improves immunity to a particular disease or infectious agent. One or more vaccine antigens currently being marketed or in development may be encoded by the RNA. Vaccine antigens encoded in the RNA may be utilized to treat conditions or diseases in many therapeutic areas such as, but not limited to, cancer, allergy and infectious disease. In some embodiments, a vaccine may be a personalized vaccine in the form of a concatemer or individual RNAs encoding peptide epitopes or a combination thereof.

A multivalent RNA composition as disclosed herein may be designed to encode on or more antimicrobial peptides (AMP) or antiviral peptides (AVP). AMPs and AVPs have been isolated and described from a wide range of animals such as, but not limited to, microorganisms, invertebrates, plants, amphibians, birds, fish, and mammals. The anti-microbial polypeptides may block cell fusion and/or viral entry by one or more enveloped viruses (e.g., HIV, HCV). For example, the anti-microbial polypeptide can comprise or consist of a synthetic peptide corresponding to a region, e.g., a consecutive sequence of at least about 5, 10, 15, 20, 25, 30, 35, 40, 45, 50, 55, or 60 amino acids of the transmembrane subunit of a viral envelope protein, e.g., HIV-1 gp120 or gp41. The amino acid and nucleotide sequences of HIV-1 gp120 or gp41 are described in, e.g., Kuiken et al., (2008). “HIV Sequence Compendium,” Los Alamos National Laboratory.

In some embodiments, RNA transcripts are used as radiolabeled RNA probes. In some embodiments, RNA transcripts are used for non-isotopic RNA labeling. In some embodiments, RNA transcripts are used as guide RNA (gRNA) for gene targeting. In some embodiments, RNA transcripts (e.g., mRNA) are used for in vitro translation and micro injection. In some embodiments, RNA transcripts are used for RNA structure, processing and catalysis studies. In some embodiments, RNA transcripts are used for RNA amplification. In some embodiments, RNA transcripts are used as anti-sense RNA for gene expression experiment. Other applications are encompassed by the present disclosure.

### EXAMPLES

### Example 1

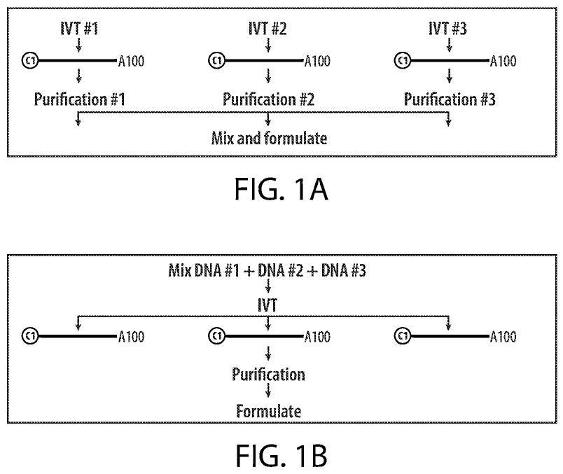

This example describes methods for producing multivalent RNA compositions. In some embodiments, methods described herein are advantageous over previously described multivalent RNA composition production methods, in which each RNA of a multivalent RNA composition is separately transcribed, purified, mixed, and then formulated into a multivalent composition (FIG. 1A). 2 or more DNAs were in vitro transcribed (IVT) in a single reaction and the resulting multivalent RNA composition was purified and formulated without further mixing of RNAs (FIG. 1B). Without being bound by theory, it is believed that optimizing the amount of each DNA in the in vitro transcription reaction based on the polyA-tailing efficiency of each DNA can result in improved yields (e.g., yields having increased purity, for example as measured by polyA-tailing efficiency). These methods have the potential to reduce the number of manufactured batches, depending on the batch size and number of mRNAs that make up the multivalent construct. For example, a multivalent construct that consists of three mRNAs and has a 30 g batch size deliverable would require three 10 g batches to be made and purified prior to formulation using previously described methods. Per the methods described in this document, the same multivalent construct can be made in one 30 g batch, if all the DNAs are mixed together at a pre-defined ratio prior to initiating in vitro transcription. Such a process can require ⅔ less GMP suite time, thereby increasing turn-around-time and capacity.

### Example 2

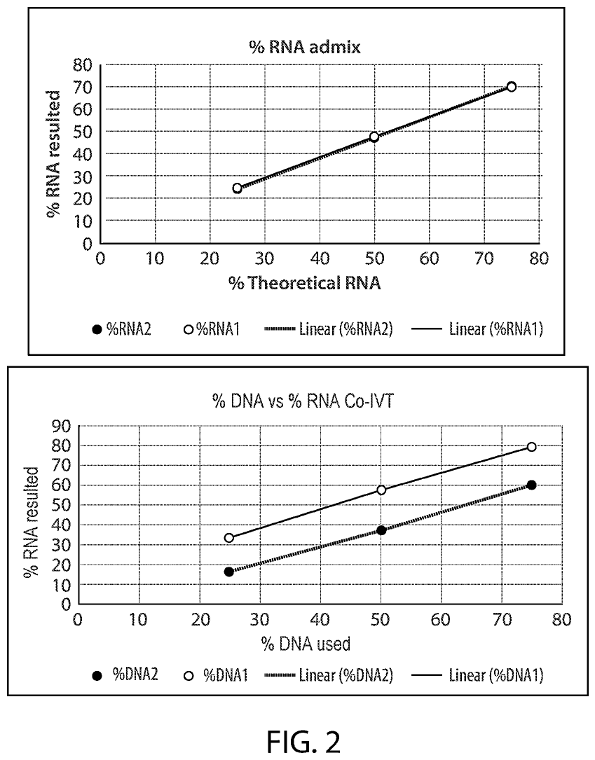

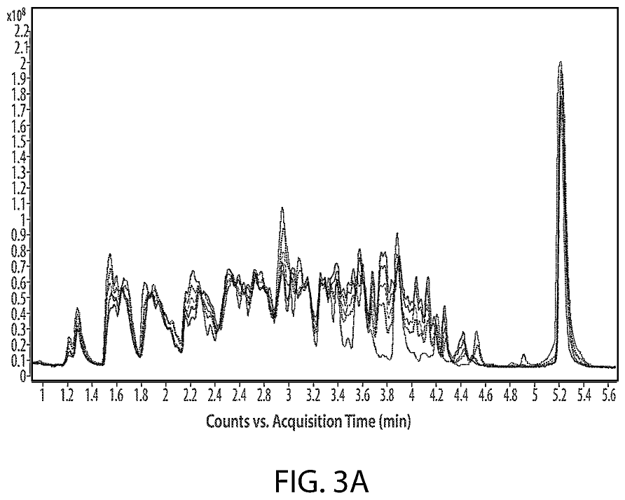

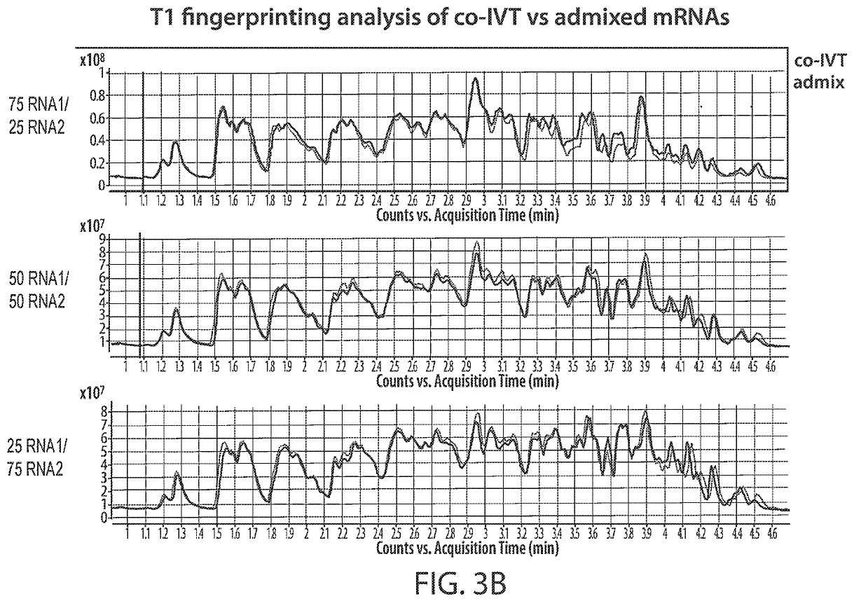

This example describes simultaneous in vitro transcription of the following DNA (e.g., plasmid) expression constructs: DNA2 having a length of ˜2600 nt and DNA1 having a length of ˜1000 nt. Various ratios of DNA plasmid were combined in the co-multivalent in vitro transcription reaction in various ratios (e.g., 100:0, 75:25, 50:50, 25:75, 0:100). As compared to a control (admixed composition prepared by separately transcribing each construct and then mixing them together), the DNA1 constructs produced less RNA than expected and the DNA2 constructs produced more RNA than expected during the co-in vitro transcription reaction (FIG. 2). No length bias over different input amounts of DNA was observed. Moreover, RNase T1 fingerprinting showed that co-IVT RNA and admixed RNA have the same RNase T1 fingerprint (FIGS. 3A-3B). No effect on capping efficiency was observed.

### Example 3

This example describes two co-transcription reactions: i) co-IVT of DNA1 and DNA2 to produce RNA1 and RNA2, and ii) co-IVT of DNA3 and DNA4 to produce RNA3 and RNA4.

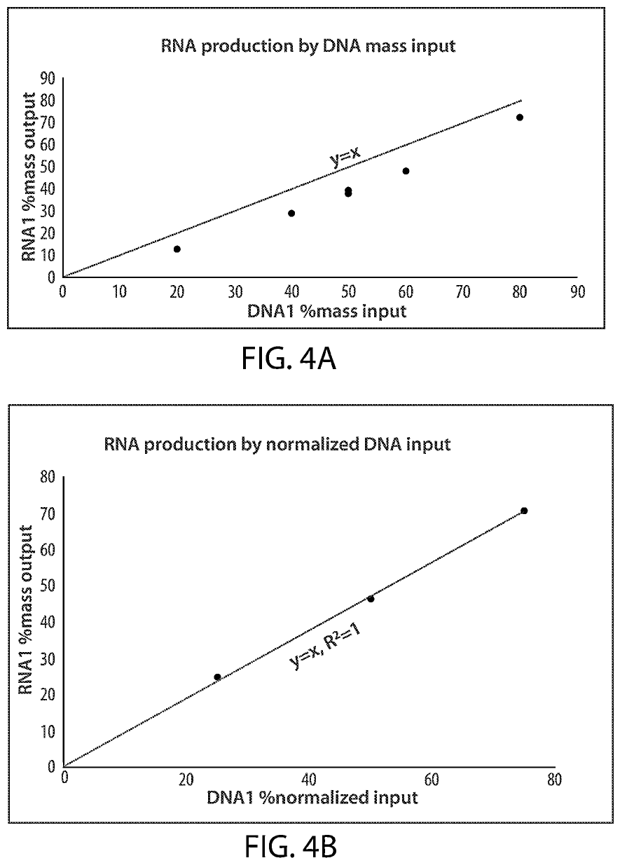

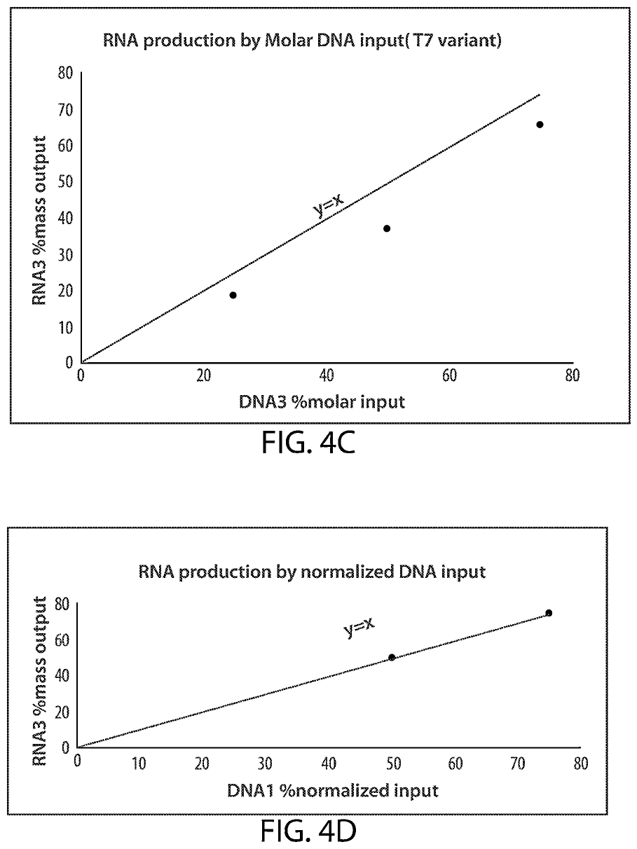

FIGS. 4A-4D show representative data for normalization of input DNA. FIG. 4A shows a linear graph showing the production of RNA1 in a non-normalized mass ratio input of DNA1 and DNA2 in the IVT reaction producing RNA1 and RNA2. Y=X dotted lines depicts no compositional bias between DNA input and RNA output by mass ratio. Data indicate a compositional bias by mass ratio, as the dots do not fall within or close to the dotted line. FIG. 4B shows a linear graph showing the production of RNA1 in a normalized molar ratio input of DNA1 and DNA2 IVT reaction producing RNA1 and RNA2. Y=X dotted lines depicts no compositional bias between DNA input and RNA output by molar ratio. Data indicate no compositional bias, as the dots of normalized DNA input fall within or close to the dotted line. FIG. 4C shows a linear graph showing the production of RNA3 in a non-normalized molar ratio input of DNA3 and DNA4 in a IVT reaction using a T7 polymerase variant producing RNA1 and RNA2. Y=X dotted lines depicts no compositional bias between DNA input and RNA output by mass ratio. Data indicate a compositional bias by molar ratio, as the dots do not fall within or close to the dotted line. FIG. 4D shows a linear graph showing the production of RNA3 in a normalized molar ratio input of DNA3 and DNA4 IVT reaction using a T7 polymerase variant producing RNA1 and RNA2. Y=X black dotted lines depicts no compositional bias between DNA input and RNA output by molar ratio. Data indicate no compositional bias, as the dots of normalized DNA input fall within or close to the dotted line.

### Example 4

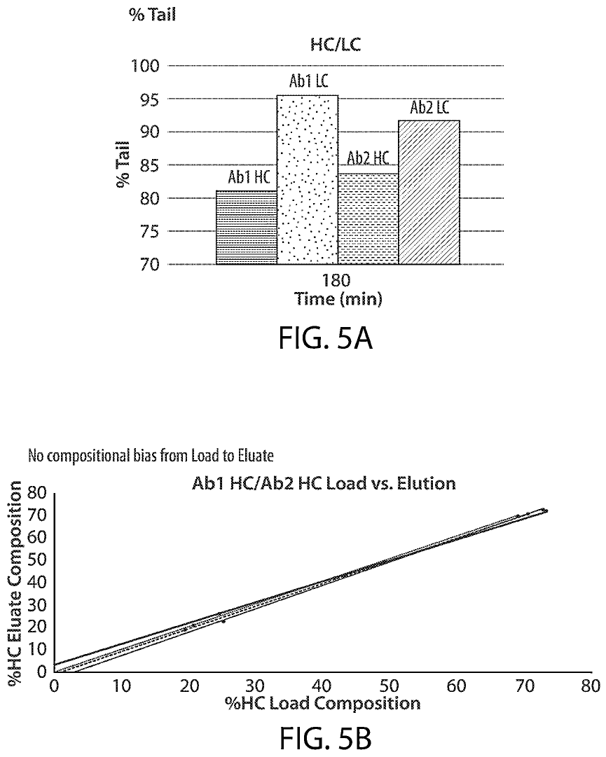

This example describes co-transcription of the heavy chains (HC) and light chains (LC) for two different monoclonal antibodies referred to as Antibody 1 (“Ab1”) (HC and LC) and Antibody B (“Ab2”) (HC and LC). For the co-IVT of each antibody RNA, the molar concentration of output RNA for a pre-determined ratio (e.g., 25:75, 50:50, 75:25, etc.) was used to calculate the molar amount of the corresponding input DNA construct, after which the plasmid mass of each corresponding input DNA construct was determined. FIG. 5A shows representative data indicating that polyA tail percentage is higher in the LC-encoding RNA than the HC-encoding RNA for both antibodies. FIG. 5B shows representative data indicating that the compositional bias not due to purification technique, which in this instance was a dT column.

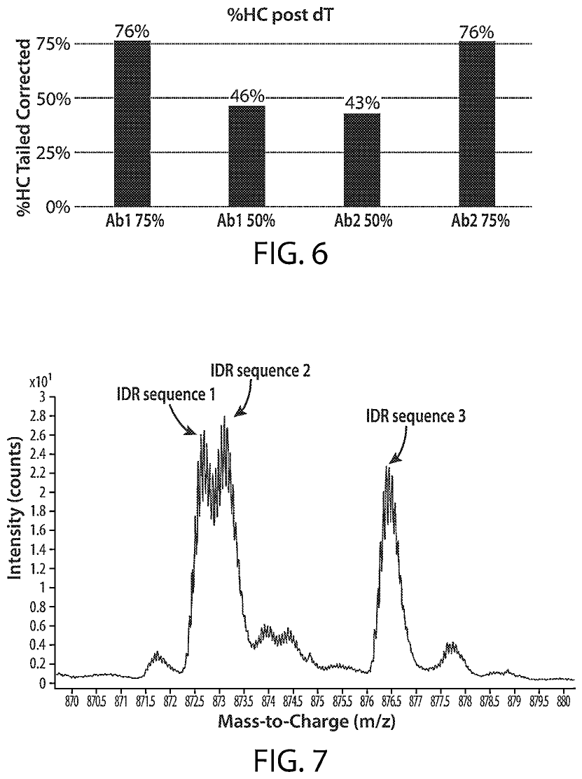

Processes to remove compositional bias when performing co-IVT with T7 RNA polymerase variants were investigated. Briefly, prior to calculating the molar amount of input DNA (and subsequently input DNA % mass) a normalizing step was added to account for differences in polyA tailing efficiency of the RNAs. It was observed that normalizing the amount of desired RNA (e.g., according to a pre-determined ratio) to the highest polyA tailing efficiency of the DNAs being used for the co-IVT resulted in removal of compositional bias from the co-IVT reactions. For example, in this antibody example, the amounts of total HC RNA for a given pre-defined ratio of HC:LC were normalized to the polyA tailing efficiency of the LC RNAs because the LC RNA for both Ab1 and Ab2 were polyA tailed more efficiently (see FIG. 5A). FIG. 6 shows representative data indicating that normalizing the input DNA mass to the highest efficiency polyA tailing RNA resulted in production of multivalent antibody RNA compositions not only having the correct ratio of HC:LC but also that the RNAs in those compositions were polyA-tailed.

### Example 5

This example describes methods of analyzing RNA fragments produced by RNase H-mediated cleavage. IDR sequences may be included in nucleic acids to identify the presence of a nucleic acid in a composition, quantify the amount of mRNA (e.g., various species of mRNA in a multivalent composition), and/or to differentiate one nucleic acid from others in a multivalent mixture. Nucleic acids containing different IDR sequences can be differentiated by molecular biology methods, such as sequencing and PCR. An alternative method of differentiating nucleic acids with distinct IDR sequences is by mass spectrometry. However, mass spectrometry-based methods utilize nucleic acids, or the fragments to be analyzed, that differ in mass, as distinct sequences may be difficult to differentiate if they are similar in mass (FIG. 7).

It was observed that homopolymeric repeats in sequences of mRNAs bound by DNA nucleotides of the RNase H guide oligonucleotides reduced the specificity of DNA binding and mRNA cleavage. Off-target binding of DNA nucleotides of the RNase H guide results in off-target cleavage of mRNA. Non-specific binding of DNA nucleotides of an RNase H guide can thus cause a single mRNA species to be cleaved into one of multiple fragments, reducing the usefulness of cleavage fragment analysis for quantification of mRNA species in a multivalent mixture. To resolve these difficulties, mRNAs were designed with recognition sequences that lacked homopolymeric repeats in the sequence bound by DNA nucleotides of the RNase H guide, to reduce the frequency of off-target cleavage. Exemplary recognition sequences are shown below, in Table 1, as SEQ ID NO: 21 and SEQ ID NO: 22.

It was also determined that IDR sequences that are sequence isomers, wherein each sequence contains the same number of a given base but in a different arrangement (e.g., AGUU and UGAU) have identical masses. The same difficulty in resolving fragments occurred when IDR sequences had similar molecular masses, even if sequences differed. To resolve this difficulty, different RNA IDR sequences, each with a different mass, were designed. Exemplary IDR sequences are shown in Table 2, though other pairs of IDR sequences that are not sequence isomers and differ in mass can be used to distinguish mRNA species in a multivalent mRNA mixture.

RNase H guides, each with a different sequence that was complementary to a sequence near the location of the RNA IDR sequence, were generated. Exemplary RNA sequences flanking the RNA IDR sequence, and sequences of RNase H guides, are shown in Table 1.

TABLE 1
Sequences of RNase H guides and
mRNA regions for targeted mRNA cleavage.

| | | SEQ |
|---|---|---|
| | | ID |
| | Sequence (5′→3′) | NO: |
| Sequence 5′ to | AGCCCCUCCUCCCCUUCCUGCA | 21 |
| RNA IDR sequence | | |
| Sequence 3′ to | GUGGUCUUUGAAUAAAGUCUGA | 22 |
| RNA IDR sequence | | |
| 3′UTR of test | UGAUAAUAGGCUGGAGCCUCGG | 23 |
| mRNA | UGGCCUAGCUUCUUGCCCCUUG | |
| | GGCCUCCCCCCAGCCCCUCCUC | |
| | CCCUUCCUGCAAGUGGUCACCC | |
| | GUACCCCCGUGGUCUUUGAAUA | |
| | AAGUCUGAGUGGGCGGC | |
| RNase H guide 1 | mUmGCAGGmAmAmGmGmGmGmA | 24 |
| | mGmGmAmGmGmGmGmCmU | |
| RNase H guide 2 | mUmCmAmGmAmCmUmUmUmAmU | 25 |
| | mUmCmAmAmAmGmACCAC | |
| RNase H guide 3 | mUmCmAmGmAmCmUmUmUmAmU | 26 |
| | mUmCmAmAmAmGACCAmC | |
| RNase H guide 4 | mUmCmAmGmAmCmUmUmUmAmU | 27 |
| | mUmCmAmAmAGACCmAmC | |
| RNase H guide 5 | mUmCmAmGmAmCmUmUmUmAmU | 28 |
| | mUmCAAAGmAmCmCmAmC | |
| RNase H guide 6 | mUmCmAmGmAmCmUmUmUmAmU | 29 |
| | mUCAAAmGmAmCmCmAmC | |
| RNase H guide 7 | mUmGmCmAmGGAAGmGmGmGmA | 30 |
| | mGmGmAmGmGmGmGmCmU | |
| RNase H guide 8 | mUmCmAmGmAmCmUmUmUmAmU | 31 |
| | mUmCmAmAmAmGACCAmC | |

mN = 2′-O-methyl RNA nucleotide.

TABLE 2
Exemplary RNA IDR sequences.

| IDR sequence | Sequence (5′→3′) |
|---|---|
| 0 | — |
| 1 | A |
| 2A | GG |
| 2B | CC |
| 3A | GGA |
| 3B | CCA |
| 4 | GGAA |
| 5 | GGUUA |
| 6 | GGACCA |
| 7 | GGCCAAA |
| 8 | GGCCAAGA |
| 9 | GGCCAAGGA |
| 11C | CCCGUACCCCC |
| | (SEQ ID NO: 1) |

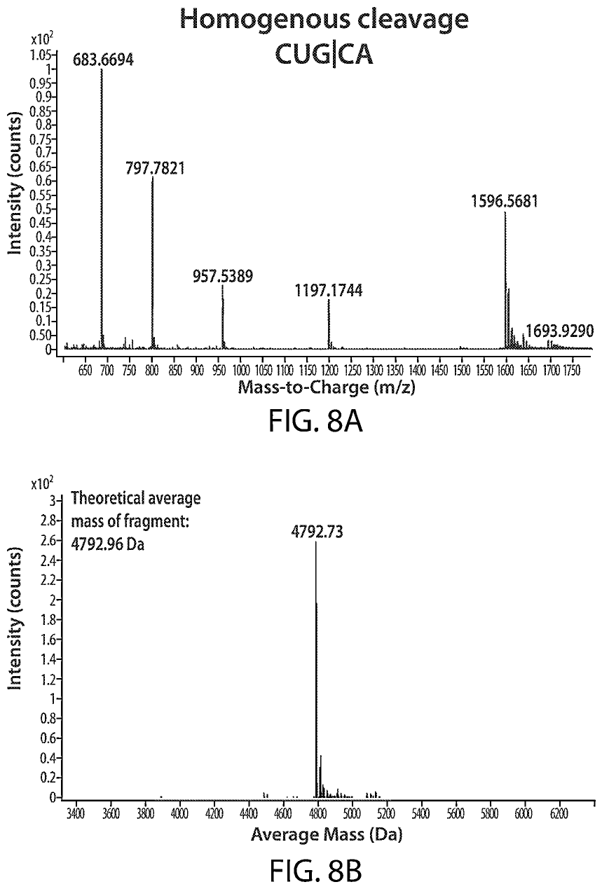

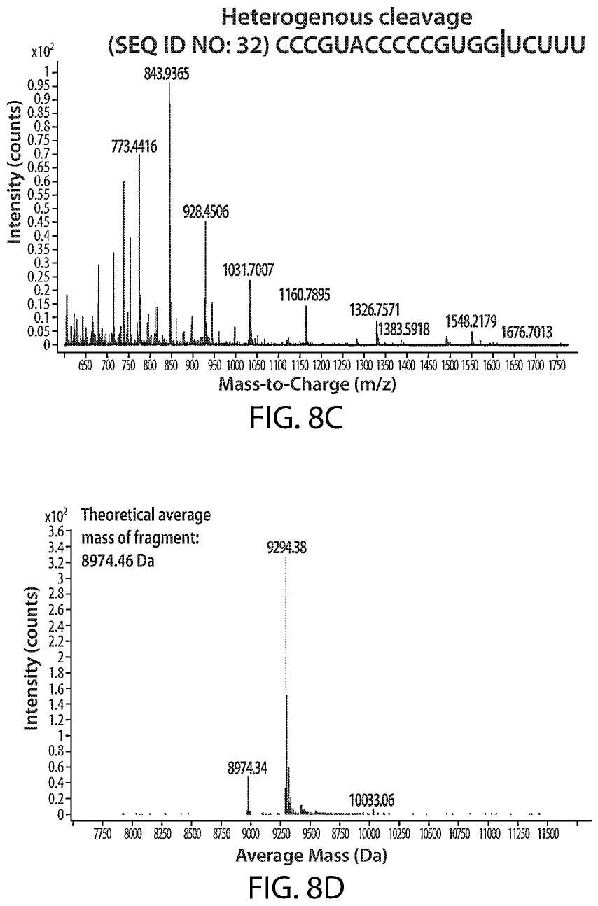

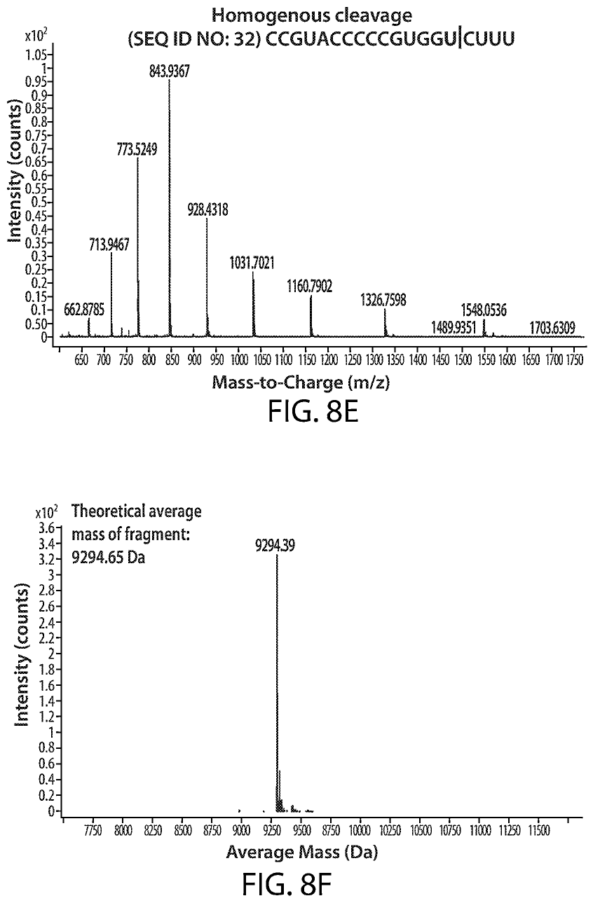

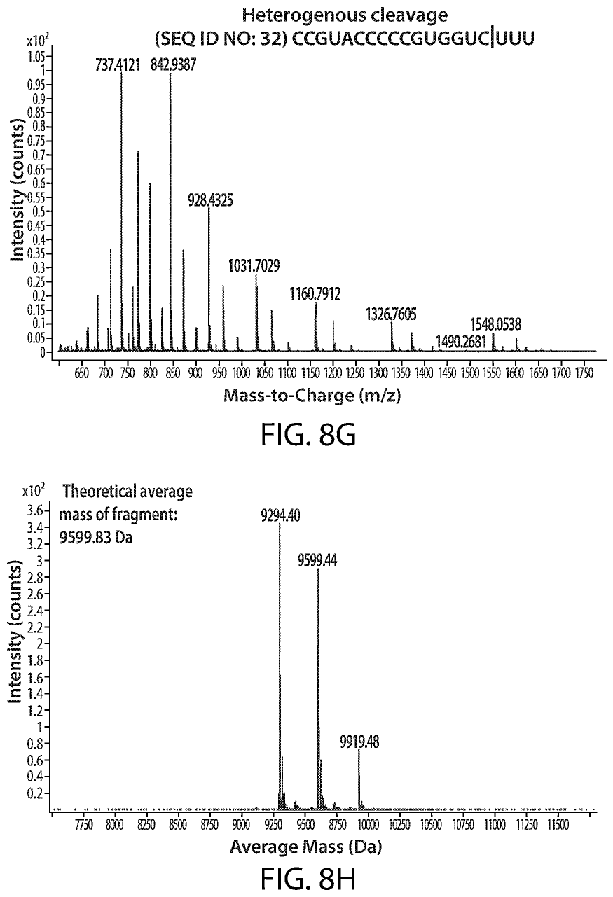

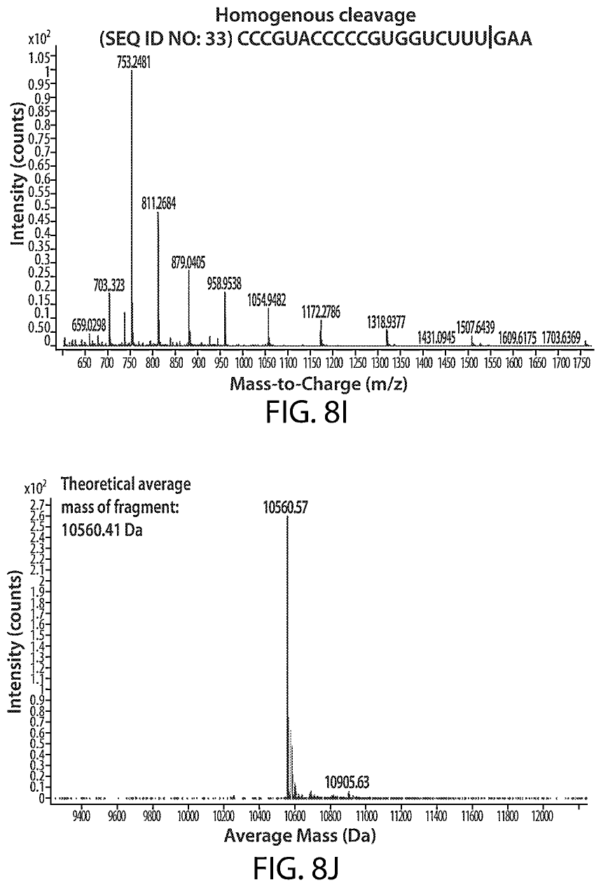

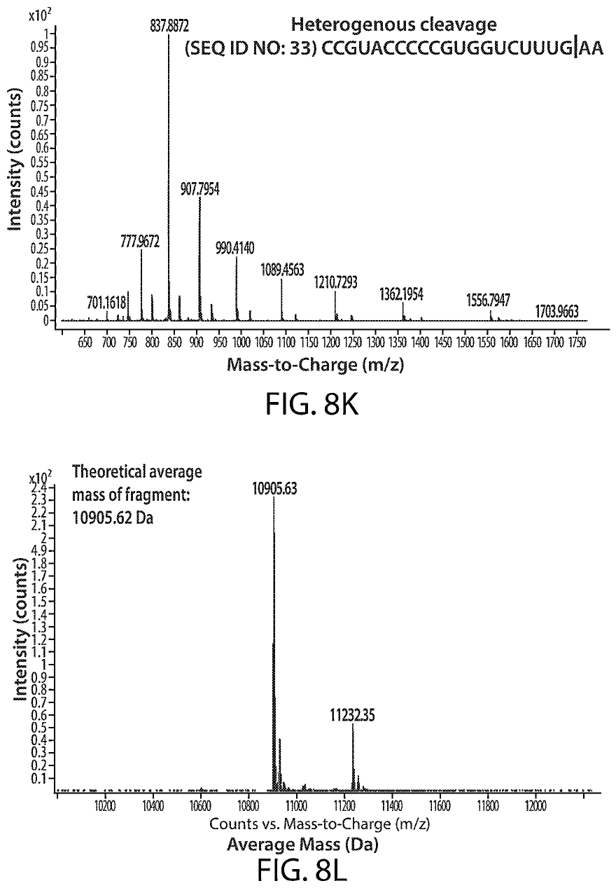

To test the specificity of binding and cleavage for each RNase H guide sequence, each RNase H guide was independently incubated with an identical mRNA, containing the IDR sequence AGTGGTCA, to allow hybridization, and RNase H was added to cleave the RNA in each DNA:RNA hybrid. Mass spectrometry was then used to analyze the composition of RNA fragments produced by RNase H cleavage. As shown in FIGS. 8A-8B, 8E-8F, and 8I-8J, DNA guides 1, 3, or 5 produced a homogenous population of RNA fragments. By contrast, as shown in FIGS. 8C-8D, 8G-8H, and 8K-8L, RNase H guides 2, 4, and 6, produced heterogenous population of RNA fragments, due to some amount of off-target cleavage. These results indicate that RNA fragments, such as those cleaved from the 3′ or 5′ end of an mRNA, can be distinguished by mass spectrometry analysis, and that the specificity of RNase cleavage can be determined by analysis of such fragments.

### Example 6

To confirm protein expression from mRNAs containing distinct sequences in their 3′ UTRs, individual mRNA species containing distinct IDR sequences in the 3′ UTR, as well as mRNAs that did not contain an IDR sequence, were transfected into separate populations of HeLa cells, and into separate populations HEK293 cells. In both cell types, expression of the encoded protein did not differ between mRNAs containing distinct IDR sequences in their 3′ UTRs. Additionally, expression from the mRNAs containing IDR sequences was unchanged relative to expression from the mRNA containing no IDR sequence. These results indicate that sequence changes within the 3′ UTR, such as the addition of IDR sequences that are useful for differentiating mRNAs, or modification of those IDR sequences, do not negatively affect translation of the mRNAs in cells.

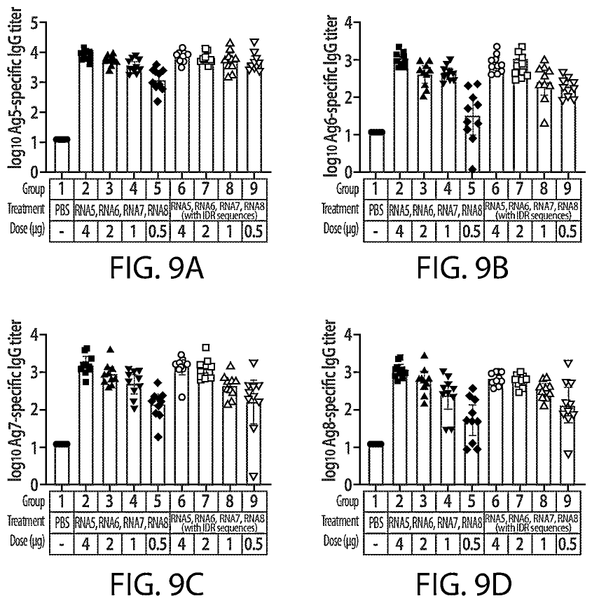

To determine whether the inclusion of IDR sequences on the 3′ UTRs of mRNAs in a multivalent mixture affected the immunogenicity of compositions containing such mRNAs, mice were immunized by administering one of two different types lipid nanoparticle compositions containing a quadrivalent mRNA mixture. Each composition contained four mRNA species, RNA5 encoding Ag5, RNA6 encoding Ag6, RNA7 encoding Ag7, and RNA8 encoding Ag8, at a 1:1:1:1 ratio of each mRNA. In one composition type, the mRNAs did not comprise IDR sequences. In another composition type, each mRNA of the composition contained a distinct IDR sequence in the 3′ UTR. Each composition was administered to mice at a dose of 4 μg, 2 μg, 1 μg, or 0.5 μg mRNA per mouse. 21 days after administration, sera were collected from mice, and titers of Ag5, Ag6, Ag7, and Ag8-specific IgG were measured in each group. The results of these experiments are shown in FIGS. 9A-9D. Antigen-specific IgG titers were lower in animals immunized with lower doses of mRNA compositions, but IgG titers against each of the four antigens were similar in mice immunized with equivalent doses of a quadrivalent composition, regardless of whether mRNAs of the composition comprised IDR sequences in the 3′ UTR. These results indicate that identifying sequences can be added to the 3′ UTR of mRNAs to facilitate identification and analysis of mRNA species in a multivalent (e.g, quadrivalent) mRNA mixture, without affecting the ability of such mRNA compositions to elicit an immune response to antigens encoded by the mRNAs.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B): in another embodiment, to B only (optionally including elements other than A); in yet another embodiment, to both A and B (optionally including other elements); etc. As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc. Each possibility represents a separate embodiment of the present invention.

It should be understood that, unless clearly indicated to the contrary, the disclosure of numerical values and ranges of numerical values in the specification includes both i) the exact value(s) or range specified, and ii) values that are “about” the value(s) or ranges specified (e.g., values or ranges falling within a reasonable range (e.g., about 10% similar)) as would be understood by a person of ordinary skill in the art.

It should also be understood that, unless clearly indicated to the contrary, in any methods disclosed herein that include more than one step or act, the order of the steps or acts of the method is not necessarily limited to the order in which the steps or acts of the method are disclosed.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method for analyzing a multivalent RNA composition, the method comprising:
(i) contacting a multivalent RNA composition, comprising a first RNA species and a second RNA species, with two or more RNase H guide oligonucleotides;
(ii) digesting the first RNA species and the second RNA species with an RNase H enzyme to release a plurality of first RNA fragments and second RNA fragments; and
(iii) measuring a presence and/or amount of the released first RNA fragments and second RNA fragments.

2. The method of claim 1, wherein the first RNA species comprises a first identifying sequence and the second RNA species comprises a second identifying sequence, wherein the first and second identifying sequences are different.

3. The method of claim 2, wherein each of the first and second identifying sequences has a nucleotide length that is independently selected from between 1 to 25 nucleotides.

4. The method of claim 2 or 3, wherein the first identifying sequence is not a sequence isomer of the second identifying sequence.

5. The method of any one of claims 2-4, wherein the first and second identifying sequences have different nucleotide lengths.

6. The method of any one of claims 2-5, wherein the first identifying sequence has a first identifying mass equal to a mass of an RNA consisting of the first identifying sequence, wherein the second identifying sequence has a second identifying mass equal to a mass of an RNA consisting of the second identifying sequence, wherein the first and second identifying masses are different.

7. The method of claim 6, wherein the first and second identifying masses differ by 9 Da or more, 25 Da or more, 50 Da or more, 75 Da or more, or 100 Da or more.

8. The method of any one of claims 2-7, wherein the measuring comprises detecting the released first RNA fragments and second RNA fragments by LC-MS.

9. The method of any one of claims 2-8, wherein the measuring comprises detecting the released first RNA fragments and second RNA fragments by LC-UV.

10. The method of claim 8 or 9, further comprising calculating a ratio between the amounts of the released first RNA fragments and second RNA fragments.

11. The method of any one of claims 2-10, wherein the first RNA species comprises a first 5′ UTR, wherein the second RNA species comprises a second 5′ UTR, wherein each of the two or more RNase H guide oligonucleotides is capable of hybridizing with a nucleotide sequence in the first 5′ UTR and the second 5′ UTR.

12. The method of claim 11, wherein the method comprises cleaving the first 5′ UTR to release the first RNA fragment, and cleaving the second 5′ UTR to release the second RNA fragment,
wherein the first RNA fragment comprises a first cap and the second RNA fragment comprises a second cap.

13. The method of claim 12, wherein the first RNA fragment comprises the first identifying sequence and the second RNA fragment comprises the second identifying sequence.

14. The method of claim 11, wherein the method comprises cleaving the first 5′ UTR to release the first RNA fragment, and cleaving the second 5′ UTR to release the second RNA fragment,
wherein the first RNA fragment comprises the first identifying sequence and the second RNA fragment comprises the second identifying sequence.

15. The method of claim 14, wherein the method comprises:
(i) cleaving the first 5′ UTR at a position upstream from the first identifying sequence and at a position downstream of the first identifying sequence to release the first RNA fragment, wherein the first RNA fragment comprises the first identifying sequence; and
(ii) cleaving the second 5′ UTR at a position upstream from the second identifying sequence and at a position downstream from the second identifying sequence to release the second RNA fragment, wherein the second RNA fragment comprises the second identifying sequence.

16. The method of any one of claims 2-10, wherein the first RNA species comprises a first 3′ UTR, wherein the second RNA species comprises a second 3′ UTR, wherein each of the two or more RNase H guide oligonucleotides is capable of hybridizing with a nucleotide sequence in the first 3′ UTR and the second 3′ UTR.

17. The method of claim 16, wherein the method comprises cleaving the first 3′ UTR to release the first RNA fragment, and cleaving the second 3′ UTR to release the second RNA fragment,
wherein the first RNA fragment comprises a first poly(A) tail and the second RNA fragment comprises a second poly(A) tail.

18. The method of claim 17, wherein the first RNA fragment comprises the first identifying sequence and the second RNA fragment comprises the second identifying sequence.

19. The method of claim 16, wherein the method comprises cleaving the first 3′ UTR to release the first RNA fragment, and cleaving the second 3′ UTR to release the second RNA fragment,
wherein the first RNA fragment comprises the first identifying sequence and the second RNA fragment comprises the second identifying sequence.

20. The method of claim 19, wherein the method comprises:
(i) cleaving the first 3′ UTR at a position upstream from the first identifying sequence and at a position downstream of the first identifying sequence to release the first RNA fragment, wherein the first RNA fragment comprises the first identifying sequence; and
(ii) cleaving the second 3′ UTR at a position upstream from the second identifying sequence and at a position downstream from the second identifying sequence to release the second RNA fragment, wherein the second RNA fragment comprises the second identifying sequence.

21. The method of claim 15 or 20, wherein the method comprises contacting the multivalent RNA composition with a first and second RNase H guide oligonucleotide,
wherein the first RNase H guide oligonucleotide is capable of hybridizing with a sequence upstream from the identifying sequence,
wherein the second RNase H guide oligonucleotide is capable of hybridizing with a sequence downstream from the identifying sequence.

22. The method of any one of claims 2-21, wherein the nucleotide sequences of the released first and second RNA fragments are identical except for the first identifying sequence in the first RNA fragment and the second identifying sequence in the second RNA fragment.

23. The method of any one of claims 1-22, wherein the each of the two or more RNase H guide oligonucleotides comprises a nucleotide sequence represented by the formula:
[R]ₚD₁D₂D₃D₄[R]_{q}
wherein each R is an RNA nucleotide, each D is a DNA nucleotide, and each of p and q are independently an integer between 1 and 50.

24. The method of claim 23, wherein one or more RNA nucleotides of the two or more RNase H guide oligonucleotides are modified RNA nucleotides.

25. The method of claim 24, wherein each RNA nucleotide of the two or more RNase H guide oligonucleotides is a modified RNA nucleotide.

26. The method of claim 23 or 24, wherein one or more modified RNA nucleotides of the two or more RNase H guide oligonucleotides are 2′-O-methyl RNA nucleotides.

27. The method of claim 26, wherein each RNA nucleotide of the two or more RNase H guide oligonucleotides is a 2′-O-methyl RNA nucleotide.

28. The method of claim 25 or 26, wherein one or more modified RNA nucleotides of the two or more RNase H guide oligonucleotides comprises:
(a) a modified nucleobase selected from the group consisting of xanthine, allyaminouracil, allyaminothymidine, hypoxanthine, digoxigeninated adenine, digoxigeninated cytosine, digoxigeninated guanine, digoxigeninated uracil, 6-chloropurineriboside, N6-methyladenine, methylpseudouracil, 2-thiocytosine, 2-thiouracil, 5-methyluracil, 4-thiothymidine, 4-thiouracil, 5,6-dihydro-5-methyluracil, 5,6-dihydrouracil, 5-[(3-Indolyl)propionamide-N-allyl]uracil, 5-aminoallylcytosine, 5-aminoallyluracil, 5-bromouracil, 5-bromocytosine, 5-carboxycytosine, 5-carboxymethylesteruracil, 5-carboxyuracil, 5-fluorouracil, 5-formylcytosine, 5-formyluracil, 5-hydroxycytosine, 5-hydroxymethylcytosine, 5-hydroxymethyluracil, 5-hydroxyuracil, 5-iodocytosine, 5-iodouracil, 5-methoxycytosine, 5-methoxyuracil, 5-methylcytosine, 5-methyluracil, 5-propargylaminocytosine, 5-propargylaminouracil, 5-propynylcytosine, 5-propynyluracil, 6-azacytosine, 6-azauracil, 6-chloropurine, 6-thioguanine, 7-deazaadenine, 7-deazaguanine, 7-deaza-7-propargylaminoadenine, 7-deaza-7-propargylaminoguanine, 8-azaadenine, 8-azidoadenine, 8-chloroadenine, 8-oxoadenine, 8-oxoguanine, araadenine, aracytosine, araguanine, arauracil, biotin-16-7-deaza-7-propargylaminoguanine, biotin-16-aminoallylcytosine, biotin-16-aminoallyluracil, cyanine 3-5-propargylaminocytosine, cyanine 3-6-propargylaminouracil, cyanine 3-aminoallylcytosine, cyanine 3-aminoallyluracil, cyanine 5-6-propargylaminocytosine, cyanine 5-6-propargylaminouracil, cyanine 5-aminoallylcytosine, cyanine 5-aminoallyluracil, cyanine 7-aminoallyluracil, dabcyl-5-3-aminoallyluracil, desthiobiotin-16-aminoallyl-uracil, desthiobiotin-6-aminoallylcytosine, isoguanine, N1-ethylpseudouracil, N1-methoxymethylpseudouracil, N1-methyladenine, N1-methylpseudouracil, N1-propylpseudouracil, N2-methylguanine, N4-biotin-OBEA-cytosine, N4-methylcytosine, N6-methyladenine, O6-methylguanine, pseudoisocytosine, pseudouracil, thienocytosine, thienoguanine, thienouracil, xanthosine, 3-deazaadenine, 2,6-diaminoadenine, 2,6-daminoguanine, 5-carboxamide-uracil, 5-ethynyluracil, N6-isopentenyladenine (i6A), 2-methyl-thio-N6-isopentenyladenine (ms2i6A), 2-methylthio-N6-methyladenine (ms2m6A), N6-(cis-hydroxyisopentenyl)adenine (io6A), 2-methylthio-N6-(cis-hydroxyisopentenyl)adenine (ms2io6A), N6-glycinylcarbamoyladenine (g6A), N6-threonylcarbamoyladenine (t6A), 2-methylthio-N6-threonyl carbamoyladenine (ms2t6A), N6-methyl-N6-threonylcarbamoyladenine (m6t6A), N6-hydroxynorvalylcarbamoyladenine (hn6A), 2-methylthio-N6-hydroxynorvalyl carbamoyladenine (ms2hn6A), N6,N6-dimethyladenine (m62A), and N6-acetyladenine (ac6A);
(b) a modified sugar selected from the group consisting of 2′-thioribose, 2′,3′-dideoxyribose, 2′-amino-2′-deoxyribose, 2′ deoxyribose, 2′-azido-2′-deoxyribose, 2′-fluoro-2′-deoxyribose, 2′-O-methylribose, 2′-O-methyldeoxyribose, 3′-amino-2′,3′-dideoxyribose, 3′-azido-2′,3′-dideoxyribose, 3′-deoxyribose, 3′-O-(2-nitrobenzyl)-2′-deoxyribose, 3′-O-methylribose, 5′-aminoribose, 5′-thioribose, 5-nitro-1-indolyl-2′-deoxyribose, 5′-biotin-ribose, 2′-O,4′-C-methylene-linked, 2′-O,4′-C-amino-linked ribose, and 2′-O,4′-C-thio-linked ribose; and/or
(c) a modified phosphate selected from the group consisting of phosphorothioate (PS), thiophosphate, 5′-O-methylphosphonate, 3′-O-methylphosphonate, 5′-hydroxyphosphonate, hydroxyphosphanate, phosphoroselenoate, selenophosphate, phosphoramidate, carbophosphonate, methylphosphonate, phenylphosphonate, ethylphosphonate, H-phosphonate, guanidinium ring, triazole ring, boranophosphate (BP), methylphosphonate, and guanidinopropyl phosphoramidate.

29. The method of any one of claims 23-28, wherein one or more DNA nucleotides of the two or more RNase H guide oligonucleotides are modified DNA nucleotides.

30. The method of claim 29, wherein each DNA nucleotide of the two or more RNase H guide oligonucleotides is a modified DNA nucleotide.

31. The method of claim 29 or 30, wherein one or more modified DNA nucleotides of the two or more RNase H guide oligonucleotides are 5-nitroindole, Inosine, 4-nitroindole, 6-nitroindole, 3-nitropyrrole, a 2-6-diaminopurine, 2-amino-adenine, or 2-thio-thiamine DNA nucleotides.

32. The method of any one of claims 1-31, wherein each of the two or more RNase H guide oligonucleotides does not comprise a nucleotide sequence comprising 6 or more, 5 or more, or 4 or more consecutive DNA nucleotides having the same nucleobase.

33. The method of any one of claims 1-32, wherein one or more of the RNAs is an mRNA.

34. The method of claim 33, wherein each of the RNAs are mRNAs.

35. The method of any one of claims 1-34, wherein one or more of the RNAs are in vitro transcribed (IVT) mRNAs.

36. The method of claim 35, wherein each of the RNAs are IVT mRNAs.

37. An RNA composition comprising two or more RNA species, wherein the first RNA species comprises a first identifying sequence and the second RNA species comprises a second identifying sequence, wherein the first and second identifying sequences are different.

38. The RNA composition of claim 37, wherein each of the first and second identifying sequences has a nucleotide length that is independently selected from between 1 to 25 nucleotides.

39. The RNA composition of claim 37 or 38, wherein the first identifying sequence is not a sequence isomer of the second identifying sequence.

40. The RNA composition of any one of claims 37-39, wherein the first and second identifying sequences have different nucleotide lengths.

41. The RNA composition of any one of claims 37-40, wherein the first identifying sequence has a first identifying mass equal to a mass of an RNA consisting of the first identifying sequence, wherein the second identifying sequence has a second identifying mass equal to a mass of an RNA consisting of the second identifying sequence, wherein the first and second identifying masses are different.

42. The RNA composition of claim 41, wherein the first and second identifying masses differ by 9 Da or more, 25 Da or more, 50 Da or more, 75 Da or more, or 100 Da or more.

43. The RNA composition of any one of claims 37-42, wherein one or more of the RNAs is an mRNA.

44. The RNA composition of claim 43, wherein each of the RNAs are mRNAs.

45. The RNA composition of any one of claims 37-44, wherein one or more of the RNAs are in vitro transcribed (IVT) mRNAs.

46. The RNA composition of claim 45, wherein each of the RNAs are IVT mRNAs.

47. The RNA composition of any one of claims 37-46, or the method of any one of claims 1-35, wherein the RNA composition comprises 2, 3, 4, 5, 6, 7, 8, 9, or 10 RNA species.

48. The RNA composition of any one of claims 37-47, or the method of any one of claims 1-35, wherein each RNA species comprises an open reading frame encoding a therapeutic peptide or therapeutic protein.

49. The RNA composition of any one of claims 37-48, or the method of any one of claims 1-35, wherein each RNA species comprises an open reading frame encoding an antigenic peptide or antigenic protein.

50. The RNA composition of any one of claims 37-49, or the method of any one of claims 1-35, wherein at least 80%, at least 85%, at least 90%, at least 95%, at least 96%, at least 97%, at least 98%, at least 99%, or up to 100% of RNAs of the RNA composition comprise a poly(A) tail.

51. The RNA composition of any one of claims 37-50, or the method of any one of claims 1-35, wherein the amount of each RNA species in the RNA composition is between 0.2 times to 5 times, 0.3 times to 3 times, or 0.5 times to 2 times, 0.75 times to 1.4 times, 0.8 times to 1.25 times, or 0.9 to 1.15 times the amount of each other RNA species in the RNA composition.

52. A pharmaceutical composition comprising:
(a) the RNA composition of any one of claims 37-51; and
(b) one or more pharmaceutically acceptable excipients.

53. The pharmaceutical composition of claim 52, wherein the RNAs of the RNA composition are packaged in a lipid-based particle.

54. The pharmaceutical composition of claim 53, wherein the lipid-based particle is a liposome or a lipid nanoparticle.

55. A method for producing a multivalent RNA composition, the method comprising:
(a) combining a linearized first DNA molecule encoding a first RNA and a linearized second DNA molecule encoding a second RNA into a single reaction vessel, wherein the first DNA molecule and the second DNA molecule are obtained from different sources; and
(b) simultaneously in vitro transcribing the linearized first DNA molecule and the linearized second DNA molecule to obtain a multivalent RNA composition.

56. A method for producing a multivalent RNA composition, the method comprising:
(a) producing a first DNA molecule in a first bacterial cell culture,
(b) producing a second DNA molecule in a second bacterial cell culture, wherein the first bacterial cell culture and second bacterial cell culture are not co-cultured,
(c) purifying and linearizing the first DNA molecule and second DNA molecule;
(d) combining the purified and linearized first DNA molecule and the purified and linearized second DNA molecule into a single IVT reaction mixture, and then
(e) simultaneously in vitro transcribing the first and second DNA molecules to obtain a multivalent RNA composition.

57. A method for producing a multivalent RNA composition, the method comprising:
(a) simultaneously in vitro transcribing at least two DNA molecules in a reaction mixture comprising:
(i) a first population of DNA molecules encoding a first RNA; and
(ii) a second population of DNA molecules encoding a second RNA that is different than the first RNA,
wherein the amounts of the first and second populations of DNA molecules present in the reaction mixture prior to the start of the IVT are normalized; and
(b) obtaining a multivalent RNA composition.

58. A method for producing a multivalent RNA composition, the method comprising:
(a) simultaneously in vitro transcribing at least two DNA molecules in a reaction mixture comprising:
(i) a first population of DNA molecules encoding a first RNA; and
(ii) a second population of DNA molecules encoding a second RNA that is different than the first RNA; and
(b) obtaining a multivalent RNA composition having a pre-defined ratio of the first RNA to the second RNA produced by the IVT of step (a), wherein the multivalent RNA composition comprises >40% polyA-tailed RNAs.

59. A method for producing a multivalent RNA composition, the method comprising:
(a) simultaneously in vitro transcribing at least two DNA molecules in a reaction mixture comprising:
(i) a first population of DNA molecules encoding a first RNA; and
(ii) a second population of DNA molecules encoding a second RNA that is different than the first RNA by at least 100 nucleotides in length; and
(b) obtaining a multivalent RNA composition having a pre-defined ratio of the first RNA to the second RNA produced by the IVT of step (a).

60. The method of any one of claims 55-59, wherein the first and/or second population of DNA molecules comprises plasmid DNA (pDNA), chemically-synthesized DNA, or complementary DNA (cDNA).

61. The method of any one of claims 55-60, wherein the IVT comprises co-transcriptional capping.

62. The method of any one of claims 55-61, wherein the first RNA and/or the second RNA comprises a 5′ cap.

63. The method of any one of claims 55-62, wherein at least 75% of the first RNAs each comprise a polyA tail.

64. The method of any one of claims 55-63, wherein at least 75% of the second RNAs each comprise a polyA tail.

65. The method of any one of claims 55-64, wherein the first RNA and/or the second RNA comprises messenger RNA (mRNA).

66. The method of any one of claims 55-65, wherein the first RNA and/or second RNA encodes a therapeutic peptide or therapeutic protein.

67. The method of any one of claims 55-65, wherein the first RNA and/or second RNA encodes an antigenic peptide or antigenic protein.

69. The method of any one of claims 57-67, wherein the normalization is based on molar mass, degradation rate (e.g., of the input DNA and/or output RNA), nucleotide content, purity, and/or polyA-tailing efficiency.

69. The method of any one of claims 55-68, wherein the molar amounts of the first and second populations of DNA molecules are normalized according to the higher polyA-tailing efficiency between the first DNA population and second DNA population.

70. The method of any one of claims 55-69, wherein the reaction mixture further comprises 1, 2, 3, 4, 5, 6, 7, 8, 9, or 10 additional DNA populations.

71. The method of claim 70, wherein the multivalent RNA composition comprises 1, 2, 3, 4, 5, 6, 7, 8, 9, or 10 additional RNAs.

72. The method of claim 70 or 71, wherein each of the additional RNAs encodes a therapeutic peptide or therapeutic protein.

73. The method of any one of claims 55-72, further comprising a step of purifying the multivalent RNA composition from the reaction mixture.

74. The method of claim 73, wherein the purifying comprises chromatography or gel electrophoresis.

75. The method of claim 73 or 74, wherein the purifying comprises column chromatography.

76. The method of any one of claims 55-75, wherein the first RNAs and/or the second RNAs comprise a 5′ untranslated region (5′ UTR).

77. The method of any one of claims 55-76, wherein the first RNAs and/or the second RNAs comprise a 3′ untranslated region (3′ UTR).

78. The method of any one of claims 55-77, wherein the multivalent RNA composition has a pre-defined RNA ratio of the first RNA to the second RNA.

79. A multivalent RNA composition produced by the method of any one of claims 55-78.

80. A pharmaceutical composition comprising:
(a) the multivalent RNA composition of claim 79; and
(b) one or more pharmaceutically acceptable excipients.

81. The pharmaceutical composition of claim 80, wherein the RNAs of the multivalent RNA composition are packaged in a lipid-based particle.

82. The pharmaceutical composition of claim 81, wherein the lipid-based particle is a liposome or a lipid nanoparticle.
